SEQUENCE LISTING (37 sequences)

SEQ ID NO: 1 - DNA, 796 bp, Homo sapiens
gtgcaatggctagtactatgtgtcaacttgtctaggctatactgctcagctgtgtggtcaaacagtagtctagatgttgctgtgaaggtattttgtagatgtgatcaacatttacaatcagttgattttaagtaaagcagtttaacttccataatgtggatgggcctcatccaattagttgaaggtgttaagagaaaagaccaaggtttcctggaaaaggaattctaccacaagactaacataaaaatgcgctgtgagtttctagcctgctggcctgccttcactgtcctgggggaggcttggagagaccaggtggactggagtagactgttgagagacgctggtctggtgaagatgtccaggaaaccacgagcctccagcccattgtccaacaaccacccaccaacaccaaagaggttcccaagacaacccggaagggaaaagggacccatcaaggaagttccaggaacaaaaggctctccctaaaagaccgccgcttcaaaaaaacctgaggaatggagtgggccaacactatccagccactctgaccagccgaacgaggaactcaatcaaaatgagccatagcgggaccacaagggcaaggagaccaccaccttctccagtctctcttcggacagccagtaattcccgggcaaggccagagacttcaagtctatctgaaaagtctccagaggtctaaccccagataaatagccaacagggtgtagagtacattttacaccccaaagagtgtgccccatggtgatgaaaataaagtgaacatgttgcaaaatga

SEQ ID NO: 2 - DNA, 773 bp, Homo sapiens
tctgcctccgcatcctcgtgggccctgaccttctctctgagagccgggcagaggctccggagccatgcaggccgaaggccagggcacagggggttcgacgggcgatgctgatggcccaggaggccctggcattcctgatggcccagggggcaatgctggcggcccaggagaggcgggtgccacgggcggcagaggtccccggggcgcaggggcagcaagggcctcggggccgagaggaggcgccccgcggggtccgcatggcggtgccgcttctgcgcaggatggaaggtgcccctgcggggccaggaggccggacagccgcctgcttcagttgcacatcacgatgcctttctcgtcgcccatggaagcggagctggtccgcaggatcctgtcccgggatgccgcacctctcccccgaccaggggcggttctgaaggacttcaccgtgtccggcaacctactgtttatccgactgactgctgcagaccaccgccaactgcagctctccatcagctcctgtctccagcagctttccctgttgatgtggatcacgcagtgctttctgcccgtgtttttggctcaggctccctcagggcagaggcgctaagcccagcctggcgccccttcctaggtcatgcctcctcccctagggaatggtcccagcacgagtggccagttcattgtgggggcctgattgtttgtcgctggaggaggacggcttacatgtttgtttctgtagaaaataaagctgagctacgattccgaaaaaaaaa

SEQ ID NO: 3 - DNA, 2188 bp, Homo sapiens
ggcagtcctagtacacaagacacgtacttgccatcactaaaacattgcctacccacatgatgccctctctccacaaaattgatccctggtgaggagtaactattgaccgcccactgagtggtgactccttatgctaagcaccacgaatacaagggcaggagcaggatttttggttgcacagccggtgtttcaggcacccatcctcccagggaacccaagagaagagccctgtatctccttagtaaatgcagaacccaagctcccaagccattttccccaggccatcctaatggctccctcagccttgatgggagaagccacgatgttcccacccatcgccttttagtagccacaagaacctgcccgcccacccactgcacgcccattggttggacagtggtagaggcgggccctgacgagcgcatgctcagagggagaagtcagcggagaagctgggagctcctttggaggctgcggtgtccctgactctcctgaggggggctcactaacgggtgggtcccatgccacagtgtctgaggagaagcaggaagtgagtccctgaggagacgccgtgacctgagggcttcccttactgaggaggcctcgtgcttcatctgccacaagcggtgcccaggccggtggtgacaactgggacgatgctcaggcttctgagacttgctttggccttctacgggaggacggccgaccctgcagagcgacagggcccacagcagcagggcctcccacaaggtgacacccagttgacaactgtgcagggagttgtcacaagtttctgtggtgattatggcatgattgatgagtcgatctacttcagtagtgatgttgtgactggcaacgtgcctctaaaagttggacaaaaagttaatgtggttgtggaagaagataaaccacattatggattgagagcaatcaaggtggatgttgtgcctcgccatctttatggtgctggaccctcagactcaggaaccagagttttaattggatgtgttacttctataaatgaagataatatttatattagtaacagcatttatttttccatagccattgtttctgaagattttgtgccttataagggtgacttgttagaagttgaatattccactgagccaggcatctcaaacatcaaggcaacttctgtgaagcccatccgttgtattcatacggaagaggtctgcattactagcgtacatggaagaaatggggtgatagattatactatttttttcaccttggattctgtgaaacttcctgatggatacgtacctcaagtagatgatatcgtcaatgtggtcatggtggagagcattcagttctgctttatttggagagcgatttctatcaccccagtgcataaatcgtaatgacaaagcatttttattctgtttatctttccttttatgagcagtaaaggggctggtttaacttaaaaggttagcttagtaagcctaaatagtattttatatatgacttttctggcaaatctaattgagacactggccagtccaactggaccaggaacccagcttagggaaataacttattaattaaaaagcatgctaaattagcttgctagtcactggaggaaaggagttcttaattaaaatgaacacggccattaaatttgaattccatatttcccattagcagcagcggattccaggatgacggaggcctgggacggcccaaaagggaacgtcggagccaaagcatttaactgaaaaggcatcagggacagcatgttaaaggcatgatttaaagttacaatttgacttcagttttgagccccgttatgctgcctgtacaacctgtattgttccatagcctctttcatcttctgtcacccacataacttgcggtgtttgttgttttacttgttccttcttccccatgctagaatgtaaactccacaatggcaggatgtcttttaatctggtttttttttttcttttttctttctttttttttttctgttgtcattaaagcatcaccaagcacttagaagagtgcctggcagactcaatgagcagataaaatgttattaagttcaatgactgaatgactgggccagcaatgatacttacctgtggttcattcatcactggttccttaggaataaaattgttgagcaatgcactaaaaaaaaaaaaaaaaaaaaaaaaaaaaa

SEQ ID NO: 4 - DNA, 830 bp, Homo sapiens
cacccgcctttcactatccgccattcttgtcacctcagctgctgccctcgctaccgcaccgacttcgcccgtgtgctcgcctgcacttgcgctgcccgccatggccaccgcccagccgtcgcaggtgcgccagaagtacgacaccaactgcgacgccgccatcaacagccacatcacgctggagctctacacctcctacctgtacctgtctatggccttctacttcaaccgggacgacgtggccctggagaacttcttccgctacttcctgcgcctgtcggacgacaaaatggagcatgcccagaagctgatgaggctgcagaacctgcgcggtggccacatctgccttcacgatatcaggaagccagagtgccaaggctgggagagcgggctcgtggccatggagtccgccttccacctggagaagaacgtcaaccagagcctgctggatctgtaccagctggccgtggagaagggcgacccccagctgtgccacttcctggagagccactacctgcacgagcaagtcaagaccatcaaagagctgggtggctacgtgagcaacctgcgcaagatttgttccccggaagccggcctggctgagtacctgttcgacaagctcaccctgggcggccgcgtcaaagagacttgagcccagatgggccccacagccacggggtcccttccctgggtcaggccactaggcggggcgtgcatgttgccctttcagaacgttctcttcagttttatctttcagttttaccattgttagcaaaaaagttatctggttctcaaagcaataaaggtgtccataaaaaaaaaaaaaaaaa

SEQ ID NO: 5 - DNA, 3157 bp, Homo sapiens
actgggcgtcttctgcccggccccttcgcccacgtgaagaacgccagggagctgtgaggcagtgctgtgtggttcctgccgtccggactctttttcctctactgagattcatctgtgtgaaatatgagttggcgaggaagatcgacctattattggcctagaccaaggcgctatgtacagcctcctgaaatgattgggcctatgcggcccgagcagttcagtgatgaagtggaaccagcaacacctgaagaaggggaaccagcaactcaacgtcaggatcctgcagctgctcaggagggagaggatgagggagcatctgcaggtcaagggccgaagcctgaagctgatagccaggaacagggtcacccacagactgggtgtgagtgtgaagatggtcctgatgggcaggagatggacccgccaaatccagaggaggtgaaaacgcctgaagaagagatgaggtctcactatgttgcccagactgggattctctggcttttaatgaacaattgcttcttaaatctttccccacggaaaccttgagtgactgaaatatcaaatggcaagagaccgtttagttcctatcatctgtggcatgtgaagggcaatcacagtgttaaaagaagacatgctgaaatgttgcaggctgctcctatgttggaaaattcttcattgaagttctcccaataaagctttacagccttctgcaaagaagtcttgtgaatcttttgtcaattttatttctagctatttgatgctgtgaaatgtttcattctttgcaattttgtattctatctccttgagctgtgtgtagaggcataattctcatgtattgattttctatccagcaaccttgttaaatatgcttatgaattttaaaagtttacttctaggttttttcagttttcaacctacagaatcatatcatttttgaataagaacaattttgtttctgccttttttgtttgttttttcttttgtatttttcgtagaggtgggattttggcctgtgtcctaggctttttttgaactcctgagcgcaagtaatccactctccttggcctttcaaagtgttgggattacaggcatgggccaccgtgctggtcctgtttttgccattttaaacccttttatttccttttctgattttatggcattgagcagatctaccggatacaattgtgatagtggaaatttttgtgttattcctgatgagaaatggaaaaatttcaacatttcacgacaatatttagtgtactttttttgtagatggactttttcagagtaagtcaagccattctgttttagtttgttgagagtgttcattttgaatatatgttgaatttcatcaaacactgacctgagtcatcttaaaacatgtgaattgagatttctttgctactaagaaagtgagcgggcactctgcttcatgtttacttttgtcatgttgcatgaaaaacattttgcttcatgtttgattctgtatgttgaaaactgaaatcatctattgtgattaccacagggttttttcccccagtaatctgtttatgtagtcaattacgttgataaatctgtactttttaaattttaacaattgagacaggtctcactctgtcacccatgctgactgcagtagtgtgatcacagcttactgcaacctcaacctcctgggctcaggcgatcctcccacctcaccctcctcagtagctaggactataggtacatgccaccatgccaagctaatttttctgttttagagatggtattttttcatgttgcccaggctggttttatactcctaatctcaagccatccactagcctcagcctcccaaagtgctgggattacaggaatgagtcacggtgcctggaaattttgtacatttaaaccaacaatttggtcatgggtaatctatgtcctaataatttatttaaggatttttatgtatatcctcatgagtgacattacctgtacttttatttcatatgctcatttgttggacgttgttatcaaggttcctctagcttcataaaacgggttggtatgtaaaacctctttttccattcactggaactcaagtctcctcaaggctgtgagtaatgcagggctaggctttcccataatgagcttttctagaatgcttctctcagatttggaccctacttaaacagcagtgaccaaacgggcagctccaggtacctatcccctcaaactttgtgagggtcgcgttctctgaagatgcctcttcaatttgaaagctatctgttcctgtttctctgctgatattaactctctgtgcacagaaagttaaacgtcactggttattaatttccctagattttgatctgtgctgtgtggctgagaatgggctgactgaccctagatctgtgtataattatgacaatggctccatttatttttaaaataagaggaattattataaaattcctatttactggatgtgtactatctatgaattacttctttgtgctaggttgtgtacatgtatgacctctttagatcctcacaagataaggcagaattttcatgaaattgatgactgactccagtaagaagcagatttggggggatttcaatttctaagctcaaagcccttgcacttttctcaaagtaaagcttttgaaagtgttaaatgtacacgaactgatggtgtatatgatgattttagttgtaatctgatgttttctttaaaaatttacatatacaaaagtgtttgactcaaaaggctttgttcttcccttaaaggaagcatctaccaaaatgtggcacacaaaccttgcatggtgtctctagggcctcctaccccgcatttcccctgctttttctcttgttctgactgaaaaacaaagtgctttgactgtgctgtgacccagccagctgcatgtttacccagcatgcttgaacccaagctggagccttgaacataaaggtgtttaagttgttgctcaaaatatggaaagaatctagctctggccttgaaccaaatcccttaaactctcctataaaactccataacctgaccccctcagtgcggatatacctaggcatgacattcttgttgcctgttgcgaggatgctttagcctactctaagttctcccaataaatgctttggactgataaaaaaaaaaaaaaaa

SEQ ID NO: 6 - DNA, 528 bp, Homo sapiens
ctgtgaggcagtgctgtgtggttcctgccgtccggactctttttcctctactgagattcatctgtgtgaaatatgagttggcgaggaagatcgacctatcggcctagaccaagacgctacgtagagcctcctgaaatgattgggcctatgcggcccgagcagttcagtgatgaagtggaaccagcaacacctgaagaaggggaaccagcaactcaacgtcaggatcctgcagctgctcaggagggagaggatgagggagcatctgcaggtcaagggccgaagcctgaagctgatagccaggaacagggtcacccacagactgggtgtgagtgtgaagatggtcctgatgggcaggagatggacccgccaaatccagaggaggtgaaaacgcctgaagaaggtgaaaagcaatcacagtgttaaaagaagacacgttgaaatgatgcaggctgctcctatgttggaaatttgttcattaaaattctcccaataaagctttacagccttctgcaaagaaaaaaaaaaaaa

SEQ ID NO: 7 - DNA, 1318 bp, Homo sapiens
cgtgcgtgtctcgagtcgcacggagggcaaccgtcgacgggcttagcgcctcaactgtcgttggtgtatttttctggtgtcacttctgtgccttccttcaaaggtggtgctttgtccctgtgggtcatctgtactgattgcgccaagcaaagcatttgttctccaaatgtcaactgtcaaggagcagctaattgagaagctaattgaggatgatgaaaactcccagtgtaaaattactattgttggaactggtgccgtaggcatggcttgtgctattagtatcttactgaaggatttggctgatgaacttgcccttgttgatgttgcattggacaaactgaagggagaaatgatggatcttcagcatggcagtcttttctttagtacttcaaagattacttctggaaaagattacagtgtatctgcaaactccagaatagttattgtcacagcaggtgcaaggcagcaggagggagaaactcgccttgccctggtccaacgtaatgtggctataatgaaatcaatcattcctgccatagtccattatagtcctgattgtaaaattcttgttgtttcaaatccagtggatattttgacatatatagtctggaagataagtggcttacctgtaactcgtgtaattggaagtggttgtaatctagactctgcccgtttccgttacctaattggagaaaagttgggtgtccaccccacaagctgccatggttggattattggagaacatggtgattctagtgtgcccttatggagtggggtgaatgttgctggtgttgctctgaagactctggaccctaaattaggaacggattcagataaggaacactggaaaaatatccataaacaagttattcaaagtgcctatgaaattatcaagctgaaggggtatacctcttgggctattggactgtctgtgatggatctggtaggatccattttgaaaaatcttaggagagtgcacccagtttccaccatggttaagggattatatggaataaaagaagaactctttctcagtatcccttgtgtcttggggcggaatggtgtctcagatgttgtgaaaattaacttgaattctgaggaggaggcccttttcaagaagagtgcagaaacactttggaatattcaaaaggatctaatattttaaattaaagccttctaatgttccactgtttggagaacagaagatagcaggctgtgtattttaaattttgaaagtattttcatttgatctttaaaaaataaaaacaaattggagacctgtgaaaaaaaaaaaaaaaaaaaaaaaaaaaaaaaaaaa

SEQ ID NO: 8 - DNA, 1755 bp, Homo sapiens
agagagaagcgaggtttccattctgagggacggcgtagagttcggccgaaggaacctgacccaggctctgtgaggaggcaaggttttcaggggacaggccaacccagaggacaggattccctggaggccacagaggagcaccaaggagaagatctgcctgtgggtcttcattgcccagctcctgcccacactcctgcctgctgccctgacgagagtcatcatgtctcttgagcagaggagtctgcactgcaagcctgaggaagcccttgaggcccaacaagaggccctgggcctggtgtgtgtgcaggctgccacctcctcctcctctcctctggtcctgggcaccctggaggaggtgcccactgctgggtcaacagatcctccccagagtcctcagggagcctccgcctttcccactaccatcaacttcactcgacagaggcaacccagtgagggttccagcagccgtgaagaggaggggccaagcacctcttgtatcctggagtccttgttccgagcagtaatcactaagaaggtggctgatttggttggttttctgctcctcaaatatcgagccagggagccagtcacaaaggcagaaatgctggagagtgtcatcaaaaattacaagcactgttttcctgagatcttcggcaaagcctctgagtccttgcagctggtctttggcattgacgtgaaggaagcagaccccaccggccactcctatgtccttgtcacctgcctaggtctctcctatgatggcctgctgggtgataatcagatcatgcccaagacaggcttcctgataattgtcctggtcatgattgcaatggagggcggccatgctcctgaggaggaaatctgggaggagctgagtgtgatggaggtgtatgatgggagggagcacagtgcctatggggagcccaggaagctgctcacccaagatttggtgcaggaaaagtacctggagtaccggcaggtgccggacagtgatcccgcacgctatgagttcctgtggggtccaagggccctcgctgaaaccagctatgtgaaagtccttgagtatgtgatcaaggtcagtgcaagagttcgctttttcttcccatccctgcgtgaagcagctttgagagaggaggaagagggagtctgagcatgagttgcagccaaggccagtgggagggggactgggccagtgcaccttccagggccgcgtccagcagcttcccctgcctcgtgtgacatgaggcccattcttcactctgaagagagcggtcagtgttctcagtagtaggtttctgttctattgggtgacttggagatttatctttgttctcttttggaattgttcaaatgtttttttttaagggatggttgaatgaacttcagcatccaagtttatgaatgacagcagtcacacagttctgtgtatatagtttaagggtaagagtcttgtgttttattcagattgggaaatccattctattttgtgaattgggataataacagcagtggaataagtacttagaaatgtgaaaaatgagcagtaaaatagatgagataaagaactaaagaaattaagagatagtcaattcttgccttatacctcagtctattctgtaaaatttttaaagatatatgcatacctggatttccttggcttctttgagaatgtaagagaaattaaatctgaataaagaattcttcctgttaaaaaaaaaaaaaaaaaaaaaaaaaaaaaaaaaaaaaaaaaaaaaa

SEQ ID NO: 9 - DNA, 1753 bp, Homo sapiens
gagattctcgccctgagcaacgagcgacggcctgacgtcggcggagggaagccggcccaggctcggtgaggaggcaaggttctgaggggacaggctgacctggaggaccagaggcccccggaggagcactgaaggagaagatctgccagtgggtctccattgcccagctcctgcccacactcccgcctgttgccctgaccagagtcatcatgcctcttgagcagaggagtcagcactgcaagcctgaagaaggccttgaggcccgaggagaggccctgggcctggtgggtgcgcaggctcctgctactgaggagcaggaggctgcctcctcctcttctactctagttgaagtcaccctgggggaggtgcctgctgccgagtcaccagatcctccccagagtcctcagggagcctccagcctccccactaccatgaactaccctctctggagccaatcctatgaggactccagcaaccaagaagaggaggggccaagcaccttccctgacctggagtccgagttccaagcagcactcagtaggaaggtggccgagttggttcattttctgctcctcaagtatcgagccagggagccggtcacaaaggcagaaatgctggggagtgtcgtcggaaattggcagtatttctttcctgtgatcttcagcaaagcttccagttccttgcagctggtctttggcatcgagctgatggaagtggaccccatcggccacttgtacatctttgccacctgcctgggcctctcctacgatggcctgctgggtgacaatcagatcatgcccaaggcaggcctcctgataatcgtcctggccataatcgcaagagagggcgactgtgcccctgaggagaaaatctgggaggagctgagtgtgttagaggtgtttgaggggagggaagacagtatcttgggggatcccaagaagctgctcacccaacatttcgtgcaggaaaactacctggagtaccggcaggtccccggcagtgatcctgcatgttatgaattcctgtggggtccaagggccctcgttgaaaccagctatgtgaaagtcctgcaccatatggtaaagatcagtggaggacctcacatttcctacccacccctgcatgagtgggttttgagagagggggaagagtgagtctgagcacgagttgcagccagggccagtgggagggggtctgggccagtgcaccttccggggccgcatcccttagtttccactgcctcctgtgacgtgaggcccattcttcactctttgaagcgagcagtcagcattcttagtagtgggtttctgttctgttggatgactttgagattattctttgtttcctgttggagttgttcaaatgttccttttaacggatggttgaatgagcgtcagcatccaggtttatgaatgacagtagtcacacatagtgctgtttatatagtttaggagtaagagtcttgttttttactcaaattgggaaatccattccattttgtgaattgtgacataataatagcagtggtaaaagtatttgcttaaaattgtgagcgaattagcaataacatacatgagataactcaagaaatcaaaagatagttgattcttgccttgtacctcaatctattctgtaaaattaaacaaatatgcaaaccaggatttccttgacttctttgagaatgcaagcgaaattaaatctgaataaataattcttcctcttcaaaaaaaaaaaaaaaaaaaaaaaaaaaaaa

SEQ ID NO: 10 - DNA, 1727 bp, Homo sapiens
ggttagagagaagcgagctgctctgtctgaccagcagcttgggattggcggagggaagcgggccaggccctgtgaggagtcaaggttctgagcagacaggccaaccggaggacaggattccctggaggccacagaggagcaccaaggagaagatctgcctgtgggtccccattgcccagcttttgcctgcactcttgcctgctgccctgaccagagtcatcatgtcttctgagcagaagagtcagcactgcaagcctgaggaaggcgttgaggcccaagaagaggccctgggcctggtgggtgcacaggctcctactactgaggagcaggaggctgctgtctcctcctcctctcctctggtccctggcaccctggaggaagtgcctgctgctgagtcagcaggtcctccccagagtcctcagggagcctctgccttacccactaccatcagcttcacttgctggaggcaacccaatgagggttccagcagccaagaagaggaggggccaagcacctcgcctgacgcagagtccttgttccgagaagcactcagtaacaaggtggatgagttggctcattttctgctccgcaagtatcgagccaaggagctggtcacaaaggcagaaatgctggagagagtcatcaaaaattacaagcgctgctttcctgtgatcttcggcaaagcctccgagtccctgaagatgatctttggcattgacgtgaaggaagtggaccccgccagcaacacctacacccttgtcacctgcctgggcctttcctatgatggcctgctgggtaataatcagatctttcccaagacaggccttctgataatcgtcctgggcacaattgcaatggagggcgacagcgcctctgaggaggaaatctgggaggagctgggtgtgatgggggtgtatgatgggagggagcacactgtctatggggagcccaggaaactgctcacccaagattgggtgcaggaaaactacctggagtaccggcaggtacccggcagtaatcctgcgcgctatgagttcctgtggggtccaagggctctggctgaaaccagctatgtgaaagtcctggagcatgtggtcagggtcaatgcaagagttcgcattgcctacccatccctgcgtgaagcagctttgttagaggaggaagagggagtctgagcatgagttgcagccagggctgtggggaaggggcagggctgggccagtgcatctaacagccctgtgcagcagcttcccttgcctcgtgtaacatgaggcccattcttcactctgtttgaagaaaatagtcagtgttcttagtagtgggtttctattttgttggatgacttggagatttatctctgtttccttttacaattgttgaaatgttccttttaatggatggttgaattaacttcagcatccaagtttatgaatcgtagttaacgtatattgctgttaatatagtttaggagtaagagtcttgttttttattcagattgggaaatccgttctattttgtgaatttgggacataataacagcagtggagtaagtatttagaagtgtgaattcaccgtgaaataggtgagataaattaaaagatacttaattcccgccttatgcctcagtctattctgtaaaatttaaaaaatatatatgcatacctggatttccttggcttcgtgaatgtaagagaaattaaatctgaataaataattctttctgttaa

SEQ ID NO: 11 - DNA, 1721 bp, Homo sapiens
ttagagagaagcgagctgctctgaccagccgcttgggattggcggagggaagcgggccaggccctgtgaggagtcaaggttctgagcagacaggccaaccggaggacaggattccctggaggccacagaggagcaccaaggagaagatctgcctgtgggtccccattgcccagcttttgcctgcactcttgcctgctgccctgaccagagtcatcatgtcttctgagcagaagagtcagcactgcaagcctgaggaaggcgttgaggcccaagaagaggccctgggcctggtgggtgcacaggctcctactactgaggagcaggaggctgctgtctcctcctcctctcctctggtccctggcaccctggaggaagtgcctgctgctgagtcagcaggtcctccccagagtcctcagggagcctctgccttacccactaccatcagcttcacttgctggaggcaacccaatgagggttccagcagccaagaagaggaggggccaagcacctcgcctgacgcagagtccttgttccgagaagcactcagtaacaaggtggatgagttggctcattttctgctccgcaagtatcgagccaaggagctggtcacaaaggcagaaatgctggagagagtcatcaaaaattacaagcgctgctttcctgtgatcttcggcaaagcctccgagtccctgaagatgatctttggcattgacgtgaaggaagtggaccccgccagcaacacctacacccttgtcacctgcctgggcctttcctatgatggcctgctgggtaataatcagatctttcccaagacaggccttctgataatcgtcctgggcacaattgcaatggagggcgacagcgcctctgaggaggaaatctgggaggagctgggtgtgatgggggtgtatgatgggagggagcacactgtctatggggagcccaggaaactgctcacccaagattgggtgcaggaaaactacctggagtaccggcaggtacccggcagtaatcctgcgcgctatgagttcctgtggggtccaagggctctggctgaaaccagctatgtgaaagtcctggagcatgtggtcagggtcaatgcaagagttcgcattgcctacccatccctgcgtgaagcagctttgttagaggaggaagagggagtctgagcatgagttgcagccagggctgtggggaaggggcagggctgggccagtgcatctaacagccctgtgcagcagcttcccttgcctcgtgtaacatgaggcccattcttcactctgtttgaagaaaatagtcagtgttcttagtagtgggtttctattttgttggatgacttggagatttatctctgtttccttttacaattgttgaaatgttccttttaatggatggttgaattaacttcagcatccaagtttatgaatcgtagttaacgtatattgctgttaatatagtttaggagtaagagtcttgttttttattcagattgggaaatccgttctattttgtgaatttgggacataataacagcagtggagtaagtatttagaagtgtgaattcaccgtgaaataggtgagataaattaaaagatacttaattcccgccttatgcctcagtctattctgtaaaatttaaaaaatatatatgcatacctggatttccttggcttcgtgaatgtaagagaaattaaatctgaataaataattctttctgttaa

SEQ ID NO: 12 - DNA, 1712 bp, Homo sapiens
cgagctgctgtctgaccagcagcttgggattggtggaaggaagcaggccaggccctgtgaggagtcaaggttctgagcagacaggccaaccggaggacaggattccctggaggccacagaggagcaccaaggagaagatctgcctgtgggtccccattgcccagcttttgcctgcactcttgcctgctgccctgaccagagtcatcatgtcttctgagcagaagagtcagcactgcaagcctgaggaaggcgttgaggcccaagaagaggccctgggcctggtgggtgcacaggctcctactactgaggagcaggaggctgctgtctcctcctcctctcctctggtccctggcaccctggaggaagtgcctgctgctgagtcagcaggtcctccccagagtcctcagggagcctctgccttacccactaccatcagcttcacttgctggaggcaacccaatgagggttccagcagccaagaagaggaggggccaagcacctcgcctgacgcagagtccttgttccgagaagcactcagtaacaaggtggatgagttggctcattttctgctccgcaagtatcgagccaaggagctggtcacaaaggcagaaatgctggagagagtcatcaaaaattacaagcgctgctttcctgtgatcttcggcaaagcctccgagtccctgaagatgatctttggcattgacgtgaaggaagtggaccccgccagcaacacctacacccttgtcacctgcctgggcctttcctatgatggcctgctgggtaataatcagatctttcccaagacaggccttctgataatcgtcctgggcacaattgcaatggagggcgacagcgcctctgaggaggaaatctgggaggagctgggtgtgatgggggtgtatgatgggagggagcacactgtctatggggagcccaggaaactgctcacccaagattgggtgcaggaaaactacctggagtaccggcaggtacccggcagtaatcctgcgcgctatgagttcctgtggggtccaagggctctggctgaaaccagctatgtgaaagtcctggagcatgtggtcagggtcaatgcaagagttcgcattgcctacccatccctgcgtgaagcagctttgttagaggaggaagagggagtctgagcatgagttgcagccagggctgtggggaaggggcagggctgggccagtgcatctaacagccctgtgcagcagcttcccttgcctcgtgtaacatgaggcccattcttcactctgtttgaagaaaatagtcagtgttcttagtagtgggtttctattttgttggatgacttggagatttatctctgtttccttttacaattgttgaaatgttccttttaatggatggttgaattaacttcagcatccaagtttatgaatcgtagttaacgtatattgctgttaatatagtttaggagtaagagtcttgttttttattcagattgggaaatccgttctattttgtgaatttgggacataataacagcagtggagtaagtatttagaagtgtgaattcaccgtgaaataggtgagataaattaaaagatacttaattcccgccttatgcctcagtctattctgtaaaatttaaaaaatatatatgcatacctggatttccttggcttcgtgaatgtaagagaaattaaatctgaataaataattctttctgttaa

SEQ ID NO: 13 - DNA, 1949 bp, Homo sapiens
aataaaggggtctgagccggtcgcctgagcctgaaaagtgctgtcacgtcagcggaaggaggcgtcccagatcttctcagctgtcttggtgccagccttcctagtcttcctacccacactcctacctgctgtcacaggccacagccatcatgcctcggggtcacaagagtaagctccgtacctgtgagaaacgccaagagaccaatggtcagccacagggtctcacgggtccccaggccactgcagagaagcaggaagagtcccactcttcctcatcctcttctcgcgcttgtctgggtgattgtcgtaggtcttctgatgcctccattcctcaggagtctcagggagtgtcacccactgggtctcctgatgcagttgtttcatattcaaaatccgatgtggctgccaacggccaagatgagaaaagtccaagcacctcccgtgatgcctccgttcctcaggagtctcagggagcttcacccactggctctcctgatgcaggtgtttcaggctcaaaatatgatgtggctgccaacggccaagatgagaaaagtccaagcacttcccatgatgtctccgttcctcaggagtctcagggagcttcacccactggctcgcctgatgcaggtgtttcaggctcaaaatatgatgtggctgccgagggtgaagatgaggaaagtgtaagcgcctcacagaaagccatcatttttaagcgcttaagcaaagatgctgtaaagaagaaggcgtgcacgttggcgcaattcctgcagaagaagtttgagaagaaagagtccattttgaaggcagacatgctgaagtgtgtccgcagagagtacaagccctacttccctcagatcctcaacagaacctcccaacatttggtggtggcctttggcgttgaattgaaagaaatggattccagcggcgagtcctacacccttgtcagcaagctaggcctccccagtgaaggaattctgagtggtgataatgcgctgccgaagtcgggtctcctgatgtcgctcctggttgtgatcttcatgaacggcaactgtgccactgaagaggaggtctgggagttcctgggtctgttggggatatatgatgggatcctgcattcaatctatggggatgctcggaagatcattactgaagatttggtgcaagataagtacgtggtttaccggcaggtgtgcaacagtgatcctccatgctatgagttcctgtggggtccacgagcctatgctgaaaccaccaagatgagagtcctgcgtgttttggccgacagcagtaacaccagtcccggtttatacccacatctgtatgaagacgctttgatagatgaggtagagagagcattgagactgagagcttaaggcagggctggcactatttccttggccagggtaccttatggggccatatcctacagatcctcccatttctagggaggtctgaagtagaattttcactttatgttagaagagagtagtgagctttctaagtagtgcagtatagtagaggctggagggaacaagatatgtatctttcttttgttacacatgagtaacttgcagatttatgttttatctctgtcagttatcaacattgttcctgttaagtgaaggtttattttgcttcagattatacaattatcaataacatagctctcacattcatggctgtttaaccaatctgaaagttacggtttgggaattaataaaacaaagtcatacaacacattttctttgtaattgagaactagataacatggtaacagagaattgattttcatatgaatcttaactccacagtaaaatagttgacatcataatatgaagagaaagaaaaggaaaaacagaaatgtaaaagttgtttaattcttggtttgcctaattcgttttcctatttcttttcatacaaataaaggatacctggatttatttaggtta

SEQ ID NO: 14 - DNA, 1365 bp, Homo sapiens
cactgcttgagccgctgagagggtggcgacgtcggggccatggggctgggcccggtcttcctgcttctggctggcatcttcccttttgcacctccgggagctgctgctgagccccacagtcttcgttataacctcacggtgctgtcctgggatggatctgtgcagtcagggtttctcactgaggtacatctggatggtcagcccttcctgcgctgtgacaggcagaaatgcagggcaaagccccagggacagtgggcagaagatgtcctgggaaataagacatgggacagagagaccagagacttgacagggaacggaaaggacctcaggatgaccctggctcatatcaaggaccagaaagaaggcttgcattccctccaggagattagggtctgtgagatccatgaagacaacagcaccaggagctcccagcatttctactacgatggggagctcttcctctcccaaaacctggagactaaggaatggacaatgccccagtcctccagagctcagaccttggccatgaacgtcaggaatttcttgaaggaagatgccatgaagaccaagacacactatcacgctatgcatgcagactgcctgcaggaactacggcgatatctaaaatccggcgtagtcctgaggagaacagtgccccccatggtgaatgtcacccgcagcgaggcctcagagggcaacattaccgtgacatgcagggcttctggcttctatccctggaatatcacactgagctggcgtcaggatggggtatctttgagccacgacacccagcagtggggggatgtcctgcctgatgggaatggaacctaccagacctgggtggccaccaggatttgccaaggagaggagcagaggttcacctgctacatggaacacagcgggaatcacagcactcaccctgtgccctctgggaaagtgctggtgcttcagagtcattggcagacattccatgtttctgctgttgctgctgctgctatttttgttattattattttctatgtccgttgttgtaagaagaaaacatcagctgcagagggtccagagctcgtgagcctgcaggtcctggatcaacacccagttgggacgagtgaccacagggatgccacacagctcggatttcagcctctgatgtcagatcttgggtccactggctccactgagggcgcctagactctacagccaggcagctgggattcaattccctgcctggatctcacgagcactttccctcttggtgcctcagtttcctgacctatgaaacagagaaaataaaagcacttatttattgttgttggaggctgcaaaatgttagtagatatgaggcgtttgcagctgtaccatatt

SEQ ID NO: 15 - DNA, 3690 bp, Homo sapiens
gtgctgggctgttcgtctcttctatgtgctgatttcctgggttactttgggtcttccttttctttctcccttttaccctgtctcctttcttgaggctgatcgatcacagccaggcctctccattctatttacccagcgttttccttctctccagttagtggggtagatgaacgccctgtgtttataaggtgcctcccaggagcctgagacctgtgagaagaatggggggtggaggtgggggagactcgtcacgaagggagaccttggagcttcgagggtgggaatgttcttattagattcttcatctctgttgacacaaacatgtaggagaagctggagaacatagacagggatgaggttttatttatttattgttcctggtcactgtctctttgaggattggtatctctgctccagaaaagatggcagcctctttcttctctgattttggtcttatgtggtatctggaggagctcaaaaaggaggagttcaggaaatttaaagaacatctcaagcaaatgactttgcagcttgaactcaagcagattccctggactgaggtcaaaaaagcatcccgggaagaacttgcaaacctcttgatcaagcactatgaagaacaacaagcttggaacataaccttaagaatctttcaaaagatggatagaaaggatctctgcatgaaggtcatgagggagagaacaggatacacaaagacctatcaagctcacgcaaagcagaaattcagccgcttatggtccagcaagtctgtcactgagattcacctatactttgaggaggaagtcaagcaagaagaatgtgaccatttggaccgcctttttgctcccaaggaagctgggaaacagccacgtacagtgatcattcaaggaccacaaggaattggaaaaacgacactcctgatgaagctgatgatggcctggtcggacaacaagatctttcgggataggttcctgtacacgttctatttctgctgcagagaactgagggagttgccgccaacgagtttggctgacttgatttccagagagtggcctgaccccgctgctcctataacagagatcgtgtctcaaccggagagactcttgttcgtcatcgacagcttcgaagagctgcagggcggcttgaacgaacccgattcggatctgtgtggtgacttgatggagaaacggccggtgcaggtgcttctgagcagtttgctgaggaagaagatgctcccggaggcctccctgctcatcgctatcaaacccgtgtgcccgaaggagctccgggatcaggtgacgatctcagaaatctaccagccccggggattcaacgagagtgataggttagtgtatttctgctgtttcttcaaagacccgaaaagagccatggaagccttcaatcttgtaagagaaagtgaacagctgttttccatatgccaaatcccgctcctctgctggatcctgtgtaccagtctgaagcaagagatgcagaaaggaaaagacctggccctgacctgccagagcactacctctgtgtactcctctttcgtctttaacctgttcacacctgagggtgccgagggcccgactccgcaaacccagcaccagctgaaggccctgtgctccctggctgcagagggtatgtggacagacacatttgagttttgtgaagacgacctccggagaaatggggttgttgacgctgacatccctgcgctgctgggcaccaagatacttctgaagtacggggagcgtgagagctcctacgtgttcctccacgtgtgtatccaggagttctgtgccgccttgttctatttgctcaagagccaccttgatcatcctcacccagctgtgagatgtgtacaggaattgctagttgccaattttgaaaaagcaaggagagcacattggatttttttggggtgttttctaactggccttttaaataaaaaggaacaagaaaaactggatgcgttttttggcttccaactgtcccaagagataaagcagcaaattcaccagtgcctgaagagcttaggggagcgtggcaatcctcagggacaggtggattccttggcgatattttactgtctctttgaaatgcaggatcctgcctttgtgaagcaggcagtgaacctcctccaagaagctaactttcatattattgacaacgtggacttggtggtttctgcctactgcttaaaatactgctccagcttgaggaaactctgtttttccgttcaaaatgtctttaagaaagaggatgaacacagctctacgtcggattacagcctcatctgttggcatcacatctgctctgtgctcaccaccagcgggcacctcagagagctccaggtgcaggacagcaccctcagcgagtcgacctttgtgacctggtgtaaccagctgaggcatcccagctgtcgccttcagaagcttggaataaataacgtttccttttctggccagagtgttctgctctttgaggtgctcttttatcagccagacttgaaatacctgagcttcaccctcacgaaactctctcgtgatgacatcaggtccctctgtgatgccttgaactacccagcaggcaacgtcaaagagctagcgctggtaaattgtcacctctcacccattgattgtgaagtccttgctggccttctaaccaacaacaagaagctgacgtatctgaatgtatcctgcaaccagttagacacaggcgtgccccttttgtgtgaagccctgtgcagcccagacacggtcctggtatacctgatgttggctttctgccacctcagcgagcagtgctgcgaatacatctctgaaatgcttctgcgtaacaagagcgtgcgctatctagacctcagtgccaatgtcctgaaggacgaaggactgaaaactctctgcgaggccttgaaacatccggactgctgcctggattcactgtgtttggtaaaatgttttatcactgctgctggctgtgaagacctcgcctctgctctcatcagcaatcaaaacctgaagattctgcaaattgggtgcaatgaaatcggagatgtgggtgtgcagctgttgtgtcgggctctgacgcatacggattgccgcttagagattcttgggttggaagaatgtgggttaacgagcacctgctgtaaggatctcgcgtctgttctcacctgcagtaagaccctgcagcagctcaacctgaccttgaacaccttggaccacacaggggtggttgtactctgtgaggccctgagacacccagagtgtgccctgcaggtgctcgggctgagaaaaactgattttgatgaggaaacccaggcacttctgacggctgaggaagagagaaatcctaacctgaccatcacagacgactgtgacacaatcacaagggtagagatctgattgcgaggaacctgggctctgactcgaacacctgcaaaggacagggactgggaccgttacttacatgacactgcacccaggagatacaaatcattgatactctgagttgtgagatttctggcaccccattcatagatttgatatgatacacgtggtttttatgtgctctgtggccttggatgagtcactgaaaggccttcatggtctctcggtctcacaaggacctcttaacccctcaataaagtgttacatttctaaacattggaaaaaaaaaaaaaaaaaa

SEQ ID NO: 16 - DNA, 752 bp, Homo sapiens
atcctcgtgggccctgaccttctctctgagagccgggcagaggctccggagccatgcaggccgaaggccggggcacagggggttcgacgggcgatgctgatggcccaggaggccctggcattcctgatggcccagggggcaatgctggcggcccaggagaggcgggtgccacgggcggcagaggtccccggggcgcaggggcagcaagggcctcggggccgggaggaggcgccccgcggggtccgcatggcggcgcggcttcagggctgaatggatgctgcagatgcggggccagggggccggagagccgcctgcttgagttctacctcgccatgcctttcgcgacacccatggaagcagagctggcccgcaggagcctggcccaggatgccccaccgcttcccgtgccaggggtgcttctgaaggagttcactgtgtccggcaacatactgactatccgactgactgctgcagaccaccgccaactgcagctctccatcagctcctgtctccagcagctttccctgttgatgtggatcacgcagtgctttctgcccgtgtttttggctcagcctccctcagggcagaggcgctaagcccagcctggcgccccttcctaggtcatgcctcctcccctagggaatggtcccagcacgagtggccagttcattgtgggggcctgattgtttgtcgctggaggaggacggcttacatgtttgtttctgtagaaaataaaactgagctacgaaaaa

SEQ ID NO: 17 - DNA, 1899 bp, Homo sapiens
agcgcgcgactttttgaaagccaggagggttcgaattgcaacggcagctgccgggcgtatgtgttggtgctagaggcagctgcagggtctcgctgggggccgctcgggaccaattttgaagaggtacttggccacgacttattttcacctccgacctttccttccaggcggtgagactctggactgagagtggctttcacaatggaagggatcagtaatttcaagacaccaagcaaattatcagaaaaaaagaaatctgtattatgttcaactccaactataaatatcccggcctctccgtttatgcagaagcttggctttggtactggggtaaatgtgtacctaatgaaaagatctccaagaggtttgtctcattctccttgggctgtaaaaaagattaatcctatatgtaatgatcattatcgaagtgtgtatcaaaagagactaatggatgaagctaagattttgaaaagccttcatcatccaaacattgttggttatcgtgcttttactgaagccaatgatggcagtctgtgtcttgctatggaatatggaggtgaaaagtctctaaatgacttaatagaagaacgatataaagccagccaagatccttttccagcagccataattttaaaagttgctttgaatatggcaagagggttaaagtatctgcaccaagaaaagaaactgcttcatggagacataaagtcttcaaatgttgtaattaaaggcgattttgaaacaattaaaatctgtgatgtaggagtctctctaccactggatgaaaatatgactgtgactgaccctgaggcttgttacattggcacagagccatggaaacccaaagaagctgtggaggagaatggtgttattactgacaaggcagacatatttgcctttggccttactttgtgggaaatgatgactttatcgattccacacattaatctttcaaatgatgatgatgatgaagataaaacttttgatgaaagtgattttgatgatgaagcatactatgcagcgttgggaactaggccacctattaatatggaagaactggatgaatcataccagaaagtaattgaactcttctctgtatgcactaatgaagaccctaaagatcgtccttctgctgcacacattgttgaagctctggaaacagatgtctagtgatcatctcagctgaagtgtggcttgcgtaaataactgtttattccaaaatatttacatagttactatcagtagttattagactctaaaattggcatatttgaggaccatagtttcttgttaacatatggataactatttctaatatgaaatatgcttatattggctataagcacttggaattgtactgggttttctgtaaagttttagaaactagctacataagtactttgatactgctcatgctgacttaaaacactagcagtaaaacgctgtaaactgtaacattaaattgaatgaccattacttttattaatgatctttcttaaatattctatattttaatggatctactgacattagcactttgtacagtacaaaataaagtctacatttgtttaaaacactgaaccttttgctgatgtgtttatcaaatgataactggaagctgaggagaatatgcctcaaaaagagtagctccttggatacttcagactctggttacagattgtcttgatctcttggatctcctcagatctttggtttttgctttaatttattaaatgtattttccatactgagtttaaaatttattaatttgtaccttaagcatttcccagctgtgtaaaaacaataaaactcaaataggatgataaagaataaaggacactttgggtaccagaaaaaaaaaaaaaaaaaaaaaaaaaaaaaaaaaaaaaaaaaaaaaaa

SEQ ID NO: 18 - DNA, 2195 bp, Homo sapiens
cccagcgctcctccccgcaaatgatcccgccccaggggcctatcccagtccccccagtgcctttggttgctggagggaagaacacaatggatctggtgctaaaaagatgccttcttcatttggctgtgataggtgctttgctggctgtgggggctacaaaagtacccagaaaccaggactggcttggtgtctcaaggcaactcagaaccaaagcctggaacaggcagctgtatccagagtggacagaagcccagagacttgactgctggagaggtggtcaagtgtccctcaaggtcagtaatgatgggcctacactgattggtgcaaatgcctccttctctattgccttgaacttccctggaagccaaaaggtattgccagatgggcaggttatctgggtcaacaataccatcatcaatgggagccaggtgtggggaggacagccagtgtatccccaggaaactgacgatgcctgcatcttccctgatggtggaccttgcccatctggctcttggtctcagaagagaagctttgtttatgtctggaagacctggggccaatactggcaagttctagggggcccagtgtctgggctgagcattgggacaggcagggcaatgctgggcacacacaccatggaagtgactgtctaccatcgccggggatcccggagctatgtgcctcttgctcattccagctcagccttcaccattactgaccaggtgcctttctccgtgagcgtgtcccagttgcgggccttggatggagggaacaagcacttcctgagaaatcagcctctgacctttgccctccagctccatgaccccagtggctatctggctgaagctgacctctcctacacctgggactttggagacagtagtggaaccctgatctctcgggcacttgtggtcactcatacttacctggagcctggcccagtcactgcccaggtggtcctgcaggctgccattcctctcacctcctgtggctcctccccagttccaggcaccacagatgggcacaggccaactgcagaggcccctaacaccacagctggccaagtgcctactacagaagttgtgggtactacacctggtcaggcgccaactgcagagccctctggaaccacatctgtgcaggtgccaaccactgaagtcataagcactgcacctgtgcagatgccaactgcagagagcacaggtatgacacctgagaaggtgccagtttcagaggtcatgggtaccacactggcagagatgtcaactccagaggctacaggtatgacacctgcagaggtatcaattgtggtgctttctggaaccacagctgcacaggtaacaactacagagtgggtggagaccacagctagagagctacctatccctgagcctgaaggtccagatgccagctcaatcatgtctacggaaagtattacaggttccctgggccccctgctggatggtacagccaccttaaggctggtgaagagacaagtccccctggattgtgttctgtatcgatatggttccttttccgtcaccctggacattgtccagggtattgaaagtgccgagatcctgcaggctgtgccgtccggtgagggggatgcatttgagctgactgtgtcctgccaaggcgggctgcccaaggaagcctgcatggagatctcatcgccagggtgccagccccctgcccagcggctgtgccagcctgtgctacccagcccagcctgccagctggttctgcaccagatactgaagggtggctcggggacatactgcctcaatgtgtctctggctgataccaacagcctggcagtggtcagcacccagcttatcatgcctggtcaagaagcaggccttgggcaggttccgctgatcgtgggcatcttgctggtgttgatggctgtggtccttgcatctctgatatataggcgcagacttatgaagcaagacttctccgtaccccagttgccacatagcagcagtcactggctgcgtctaccccgcatcttctgctcttgtcccattggtgagaacagccccctcctcagtgggcagcaggtctgagtactctcatatgatgctgtgattttcctggagttgacagaaacacctatatttcccccagtcttccctgggagactactattaactgaaataaatactcagagcctgaaaaaaaaaaaaaaa

SEQ ID NO: 19 - DNA, 384 bp, Homo sapiens
aagcctgccactgacattgaagaaccaatatatacaatggacaaacaatccagtgccggcggggtgaagaggagcgtcccctgtgattccaacgaggccaacgagatgatgccggagaccccaactggggactcagacccgcaacctgctcctaaaaaaatgaaaacatctgagtcctcgaccatactagtggttcgctacaggaggaactttaaaagaacatctccagaggaactgctgaatgaccacgcccgagagaacagaatcaaccccctccaaatggaggaggaggaattcatggaaataatggttgaaatacctgcaaagtagcaagaagctacatctctcaaccttgggcaatgaaaataaagtttgagaagctga

SEQ ID NO: 20 - DNA, 472 bp, Homo sapiens
gtcaccaggagggtatgcatagggagggcaagagctctgggccactgcgaagattcaaaagctccaaaaacctactgtagacatcgaagaaccaatatatacaatgggccaacaatccagtgtccgcaggctgaagaggagcgtcccctgtgaatccaacgaggccaacgaggccaatgaggccaacaagacgatgccggagaccccaactggggactcagacccgcaacctgctcctaaaaaaatgaaaacatctgagtcctcgaccatactagtggttcgctacaggaggaacgtgaaaagaacatctccagaggaactggtgaatgaccacgcccgagagaacagaatcaaccccgaccaaatggaggaggaggaattcatagaaataacgactgaaagacctaaaaagtagcaagaagctacatccctcaaacttcggcaatgaaaataaagtttgagaagctgaaaa

SEQ ID NO: 21 - DNA, 1466 bp, Homo sapiens
gcatgctctgactttctctctctttcgattcttccatactcagagtacgcacggtctgattttctctttggattcttccaaaatcagagtcagactgctcccggtgccatgaacggagacgacgcctttgcaaggagacccacggttggtgctcaaataccagagaagatccaaaaggccttcgatgatattgccaaatacttctctaaggaagagtgggaaaagatgaaagcctcggagaaaatcttctatgtgtatatgaagagaaagtatgaggctatgactaaactaggtttcaaggccaccctcccacctttcatgtgtaataaacgggccgaagacttccaggggaatgatttggataatgaccctaaccgtgggaatcaggttgaacgtcctcagatgactttcggcaggctccagggaatctccccgaagatcatgcccaagaagccagcagaggaaggaaatgattcggaggaagtgccagaagcatctggcccacaaaatgatgggaaagagctgtgccccccgggaaaaccaactacctctgagaagattcacgagagatctggaaatagggaggcccaagaaaaggaagagagacgcggaacagctcatcggtggagcagtcagaacacacacaacattggtcgattcagtttgtcaacttctatgggtgcagttcatggtacccccaaaacaattacacacaacagggacccaaaaggggggaacatgcctggacccacagactgcgtgagagaaaacagctggtgatttatgaagagatcagcgaccctgaggaagatgacgagtaactcccctcagggatacgacacatgcccatgatgagaagcagaacgtggtgacctttcacgaacatgggcatggctgcggacccctcgtcatcaggtgcatagcaagtgaaagcaagtgttcacaacagtgaaaagttgagcgtcatttttcttagtgtgccaagagttcgatgttagcgtttacgttgtattttcttacactgtgtcattctgttagatactaacattttcattgatgagcaagacatacttaatgcatattttggtttgtgtatccatgcacctaccttagaaaacaagtattgtcggttacctctgcatggaacagcattaccctcctctctccccagatgtgactactgagggcagttctgagtgtttaatttcagattttttcctctgcatttacacacacacgcacacaaaccacaccacacacacacacacacacacacacacacacacacacacacaccaagtaccagtataagcatctgccatctgcttttcccattgccatgcgtcctggtcaagctcccctcactctgtttcctggtcagcatgtactcccctcatccgattcccctgtagcagtcactgacagttaataaacctttgcaaacgttcaaaaaaaaaaaaaaaaaa

SEQ ID NO: 22 - DNA, 1250 bp, Homo sapiens
acacgccgatttgcccttttgattcttccacaatcagggtgagactgctcccagtgccatgaacggagacgacgcctttgcaaggagacccagggatgatgctcaaatatcagagaagttacgaaaggccttcgatgatattgccaaatacttctctaagaaagagtgggaaaagatgaaatcctcggagaaaatcgtctatgtgtatatgaagctaaactatgaggtcatgactaaactaggtttcaaggtcaccctcccacctttcatgcgtagtaaacgggctgcagacttccacgggaatgattttggtaacgatcgaaaccacaggaatcaggttgaacgtcctcagatgactttcggcagcctccagagaatcttcccgaagatcatgcccaagaagccagcagaggaagaaaatggtttgaaggaagtgccagaggcatctggcccacaaaatgatgggaaacagctgtgccccccgggaaatccaagtaccttggagaagattaacaagacatctggacccaaaagggggaaacatgcctggacccacagactgcgtgagagaaagcagctggtggtttatgaagagatcagcgaccctgaggaagatgacgagtaactcccctcggggatatgacacatgcccatgatgagaagcagaacgtggtgacctttcacgaacatgggcatggctgcggacccctcgtcatcaggtgcatagcaagtgaaagcaagtgttcacaacagtgaaaagttgagcgtcatttttcttagtgtgccaagagttcgatgttggcgtttccgctgtattttcttgcagtgtgccattctgttagacattagcgttttcgttgatgagcaagacatgcttaatgcatatttcggcttgtgtatccatgcacctacctcagaaaacaagtattgtcaggtattctctccatagaacagcactaccctcctctctccccagatgtgactactgaggggaggtctgagtgtttaatttccgattttttcctctgcatttacacacacaccacacacgcacacacacacaccaagtaccagtataagcatctcccatctgcttttctccattgccatgcgacctggtcaagcccccctcactctgtttcctgttcagcatgtactcccctcatccgattccgttgtatcagtcactgacagttaataaacctttgcaaacgttcaaaaaaaaaaaaaaaaaaaaaaaaaaaaaaaaa

SEQ ID NO: 23 - DNA, 3685 bp, Homo sapiens
agcacagagataacggccagccctgggcgaaggttaccaccaaagaatatgattttggtctaggggctccagtttctgaagctgaaaactaccagaatactctccagctagaacaagaagtgagaaaccaagatagattcatctcgacactgaaattacaggcatttggatacctttgtgatagaaaaggcccttttcttcatccattgatggtttgatagtgggctgggaaggaaagctgtgttcctccacattaggcagcaaatacttgattgatttgatatttagcttagtcaaattgaagatctcaaacagacaaatcatggcttggaagaatatgttaggaaactcttggatagtaaggaggtggtaagcagtcaagtagatgatttaaccagccacaatgagcatctttgtaaagaattgattaaaattgaccaactagcagagcaactcgaaaaagagaaaaattttgtggtggattccgccaacaaggaacttgaagaagccaagattgatctcatttgccagcaaaataatattatagtattggaagatacaataaaaaggcttaaatctacttgattcctttgtcaagactttggaagcagacaaagatcactataagagtgaagctcaacatttgagaaagatgatgcgaagtaggtctaaaagtccaagacgcccatcaccaactgcccggggtgcaaactgtgatgtagaacttttgaagacaacaacaagagatcgtgaagaacttaaatgcatgctggaaaaatatgagcgccatttggcagaaattcagggtaatgtcaaggttcttaaatctgagagagacaagatcttccttctttatgaacaggcacaggaagaaattacccgacttcgacgagaaatgatgaaaagctgtaagagtcctaaatcaacaacggcacatgctattctccggcgagtggagactgaaagagatgtagcctttactgatttacgaagaatgaccacagaacgagatagtctaagggagaggctaaagattgctcaagagacagcatttaatgagaaggctcacctggaacaaaggatagaggagctggagtgtacagttcataatcttgatgatgaacgtatggagcaaatgtcaaatatgactttgatgaaggaaaccataagcactgtggaaaaagaaatgaaatcactagcaagaaaggcaatggataccgaaagtgaacttggcagacaaaaagcagagaataattctttgagacttttgtatgaaaacacagaaaaagatctttctgatactcagcgacaccttgctaagaaaaaatatgagctacagcttactcaggagaaaattatgtgcttggatgaaaaaattgataactttacaaggcaaaatattgcacagcgagaagaaatcagcattcttggtggaaccctcaatgatctggctaaagaaaaggaatgcctgcaagcatgtttggataaaaaatctgagaatattgcatcccttggagagagtttggcaatgaaagaaaagaccatttcaggcatgaagaatatcattgctgagatggaacaggcatcaagacagtgtactgaggccctaattgtgtgtgaacaagacgtttccagaatgcgtcggcaattggatgagacaaatgatgagctggcccagatcgccagggaaagagatatcttggctcatgacaatgacaatctccaggaacagtttgctaaagctaaacaagaaaaccaggcactgtccaaaaaattgaatgacactcataatgaacttaatgacataaaacagaaggttcaagatactaatttggaggttaacaagctgaagaatatattaaagtctgaagaatctgagaaccggcaaatgatggaacaacttcgaaaagccaatgaagatgctgaaaactgggaaaataaagcccgtcaatcagaggcagataacaataccctcaaactggaacttatcactgctgaggcagagggtaacagattaaaagaaaaagtagattccctcaacagagaggttgagcaacacttaaatgcagaaaggtcttacaagtcccagatttctaccttacataaatctgttgtaaaaatggaagaggagcttcagaaggttcagtttgaaaaagtgtccgctcttgcagatttgtcttctactagggaactctgtattaaacttgactcaagcaaagaacttcttaatcgacagctggttgctaaagatcaagaaatagaaatgagggagaatgagttagattctgctcattctgaaattgaactcctgaggagtcagatggcaaatgagagaatctccatgcagaatctagaagctttgctggtggccaatcgagacaaagaatatcagtctcagatagcacttcaagaaaaagaatctgaaattcagcttcttaaagaacacctttgtttggcagaaaataaaatggccatccagagtagagatgtggcccagttcagaaatgttgtcacacaattggaagctgatttagacattaccaaaagacaactaggaacagagcgctttgaaagggagagggccgtacaagaacttcgccgccaaaattattcaagtaatgcttatcatatgagttctacaatgaagccaaatacaaaatgtcattcaccagaacgtgctcaccatcgatctcctgaccgaggcctagatcgatcattagaagagaatctttgctacagagatttctgacacctgaaatgattcttcacatccctgagaaaggtcaaagttacaaactgatttttttttttttgctacatgagtgcatttatcttttaaatgcttggcaatgttaaatgtatttattaactttgtgtctctgaatctctgttctaatgtgccatgttgcagtgatctgagatgacttataaaaacaaaaatgtatatggctctttctatccatgcagtgatagtgagtgtaaaatctgcttacttcactattgaacactattctgttcactatctggagtaaataaagaagcttattaaaacagggaaaagtgtttttacaaaactgctttcctttcctttcttggtatctcaaataattggttggttaattttttttctttgtgatttgtgttttcatggctggaaaatctgtgccaaaatgggtacaacttactgatactggtctgggatctgatattccattttggaaccaaccttacatgaattccatgccataatctgatattgaagcagcagaaagtaggagtgttgtcagggttttaaaacctatagtattgtaccactcaaagactaagtaacatacatattctacaattgtgtttactactactgcttttagaaaggtaggtgatggtatatctgtcaagaatgtaagattttattaaaaatctttatttttaataagttttctttcaagtactgcaatcaacttaattgcgattttattttattgtctgtggcagctgtctgtaatctggagtaataagataagtgatgtttccttcctcccccaatttcctaaatctcttaccttatctatccataggctttgatgtaatgttcatattagagcctgaatgctactgtattttactgatataaactatttgtattcttgccattttaataaaaaaaggcttgtcattaatagta

SEQ ID NO: 24 - DNA, 1459 bp, Homo sapiens
ccggggcggggtcccggacccccaccctaaggcggagtgacgcccgcagtcacttcacaaggcaaaaattgttacggggcaataaaaggcacaacagcggccaatgtctggcagtgggcacatgggggtgcgggggtgtaggtgccaagcgccatggcttagacccgagattggagtccggccgccccccgacagcagccgcctcctgccccccgtgcgccctaggcgccaccatgtcgggagacaaacttctgagcgaactcggttataagctgggccgcacaattggagagggcagctactccaaggtgaaggtggccacatccaagaagtacaagggtaccgtggccatcaaggtggtggaccggcggcgagcgcccccggacttcgtcaacaagttcctgccgcgagagctgtccatcctgcggggcgtgcgacacccgcacatcgtgcacgtcttcgagttcatcgaggtgtgcaacgggaaactgtacatcgtgatggaagcggccgccaccgacctgctgcaagccgtgcagcgcaacgggcgcatccccggagttcaggcgcgcgacctctttgcgcagatcgccggcgccgtgcgctacctgcacgatcatcacctggtgcaccgcgacctcaagtgcgaaaacgtgctgctgagcccggacgagcgccgcgtcaagctcaccgacttcggcttcggccgccaggcccatggctacccagacctgagcaccacctactgcggctcagccgcctacgcgtcacccgaggtgctcctgggcatcccctacgaccccaagaagtacgatgtgtggagcatgggcgtcgtgctctacgtcatggtcaccgggtgcatgcccttcgacgactcggacatcgccggcctgccccggcgccagaaacgcggcgtgctctatcccgaaggcctcgagctgtccgagcgctgcaaggccctgatcgccgagctgctgcagttcagcccgtccgccaggccctccgcgggccaggtagcgcgcaactgctggctgcgcgccggggactccggctagaagccgggtggttccagccattcctgcagccaagggcactgggccagggcggcgcacgcgcaagaggcgcgcttcgagggaatatgcgaagctgccgcgtgctgctgcacatgcgctttttcccttccgcttccctccctttcttcccacgggggagtccgcagttgcccttgttcggaatccacgttccccgcgatcccgggagctggaggcgcatgcgcatccgcgattccctgcgaccaggccccgagagggcgagaccagaggggacggaagcattgcgcctgcgcggaactctcagcctctgcgcggagggcgtcccttcccaaccagccgtgggtgccaggttcccggttggaacctgcaataaactcgctgttcctcgcaaaaaaaaaaaaaaaaaa

SEQ ID NO: 25 - DNA, 2082 bp, Homo sapiens
atcactgtagtagtagctggaaagagaaatctgtgactccaattagccagttcctgcagaccttgtgaggactagaggaagaatgctcctggctgttttgtactgcctgctgtggagtttccagacctccgctggccatttccctagagcctgtgtctcctctaagaacctgatggagaaggaatgctgtccaccgtggagcggggacaggagtccctgtggccagctttcaggcagaggttcctgtcagaatatccttctgtccaatgcaccacttgggcctcaatttcccttcacaggggtggatgaccgggagtcgtggccttccgtcttttataataggacctgccagtgctctggcaacttcatgggattcaactgtggaaactgcaagtttggcttttggggaccaaactgcacagagagacgactcttggtgagaagaaacatcttcgatttgagtgccccagagaaggacaaattttttgcctacctcactttagcaaagcataccatcagctcagactatgtcatccccatagggacctatggccaaatgaaaaatggatcaacacccatgtttaacgacatcaatatttatgacctctttgtctggatgcattattatgtgtcaatggatgcactgcttgggggatctgaaatctggagagacattgattttgcccatgaagcaccagcttttctgccttggcatagactcttcttgttgcggtgggaacaagaaatccagaagctgacaggagatgaaaacttcactattccatattgggactggcgggatgcagaaaagtgtgacatttgcacagatgagtacatgggaggtcagcaccccacaaatcctaacttactcagcccagcatcattcttctcctcttggcagattgtctgtagccgattggaggagtacaacagccatcagtctttatgcaatggaacgcccgagggacctttacggcgtaatcctggaaaccatgacaaatccagaaccccaaggctcccctcttcagctgatgtagaattttgcctgagtttgacccaatatgaatctggttccatggataaagctgccaatttcagctttagaaatacactggaaggatttgctagtccacttactgggatagcggatgcctctcaaagcagcatgcacaatgccttgcacatctatatgaatggaacaatgtcccaggtacagggatctgccaacgatcctatcttccttcttcaccatgcatttgttgacagtatttttgagcagtggctccgaaggcaccgtcctcttcaagaagtttatccagaagccaatgcacccattggacataaccgggaatcctacatggttccttttataccactgtacagaaatggtgatttctttatttcatccaaagatctgggctatgactatagctatctacaagattcagacccagactcttttcaagactacattaagtcctatttggaacaagcgagtcggatctggtcatggctccttggggcggcgatggtaggggccgtcctcactgccctgctggcagggcttgtgagcttgctgtgtcgtcacaagagaaagcagcttcctgaagaaaagcagccactcctcatggagaaagaggattaccacagcttgtatcagagccatttataaaaggcttaggcaatagagtagggccaaaaagcctgacctcactctaactcaaagtaatgtccaggttcccagagaatatctgctggtatttttctgtaaagaccatttgcaaaattgtaacctaatacaaagtgtagccttcttccaactcaggtagaacacacctgtctttgtcttgctgttttcactcagcccttttaacattttcccctaagcccatatgtctaaggaaaggatgctatttggtaatgaggaactgttatttgtatgtgaattaaagtgctcttattttaaaaaattgaaataattttgatttttgccttctgattatttaaagatctatatatgttttattggccccttctttattttaataaaacagtgagaaatctaaaaaaaaaaaaaaaaaa

SEQ ID NO: 26 - DNA, 720 bp, Homo sapiens
accagggaggggagggaggactgcatgacgcgggatggggtggggcgaggcggggcactgcagcacacaacgcaggcaccgacttcagtgtgcatgttccttggacacctgcctcagtgtgcatgttcactgggcatcttcccttcgacccctttgcccacgtggtgaccgctggggagctgtgagagtgtgaggggcacgttccagccgtctggactctttctctcctactgagacgcagcctataggtccgcaggccagtcctcccaggaactgaaatagtgaaatatgagttggcgaggaagatcaacatataggcctaggccaagaagaagtttacagcctcctgagctgattggggctatgcttgaacccactgatgaagagcctaaagaagagaaaccacccactaaaagtcggaatcctacacctgatcagaagagagaagatgatcagggtgcagctgagattcaagtgcctgacctggaagccgatctccaggagctatgtcagacaaagactggggatggatgtgaaggtggtactgatgtcaaggggaagattctaccaaaagcagagcactttaaaatgccagaagcaggtgaagggaaatcacaggtttaaaggaagataagctgaaacaacacaaactgtttttatattagatattttactttaaaatatcttaataaagttttaagcttttctccaaaaaaaaaa

SEQ ID NO: 27 - DNA, 2411 bp, Homo sapiens
cttttcaggagtcccttctgttatttagcaagacacactactacaatggagaaaaaagatgccccctccttcaagccctgagatcttcaggactttggcgaaaagtctgcgcccgaagagacccaggaaggattcttggaattgtagtccaaaggcatcccgccttctgcgcagactcacaagtccctgtggacggaattcttgaagtgtagcgccgctcagtccttccaccggaagtgtccgatcggaatcagccctgtccgagaggtgagtccgggtttggggatccagatgtccagccccgtgtccccctccaaacatccagtccctctcatattgcctttgaaattagcagcctctgggtgaccagaccttggccctcagaggaatcccggagaaaggtagaaccagcttcggcgttgggaacgcaggcgcgcttacgcatttagtgagggtttggcggtctccatagttaccgccgccgcgcgtgacgtcatagtggagcgctgagggcttggtggcgtggggtgggggctgtcctactgatcctgaatttggggtcactggtaagaggagttgcccattccagccaggtggaacggggaggggtgccacatgtctcagatctgccattgtctgcgaaaagaaactgctgcgaggaccatccccaatcccctgcttcccttgggaagagtaaccgccgttttgtaggacacttggggacaaccccgcttgtcctgaaatttattgacacggtaaatagtatttcctgtgtgccgaggatgcagttaaaccaacactgaccccctgcccttgagaaacacaagatggctacagaaccaaagaaagctgcagcccagaactctccagaggatgaaggacttctgatagtgaagatagaagaggaagaatttatccatgggcaggacacttgcttacagagaagtgaactccttaagcaggagctctgcaggcagctttttaggcagttctgctaccaggattctcctggacctcgcgaggcactgagccgcctccgggagctctgctgtcagtggctgaagccagagatccataccaaggaacagattctggaactgctggtgctagagcagttcctgaccatcctgccaggagatttgcaggcctgggtacatgaacattacccagagagtggagaggaggcagtgaccatactagaagatttggagagaggcactgatgaagcagtactccaggttcaagcccatgaacatggacaagaaatattccagaaaaaagtgtcacctcctggaccagcacttaatgtcaagttacagccagtggagaccaaggcccattttgattcatcagaaccccagctcctatgggactgtgataatgagagtgaaaacagtagatccatgccaaagctggaaatttttgaaaaaattgaatcacagagaattatatctggaagaatctcaggatacatatcagaagcatctggtgagtctcaagacatctgtaagtctgcaggcagggtaaagagacaatgggaaaaagaatcaggggagtctcagagactctcgtctgcccaggatgaaggttttggtaaaatcctcacccacaaaaatacagtcagaggtgaaataataagccacgatggatgtgagaggagattaaatctgaactcaaatgaattcacacaccagaaatcttgtaaacatggtacctgtgaccagagcttcaaatggaactcagattttattaaccatcaaataatttatgctggagaaaaaaatcaccaatatggaaaatctttcaagagcccaaaacttgctaaacatgcagcagttttcagtggagataaaactcatcagtgtaatgaatgtgggaaagctttcaggcacagctcaaaacttgctaggcatcagagaatccacactggagagagatgctatgaatgtaatgaatgtgggaaaagctttgcagagagctcagatcttactagacatcggcgaattcacactggggaaagaccctttggttgcaaagaatgtgggagagcattcaacctgaactcacatcttatcaggcatcagagaattcacaccagagagaaaccctacgagtgtagtgaatgtgggaaaaccttccgagtgagctcacatcttattcgacactttagaattcacactggagaaaaaccctatgaatgcagtgagtgtggaagagccttcagtcagagctcaaaccttagtcaacaccagagaattcacatgagggaaaacctattaatgtaaggaacttaaatttgtaagtaaatgctgaggaaatggcacaatatgaaaaatattaaataaaaaataaatattgggcaagatggaagactgaaaaaaaaaaaaaaaaaaaaaaaaaaaaaaaaaaaa

SEQ ID NO: 28 - DNA, 5916 bp, Homo sapiens
gcccctccctccgcccgcccgccggcccgcccgtcagtctggcaggcaggcaggcaatcggtccgagtggctgtcggctcttcagctctcccgctcggcgtcttccttcctcctcccggtcagcgtcggcggctgcaccggcggcggcgcagtccctgcgggaggggcgacaagagctgagcggcggccgccgagcgtcgagctcagcgcggcggaggcggcggcggcccggcagccaacatggcggcggcggcggcggcgggcgcgggcccggagatggtccgcgggcaggtgttcgacgtggggccgcgctacaccaacctctcgtacatcggcgagggcgcctacggcatggtgtgctctgcttatgataatgtcaacaaagttcgagtagctatcaagaaaatcagcccctttgagcaccagacctactgccagagaaccctgagggagataaaaatcttactgcgcttcagacatgagaacatcattggaatcaatgacattattcgagcaccaaccatcgagcaaatgaaagatgtatatatagtacaggacctcatggaaacagatctttacaagctcttgaagacacaacacctcagcaatgaccatatctgctattttctctaccagatcctcagagggttaaaatatatccattcagctaacgttctgcaccgtgacctcaagccttccaacctgctgctcaacaccacctgtgatctcaagatctgtgactttggcctggcccgtgttgcagatccagaccatgatcacacagggttcctgacagaatatgtggccacacgttggtacagggctccagaaattatgttgaattccaagggctacaccaagtccattgatatttggtctgtaggctgcattctggcagaaatgctttctaacaggcccatctttccagggaagcattatcttgaccagctgaaccacattttgggtattcttggatccccatcacaagaagacctgaattgtataataaatttaaaagctaggaactatttgctttctcttccacacaaaaataaggtgccatggaacaggctgttcccaaatgctgactccaaagctctggacttattggacaaaatgttgacattcaacccacacaagaggattgaagtagaacaggctctggcccacccatatctggagcagtattacgacccgagtgacgagcccatcgccgaagcaccattcaagttcgacatggaattggatgacttgcctaaggaaaagctcaaagaactaatttttgaagagactgctagattccagccaggatacagatcttaaatttgtcaggacaagggctcagaggactggacgtgctcagacatcggtgttcttcttcccagttcttgacccctggtcctgtctccagcccgtcttggcttatccactttgactcctttgagccgtttggaggggcggtttctggtagttgtggcttttatgctttcaaagaatttcttcagtccagagaattcctcctggcagccctgtgtgtgtcacccattggtgacctgcggcagtatgtacttcagtgcacctactgcttactgttgctttagtcactaattgctttctggtttgaaagatgcagtggttcctccctctcctgaatccttttctacatgatgccctgctgaccatgcagccgcaccagagagagattcttccccaattggctctagtcactggcatctcactttatgatagggaaggctactacctagggcactttaagtcagtgacagccccttatttgcacttcaccttttgaccataactgtttccccagagcaggagcttgtggaaataccttggctgatgttgcagcctgcagcaagtgcttccgtctccggaatccttggggagcacttgtccacgtcttttctcatatcatggtagtcactaacatatataaggtatgtgctattggcccagcttttagaaaatgcagtcatttttctaaataaaaaggaagtactgcacccagcagtgtcactctgtagttactgtggtcacttgtaccatatagaggtgtaacacttgtcaagaagcgttatgtgcagtacttaatgtttgtaagacttacaaaaaaagatttaaagtggcagcttcactcgacatttggtgagagaagtacaaaggttgcagtgctgagctgtgggcggtttctggggatgtcccagggtggaactccacatgctggtgcatatacgcccttgagctacttcaaatgtgggtgtttcagtaaccacgttccatgcctgaggatttagcagagaggaacactgcgtctttaaatgagaaagtatacaattctttttccttctacagcatgtcagcatctcaagttcatttttcaacctacagtataacaatttgtaataaagcctccaggagctcatgacgtgaagcactgttctgtcctcaagtactcaaatatttctgatactgctgagtcagactgtcagaaaaagctagcactaactcgtgtttggagctctatccatattttactgatctctttaagtatttgttcctgccactgtgtactgtggagttgactcggtgttctgtcccagtgcggtgcctcctcttgacttccccactgctctctgtggtgagaaatttgccttgttcaataattactgtaccctcgcatgactgttacagctttctgtgcagagatgactgtccaagtgccacatgcctacgattgaaatgaaaactctattgttacctctgagttgtgttccacggaaaatgctatccagcagatcatttaggaaaaataattctatttttagcttttcatttctcagctgtccttttttcttgtttgatttttgacagcaatggagaatgggttatataaagactgcctgctaatatgaacagaaatgcatttgtaattcatgaaaataaatgtacatcttctatcttcacattcatgttaagattcagtgttgctttcctctggatcagcgtgtctgaatggacagtcaggttcaggttgtgctgaacacagaaatgctcacaggcctcactttgccgcccaggcactggcccagcacttggatttacataagatgagttagaaaggtacttctgtagggtcctttttacctctgctcggcagagaatcgatgctgtcatgttcctttattcacaatcttaggtctcaaatattctgtcaaaccctaacaaagaagccccgacatctcaggttggattccctggttctctctaaagagggcctgcccttgtgccccagaggtgctgctgggcacagccaagagttgggaagggccgccccacagtacgcagtcctcaccacccagcccagggtgctcacgctcaccactcctgtggctgaggaaggatagctggctcatcctcggaaaacagacccacatctctattcttgccctgaaatacgcgcttttcacttgcgtgctcagagctgccgtctgaaggtccacacagcattgacgggacacagaaatgtgactgttaccggataacactgattagtcagttttcatttataaaaaagcattgacagttttattactcttgtttctttttaaatggaaagttactattataaggttaatttggagtcctcttctaaatagaaaaccatatccttggctactaacatctggagactgtgagctccttcccattccccttcctggtactgtggagtcagattggcatgaaaccactaacttcattctagaatcattgtagccataagttgtgtgctttttattaatcatgccaaacataatgtaactgggcagagaatggtcctaaccaaggtacctatgaaaagcgctagctatcatgtgtagtagatgcatcattttggctcttcttacatttgtaaaaatgtacagattaggtcatcttaattcatattagtgacacggaacagcacctccactatttgtatgttcaaataagctttcagactaatagcttttttggtgtctaaaatgtaagcaaaaaattcctgctgaaacattccagtcctttcatttagtataaaagaaatactgaacaagccagtgggatggaattgaaagaactaatcatgaggactctgtcctgacacaggtcctcaaagctagcagagatacgcagacattgtggcatctgggtagaagaatactgtattgtgtgtgcagtgcacagtgtgtggtgtgtgcacactcattccttctgctcttgggcacaggcagtgggtgtagaggtaaccagtagctttgagaagctacatgtagctcaccagtggttttctctaaggaatcacaaaagtaaactacccaaccacatgccacgtaatatttcagccattcagaggaaactgttttctctttatttgcttatatgttaatatggtttttaaattggtaacttttatatagtatggtaacagtatgttaatacacacatacatacgcacacatgctttgggtccttccataatacttttatatttgtaaatcaatgttttggagcaatcccaagtttaagggaaatatttttgtaaatgtaatggttttgaaaatctgagcaatccttttgcttatacatttttaaagcatttgtgctttaaaattgttatgctggtgtttgaaacatgatactcctgtggtgcagatgagaagctataacagtgaatatgtggtttctcttacgtcatccaccttgacatgatgggtcagaaacaaatggaaatccagagcaagtcctccagggttgcaccaggtttacctaaagcttgttgccttttcttgtgctgtttatgcgtgtagagcactcaagaaagttctgaaactgctttgtatctgctttgtactgttggtgccttcttggtattgtaccccaaaattctgcatagattatttagtataatggtaagttaaaaaatgttaaaggaagattttattaagaatctgaatgtttattcattatattgttacaatttaacattaacatttatttgtggtatttgtgatttggttaatctgtataaaaattgtaagtagaaaggtttatatttcatcttaattcttttgatgttgtaaacgtactttttaaaagatggattatttgaatgtttatggcacctgacttgtaaaaaaaaaaaactacaaaaaaatccttagaatcattaaattgtgtccctgtattaccaaaataacacagcaccgtgcatgtatagtttaattgcagtttcatctgtgaaaacgtgaaattgtctagtccttcgttatgttccccagatgtcttccagatttgctctgcatgtggtaacttgtgttagggctgtgagctgttcctcgagttgaatggggatgtcagtgctcctagggttctccaggtggttcttcagaccttcacctgtgggggggggggtaggcggtgcccacgcccatctcctcatcctcctgaacttctgcaaccccactgctgggcagacatcctgggcaaccccttttttcagagcaagaagtcataaagataggatttcttggacatttggttcttatcaatattgggcattatgtaatgacttatttacaaaacaaagatactggaaaatgttttggatgtggtgttatggaaagagcacaggccttggacccatccagctgggttcagaactaccccctgcttataactgcggctggctgtgggccagtcattctgcgtctctgctttcttcctctgcttcagactgtcagctgtaaagtggaagcaatattacttgccttgtatatggtaaagattataaaaatacatttcaactgttcagcatagtacttcaaagcaagtactcagtaaatagcaagtctttttaaa

SEQ ID NO: 29 - DNA, 2949 bp, Homo sapiens
cgcctcccttccccctccccgcccgacagcggccgctcgggccccggctctcggttataagatggcggcgctgagcggtggcggtggtggcggcgcggagccgggccaggctctgttcaacggggacatggagcccgaggccggcgccggcgccggcgccgcggcctcttcggctgcggaccctgccattccggaggaggtgtggaatatcaaacaaatgattaagttgacacaggaacatatagaggccctattggacaaatttggtggggagcataatccaccatcaatatatctggaggcctatgaagaatacaccagcaagctagatgcactccaacaaagagaacaacagttattggaatctctggggaacggaactgatttttctgtttctagctctgcatcaatggataccgttacatcttcttcctcttctagcctttcagtgctaccttcatctctttcagtttttcaaaatcccacagatgtggcacggagcaaccccaagtcaccacaaaaacctatcgttagagtcttcctgcccaacaaacagaggacagtggtacctgcaaggtgtggagttacagtccgagacagtctaaagaaagcactgatgatgagaggtctaatcccagagtgctgtgctgtttacagaattcaggatggagagaagaaaccaattggttgggacactgatatttcctggcttactggagaagaattgcatgtggaagtgttggagaatgttccacttacaacacacaactttgtacgaaaaacgtttttcaccttagcattttgtgacttttgtcgaaagctgcttttccagggtttccgctgtcaaacatgtggttataaatttcaccagcgttgtagtacagaagttccactgatgtgtgttaattatgaccaacttgatttgctgtttgtctccaagttctttgaacaccacccaataccacaggaagaggcgtccttagcagagactgccctaacatctggatcatccccttccgcacccgcctcggactctattgggccccaaattctcaccagtccgtctccttcaaaatccattccaattccacagcccttccgaccagcagatgaagatcatcgaaatcaatttgggcaacgagaccgatcctcatcagctcccaatgtgcatataaacacaatagaacctgtcaatattgatgacttgattagagaccaaggatttcgtggtgatggaggatcaaccacaggtttgtctgctaccccccctgcctcattacctggctcactaactaacgtgaaagccttacagaaatctccaggacctcagcgagaaaggaagtcatcttcatcctcagaagacaggaatcgaatgaaaacacttggtagacgggactcgagtgatgattgggagattcctgatgggcagattacagtgggacaaagaattggatctggatcatttggaacagtctacaagggaaagtggcatggtgatgtggcagtgaaaatgttgaatgtgacagcacctacacctcagcagttacaagccttcaaaaatgaagtaggagtactcaggaaaacacgacatgtgaatatcctactcttcatgggctattccacaaagccacaactggctattgttacccagtggtgtgagggctccagcttgtatcaccatctccatatcattgagaccaaatttgagatgatcaaacttatagatattgcacgacagactgcacagggcatggattacttacacgccaagtcaatcatccacagagacctcaagagtaataatatatttcttcatgaagacctcacagtaaaaataggtgattttggtctagctacagtgaaatctcgatggagtgggtcccatcagtttgaacagttgtctggatccattttgtggatggcaccagaagtcatcagaatgcaagataaaaatccatacagctttcagtcagatgtatatgcatttggaattgttctgtatgaattgatgactggacagttaccttattcaaacatcaacaacagggaccagataatttttatggtgggacgaggatacctgtctccagatctcagtaaggtacggagtaactgtccaaaagccatgaagagattaatggcagagtgcctcaaaaagaaaagagatgagagaccactctttccccaaattctcgcctctattgagctgctggcccgctcattgccaaaaattcaccgcagtgcatcagaaccctccttgaatcgggctggtttccaaacagaggattttagtctatatgcttgtgcttctccaaaaacacccatccaggcagggggatatggtgcgtttcctgtccactgaaacaaatgagtgagagagttcaggagagtagcaacaaaaggaaaataaatgaacatatgtttgcttatatgttaaattgaataaaatactctctttttttttaaggtgaaccaaagaacacttgtgtggttaaagactagatataatttttccccaaactaaaatttatacttaacattggatttttaacatccaagggttaaaatacatagacattgctaaaaattggcagagcctcttctagaggctttactttctgttccgggtttgtatcattcacttggttattttaagtagtaaacttcagtttctcatgcaacttttgttgccagctatcacatgtccactagggactccagaagaagaccctacctatgcctgtgtttgcaggtgagaagttggcagtcggttagcctgggttagataaggcaaactgaacagatctaatttaggaagtcagtagaatttaataattctattattattcttaataatttttctataactatttctttttataacaatttggaaaatgtggatgtcttttatttccttgaagcaataaactaagtttctttttataaaaa

SEQ ID NO: 30 - DNA, 2333 bp, Homo sapiens
tgcggagcttcgtgatgcacgccccgatgcctgcggggctataaaaacgctcgcaagcgccaagtctcctcaggagccgccggcaagggggcaacgaggaagctcttaagagcgcggccggaaagcagttgagttacagacatcctgccaaaatgatttcttcaaagcccagacttgtcgtaccctatggcctcaagactctgctcgagggaattagcagagctgttctcaaaaccaacccatcaaacatcaaccagtttgcagcagcttattttcaagaacttactatgtatagagggaatactactatggatataaaagatctggttaaacaatttcatcagattaaagtagagaaatggtcagaaggaacgacaccacagaagaaattagaatgtttaaaagaaccaggaaaaacatctgtagaatctaaagtacctacccagatggaaaaatctacagacacagacgaggacaatgtaaccagaacagaatatagtgacaaaaccacccagtttccatcagtttatgctgtgccaggcactgagcaaacggaagcagttggtggtctttcttccaaaccagccacccctaagactactaccccaccctcatcaccacctccaacagctgtctcaccagagtttgcctacgtcccagctgacccagctcagcttgctgctcagatgttaggtaaagtttcatctattcattctgatcaatctgatgtgttaatggtggatgtggcaaccagtatgcctgttgttatcaaggaggtgccaagctcagaggctgctgaagatgtcatggtggctgctcctcttgtgtgttctggaaaggtgctagaagtgcaggttgtgaaccaaacatctgtccatgtagatttgggttctcaacctaaagaaaatgaggctgaaccatcaacggcttcctcagtccccttgcaggatgaacaagaacctcctgcttatgatcaagctcctgaggtcactttgcaggctgatattgaggttatgtcaactgttcatatatcatctgtctataacgatgtgcctgtgactgaaggagttgtttatatcgagcaactgccagaacaaatagttatcccttttactgatcaagttgcttgtcttaaagaaaatgagcagtcaaaagaaaatgagcagtcaccacgagttagtcccaaatctgtagtagaaaagaccacctctggcatgtctaaaaaatctgtagagtctgtaaaacttgcacagttggaggagaatgcaaaatattcctcagtatatatggaggcagaagcaacagctctgctctctgacacatctttgaaaggtcagcctgaggtacctgcacaactcctggatgcagaaggtgctatcaaaataggctctgaaaaatctctgcaccttgaagtggagatcacttcaatagtctctgacaatactgggcaggaggagtctggggaaaactctgtaccccaggagatggaaggcaaacctgtgctctctggggaagctgcagaagcagtgcactcaggtacatctgtaaagtcatctagtggccccttccctcctgctccagaaggccttactgcaccagaaattgaaccagaaggggaatcaacagctgaataaggtttgatgaagccagcaatggcaacaagtgaacgaggacaaccaccaccatgttctaacatgtggaccctttattgtctaactgataagaatcaacaaggtcacccatcaccgccacctgcacctgggccttttccccaagcaaccctctatttacctaatcctaaggatccacagtttcagcagcatccaccaaaagtcacttttccaacttatgtgatgggcgacaccaagaagaccagtgccccaccttttatcttagtaggctcaaatgttcaggaagcacagggatggaaacctcttcctggacatgctgtcgtttcacagtcagatgtcttgagatatgttgcaatgcaagtgcccattgctgttcctgcagatgagaaataccagaaacataccctaagtccccagaatgctaatcctccaagtggacaagatgtccccaggccaaaaagccctgttttcctttctgttgctttcccagtagaagatgtagctaaaaaaagttcaggatctggtgacaaatgtgctccctttggaagttacggtattgctggggaggtaaccgtgactactgctcacaaacgtcgcaaagcagaaactgaaaactgatccagaaatgacgctgtctgggtcaacatttcagggaggagtctgccaccagtgtaatgtatcaataaacttcatgcaagcata

SEQ ID NO: 31 - DNA, 2219 bp, Homo sapiens
gcacaaccagaatttgccaaaacaggaaataggtgtttcatatatacggctctaaccttctctctctgcaccttccttctgtcaatagatgaaacaaatacttcatcctgctctggaaaccactgcaatgacattattcccagtgctgttgttcctggttgctgggctgcttccatcttttccagcaaatgaagataaggatcccgcttttactgctttgttaaccacccaaacacaagtgcaaagggagattgtgaataagcacaatgaactgaggagagcagtatctccccctgccagaaacatgctgaagatggaatggaacaaagaggctgcagcaaatgcccaaaagtgggcaaaccagtgcaattacagacacagtaacccaaaggatcgaatgacaagtctaaaatgtggtgagaatctctacatgtcaagtgcctccagctcatggtcacaagcaatccaaagctggtttgatgagtacaatgattttgactttggtgtagggccaaagactcccaacgcagtggttggacattatacacaggttgtttggtactcttcatacctcgttggatgtggaaatgcctactgtcccaatcaaaaagttctaaaatactactatgtttgccaatattgtcctgctggtaattgggctaatagactatatgtcccttatgaacaaggagcaccttgtgccagttgcccagataactgtgacgatggactatgcaccaatggttgcaagtacgaagatctctatagtaactgtaaaagtttgaagctcacattaacctgtaaacatcagttggtcagggacagttgcaaggcctcctgcaattgttcaaacagcatttattaaatacgcattacacaccgagtagggctatgtagagaggagtcagattatctacttagatttggcatctacttagatttaacatatactagctgagaaattgtaggcatgtttgatacacatttgatttcaaatgtttttcttctggatctgctttttattttacaaaaatatttttcatacaaatggttaaaaagaaacaaaatctataacaacaactttggatttttatatataaactttgtgatttaaatttactgaatttaattagggtgaaaattttgaaagttgtattctcatatgactaagttcactaaaaccctggattgaaagtgaaaattatgttcctagaacaaaatgtacaaaaagaacaatataattttcacatgaacccttggctgtagttgcctttcctagctccactctaaggctaagcatcttcaaagacgttttcccatatgctgtcttaattcttttcactcattcacccttcttcccaatcatctggctggcatcctcacaattgagttgaagctgttcctcctaaaacaatcctgacttttattttgccaaaatcaatacaatcctttgaattttttatctgcataaattttacagtagaatatgatcaaaccttcatttttaaacctctcttctctttgacaaaacttccttaaaaaagaatacaagataatataggtaaataccctccactcaaggaggtagaactcagtcctctcccttgtgagtcttcactaaaatcagtgactcacttccaaagagtggagtatggaaagggaaacatagtaactttacaggggagaaaaatgacaaatgacgtcttcaccaagtgatcaaaattaacgtcaccagtgataagtcattcagatttgttctagataatctttctaaaaattcataatcccaatctaattatgagctaaaacatccagcaaactcaagttgaaggacattctacaaaatatccctggggtattttagagtattcctcaaaactgtaaaaatcatggaaaataagggaatcctgagaaacaatcacagaccacatgagactaaggagacatgtgagccaaatgcaatgtgcttcttggatcagatcctggaacagaaaaagatcagtaatgaaaaaactgatgaagtctgaatagaatctggagtatttttaacagtagtgttgatttcttaatcttgataaatatagcagggtaatgtaagatgataacgttagagaaactgaaactgggtgagggctatctaggaattctctgtactatcttaccaaattttcggtaagtctaagaaagcaatgcaaaataaaaagtgtcttgaaaaaaaa

SEQ ID NO: 32 - DNA, 3932 bp, Homo sapiens
tcccagacagaacctactatgtgcggcggcagctggggcgggaaggcgggagctgggggcgctgggggcgctgcggccgctgcggccgctgcagccgctgcagcgccagggtccacctggtcggctgcacctgtggaggaggaggtggatttcaggcttcccgtagactggaagaatcggctcaaaaccgcttgcctcgcaggggctgagctggaggcagcgaggccgcccgacgcaggcttccggcgagacatggcagggcaaggatggcagcccggcggcagggcctggcgaggagcgcgagcccgcggccgcagttcccaggcgtctgcgggcgcgagcacgccgcgaccctgcgtgcgccggggcgggggggcggggcctcgcctgcacaaatggggacgaggggggcggggcggccacaatttcgcgccaaacttgaccgcgcgttctgctgtaacgagcgggctcggaggtcctcccgctgctgtcatggttggttcgctaaactgcatcgtcgctgtgtcccagaacatgggcatcggcaagaacggggacctgccctggccaccgctcaggaatgaattcagatatttccagagaatgaccacaacctcttcagtagaaggtaaacagaatctggtgattatgggtaagaagacctggttctccattcctgagaagaatcgacctttaaagggtagaattaatttagttctcagcagagaactcaaggaacctccacaaggagctcattttctttccagaagtctagatgatgccttaaaacttactgaacaaccagaattagcaaataaagtagacatggtctggatagttggtggcagttctgtttataaggaagccatgaatcacccaggccatcttaaactatttgtgacaaggatcatgcaagactttgaaagtgacacgttttttccagaaattgatttggagaaatataaacttctgccagaatacccaggtgttctctctgatgtccaggaggagaaaggcattaagtacaaatttgaagtatatgagaagaatgattaatatgaaggtgttttctagtttaagttgttccccctccctctgaaaaaagtatgtatttttacattagaaaaggttttttgttgactttagatctataattatttctaagcaactagtttttattccccactactcttgtctctatcagataccatttatgagacattcttgctataactaagtgcttctccaagaccccaactgagtccccagcacctgctacagtgagctgccattccacacccatcacatgtggcactcttgccagtccttgacattgtcgggcttttcacatgttggtaatatttattaaagatgaagatccacatacccttcaactgagcagtttcactagtggaaataccaaaagcttcctacgtgtatatccagaggtttgtagataaatgttgccaccttgtttgtaacagtgaaaaattgaaaacaacctggaagtccagtgatgggaaaatgagtatgtttctgtcttagattggggaacccaaagcagattgcaagactgaaatttcagtgaaagcagtgtatttgctaggtcataccagaaatcatcaattgaggtacggagaaactgaactgagaaggtaagaaaagcaatttaaagtcagcgagcaggttctcattgataacaagctccatactgctgagatacagggaaatggaggggggaaagctggagtattgatcccgcccccctccttggttgtcagctccctgtcctgtgtgtgggcggaacatagtccagctgctctatagcaagtctcaggtgtttgcagtaagaagctgctggcatgcacgggaacagtgaatgccaaacacttaaagcaattcgatgtttaagtatgtaagttcttttttttttagacagcgtttcgctcttgttgcccaggctagcatgcaatggtgtgacctcggcttactgcaacctccgccttcccagattcaagcgattctcctgcctcaggctcccaagtagctaggaccaggtgcgcgccaccacgcccggctaatttttgtattttgtatttttagtagagatggggtttcaccatgttggtcaggctagtctcgaactcgtgaccgcaagcgattcacccacctcagcctcccaaagtgctgggattaccggcttgagccaccacacccggcacatcttcattctttttatgtagtaaaaagtataaggccacacatggtttatttgaagtattttataatttaaaaaaatacagaagcaggaaaaccaattataagttcaagtgagggatgatggttgcttgaaccaaagggttgcatgtagtaagaaattgtgatttaagatatattttaaagttataagtagcaggatattctgatggagtttgactttggttttgggcccagggagtttcagatgcctttgagaaatgaatgaagtagagagaaaataaaagaaaaaccagccaggcacagtggctcacacctgtaatcccagcgctttgggaggctaaggcaggcagatcacttgagaccagcttgggcaacatggcaaagccccatctctacaaaaaacacaaaaattagctgggcattgtggcgcacacctgtattcccatctagtcaggaagctgagatggaagaattaattgagcccacgagttcaaggctgcagtgagtcgtgattgtgccactgcactccagccggggtgacagaagagaccttgtctcgaaaaggaatctgaaaacaatggaaccatgccttcataattctagaaagttattttcaactgataaatctatattcacccaaataatcaagggtgaaggtaaaataatacatttttagacaagcaaagactcaggggttacctccatgtgccctttttagggaagctgttggagaaaatactccagcaaaatgaaggagtacacaaaccagagaatgacatgaatccagcaaataggatccaacacaggcaatattccagctatggagctagctttaaaaaggaacagtaaaaatattaatcggttagctgggtggaatggcccatgcctgtagtcccagctactcaggaggctcagcagcaggacgacttgagcccaagagttccagaccagcctggccaccttagtgagatcccttctcttaaaaataataacttattgccagatttggggcatttggaaagaagttcattgaagataaagcaaaagtaaaaaaaaaaaaaaaaaaaacaaggggaaagggttggttaggcaatcattctagggcagaaagaagtacaggataggaagagcataatacactgtttttctcaacaaggagcagtatgtacacagtcataatgatgtgactgcttagcccctaaatatggtaactactctgggacaatatgggaggaaaagtgaagattgtgatggtgtaagagctaaatcctcatctgtcatatccagaaatcactatataatatataataatgaaatgactaagttatgtgaggaaaaaaacagaagacattgctaaaagagttaaaagtcattgctctggagaattaggagggatggggcaggggactgttaggatgcattataaactgaaaagcctttttaaaattttatgtattaatatatgcattcacttgaaaaactaaaaaaaaacaataatttggaaaaacccatgaaggtaactaacggaaggaaaaactaagagaatgaaaagtatttgcctctggaaagaacaactggcaggactgttgttttcattgtaagacttttggagccatttaattgtacttaaccattttcatctatttctttaataagaacaattccatcttaataaagagttacacttgttaataagtaaaaaaaaaaaaaaa

SEQ ID NO: 33 - DNA, 3486 bp, Homo sapiens
gcgctgccgccagcgaggcccggggaggccgcggcggaggcggaggcccggcgccctgggcggcgccctgtccccgaagtccgtcctccccgttaggtggcgagcgcccgaggggaggggacagccgggcaagcaggaagctgcggcttaaaagggcaacccgcgcgggacccttcctccctagtcgcggggagtctgagaaagcgcgcctgtttcgcgaccatcacgcacctcccctccgcttgtggccatgtaccgctacctgggcgaagcgctgttgctgtcccgggccgggcccgctgccctgggctcggcgtccgccgactcggccgcgttgctgggctgggcccggggacagcccgccgccgccccgcagccggggctggcattggccgcccggcgccactacagcgaggcggtggccgaccgcgaggacgaccccaacttcttcaagatggtggagggcttcttcgatcgcggcgccagcatcgtggaggacaagctggtggaggacctgaggacccgggagagcgaggagcagaagcggaaccgggtgcgcggcatcctgcggatcatcaagccctgcaaccatgtgctgagtctctccttccccatccggcgcgacgacggctcctgggaggtcatcgaaggctaccgggcccagcacagccagcaccgcacgccctgcaagggaggtatccgttacagcactgatgtgagtgtagatgaagtaaaagctttggcttctctgatgacatacaagtgtgcagtggttgatgtgccgtttgggggtgctaaagctggtgttaagatcaatcccaagaactatactgataatgaattggaaaagatcacaaggaggttcaccatggagctagcaaaaaagggctttattggtcctggcattgatgtgcctgctccagacatgagcacaggtgagcgggagatgtcctggatcgctgatacctatgccagcaccatagggcactatgatattaatgcacacgcctgtgttactggtaaacccatcagccaagggggaatccatggacgcatctctgctactggccgtggtgtcttccatgggattgaaaatttcatcaatgaagcttcttacatgagcattttaggaatgacaccagggtttggagataaaacatttgttgttcagggatttggtaatgtgggcctacactctatgagatatttacatcgttttggtgctaaatgtattgctgttggtgagtctgatgggagtatatggaatccagatggtattgacccaaaggaactggaagacttcaaattgcaacatgggtccattctgggcttccccaaggcaaagccctatgaaggaagcatcttggaggccgactgtgacatactgatcccagctgccagtgagaagcagttgaccaaatccaacgcacccagagtcaaagccaagatcattgctgaaggtgccaatgggccaacaactccagaagctgacaagatcttcctggagagaaacattatggttattccagatctctacttgaatgctggaggagtgacagtatcttactttgagtggctgaagaatctaaatcatgtcagctatggccgtttgaccttcaaatatgaaagggattctaactaccacttgctcatgtctgttcaagagagtttagaaagaaaatttggaaagcatggtggaactattcccattgtacccacggcagagttccaagacaggatatcgggtgcatctgagaaagacatcgtgcactctggcttggcatacacaatggagcgttctgccaggcaaattatgcgcacagccatgaagtataacctgggattggacctgagaacagctgcctatgttaatgccattgagaaagtcttcaaagtgtacaatgaagctggtgtgaccttcacatagatggatcatggctgacttcctcactatcctcttcacatgtaacttctgcagacctatcacaagtttacatgtaaccacagaaatccctttctctcctgactcattaataatggataccattctcaacaagtcaatccaagtcagcccgttaaggagaaagaaattaaggttagcggatcatgtacaagctgagtgtgaaagtagaaatcacctacaccagagagccattttggtattttgcctttaaataaaaagcctcctttatctggctgtgcagccttgctctgtggcttttcccaacacaatcagtgctagtgctggggaggaacagtcaagagcagtcagttgcttgcttattttttctggatgagtctgggacacactgtaactttaacacatttaagaagtaggtgtgtggccttttcagaaggtggcatggtcctcaagtgagttcttagtattttatatcagcaaaataattcaattttgcaggttgcaaacaaatataaaacctgtttctgtttatgaatattattcttttagaatagaataagtacatgctgctgtaataaaattgcctttaatcacttaacaagcctaaccttgactcaaacagtgaatgcctatagaaataataaatgaaaaaaactagtatttttatatcataaaacaatgtcatttatagcttatcattcatgtattgtccagcagacattaaaagccctgtggataattaagttatcttcatacctgcaaaatggtggaggctattttcattaaaactgtcagaatttgcttactataattatgatacagtccaaagaatgcagtcactttttatcatgttaactaattgttctcttttgaagatctatggttgactaattaaacaataattcaagtagagtgtcccagaaaaaaaccacttgggctccctgtttggagtctggctggctctgagcattgccaatggcccctactcacctgactttgtatcctctccttttagaggctttgcattctgcacccagcttcactaacagtgggctgaaaacatccttgggttgagtgtttcatttgggagttatttggccagggccttttgaacagtagtgtccccatgaagtgctagataatatatgtgtaagagtcagctttttttttttttttaactctaacacccttcagaaatttctaactactttgtaactgcatggcttaacctggtgataaaagcagttattaaaagtctacgttttccaaaacttacgtttcttttctgtgtttttacatgtggtagtttctcttttcataagttataatactgcaattggatttctgaaatgtttatagcgaccacctgtataacatttccttccactttattgtgagctgcccagattttattcttgaattgttttttttttttttgttcggtgctttacacgttcagagaaacttcccgagtaacgaactatagaaatgatccctgaaagcatagtctttattctcgaattattttgtattttattaaataatatgaacagctaaaaaaaaaa

SEQ ID NO: 34 - DNA, 1220 bp, Homo sapiens
cgctccacctctcaagcagccagcgcctgcctgaatctgttctgccccctccccacccatttcaccaccaccatgacaccgggcacccagtctcctttcttcctgctgctgctcctcacagtgcttacagttgttacgggttctggtcatgcaagctctaccccaggtggagaaaaggagacttcggctacccagagaagttcagtgcccagctctactgagaagaatgctttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtaggggcacgtcgcccgctgagctgagtggccagccagtgccattccactccactcaggttcttcagggccagagcccctgcaccctgtttgggctggtgagctgggagttcaggtgggctgctcacagcctccttcagaggccccaccaatttctcggacacttctcagtgtgtggaagctcatgtgggcccctgagggctcatgcctgggaagtgttgtggtgggggctcccaggaggactggcccagagagccctgagatagcggggatcctgaactggactgaataaaacgtggtctcccactgcgccaaaaaaaaaaaaaaaaa

SEQ ID NO: 35 - DNA, 2162 bp, Homo sapiens
cgagttccggcgaggcttcagggtacagctcccccgcagccagaagccgggcctgcagcgcctcagcaccgctccgggacaccccacccgcttcccaggcgtgacctgtcaacagcaacttcgcggtgtggtgaactctctgaggaaaaaccattttgattattactctcagacgtgcgtggcaacaagtgactgagacctagaaatccaagcgttggaggtcctgaggccagcctaagtcgcttcaaaatggaacgaaggcgtttgtggggttccattcagagccgatacatcagcatgagtgtgtggacaagcccacggagacttgtggagctggcagggcagagcctgctgaaggatgaggccctggccattgccgccctggagttgctgcccagggagctcttcccgccactcttcatggcagcctttgacgggagacacagccagaccctgaaggcaatggtgcaggcctggcccttcacctgcctccctctgggagtgctgatgaagggacaacatcttcacctggagaccttcaaagctgtgcttgatggacttgatgtgctccttgcccaggaggttcgccccaggaggtggaaacttcaagtgctggatttacggaagaactctcatcaggacttctggactgtatggtctggaaacagggccagtctgtactcatttccagagccagaagcagctcagcccatgacaaagaagcgaaaagtagatggtttgagcacagaggcagagcagcccttcattccagtagaggtgctcgtagacctgttcctcaaggaaggtgcctgtgatgaattgttctcctacctcattgagaaagtgaagcgaaagaaaaatgtactacgcctgtgctgtaagaagctgaagatttttgcaatgcccatgcaggatatcaagatgatcctgaaaatggtgcagctggactctattgaagatttggaagtgacttgtacctggaagctacccaccttggcgaaattttctccttacctgggccagatgattaatctgcgtagactcctcctctcccacatccatgcatcttcctacatttccccggagaaggaagagcagtatatcgcccagttcacctctcagttcctcagtctgcagtgcctgcaggctctctatgtggactctttatttttccttagaggccgcctggatcagttgctcaggcacgtgatgaaccccttggaaaccctctcaataactaactgccggctttcggaaggggatgtgatgcatctgtcccagagtcccagcgtcagtcagctaagtgtcctgagtctaagtggggtcatgctgaccgatgtaagtcccgagcccctccaagctctgctggagagagcctctgccaccctccaggacctggtctttgatgagtgtgggatcacggatgatcagctccttgccctcctgccttccctgagccactgctcccagcttacaaccttaagcttctacgggaattccatctccatatctgccttgcagagtctcctgcagcacctcatcgggctgagcaatctgacccacgtgctgtatcctgtccccctggagagttatgaggacatccatggtaccctccacctggagaggcttgcctatctgcatgccaggctcagggagttgctgtgtgagttggggcggcccagcatggtctggcttagtgccaacccctgtcctcactgtggggacagaaccttctatgacccggagcccatcctgtgcccctgtttcatgcctaactagctgggtgcacatatcaaatgcttcattctgcatacttggacactaaagccaggatgtgcatgcatcttgaagcaacaaagcagccacagtttcagacaaatgttcagtgtgagtgaggaaaacatgttcagtgaggaaaaaacattcagacaaatgttcagtgaggaaaaaaaggggaagttggggataggcagatgttgacttgaggagttaatgtgatctttggggagatacatcttatagagttagaaatagaatctgaatttctaaagggagattctggcttgggaagtacatgtaggagttaatccctgtgtagactgttgtaaagaaactgttgaaaataaagagaagcaatgtgaagcaaaaaaaaaaaaaaaaaa

SEQ ID NO: 36 - DNA, 2520 bp, Homo sapiens
ggatggttgtctattaacttgttcaaaaaagtatcaggagttgtcaaggcagagaagagagtgtttgcaaaagggggaaagtagtttgctgcctctttaagactaggactgagagaaagaagaggagagagaaagaaagggagagaagtttgagccccaggcttaagcctttccaaaaaataataataacaatcatcggcggcggcaggatcggccagaggaggagggaagcgctttttttgatcctgattccagtttgcctctctctttttttcccccaaattattcttcgcctgattttcctcgcggagccctgcgctcccgacacccccgcccgcctcccctcctcctctccccccgcccgcgggccccccaaagtcccggccgggccgagggtcggcggccgccggcgggccgggcccgcgcacagcgcccgcatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgtgagggccggacagcgaactggaggggggagaaattttcaaagaaaaacgagggaaatgggaggggtgcaaaagaggagagtaagaaacagcatggagaaaacccggtacgctcaaaaagaaaaaggaaaaaaaaaaatcccatcacccacagcaaatgacagctgcaaaagagaacaccaatcccatccacactcacgcaaaaaccgcgatgccgacaagaaaacttttatgagagagatcctggacttctttttgggggactatttttgtacagagaaaacctggggagggtggggagggcgggggaatggaccttgtatagatctggaggaaagaaagctacgaaaaactttttaaaagttctagtggtacggtaggagctttgcaggaagtttgcaaaagtctttaccaataatatttagagctagtctccaagcgacgaaaaaaatgttttaatatttgcaagcaacttttgtacagtatttatcgagataaacatggcaatcaaaatgtccattgtttataagctgagaatttgccaatatttttcaaggagaggcttcttgctgaattttgattctgcagctgaaatttaggacagttgcaaacgtgaaaagaagaaaattattcaaatttggacattttaattgtttaaaaattgtacaaaaggaaaaaattagaataagtactggcgaaccatctctgtggtcttgtttaaaaagggcaaaagttttagactgtactaaattttataacttactgttaaaagcaaaaatggccatgcaggttgacaccgttggtaatttataatagcttttgttcgatcccaactttccattttgttcagataaaaaaaaccatgaaattactgtgtttgaaatattttcttatggtttgtaatatttctgtaaatttattgtgatattttaaggttttcccccctttattttccgtagttgtattttaaaagattcggctctgtattatttgaatcagtctgccgagaatccatgtatatatttgaactaatatcatccttataacaggtacattttcaacttaagtttttactccattatgcacagtttgagataaataaatttttgaaatatggacactgaaaaaaaaaa

SEQ ID NO: 37 - DNA, 1791 bp, Homo sapiens
tttgttggaagtggagagtggagggtcagaagggagtggaccagttcaggtcccagagggaatcctccctccctctgagccgtctttcttctcctccctatttcgcagatatcccgagattaggtccccagcttccaaagagaggatcagaatgtctcaggataatgacacattgatgagagacatcctggggcatgagctcgctgctatgaggctgcagaagctggaacagcagcggcggctgtttgaaaagaagcagcgacagaagcgccaggagctcctcatggttcaggccaatcctgacgcttccccgtggctttggcgctcttgtctgcgggaggagcgccttttaggtgacagaggccttgggaaccctttcctccggaagaaagtgtcagaggcacatctgccctctggcatccacagtgccctgggcaccgtgagctgtggtggagacggcaggggcgagcgcggcctcccgacaccgcggacagaagcagtgttcaggaatctcggtctccagtcccctttcttatcctggctcccagacaattccgatgcagaattggaggaagtctccgtggagaatggttccgtctctcccccaccttttaaacagtctccgagaatccgacgcaagggttggcaagcccaccaacgacctgggacccgtgcagagggtgagagtgactcccaggatatgggagatgcacacaagtcacccaatatgggaccaaaccctggaatggatggtgactgtgtatatgaaaacttggccttccaaaaggaagaagacttggaaaagaagagagaggcctctgagtctacagggacgaactcctcagcagcacacaacgaagagttgtccaaggccctgaaaggcgagggtggcacggacagcgaccatatgaggcacgaagcctccttggcaatccgctccccctgccctgggctggaggaggacatggaagcctacgtgctgcggccagcgctcccgggcaccatgatgcagtgctacctcacccgtgacaagcacggcgtggacaagggcttgttccccctctactacctctacctggagacctctgacagcctgcagcgcttcctcctggctgggcgaaagagaagaaggagcaaaacttctaattacctcatctccctggatcctacacacctatctcgggacggggacaatttcgtgggcaaagtcagatccaatgtcttcagcaccaagttcaccatctttgacaatggggtgaatcctgaccgggagcatttaaccaggaatactgcccggatcagacaggagctgggggctgtgtgttatgagcccaacgtcttaggatacctggggcctcggaaaatgactgtgattctcccaggaaccaacagccagaaccagcgaatcaatgtccagccactaaatgaacaggagtcgctactgagtcgttaccaacgtggggacaaacaagggttgcttttgttgcacaacaaaaccccgtcgtgggacaaggagaacggtgtctacacgctcaatttccatggtcgagtcactcgggcttcggtgaagaacttccaaatcgtggatcccaaacaccaagaacatctggtgctccagttcggccgagtgggcccagacacattcaccatggacttctgctttccatttagcccgctccaggccttcagcatctgcttgtccagtttcaattagaagctggctgttgaataactcaataaaataccatacccttgccagcaaaaaaaaaaaaaaaaaaaaaaaaaaaaaaa

(19) United States
(12) Patent Application Publication
(10) Pub. No.: US 2015/0044224 A1
(43) Pub. Date: Feb. 12, 2015

(54) MATERIALS AND METHODS FOR DIFFERENTIAL TREATMENT OF CANCER

(71) Applicants: H. LEE MOFFITT CANCER CENTER AND RESEARCH INSTITUTE, INC., TAMPA, FL (US); SERAMETRIX CORPORATION, CARLSBAD, CA (US)

(72) Inventors: Hatem Soliman, Tampa, FL (US); Phoebe Bonner-Ferraby, Cardiff-by-the-Sea, CA (US); Henry Hepburne-Scott, Reading (GB); Scott J. Antonia, Land O'Lakes, FL (US)

(21) Appl. No.: 14/381,504

(22) PCT Filed: Mar. 1, 2013
(86) PCT No.: PCT/US13/28741

§ 371 Date: Aug. 27, 2014

Related U.S. Application Data
(60) Provisional application No. 61/606,187, filed on Mar. 2, 2012.
(60) Provisional application No. 61/654,530, filed on Jun. 1, 2012.

(51) Int. Cl. G01N 33/574; C07K 16/28; C07K 16/18

(52) U.S. Cl. CPC G01N 33/57484; C07K 16/18; C07K 16/2818; G01N 2800/52
USPC 424/142.1; 424/141.1; 424/277.1; 506/9; 506/18; 506/16

(57) ABSTRACT

The present invention concerns differential therapeutic treatment of cancer patients based on prognostic antigen/antibody profiles used for predicting (prognosticating) a clinical response (efficacy) and/or adverse event to an immunotherapy for treatment of a malignancy in a subject, and for treating or delaying the onset or relapse of a malignancy in a subject.

### CROSS-REFERENCE TO RELATED APPLICATIONS

The present application claims the benefit of U.S. Provisional Application Ser. No. 61/606,187, filed Mar. 2, 2012, and U.S. Provisional Application Ser. No. 61/654,530, filed Jun. 1, 2012, which are hereby incorporated by reference herein in its entirety, including any figures, tables, nucleic acid sequences, amino acid sequences, and drawings.

### BACKGROUND OF INVENTION

Immunotherapy is emerging as a promising treatment option for patients with malignancies. Immunotherapeutics such as vaccines, immunomodulators, monoclonal antibodies, immunostimulants, dendritic cells, and viral therapies are being tested extensively. However, it is becoming increasingly clear that immunotherapies can induce unwanted immune reactions against normal tissues, involving potentially life-threatening autoimmune side effects and adverse events associated with immunotoxicity (Amos, S. M. et al., “Autoimmunity associated with immunotherapy of cancer,” Blood, Jul. 21, 2011; Epub Apr. 29, 2011; 118(3):499-509). It would be advantageous to have available a reliable tool for predicting clinical outcome and adverse events that can be incorporated into diagnostic and treatment regimens for cancer patients.

### BRIEF SUMMARY

The inventors have shown herein that the clinical outcome of an immunotherapy for a malignancy, including adverse events, may be predicted based on the profile or signature composed of the abundance of prognostic antigens and the antibody response they provoke.

The present invention concerns tumor antigen sets having prognostic value. In one aspect, the invention concerns an array comprising an array of capture probes disposed on a substrate, in which the capture probes specifically bind (1) antibodies of the antigens, or (2) two or more of the prognostic antigens (proteins) themselves, or (3) nucleic acid molecules encoding two or more of the prognostic antigens. Thus, the array can be, for example, a protein array (with antigenic epitopes disposed on the substrate), an antibody array (with antibodies or antibody fragments disposed on the substrate), or a nucleic acid array (with oligonucleotides disposed on the substrate). Another aspect of the invention concerns kits comprising the capture probes and arrays of the invention. The arrays and kits may be used to carry out prognostic methods and treatment methods of the invention. These methods of the invention include a method for predicting (prognosticating) a clinical response (efficacy) and/or adverse event to an immunotherapy for treatment of a malignancy in a subject, and a method for treating or delaying the onset or relapse of a malignancy in a subject. The arrays, kits, and methods of the invention can assist clinicians in making treatment decisions for malignancies, and can be incorporated into pharmacovigilance programs in connection with immunotherapies.

### BRIEF DESCRIPTION OF DRAWINGS

FIGS. 1A and 1B show that changes in antibody profiles after immunotherapy predict adverse events. FIG. 1A is a table showing that patients who did have a 50%+ increase in reactivity to 3+ (three or more) panel antigens did suffer autoimmune side effect (100%, n=3), and patients who did not have a 50%+ increase in reactivity to 3+ (three or more) panel antigens did not suffer autoimmune side effect (89%, n=9). FIG. 1B is a graph showing the number of panel antigens exhibiting greater than 50% after treatment versus incidence of adverse event. The panel of antigens consisted of CTAG2, MAGEA1, MAGEA3, MAGEAv2, MICA, NLRP4, SILV, SSX4, TSSK6, and XAGE-2.

FIGS. 2A and 2B show that baseline antibody profiles can predict responses to immunotherapy. A positive relationship between the number of seropositive events at baseline and the number of months' survival following immunotherapy was observed. FIG. 2B is a table showing that patients who did not test positive to 5+ panel antigens did not survive more than 300 days (100%, n=5), and patients who did test positive to 5+ panel antigens did survive more than 300 days (66%, n=3). FIG. 2B is a graph showing baseline immunity versus survival time. With the removal of one outlier, the R-squared value of the trend line was 0.82. The panel of antigens consisted of CTAG2, MAGEA1, MAGEA3, MAGEA4v3, MICA, NURP4, SILV, SSX4, TSSK6, and XAGE-2.

FIG. 3 is a graph showing the number of seropositive panel antigens of example combination A at baseline versus survival time following immunotherapy.

FIG. 4 is a graph showing the number of seropositive panel antigens of example combination B at baseline versus survival time following immunotherapy.

FIG. 5 is a graph showing the number of seropositive panel antigens of example combination C at baseline versus survival time following immunotherapy.

FIG. 6 is a graph showing the number of seropositive panel antigens of example combination D at baseline versus survival time following immunotherapy.

FIG. 7 is a graph showing the number of seropositive panel antigens of example combination E at baseline versus survival time following immunotherapy.

FIG. 8 is a graph showing the number of panel antigens of example combination F exhibiting an increase of greater than 50% seropositivity after treatment with an immunotherapy versus the incidence of an adverse event.

FIG. 9 is a graph showing the number of panel antigens of example combination G exhibiting an increase of greater than 50% seropositivity after treatment with an immunotherapy versus the incidence of an adverse event.

FIG. 10 is a graph showing the number of panel antigens of example combination H exhibiting an increase of greater than 50% seropositivity after treatment with an immunotherapy versus the incidence of an adverse event.

FIG. 11 is a graph showing the number of panel antigens of example combination I exhibiting an increase of greater than 50% seropositivity after treatment with an immunotherapy versus the incidence of an adverse event.

FIG. 12 is a graph showing the number of panel antigens of example combination J exhibiting an increase of greater than 50% seropositivity after treatment with an immunotherapy versus the incidence of an adverse event.

FIG. 13 is a graph showing survival according to antibody score.

FIG. 14 is a chart showing sites of primary melanoma.

FIG. 15 is a bar graph showing frequency distribution of antibody responses detected at baseline in the study cohort.

FIG. 16 is a Kaplan-Meier survival curve, comparing survival of radiological responders (stable disease (SD)/partial response (PR)) to non-responders (partial disease (PD)).

FIG. 17 shows Kaplan-Meier survival curves comparing survival in Ipilimumab-treated patients with an antibody response to 0 panel antigens to patients with an antibody response to 1 or more antigens.

FIG. 18 shows Kaplan-Meier survival curves comparing survival in Ipilimumab-treated patients with an antibody response to 0 panel antigens to patients with an antibody response to 2 or more antigens.

FIG. 19 shows Kaplan-Meier survival curves comparing survival in Ipilimumab-treated patients with an antibody response to 0 panel antigens, an antibody response to 1 panel antigen, an antibody response to 2 panel antigens, and an antibody response to 3 or more panel antigens.

### DETAILED DISCLOSURE

An aspect of the invention concerns an array comprising arrayed capture probes disposed on a substrate, in which the capture probes specifically bind: (1) antibodies of the antigens, or (2) two or more of the prognostic antigens (proteins) themselves, or (3) nucleic acid molecules encoding two or more of the prognostic antigens (see, for example, Berton P. and Snyder M., “Advances in functional protein microarray technology,” FEBS J, 2005; 272(21):5400-5411; Wingren C. and Borrebaeck C. A., “Antibody microarrays: current status and key technological advances,” OMICS, 2006, 10(3):411-427; Zhu H. and Snyder M., Curr. Opin. Chem. Biol., 2003, 7(1):55-63; Büssow K. et al., “Protein Array Technology: Potential Use in Medical Diagnostics,” Am. J. Pharmaceogenomics, 2001, 1(1):1-7). Thus, for example, the array can be a protein array (with antigenic epitopes disposed on the substrate), an antibody array (with antibodies or antibody fragments disposed on the substrate), or a nucleic acid array (with oligonucleotides disposed on the substrate, in which the oligonucleotides are partially or fully complementary with nucleic acid sequences encoding the prognostic antigens).

In some embodiments, the array comprises a substrate and two or more capture probes disposed thereon, wherein the two or more capture probes comprise or consist of:

(a) at least antigenic epitopes of two or more antigens selected from among BRAF, CABYR, CRISP3, CSAG2, CTAG2, CXorf48.1, DHFR, FTHL17, GAGE1, GAGE2A, GLUD1, LDHC, MAGEA1, MAGEA3, MAGEA4v2, MAGEA4v3, MAGEA4v4, MAGEB6, MAPK1, MICA, MUC1, NLRP4, NY-ESO-1, PBK, PRAME, SOX2, SILV, SPANXA1, SPANXB1, SSX2A, SSX4, TSGA10, TSSK6, TULP2, TYR, XAGE-2, and ZNF165; or

(b) antibodies, or antibody fragments, that specifically bind two or more antigens from those set forth in (a); or

(c) oligonucleotides that bind to nucleic acid sequences encoding two or more antigens from those set forth in (a).

In an alternative embodiment, the antibodies or antibody fragments of (b) specifically bind to antibodies of two or more antigens from those set forth in (a) (thus, relying on an antibody-antibody interaction).

In some embodiments, the antigens comprise or consist of the group of antigens of example combination A, example combination B, example combination C, example combination D, example combination E, example combination F, example combination G, example combination H, example combination I, or example combination J.

In some embodiments, the antigens comprise or consist of two or more of CSAG2, MAGEA1, MAGEA3, MAGEA4v2, MICA, NLRP4, SILV, SSX4, TSSK6, and XAGE-2. In some embodiments, the antigens comprise or consist of CSAG2, MAGEA1, MAGEA3, MAGEA4v2, MICA, NLRP4, SILV, SSX4, TSSK6, and XAGE-2.

In some embodiments, the antigens comprise or consist of two or more of the following antigens: BRAF, CABYR, CRISP3, CSAG2, CTAG2, CXorf48.1, DHFR, FTHL17, GAGE1, GAGE2A, GLUD1, LDHC, MAGEA1, MAGEA3, MAGEA4v2, MAGEA4v3, MAGEA4v4, MAGEB6, MAPK1, MICA, MUC1, NLRP4, NY-ESO-1, PBK, PRAME, SOX2, SILV, SPANXA1, SPANXB1, SSX2A, SSX4, TSGA10, TSSK6, TULP2, TYR, XAGE-2, and ZNF165. Thus, the antigens comprise or consist of two, three, three, four, five, six, seven, eight, nine, ten, eleven, twelve, thirteen, fourteen, fifteen, sixteen, seventeen, eighteen, nineteen, twenty, twenty one, twenty two, twenty three, twenty four, twenty five, twenty six, twenty seven, twenty eight, twenty nine, thirty, thirty one, thirty two, thirty three, thirty four, thirty five, thirty six, or all thirty seven of the aforementioned antigens.

In some embodiments, the antigens comprise or consist of two or more of the following antigens: BRAF, CABYR, CRISP3, CSAG2, CTAG2, DHFR, FTHL17, GAGE1, GLUD1, LDHC, MAGEA1, MAGEB6, MAPK1, FTHL17, SSX2, XAGE2, TULP2, PRAME, SOX2, SPANX-B1, SSX4, TSSK6, and SSX5. Thus, the antigens may comprise or consist of two, three, three, four, five, six, seven, eight, nine, ten, eleven, twelve, thirteen, fourteen, fifteen, sixteen, seventeen, eighteen, nineteen, twenty, twenty one, twenty two, or all twenty three of the aforementioned antigens.

In some embodiments, the array is a protein array in which the capture probes disposed on the substrate are amino acid sequences making up at least antigenic epitopes of two or more of the antigens of interest. Preferably, the disposed antigenic epitopes are full-length antigens.

In the various embodiments of the array of the invention, the substrate may be any solid or semi-solid carrier for supporting the capture probes, such as a particle (e.g., magnetic or latex particle), a microtiter multi-well plate, a bead, a slide, a filter, a chip, a membrane, a cuvette, or a reaction vessel.

In some embodiments, the array comprises or consists of:

(a) at least antigenic epitopes of three, four, five, six, seven, eight, nine, ten, eleven, twelve, thirteen, fourteen, fifteen, sixteen, seventeen, eighteen, nineteen, twenty, twenty one, twenty two, twenty three, twenty four, twenty five, twenty six, twenty seven, twenty eight, twenty nine, thirty, thirty one, thirty two, thirty three, thirty four, thirty five, thirty six, or thirty seven of the antigens (preferably, the full-length antigens);

(b) antibodies, or antibody fragments, that specifically bind three, four, five, six, seven, eight, nine, ten, eleven, twelve, thirteen, fourteen, fifteen, sixteen, seventeen, eighteen, nineteen, twenty, twenty one, twenty two, twenty three, twenty four, twenty five, twenty six, twenty seven twenty eight, twenty nine, thirty, thirty one, thirty two, thirty three, thirty four, thirty five, thirty six, or thirty seven of the antigens; or

(c) oligonucleotides that bind to nucleic acid sequences encoding three, four, five, six, seven, eight, nine, ten, eleven, twelve, thirteen, fourteen, fifteen, sixteen, seventeen, eighteen, nineteen, twenty, twenty one, twenty two, twenty three, twenty four, twenty five, twenty six, twenty seven twenty eight, twenty nine, thirty, thirty one, thirty two, thirty three, thirty four, thirty five, thirty six, or thirty seven of the antigens.

The array of the invention may be used for determining the level of two or more of the recited targets (biomarkers) in a biological sample taken from a subject, such as for the methods disclosed herein.

Another aspect of the invention concerns a method for determining the levels of biomarkers in a sample from a subject, comprising:

(a) determining the level of two or more biomarkers in a biological sample taken from the subject before or after initiation of the immunotherapy, and wherein the two or more biomarkers comprise or consist of:
(1) immunoglobulins to two or more antigens selected from among BRAF, CABYR, CRISP3, CSAG2, CTAG2, CXorf48.1, DHFR, FTHL17, GAGE1, GAGE2A, GLUD1, LDHC, MAGEA1, MAGEA3, MAGEA4v2, MAGEA4v3, MAGEA4v4, MAGEB6, MAPK1, MICA, MUC1, NLRP4, NY-ESO-1, PBK, PRAME, SOX2, SILV, SPANXA1, SPANXB1, SSX2A, SSX4, TSGA10, TSSK6, TULP2, TYR, XAGE-2, and ZNF165; or
(2) two or more antigens selected from those set forth in (a)(1); or
(3) nucleic acid sequences that encode two or more antigens selected from those set forth in (a)(1); or
(4) T-cells activated against two or more antigens selected from those set forth in (a)(1).

Another aspect of the invention concerns a method for predicting a clinical response (efficacy) and/or adverse event to an immunotherapy for treatment of a malignancy in a subject, comprising:

(a) determining the level of two or more biomarkers in a biological sample taken from the subject before or after initiation of the immunotherapy, and wherein the two or more biomarkers comprise or consist of:
(1) immunoglobulins to two or more antigens selected from among BRAF, CABYR, CRISP3, CSAG2, CTAG2, CXorf48.1, DHFR, FTHL17, GAGE1, GAGE2A, GLUD1, LDHC, MAGEA1, MAGEA3, MAGEA4v2, MAGEA4v3, MAGEA4v4, MAGEB6, MAPK1, MICA, MUC1, NLRP4, NY-ESO-1, PBK, PRAME, SOX2, SILV, SPANXA1, SPANXB1, SSX2A, SSX4, TSGA10, TSSK6, TULP2, TYR, XAGE-2, and ZNF165; or
(2) two or more antigens selected from those set forth in (a)(1); or
(3) nucleic acid sequences that encode two or more antigens selected from those set forth in (a)(1); or
(4) T-cells activated against two or more antigens selected from those set forth in (a)(1); and

(b) correlating the level of the two or more biomarkers in the sample with a predicted clinical response and/or likelihood of an adverse event in the subject. Correlation of the biomarker levels to the clinical response and/or likelihood of adverse event can be done by comparing the level of the two or more biomarkers in the sample to a reference level (a predetermined value) of the two or more biomarkers, wherein the relationship (an identical level or a difference (higher or lower)) between the level of the two or more biomarkers in the sample and the reference level is indicative of the clinical response and/or the likelihood of an adverse event. In some embodiments, the reference level is the level of a normal subject, or the level of a normal population of subjects. In some embodiments, the determining step of (a) comprises measuring the level of the two or more biomarkers in a biological sample taken from the subject, and the correlating step of (b) comprises comparing the measured level of the two or more biomarkers to a reference level of the two or more biomarkers, wherein the relationship (an identical level or a difference (higher or lower)) between the level of the two or more biomarkers in the sample and the reference level is indicative of the clinical response and/or the likelihood of an adverse event.

The levels of the biomarkers of the present invention can be measured using any method known in the art appropriate for the form of biomarker (e.g., antibody or nucleic acid). The “readout” of the methods and arrays of the invention (the information conveyed regarding the biomarker or biomarkers in a sample) may be qualitative (binary “yes” or “no”, e.g., reflecting the presence or absence of a biomarker in a sample, such as the presence or absence of an antibody to a tumor antigen) or quantitative.

The biomarker data obtained from the sample may be analyzed and interpreted such that a threshold or cutoff is applied. For example, the reference level may be a threshold or cutoff such that when the sample biomarker level is high compared to the threshold level, this relationship is indicative of the immunotherapy's efficacy (e.g., increased survival) and/or likelihood of an adverse event. In some embodiments, the level of the two or more biomarkers compared to a reference level of the corresponding biomarkers is high and, therefore, the subject's prognosis is indicative of a survival rate greater than that of a subject without a high level of the two or more biomarkers.

A threshold or cutoff may be applied, for example to sample raw data, such that a “hit” is determined for a particular target biomarker (e.g., antigen, antigenic epitope, antibody or antibody fragment, oligonucleotide, or other substrate). As a specific example, the threshold or cutoff may be applied to serum antibody raw data. The sum of the number of “hits” from a sample is deemed to be the Score value of subject (e.g., human patient) immunity. Intervals of scores are then applied to categorize subjects according to their anti-tumor immune status and thereby their likelihood of good clinical response to immunotherapy. For example, subjects with a Score of less than two out of a given panel of antigens will be deemed unlikely to exhibit good clinical outcome to immunotherapy, whereas subjects with a Score of two or more may be deemed good candidates for immunotherapy as they are more likely to exhibit a favorable clinical response. There may be variable intervals for binning scores, for example, a single threshold of two such that subjects are deemed to have a “low” or “high” likelihood of good clinical response, or multiple thresholds, for example three, such that subjects are categorized as having a “low”, “medium” or “high” likelihood of good clinical response to immunotherapy.

In some embodiments, the correlating step comprises determining a value (score) representative of the number of biomarker levels that meet or exceed a reference threshold level, and comparing the determined score to one or more reference scores, wherein the relationship between the determined score and the one or more reference scores is predictive of (correlates with) an adverse event or absence of an adverse event. The method may further comprise categorizing the subject (assigning a category) based on the relationship between the determined score and the reference score, wherein the assigned category is representative of the likelihood of positive clinical response to immunotherapy, or likelihood of an adverse event. The subject can be categorized into a category from among two, three, or more categories. In some embodiments, the subject is categorized into one of two categories (e.g., “low” or “high”). In some embodiments, the determined score is compared to a plurality of scores, and the method further comprises categorizing the subject based on the relationship between the determined score and the plurality of reference scores. The subject can then be categorized into one of three or more categories (e.g., “low”, “medium”, or “high”).

The determination of the level of a plurality of biomarkers may be done simultaneously or consecutively. Capture probes for a single biomarker may be arrayed on each substrate, or capture probes for two or more biomarkers may be arrayed on each substrate. Preferably, the levels of two or more biomarkers are determined within the same biological sample taken from the subject, but may be from different biological samples taken from the subject (e.g., one biomarker determined per sample). When multiple biomarkers are being assessed within different samples, the samples are preferably obtained from the subject at the same time. It should be understood that the order in which the levels of a “first”, “second”, “third” or more biomarkers are measured is not important. For example, all biomarkers may be measured concurrently. Alternatively, the second or third or subsequent biomarker may be assessed prior to the level of the first biomarker.

Although the methods of the invention require the detection of two or more biomarkers in one or more patient samples, in some embodiments 3, 4, 5, 6, 7, 8, 9, 10 or more biomarkers may be used to practice the present invention. The two or more biomarkers will be complementary biomarkers. The term “complementary” in this context is intended to mean that detection and correlation of the combination of biomarkers in a biological sample(s) results in the successful identification of a clinical response (e.g., survival) and/or likelihood of an adverse event in a greater percentage of cases than would be identified if only one of the biomarkers was used. Thus, in some cases, a more accurate determination of prognosis can be made by using at least two biomarkers from among the biomarkers disclosed herein.

In some embodiments of the methods, the antigens comprise or consist of the group of antigens of example combination A, example combination B, example combination C, example combination D, example combination E, example combination F, example combination G, example combination II, example combination I, or example combination J.

In some embodiments, the antigens comprise or consist of two or more of CSAG2, MAGEA1, MAGEA3, MAGEA4v2, MICA, NLRP4, SILV, SSX4, TSSK6, and XAGE-2. In some embodiments, the antigens comprise or consist of CSAG2, MAGEA1, MAGEA3, MAGEA4v2, MICA, NLRP4, SILV, SSX4, TSSK6, and XAGE-2.

In some embodiments, the antigens comprise or consist of two or more of the following antigens: BRAF, CABYR, CRISP3, CSAG2, CTAG2, CXorf48.1, DHFR, FTHL17, GAGE1, GAGE2A, GLUD1, LDHC, MAGEA1, MAGEA3, MAGEA4v2, MAGEA4v3, MAGEA4v4, MAGEB6, MAPK1, MICA, MUC1, NLRP4, NY-ESO-1, PBK, PRAME, SOX2, SILV, SPANXA1, SPANXB1, SSX2A, SSX4, TSGA10, TSSK6, TULP2, TYR, XAGE-2, and ZNF165. Thus, the antigens comprise or consist of two, three, three, four, five, six, seven, eight, nine, ten, eleven, twelve, thirteen, fourteen, fifteen, sixteen, seventeen, eighteen, nineteen, twenty, twenty one, twenty two, twenty three, twenty four, twenty five, twenty six, twenty seven, twenty eight, twenty nine, thirty, thirty one, thirty two, thirty three, thirty four, thirty five, thirty six, or all thirty seven of the aforementioned antigens.

In some embodiments, the antigens comprise or consist of two or more of the following antigens: BRAF, CABYR, CRISP3, CSAG2, CTAG2, DHFR, FTHL17, GAGE1, GLUD1, LDHC, MAGEA1, MAGEB6, MAPK1, FTHL17, SSX2, XAGE2, TULP2, PRAME, SOX2, SPANX-B1, SSX4, TSSK6, and SSX5. Thus, the antigens may comprise or consist of two, three, three, four, five, six, seven, eight, nine, ten, eleven, twelve, thirteen, fourteen, fifteen, sixteen, seventeen, eighteen, nineteen, twenty, twenty one, twenty two, or all twenty three of the aforementioned antigens.

In some embodiments of the methods, a significant increase in the level of two, three, or more biomarkers (e.g., 50%+) after immunotherapy is predictive of (correlates with) an adverse event. In some embodiments, lack of a significant increase (e.g., not having 50%+) in the level of two, three, or more biomarkers after immunotherapy is predictive of (correlates with) an absence of an adverse event. For example, in some embodiments, a significant increase (e.g., 50%+) in seroreactivity to two, three, or more of the antigens after immunotherapy is predictive of (correlates with) an adverse event. In some embodiments, lack of a significant increase (e.g., not having 50%+) in seroreactivity to two, three, or more antigens after immunotherapy is predictive of (correlates with) an absence of an adverse event.

In some embodiments of the methods, if the level of two, three, four, five, or more biomarkers does not reach a threshold level, the subject is predicted to have a poor clinical response, e.g., survival of 300 days or less. In some embodiments, if the level of two, three, four, five, or more biomarkers does reach a threshold level, the subject is predicted to have a positive clinical response (treatment efficacy), e.g., survival more than 300 days.

In some embodiments of the methods, the sample is obtained from the subject after initiation of the immunotherapy, and wherein the reference level is the level of the two or more biomarkers in a sample taken from the subject before initiation of the immunotherapy (thus, a comparison pre- and post-immunotherapy is made).

In some embodiments of the methods, the biomarkers comprise or consist of (a)(1), and wherein the biological sample is serum.

In some embodiments of the methods, the biomarkers comprise or consist of (a)(1) or (a)(2), and the biological sample comprises cells of the malignancy.

In some embodiments of the methods, the biomarkers comprise or consist of (a)(4), i.e., T-cells activated against two or more antigens. The quantitation of T-cells (CD8+ and/or CD4+ T-cells) activated against two or more antigens can be made, for example, by single-cell assay involving staining antigen-specific T-cells with fluorescently labeled tetrameric major histocompatibility complex (MHC)/peptide complexes (MHC tetramer technology) (see, for example, Constantin C. M. et al., “Major Histocompatibility Complex (MHC) Tetramer Technology: An Evaluation”, Biological Research for Nursing, October 2002, 4(2):115-127). Various other immunologic assays can be used to monitor a subject's antigen-specific T-cell responses including, but not limited to, enzyme-linked immunosorbent spot (ELISPOT) assay (see, for example, Gajewski T. F. et al., “Monitoring Specific T-Cell Responses to Melanoma Vaccines: ELISPOT, Tetramers, and Beyond,” Clin. Diagn. Lab. Immunol., 2000, 7(2):141-144).

In some embodiments of the methods, the malignancy is selected from among melanoma, ovarian cancer, breast cancer, lung cancer (small cell or non-small cell), esophageal cancer, sarcoma, or colorectal cancer.

In some embodiments of the methods, the clinical response is survival.

In some embodiments of the methods, the adverse event is autoimmune toxicity, including but not limited to, a gastrointestinal autoimmune side effect (colitis, stomach pain, bloating, constipation, diarrhea), dermatitis, anti-pituitary autoimmune side effect, hepatitis, inflammation of the hormone gland(s), inflammation of the eyes, inflammation of the nerves, or two or more of the foregoing.

In some embodiments of the methods, the immunotherapy is selected from among a cancer vaccine, immunomodulator, monoclonal antibody, immunostimulant, dendritic cell, viral therapy. For example, the immunotherapy may be an antibody that binds to cytotoxic T lymphocyte-associated antigen 4 (CTLA-4) (e.g., Ipilimumab), a p53 cancer vaccine, 1-methyl-D-tryptophan (1MT), or autologous dendritic cells activated against an antigen of the malignancy (for example prostatic acid phosphatase (PAP), e.g., sipuleucel-T).

In a specific embodiment of the methods, the antigens comprise or consist of two, three, three, four, five, six, seven, eight, nine, ten, eleven, twelve, thirteen, fourteen, fifteen, sixteen, seventeen, eighteen, nineteen, twenty, twenty one, twenty two, or all twenty three of: BRAE, CABYR, CRISP3, CSAG2, CTAG2, DHFR, FTHL17, GAGE1, GLUD1, LDHC, MAGEA1, MAGEB6, MAPK1, FTHL17, SSX2, XAGE2, TULP2, PRAME, SOX2, SPANX-B1, SSX4, TSSK6, and SSX5; the malignancy is selected from among melanoma, ovarian cancer, breast cancer, lung cancer (small cell or non-small cell), esophageal cancer, sarcoma, or colorectal cancer; and the immunotherapy comprises an antibody that binds to CTLA-4 (e.g., Ipilimumab).

Optionally, the method for predicting a clinical response (efficacy) and/or adverse event to an immunotherapy for treatment of a malignancy may further comprise: (c) administering an immunotherapy to the subject if it is predicted that the immunotherapy will have efficacy and/or will not result in an adverse event; or (d) withholding the immunotherapy from the subject if it is predicted that the immunotherapy will not have efficacy and/or will result in an adverse event. Optionally, the withholding step of (d) may further comprise administering a therapy other than an immunotherapy to the subject if it is predicted that the immunotherapy will not have efficacy and/or will result in an adverse event. Examples of non-immunotherapies that may be administered include chemotherapy, radiation therapy, surgery, or a combination of two or three of the foregoing.

Another aspect of the invention concerns a method for treating or delaying the onset or relapse of a malignancy in a subject, comprising carrying out the aforementioned method for predicting a clinical response (efficacy) and/or adverse event, and

(a) administering an immunotherapy to the subject if it is predicted that the immunotherapy will have efficacy and/or will not result in an adverse event; or

(b) withholding the immunotherapy from the subject if it is predicted that the immunotherapy will not have efficacy and/or will result in an adverse event. Optionally, (b) further comprises administering an alternative therapy (a therapy other than an immunotherapy, i.e., a non-immunotherapy) to the subject if it is predicted that the immunotherapy will not have efficacy (not have a positive clinical outcome) and/or will result in an adverse event. In some embodiments the alternative comprises chemotherapy, radiation therapy, surgery, or a combination of two or three of the foregoing. The prediction as to clinical response (efficacy) and/or adverse event may be made using the method described herein (i.e., the method for predicting a clinical response (efficacy) and/or adverse event to an immunotherapy for treatment of a malignancy in a subject). Thus, the prediction as to clinical response and/or adverse event may include: determining the level of two or more biomarkers in a biological sample taken from the subject before or after initiation of the immunotherapy, and wherein the two or more biomarkers comprise or consist of:
(1) immunoglobulins to two or more antigens selected from among BRAF, CABYR, CRISP3, CSAG2, CTAG2, CXorf48.1, DHFR, FTHL17, GAGE1, GAGE2A, GLUD1, LDHC, MAGEA1, MAGEA3, MAGEA4v2, MAGEA4v3, MAGEA4v4, MAGEB6, MAPK1, MICA, MUC1, NLRP4, NY-ESO-1, PBK, PRAME, SOX2, SILV, SPANXA1, SPANXB1, SSX2A, SSX4, TSGA10, TSSK6, TULP2, TYR, XAGE-2, and ZNF165; or
(2) two or more antigens selected from those set forth in (a)(1); or
(3) nucleic acid sequences that encode two or more antigens selected from those set forth in (a)(1); or
(4) T-cells activated against two or more antigens selected from those set forth in (a)(1); and
   correlating the level of the two or more biomarkers in the sample with a predicted clinical response and/or likelihood of an adverse event in the subject.

In some embodiments of the methods, the antigens comprise or consist of the group of antigens of example combination A, example combination B, example combination C, example combination D, example combination E, example combination F, example combination G, example combination H, example combination I, or example combination J.

In some embodiments, the antigens comprise or consist of two or more of CSAG2, MAGEA1, MAGEA3, MAGEA4v2, MICA, NLRP4, SILV, SSX4, TSSK6, and XAGE-2. In some embodiments, the antigens comprise or consist of CSAG2, MAGEA1, MAGEA3, MAGEA4v2, MICA, NLRP4, SILV, SSX4, TSSK6, and XAGE-2.

In some embodiments, the antigens comprise or consist of two or more of the following antigens: BRAF, CABYR, CRISP3, CSAG2, CTAG2, CXorf48.1, DHFR, FTHL17, GAGE1, GAGE2A, GLUD1, LDHC, MAGEA1, MAGEA3, MAGEA4v2, MAGEA4v3, MAGEA4v4, MAGEB6, MAPK1, MICA, MUC1, NLRP4, NY-ESO-1, PBK, PRAME, SOX2, SILV, SPANXA1, SPANXB1, SSX2A, SSX4, TSGA10, TSSK6, TULP2, TYR, XAGE-2, and ZNF165. Thus, the antigens comprise or consist of two, three, three, four, five, six, seven, eight, nine, ten, eleven, twelve, thirteen, fourteen, fifteen, sixteen, seventeen, eighteen, nineteen, twenty, twenty one, twenty two, twenty three, twenty four, twenty five, twenty six, twenty seven, twenty eight, twenty nine, thirty, thirty one, thirty two, thirty three, thirty four, thirty five, thirty six, or all thirty seven of the aforementioned antigens.

In some embodiments, the antigens comprise or consist of two or more of the following antigens: BRAF, CABYR, CRISP3, CSAG2, CTAG2, DHFR, FTHL17, GAGE1, GLUD1, LDHC, MAGEA1, MAGEB6, MAPK1, FTHL17, SSX2, XAGE2, TULP2, PRAME, SOX2, SPANX-B1, SSX4, TSSK6, and SSX5. Thus, the antigens may comprise or consist of two, three, three, four, five, six, seven, eight, nine, ten, eleven, twelve, thirteen, fourteen, fifteen, sixteen, seventeen, eighteen, nineteen, twenty, twenty one, twenty two, or all twenty three of the aforementioned antigens.

In a specific embodiment of the methods, the antigens comprise or consist of two, three, three, four, five, six, seven, eight, nine, ten, eleven, twelve, thirteen, fourteen, fifteen, sixteen, seventeen, eighteen, nineteen, twenty, twenty one, twenty two, or all twenty three of: BRAF, CABYR, CRISP3, CSAG2, CTAG2, DHFR, FTHL17, GAGE1, GLUD1, LDHC, MAGEA1, MAGEB6, MAPK1, FTHL17, SSX2, XAGE2, TULP2, PRAME, SOX2, SPANX-B1, SSX4, TSSK6, and SSX5; the malignancy is selected from among melanoma, ovarian cancer, breast cancer, lung cancer (small cell or non-small cell), esophageal cancer, sarcoma, or colorectal cancer; and the immunotherapy comprises an antibody that binds to CTLA-4 (e.g., Ipilimumab).

Another aspect of the invention concerns immunotherapeutic agent for use in treatment of a malignancy in a subject, the treatment comprising the following prior to administration of the immunotherapeutic agent:

(a) determining the level of two or more biomarkers in a biological sample taken from the subject before or after initiation of the immunotherapy, and wherein the two or more biomarkers comprise or consist of:
(1) immunoglobulins to two or more antigens selected from among BRAF, CABYR, CRISP3, CSAG2, CTAG2, CXorf48.1, DHFR, FTHL17, GAGE1, GAGE2A, GLUD1, LDHC, MAGEA1, MAGEA3, MAGEA4v2, MAGEA4v3, MAGEA4v4, MAGEB6, MAPK1, MICA, MUC1, NLRP4, NY-ESO-1, PBK, PRAME, SOX2, SILV, SPANXA1, SPANXB1, SSX2A, SSX4, TSGA10, TSSK6, TULP2, TYR, XAGE-2, and ZNF165; or
(2) two or more antigens selected from those set forth in (a)(1); or
(3) nucleic acid sequences that encode two or more antigens selected from those set forth in (a)(1); or
(4) T-cells activated against two or more antigens selected from those set forth in (a)(1); and

(b) correlating the level of the two or more biomarkers in the sample with a predicted clinical response and/or likelihood of an adverse event in the subject.

Generally, the expression level of a gene encoding an antigen may be determined at the RNA or protein level as a relative expression level. More preferably, the determination comprises contacting the sample with selective reagents (i.e., capture probes), such as probes, primers or ligands, and thereby detecting the presence, or measuring the amount, of immunoglobulin (antibody or antibody fragment), polypeptide, or nucleic acids of interest originally in the sample. The capture probes may be disposed (immobilized, deposited on, or otherwise associated with) a substrate as an array. The capture probes may be arranged on the substrate of the array in an organized (spatially arranged) or random fashion.

In some embodiments, the capture probe is an antibody or antibody fragment that specifically binds an antigen of interest. In some embodiments, the capture probe is at least an antigenic epitope of an antigen (preferably, the full-length antigen) that induces antibodies that specifically bind the antigenic epitope or antigen. In some embodiments, the capture probes are oligonucleotides that bind to nucleic acid sequences encoding the antigens of interest. In some embodiments, the capture probes comprise or consist of:

(a) at least antigenic epitopes of two or more antigens selected from among BRAF, CABYR, CRISP3, CSAG2, CTAG2, CXorf48.1, DHFR, FTHL17, GAGE1, GAGE2A, GLUD1, LDHC, MAGEA1, MAGEA3, MAGEA4V2, MAGEA4V3, MAGEA4V4, MAGEB6, MAPK1, MICA, MUC1, NLRP4, NY-ESO-1, PBK, PRAMS, SOX2, SILV, SPANXA1, SPANXB1, SSX2A, SSX4, TSGA10, TSSK6, TULP2, TYR, RAGE-2, and ZNF165; or

(b) antibodies, or antibody fragments, that specifically bind two or more antigens from those set forth in (a); or

(c) oligonucleotides that are partially or fully complementary to, and bind (hybridize) to, nucleic acid sequences encoding two or more antigens from those set forth in (a).

In some embodiments, the array comprises or consists of:

(a) at least antigenic epitopes of three, four, five, six, seven, eight, nine, ten, eleven, twelve, thirteen, fourteen, fifteen, sixteen, seventeen, eighteen, nineteen, twenty, twenty one, twenty two, twenty three, twenty four, twenty five, twenty six, twenty seven, twenty eight, twenty nine, thirty, thirty one, thirty two, thirty three, thirty four, thirty five, thirty six, or thirty seven of the antigens (preferably, the full-length antigens);

(b) antibodies, or antibody fragments, that specifically bind three, four, five, six, seven, eight, nine, ten, eleven, twelve, thirteen, fourteen, fifteen, sixteen, seventeen, eighteen, nineteen, twenty, twenty one, twenty two, twenty three, twenty four, twenty five, twenty six, twenty seven, twenty eight, twenty nine, thirty, thirty one, thirty two, thirty three, thirty four, thirty five, thirty six, or thirty seven of the antigens; or

(c) oligonucleotides that bind to nucleic acid sequences encoding three, four, five, six, seven, eight, nine, ten, eleven, twelve, thirteen, fourteen, fifteen, sixteen, seventeen, eighteen, nineteen, twenty, twenty one, twenty two, twenty three, twenty four, twenty five, twenty six, twenty seven, twenty eight, twenty nine, thirty, thirty one, thirty two, thirty three, thirty four, thirty five, thirty six, or thirty seven of the antigens. Optionally, the arrays further include capture probes directed at other targets (e.g., other tumor antigens, antibodies of other tumor antigens, nucleic acid molecules encoding other tumor antigens, or entirely different targets). Alternatively, in some embodiments, arrays do not include captures probes for any other targets.

In some embodiments, the arrays have capture probes that target no more than 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 50, 60, 70, 80, 90, or 100 molecular species in total.

The substrate bearing the capture probes is contacted with a biological sample that potentially contains the target binding partner of the capture probes (e.g., antibodies, antigens, or nucleic acid sequences (DNA or mRNA) encoding the antigens). Contacting may be performed in any suitable device. The substrate may be, for example, a plate, microtiter dish, test tube, well, glass, polymer, membrane, column, and so forth. In specific embodiments, the contacting is performed on a substrate coated with the capture probes, such as a nucleic acid array, protein array, antibody array, or a specific ligand array. The substrate may be a solid or semi-solid substrate such as any suitable support comprising glass, plastic, nylon, paper, metal, polymers and the like. The substrate may be of various forms and sizes, such as a slide, a membrane, a bead, a column, a chip, a gel, etc. The contacting may be made under any condition suitable for a detectable complex, such as a nucleic acid hybrid or an antibody-antigen complex, to be formed between the capture probe and the nucleic acids, immunoglobulins, or polypeptides of the sample.

The subject invention also concerns kits for the detection of two or more target antibodies or antigens of the invention. In one embodiment, a kit of the invention comprises, in one or more separate containers, two or more capture probes of the invention. Optionally, the two or more capture probes are attached to a substrate. The kits may include one or more arrays of the invention. Kits of the invention can also optionally comprise additional reagents. Containers in a kit of the invention can be composed of any suitable material, such as glass or plastic. In one embodiment, a kit of the invention further comprises positive or negative controls or standards that the assayed sample can be compared to. In one embodiment, a kit of the invention can optionally comprises instructions pertaining to the use of the reagents and/or methods of the invention, packaging materials, sample diluents, buffers, wash reagents, and/or additional containers.

The arrays and kits of the invention may be used to carry out prognostic methods and treatment methods of the invention.

The names, National Center for Biotechnology Information (NCBI) Reference Sequence Accession numbers, and nucleic acid sequences of the prognostic antigens (biomarkers) of the invention are provided herein. Numeric sequence identifiers assigned to nucleic acid sequences representing embodiments of these biomarkers are as follows: BRAF (SEQ ID NO:29), CABYR (SEQ ID NO:30), CRISP3 (SEQ ID NO:31), CSAG2 (SEQ ID NO:1), CTAG2 (SEQ ID NO:2), CXorf48.1 (SEQ ID NO:3), DHFR (SEQ ID NO:32), FTHL17 (SEQ ID NO:4), GAGE1 (SEQ ID NO:5), GAGE2A (SEQ ID NO:6), GLUD1 (SEQ ID NO:33), LDHC (SEQ ID NO:7), MAGEA1 (SEQ ID NO:8), MAGEA3 (SEQ ID NO:9), MAGEA4V2 (SEQ ID NO:10), MAGEA4V3 (SEQ ID NO:11), MAGEA4V4 (SEQ ID NO:12), MAGEB6 (SEQ ID NO:13), MAPK1 (SEQ ID NO:28), MICA (SEQ ID NO 14), MUC1 (SEQ ID NO:34), NLRP4 (SEQ ID NO:15), NY-ESO-1 (SEQ ID NO:16), PBK (SEQ ID NO:17), PRAME (SEQ ID NO:35), SOX2 (SEQ ID NO:36), SILV (SEQ ID NO:18), SPANXA1 (SEQ ID NO:19), SPANXB1 (SEQ ID NO:20), SSX2A (SEQ ID NO:21), SSX4 (SEQ ID NO:22), TSGA10 (SEQ ID NO:23), TSSK6 (SEQ ID NO:24), TULP2 (SEQ ID NO:37), TYR (SEQ ID NO:25), XAGE-2 (SEQ ID NO:26), and ZNF165 (SEQ ID NO:27). It should be understood, that the biomarkers used in the subject invention also include variants of these nucleic acid sequences and variant polypeptides encoded by SEQ ID NOs:1-37 or encoded by variants thereof. Preferably, the nucleic acid sequences encode functional polypeptides (functional versions of the recited polypeptide biomarkers). Variant sequences include those sequences wherein one or more nucleotides or amino acids of the sequence have been substituted, deleted, and/or inserted. Amino acids can be generally categorized in the following classes: non-polar, uncharged polar, basic, and acidic. Conservative substitutions whereby a polypeptide having an amino acid of one class is replaced with another amino acid of the same class fall within the scope of the subject invention so long as the polypeptide having the substitution still retains substantially the same functional activity as the polypeptide that does not have the substitution. Polynucleotides encoding a polypeptide having one or more amino acid substitutions in the sequence are contemplated within the scope of the present invention.

Polynucleotides and polypeptides contemplated within the scope of the subject invention can also be defined in terms of more particular identity and/or similarity ranges with those sequences of the invention specifically exemplified herein. The sequence identity will typically be greater than 60%, preferably greater than 75%, more preferably greater than 80%, even more preferably greater than 90%, and can be greater than 95%. The identity and/or similarity of a sequence can be 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% as compared to a sequence exemplified herein (e.g., compared to a sequence of SEQ ID NOs:1-37, or compared to a sequence encoded by SEQ ID NOs:1-37). Unless otherwise specified, as used herein, percent sequence identity and/or similarity of two sequences can be determined using the algorithm of Karlin and Altschul (1990) (“Methods for Assessing the Statistical Significance of Molecular Sequence Features by Using General Scoring Schemes” Proc. Natl. Acad. Sci. USA 87:2264-2268 (1990)), modified as in Karlin and Altschul (1993) (“Applications and Statistics for Multiple High-Scoring Segments in Molecular Sequences” Proc. Natl. Acad. Sci. USA 90:5873-5877 (1993)). Such an algorithm is incorporated into the NBLAST and XBLAST programs of Altschul et al. (1997) (“Gapped BLAST and PSI-BLAST: A New Generation of Protein Database Search Programs” Nucl. Acids Res. 25:3389-3402 (1997)). BLAST searches can be performed with the NBLAST program, score=100, wordlength=12, to obtain sequences with the desired percent sequence identity. To obtain gapped alignments for comparison purposes, Gapped BLAST can be used as described in Altschul et al. (1997). When utilizing BLAST and Gapped BLAST programs, the default parameters of the respective programs (NBLAST and XBLAST) can be used. See NCBI/NIH website.

The subject invention also contemplates those polynucleotide molecules having sequences which are sufficiently homologous with the polynucleotide sequences exemplified herein so as to permit hybridization with that sequence under standard stringent conditions and standard methods (Maniatis et al., 1982). As used herein, “stringent” conditions for hybridization refers to conditions wherein hybridization is typically carried out overnight at 20-25 C below the melting temperature (Tm) of the DNA hybrid in 6×SSPE, 5×Denhardt's solution, 0.1% SDS, 0.1 mg/ml denatured DNA. The melting temperature, Tm, is described by the following formula (Boltz et al., 1983):

Tm=81.5 C+16.6 Log [Na+]+0.41 (% G+C)−0.61 (% formamide)−600/length of duplex in base pairs.

Washes are typically carried out as follows:

(1) Twice at room temperature for 15 minutes in 1×SSPE, 0.1% SDS (low stringency wash).

(2) Once at Tm-20 C for 15 minutes in 0.2×SSPE, 0.1% SDS (moderate stringency wash).

As used herein, the terms “nucleic acid” and “polynucleotide” refer to a deoxyribonucleotide, ribonucleotide, or a mixed deoxyribonucleotide and ribonucleotide polymer in either single- or double-stranded form, and unless otherwise limited, would encompass known analogs of natural nucleotides that can function in a similar manner as naturally-occurring nucleotides. The polynucleotide sequences include the DNA strand sequence that is transcribed into RNA and the strand sequence that is complementary to the DNA strand that is transcribed. The polynucleotide sequences also include both full-length sequences as well as shorter sequences derived from the full-length sequences. Allelic variations of the exemplified sequences also fall within the scope of the subject invention. The polynucleotide sequence includes both the sense and antisense strands either as individual strands or in the duplex.

As used herein, the terms “administering” or “administer” are used herein to refer the introduction of a substance into cells in vitro or into the body of an individual in vivo by any route (for example, oral, nasal, ocular, rectal, vaginal and parenteral routes). Active agents, such as immunotherapeutics, may be administered individually or in combination with other immunotherapeutic or non-therapeutic agents via any route of administration, including but not limited to subcutaneous (SQ), intramuscular (IM), intravenous (IV), intraperitoneal (IP), intradermal (ID), via the nasal, ocular or oral mucosa (IN), or orally. For example, active agents such as immunotherapeutics can be administered by direct injection into or on a tumor, or systemically (e.g., into the circulatory system).

As used herein, the term “adverse event” in connection with an immunotherapy refers to autoimmune toxicity, which can include, for example, a gastrointestinal autoimmune side effect (colitis, stomach pain, bloating, constipation, diarrhea), dermatitis, anti-pituitary autoimmune side effect, hepatitis, inflammation of the hormone gland(s), inflammation of the eyes, inflammation of the nerves, or two or more of the foregoing (see, for example, Amos, S. M. et al., “Autoimmunity associated with immunotherapy of cancer,” Blood, Jul. 21, 2011; Epub Apr. 29, 2011; 118(3):499-509).

As used herein, the term “(therapeutically) effective amount” refers to an amount of the immunotherapeutic or other active agent (drug, biologic, etc.) effective to treat a disease or disorder in a mammal. In the case of a malignancy, the therapeutically effective amount of the agent may reduce (i.e., slow to some extent and preferably stop) unwanted cellular proliferation; reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; reduce signaling in the target cells, and/or relieve, to some extent, one or more of the symptoms associated with the cancer. It should be noted that a therapeutically effective amount of an immunotherapeutic may initially cause a tumor to enlarge, from lymphocyte infiltration. To the extent the administered agent directly or indirectly prevents growth of and/or kills existing cancer cells, it may be cytostatic and/or cytotoxic. For cancer therapy, efficacy of the immunotherapeutic or other agent can, for example, be measured by assessing the time to disease progression (TTP), survival, and/or determining the response rate (RR).

As used herein, the term “bind” refers to any physical attachment or close association, which may be permanent or temporary. The binding can result from hydrogen bonding, hydrophobic forces, van der Waals forces, covalent, or ionic bonding, for example. For example, the binding may be an antigen-antibody reaction, such as between a prognostic antigen of the invention and its antibody. Binding may also be hybridization at various stringencies through standard Watson and Crick type base-pairing, as between an oligonucleotide and a nucleic acid sequence encoding a prognostic antigen of the invention.

As used herein, the term “biomarker” may refer to a prognostic antigen of the invention, antibodies to the antigen, or nucleic acid sequences encoding the antigen.

As used herein, the term “sample” refers to a composition (e.g., biological composition) that potentially contains the target molecules (e.g., target antigens, antibodies to the antigens, nucleic acid molecules, T cells activated against the antigens) with which the capture probes are contacted. Thus, a sample potentially contains the target binding partner of capture probes (e.g., antibodies, antigens, or nucleic acid sequences (DNA or mRNA) encoding the antigens). Samples may be removed from the body of a subject using any method or technique. For example, blood or other fluid samples may be removed using a syringe or needle. A swab may be used to remove endothelium cells. Other samples may be removed by biopsy or tissue section.

Examples of such samples include fluids such as blood (e.g., peripheral blood), plasma, serum, saliva, urine and seminal fluid samples as well as biopsies, organs, tissues or cell samples. The sample may be treated prior to its use, e.g., in order to render nucleic acids available. The terms “cancer sample”, “malignancy sample”, or “tumor sample” refers to any sample containing tumoral cells derived from a patient. The term “normal sample” refers to any sample which does not contain any tumoral cell.

The sample may be a cellular sample (samples of intact cells, e.g., a cytology sample) or non-cellular sample. One or more samples of a malignancy may be obtained from a subject by techniques known in the art, such as biopsy. The type of biopsy utilized is dependent upon the anatomical location from which the sample is to be obtained. Methods for collecting various body samples are known in the art. Examples include fine needle aspiration (FSA), excisional biopsy, incisional biopsy, colonoscopic biopsy, punch biopsy, and bone marrow biopsy. Samples may be transferred to a glass slide for viewing under magnification. Fixative and staining solutions may be applied to the cells on the slide for preserving the specimen and/or for facilitating examination. It should be understood that the methods of the invention may include a step in which a sample is obtained directly from a subject; alternatively, a sample may be obtained or otherwise provided, e.g., by a third party.

A sample may be taken from a subject having or suspected of having cancer. A sample may also comprise proteins isolated from a tissue or cell sample from a subject. In certain aspects, the sample can be, but is not limited to tissue (e.g., biopsy, particularly fine needle biopsy, excision, or punch biopsy), blood, serum, plasma. The sample can be fresh, frozen, fixed (e.g., formalin fixed), or embedded (e.g., paraffin embedded) tissues or cells (e.g., FFPE tissue). In a particular aspect, the sample is a blood or serum sample and the level of antibodies specific for the antigens of interest is determined by contacting the sample with an array with the corresponding capture probes (e.g., antigenic epitopes or full length antigens) disposed thereon.

Mammalian species which benefit from the disclosed arrays, methods, and kits include, but are not limited to, primates, such as apes, chimpanzees, orangutans, humans, monkeys; domesticated animals (e.g., pets) such as dogs, cats, guinea pigs, hamsters, Vietnamese pot-bellied pigs, rabbits, and ferrets; domesticated farm animals such as cows, buffalo, bison, horses, donkey, swine, sheep, and goats; exotic animals typically found in zoos, such as bear, lions, tigers, panthers, elephants, hippopotamus, rhinoceros, giraffes, antelopes, sloth, gazelles, zebras, wildebeests, prairie dogs, koala bears, kangaroo, opossums, raccoons, pandas, hyena, seals, sea lions, elephant seals, otters, porpoises, dolphins, and whales. Other species that may benefit from the disclosed methods include fish, amphibians, avians, and reptiles. As used herein, the terms “patient”, “subject”, and “individual” are used interchangeably and are intended to include such human and non-human species unless specified to be human or non-human.

Patients in need of treatment using the methods of the present invention (e.g., having a malignancy) can be identified using standard techniques known to those in the medical or veterinary professions, as appropriate. A subject having a malignancy may be symptomatic or asymptomatic.

Patient responsiveness to treatment for a particular disorder can be based on a measurable parameter that is indicative of patient improvement after receiving a therapeutic treatment.

The terms “cancer” and “malignancy” are used herein interchangeably to refer to or describe the physiological condition in mammals that is typically characterized by unregulated cell growth. The cancer may be drug-resistant or drug-sensitive. The cancer may be primary or metastatic. Examples of cancer include but are not limited to, carcinoma, lymphoma, blastoma, sarcoma, and leukemia. More particular examples of such cancers include breast cancer, prostate cancer, colon cancer, squamous cell cancer, small-cell lung cancer, non-small cell lung cancer, gastrointestinal cancer, pancreatic cancer, cervical cancer, ovarian cancer, peritoneal cancer, liver cancer, e.g., hepatic carcinoma, bladder cancer, colorectal cancer, endometrial carcinoma, kidney cancer, and thyroid cancer.

Other non-limiting examples of cancers are basal cell carcinoma, biliary tract cancer; bone cancer; brain and CNS cancer; choriocarcinoma; connective tissue cancer; esophageal cancer; eye cancer; cancer of the head and neck; gastric cancer; intra-epithelial neoplasm; larynx cancer; lymphoma including Hodgkin's and Non-Hodgkin's lymphoma; melanoma; myeloma; neuroblastoma; oral cavity cancer (e.g., lip, tongue, mouth, and pharynx); retinoblastoma; rhabdomyo sarcoma; rectal cancer; cancer of the respiratory system; sarcoma; skin cancer; stomach cancer; testicular cancer; uterine cancer; cancer of the urinary system, as well as other carcinomas and sarcomas. Examples of cancer types that may potentially be sampled and treated using the arrays, kits, and methods of the invention are also listed in Table 1.

TABLE 1
Examples of Cancer Types

| | |
|---|---|
| Acute Lymphoblastic Leukemia, Adult | Hairy Cell Leukemia |
| Acute Lymphoblastic Leukemia, | Head and Neck Cancer |
| Childhood | Hepatocellular (Liver) Cancer, Adult |
| Acute Myeloid Leukemia, Adult | (Primary) |
| Acute Myeloid Leukemia, Childhood | Hepatocellular (Liver) Cancer, Childhood |
| Adrenocortical Carcinoma | (Primary) |
| Adrenocortical Carcinoma, Childhood | Hodgkin's Lymphoma, Adult |
| AIDS-Related Cancers | Hodgkin's Lymphoma, Childhood |
| AIDS-Related Lymphoma | Hodgkin's Lymphoma During Pregnancy |
| Anal Cancer | Hypopharyngeal Cancer |
| Astrocytoma, Childhood Cerebellar | Hypothalamic and Visual Pathway Glioma, |
| Astrocytoma, Childhood Cerebral | Childhood |
| Basal Cell Carcinoma | Intraocular Melanoma |
| Bile Duct Cancer, Extrahepatic | Islet Cell Carcinoma (Endocrine Pancreas) |
| Bladder Cancer | Kaposi's Sarcoma |
| Bladder Cancer, Childhood | Kidney (Renal Cell) Cancer |
| Bone Cancer, Osteosarcoma/Malignant | Kidney Cancer, Childhood |
| Fibrous Histiocytoma | Laryngeal Cancer |
| Brain Stem Glioma, Childhood | Laryngeal Cancer, Childhood |
| Brain Tumor, Adult | Leukemia, Acute Lymphoblastic, Adult |
| Brain Tumor, Brain Stem Glioma, | Leukemia, Acute Lymphoblastic, Childhood |
| Childhood | Leukemia, Acute Myeloid, Adult |
| Brain Tumor, Cerebellar Astrocytoma, | Leukemia, Acute Myeloid, Childhood |
| Childhood | Leukemia, Chronic Lymphocytic |
| Brain Tumor, Cerebral | Leukemia, Chronic Myelogenous |
| Astrocytoma/Malignant Glioma, | Leukemia, Hairy Cell |
| Childhood | Lip and Oral Cavity Cancer |
| Brain Tumor, Ependymoma, Childhood | Liver Cancer, Adult (Primary) |
| Brain Tumor, Medulloblastoma, | Liver Cancer, Childhood (Primary) |
| Childhood | Lung Cancer, Non-Small Cell |
| Brain Tumor, Supratentorial Primitive | Lung Cancer, Small Cell |
| Neuroectodermal Tumors, Childhood | Lymphoma, AIDS-Related |
| Brain Tumor, Visual Pathway and | Lymphoma, Burkitt's |
| Hypothalamic Glioma, Childhood | Lymphoma, Cutaneous T-Cell, see Mycosis |
| Brain Tumor, Childhood | Fungoides and Sézary Syndrome |
| Breast Cancer | Lymphoma, Hodgkin's, Adult |
| Breast Cancer, Childhood | Lymphoma, Hodgkin's, Childhood |
| Breast Cancer, Male | Lymphoma, Hodgkin's During Pregnancy |
| Bronchial Adenomas/Carcinoids, | Lymphoma, Non-Hodgkin's, Adult |
| Childhood | Lymphoma, Non-Hodgkin's, Childhood |
| Burkitt's Lymphoma | Lymphoma, Non-Hodgkin's During |
| Carcinoid Tumor, Childhood | Pregnancy |
| Carcinoid Tumor, Gastrointestinal | Lymphoma, Primary Central Nervous System |
| Carcinoma of Unknown Primary | Macroglobulinemia, Waldenström's |
| Central Nervous System Lymphoma, | Malignant Fibrous Histiocytoma of |
| Primary | Bone/Osteosarcoma |
| Cerebellar Astrocytoma, Childhood | Medulloblastoma, Childhood |
| Cerebral Astrocytoma/Malignant | Melanoma |
| Glioma, Childhood | Melanoma, Intraocular (Eye) |
| Cervical Cancer | Merkel Cell Carcinoma |
| Childhood Cancers | Mesothelioma, Adult Malignant |
| Chronic Lymphocytic Leukemia | Mesothelioma, Childhood |
| Chronic Myelogenous Leukemia | Metastatic Squamous Neck Cancer with |
| Chronic Myeloproliferative Disorders | Occult Primary |
| Colon Cancer | Multiple Endocrine Neoplasia Syndrome, |
| Colorectal Cancer, Childhood | Childhood |
| Cutaneous T-Cell Lymphoma, see | Multiple Myeloma/Plasma Cell Neoplasm |
| Mycosis Fungoides and Sézary | Mycosis Fungoides |
| Syndrome | Myelodysplastic Syndromes |
| Endometrial Cancer | Myelodysplastic/Myeloproliferative Diseases |
| Ependymoma, Childhood | Myelogenous Leukemia, Chronic |
| Esophageal Cancer | Myeloid Leukemia, Adult Acute |
| Esophageal Cancer, Childhood | Myeloid Leukemia, Childhood Acute |
| Ewing's Family of Tumors | Myeloma, Multiple |
| Extracranial Germ Cell Tumor, | Myeloproliferative Disorders, Chronic |
| Childhood | Nasal Cavity and Paranasal Sinus Cancer |
| Extragonadal Germ Cell Tumor | Nasopharyngeal Cancer |
| Extrahepatic Bile Duct Cancer | Nasopharyngeal Cancer, Childhood |
| Eye Cancer, Intraocular Melanoma | Neuroblastoma |
| Eye Cancer, Retinoblastoma | Non-Hodgkin's Lymphoma, Adult |
| Gallbladder Cancer | Non-Hodgkin's Lymphoma, Childhood |
| Gastric (Stomach) Cancer | Non-Hodgkin's Lymphoma During Pregnancy |
| Gastric (Stomach) Cancer, Childhood | Non-Small Cell Lung Cancer |
| Gastrointestinal Carcinoid Tumor | Oral Cancer, Childhood |
| Germ Cell Tumor, Extracranial, | Oral Cavity Cancer, Lip and |
| Childhood | Oropharyngeal Cancer |
| Germ Cell Tumor, Extragonadal | Osteosarcoma/Malignant Fibrous |
| Germ Cell Tumor, Ovarian | Histiocytoma of Bone |
| Gestational Trophoblastic Tumor | Ovarian Cancer, Childhood |
| Glioma, Adult | Ovarian Epithelial Cancer |
| Glioma, Childhood Brain Stem | Ovarian Germ Cell Tumor |
| Glioma, Childhood Cerebral | Ovarian Low Malignant Potential Tumor |
| Astrocytoma | Pancreatic Cancer |
| Glioma, Childhood Visual Pathway and | Pancreatic Cancer, Childhood |
| Hypothalamic | Pancreatic Cancer, Islet Cell |
| Skin Cancer (Melanoma) | Paranasal Sinus and Nasal Cavity Cancer |
| Skin Carcinoma, Merkel Cell | Parathyroid Cancer |
| Small Cell Lung Cancer | Penile Cancer |
| Small Intestine Cancer | Pheochromocytoma |
| Soft Tissue Sarcoma, Adult | Pineoblastoma and Supratentorial Primitive |
| Soft Tissue Sarcoma, Childhood | Neuroectodermal Tumors, Childhood |
| Squamous Cell Carcinoma, see Skin | Pituitary Tumor |
| Cancer (non-Melanoma) | Plasma Cell Neoplasm/Multiple Myeloma |
| Squamous Neck Cancer with Occult | Pleuropulmonary Blastoma |
| Primary, Metastatic | Pregnancy and Breast Cancer |
| Stomach (Gastric) Cancer | Pregnancy and Hodgkin's Lymphoma |
| Stomach (Gastric) Cancer, Childhood | Pregnancy and Non-Hodgkin's Lymphoma |
| Supratentorial Primitive | Primary Central Nervous System Lymphoma |
| Neuroectodermal Tumors, Childhood | Prostate Cancer |
| T-Cell Lymphoma, Cutaneous, see | Rectal Cancer |
| Mycosis Fungoides and Sézary | Renal Cell (Kidney) Cancer |
| Syndrome | Renal Cell (Kidney) Cancer, Childhood |
| Testicular Cancer | Renal Pelvis and Ureter, Transitional Cell |
| Thymoma, Childhood | Cancer |
| Thymoma and Thymic Carcinoma | Retinoblastoma |
| Thyroid Cancer | Rhabdomyosarcoma, Childhood |
| Thyroid Cancer, Childhood | Salivary Gland Cancer |
| Transitional Cell Cancer of the Renal | Salivary Gland Cancer, Childhood |
| Pelvis and Ureter | Sarcoma, Ewing's Family of Tumors |
| Trophoblastic Tumor, Gestational | Sarcoma, Kaposi's |
| Unknown Primary Site, Carcinoma of, | Sarcoma, Soft Tissue, Adult |
| Adult | Sarcoma, Soft Tissue, Childhood |
| Unknown Primary Site, Cancer of, | Sarcoma, Uterine |
| Childhood | Sezary Syndrome |
| Unusual Cancers of Childhood | Skin Cancer (non-Melanoma) |
| Ureter and Renal Pelvis, Transitional | Skin Cancer, Childhood |
| Cell Cancer | |
| Urethral Cancer | |
| Uterine Cancer, Endometrial | |
| Uterine Sarcoma | |
| Vaginal Cancer | |
| Visual Pathway and Hypothalamic | |
| Glioma, Childhood | |
| Vulvar Cancer | |
| Waldenström's Macroglobulinemia | |
| Wilms' Tumor | |

As used herein, the term “tumor” refers to all neoplastic cell growth and proliferation, whether malignant or benign, and all pre-cancerous and cancerous cells and tissues. For example, a particular cancer may be characterized by a solid mass tumor or non-solid tumor. The solid tumor mass, if present, may be a primary tumor mass. A primary tumor mass refers to a growth of cancer cells in a tissue resulting from the transformation of a normal cell of that tissue. In most cases, the primary tumor mass is identified by the presence of a cyst, which can be found through visual or palpation methods, or by irregularity in shape, texture or weight of the tissue. However, some primary tumors are not palpable and can be detected only through medical imaging techniques such as X-rays (e.g., mammography) or magnetic resonance imaging (MRI), or by needle aspirations. The use of these latter techniques is more common in early detection. Molecular and phenotypic analysis of cancer cells within a tissue can usually be used to confirm if the cancer is endogenous to the tissue or if the lesion is due to metastasis from another site. Some tumors are unresectable (cannot be surgically removed due to, for example the number of metastatic foci or because it is in a surgical danger zone). The treatment and prognostic methods of the invention can be utilized for early, middle, or late stage disease, and acute or chronic disease.

According to methods of the subject invention, an immunotherapy or alternative therapy can be administered to a patient by itself, or co-administered with one or more other agents such as another immunotherapeutic and/or another non-immunotherapeutic. Co-administration can be carried out simultaneously (in the same or separate formulations) or consecutively. Furthermore, immunotherapies can be administered to a patient as adjuvant therapy. For example, an immunotherapy can be administered to a patient in conjunction with chemotherapy, radiation therapy, surgery, or a combination of two or more of the foregoing.

Thus, immunotherapeutics, whether administered separately, or as a pharmaceutical composition, can include various other components as additives. Examples of acceptable components or adjuncts which can be employed in relevant circumstances include antioxidants, free radical scavenging agents, peptides, growth factors, antibiotics, bacteriostatic agents, immunosuppressives, anticoagulants, buffering agents, anti-inflammatory agents, anti-angiogenics, anti-pyretics, time-release binders, anesthetics, steroids, and corticosteroids. Such components can provide additional therapeutic benefit, act to affect the therapeutic action of the compounds of the invention, or act towards preventing any potential side effects which may be posed as a result of administration of the compounds. The immunotherapeutic agent can be conjugated to a therapeutic agent or other agent, as well.

As used herein, the term “immunotherapy” refers to the treatment of disease via the stimulation, induction, subversion, mimicry, enhancement, augmentation or any other modulation of a subject's immune system to elicit or amplify adaptive or innate immunity (actively or passively) against cancerous or otherwise harmful proteins, cells or tissues. Immunotherapies (i.e., immunotherapeutic agents) include cancer vaccines, immunomodulators, monoclonal antibodies (e.g., humanized monoclonal antibodies), immunostimulants, dendritic cells, and viral therapies, whether designed to treat existing cancers or prevent the development of cancers or for use in the adjuvant setting to reduce likelihood of recurrence of cancer. Examples of cancer vaccines include GVAX, Stimuvax, DCVax and other vaccines designed to elicit immune responses to tumor and other antigens including MUC1, NY-ESO-1, MAGE, p53 and others. Examples of immunomodulators include 1MT, Ipilimumab, Tremelimumab and/or any drug designed to de-repress or otherwise modulate cytotoxic or other T cell activity against tumor or other antigens, including, but not restricted to, treatments that modulate T-Reg cell control pathways via CTLA-4, CD80, CD86, MHC, B7-DC, B7-H1, B7-H2, B7-H3, B7-H4, CD28, other TCRs, PD-1, PDL-1, CD80, ICOS and their ligands, whether via blockade, agonist or antagonist. Examples of immunostimulants include corticosteroids and any other anti- or pro-inflammatory agent, steroidal or non-steroidal, including, but not restricted to, GM-CSF, interleukins (eg IL-2, IL-7. IL-12), cytokines such as the interferons, and others. Examples of dendritic cell (DC) therapies include modified dendritic cells and any other antigen presenting cell, autologous or xeno, whether modified by multiple antigens, whole cancer cells, single antigens, by mRNA, phage display or any other modification, including but not restricted to ex vivo-generated, antigen-loaded dendritic cells (DCs) to induce antigen-specific T-cell immunity, ex vivo gene-loaded DCs to induce humoral immunity, ex vivo-generated antigen-loaded DCs induce tumour-specific immunity, ex vivo-generated immature DCs to induce tolerance, including but not limited to Provenge and others. Examples of viral therapies include oncolytic viruses or virus-derived genetic or other material designed to elicit anti-tumor immunity and inhibitors of infectious viruses associated with tumor development, such as drugs in the Prophage series. Examples of monoclonal antibodies include Alemtuzumab, Bevacizumab, Cetuximab, Gemtuzumab ozogamicin, Rituximab, Trastuzumab, Radioimmunotherapy, Ibritumomab tiuxetan, Tositumomab/iodine tositumomab regimen. An immunotherapy may be a monotherapy or used in combination with one or more other therapies (one or more other immunotherapies or non-immunotherapies).

Enhancing or prolonging T-cell activation by monoclonal antibodies (mAbs) blocking negative signaling receptors such as CTLA-4 is an approach to overcoming tumor-induced immune tolerance. Ipilimumab and Tremelimumab inhibit CTLA-4, prolonging antitumor immune responses and leading to durable anti-tumor effects (Graziani G. et al., “Ipilimumab: A Novel Immunostimulatory Monoclonal Antibody for the Treatment of Cancer,” Pharmacol. Res., 2012, Jan., Epub 2011 Sep. 10, 65(1):9-22; and Tarhini A. A. et al., “CTLA-4 Blockade: Therapeutic Potential in Cancer Patients,” Onco. Targets Ther., 2010, 3:15-25, which are each incorporated herein by reference in their entirety). Ipilumumab has been approved by the U.S. Food and Drug Administration for the treatment of unresectable or metastatic melanoma. In some embodiments of the invention, the immunotherapy comprises an anti-CTLA-4 therapy, i.e., an agent that blocks or inhibits CTLA-4, such as an antibody that binds to CTLA-4 (e.g., Ipilimumab, which is an IgG1 isotype antibody, or Tremelimumab, which is an IgG2 isotype antibody). In some embodiments, the immunotherapy comprises an anti-CTLA-4 therapy, such as Ipilimumab or Tremelimumab, and the cancer is one selected from melanoma (unresectable, metastatic, or other melanoma), lung cancer (small-cell or non-small cell lung cancer), or prostate cancer.

As indicated above, the invention includes an array comprising capture probes disposed on a substrate, in which the capture probes specifically bind (1) antibodies of the antigens, or (2) two or more of the prognostic antigens (proteins) themselves, or (3) nucleic acid molecules encoding two or more of the prognostic antigens (see, for example, Berton P. and Snyder M., “Advances in functional protein microarray technology,” FEBS J, 2005; 272(21):5400-5411; Wingren C. and Borrebaeck C. A., “Antibody microarrys: current status and key technological advances,” OMICS, 2006, 10(3):411-427; Zhu H. and Snyder M., Curr. Opin. Chem. Biol., 2003, 7(1):55-63; Büssow K. et al., “Protein Array Technology: Potential Use in Medical Diagnostics,” Am. J. Pharmaceogenomics, 2001, 1(1):1-7). Thus, for example, the array can be a protein array (with antigenic epitopes disposed on the substrate), an antibody array (with antibodies or antibody fragments disposed on the substrate), or a nucleic acid array (with oligonucleotides disposed on the substrate, in which the oligonucleotides are partially or fully complementary with nucleic acid sequences encoding the prognostic antigens).

The substrate may be any solid or semi-solid support for supporting the capture probes, such as a particle (e.g., magnetic or latex particle), a microtiter multi-well plate (e.g., 96-well, 384-well, 1536-well, etc.), a bead, a slide, a filter, a chip, a membrane, a cuvette, or a reaction vessel. The capture probes may be manufactured synthetically directly on the substrate or be produced and subsequently immobilized or otherwise attached to the substrate using standard technologies such as pin-based spotting, liquid microdispensing, adsorption to charged or hydrophobic surfaces, covalent cross-linking or specific binding via tags (e.g., nickel chelating or streptavidin coated surfaces for plasmon resonance measurements). In the arrays of the invention, the capture probes can be in ordered arrangements on the substrates, or be randomly disposed, and can be of various densities.

Detectable labels that can be used with the present invention include, but are not limited to, enzymes, radioisotopes, chemiluminescent and bioluminescent reagents, and fluorescent moieties. Enzymes that can be used include but are not limited to lucerifase, beta-galactosidase, acetylcholinesterase, horseradish peroxidase, glucose-6-phosphate dehydrogenase, and alkaline phosphatase. If the detectable label is an enzyme, then a suitable substrate that can be acted upon by the enzyme can be used for detection and measurement of enzyme activity. In one embodiment, if the detectable label is a peroxidase, the substrate can be hydrogen peroxide (H₂O₂) and 3-3′ diaminobenzidine or 4-chloro-1-naphthol and the like. Other substrates suitable for use with other enzymes are well known in the art. An example of a luminescent material includes luminol. Examples of bioluminescent materials include, but are not limited to, luciferin, green fluorescent protein (GFP), enhanced GFP (Yang et al., 1996), and aequorin. Fluorescent moieties include, but are not limited to, umbelliferone, fluorescein, fluorescein isothiocyanate, Cascade Blue, rhodamine, dichlorotriazinylamine fluorescein, dansyl chloride, Texas Red, Oregon Green, cyanines (e.g., CY2, CY3, and CY5), allophycocyanine, or phycoerythrin. Isotopes that can be used include, but are not limited to, ¹²⁵I, ¹⁴C, ³⁵S, and ³H.

### Antibodies

Antibodies contemplated for use in the present invention can be in any of a variety of forms, including a whole immunoglobulin, an antibody fragment such as Fv, Fab, and similar fragments, a single chain antibody that includes the variable domain complementarity determining regions (CDR), and the like forms, all of which fall under the broad term “antibody,” as used herein. Antibodies useful in the arrays, kits, and methods of the present invention can be monoclonal or polyclonal antibodies, and can be from any source including, but not limited to, mouse, rabbit, goat, rat, or human. Antibodies of the invention can be conjugated to a detectable label, such as, for example, a fluorescent moiety. In one embodiment of the present invention, a detectable label can be directly bound to an antibody that binds to a prognostic antigen of the invention (or to another antibody that binds the prognostic antigen). If the detectable label is to be directly bound, the label may comprise a functional group which is capable of binding to the antibody used with the invention. Alternatively, the detectable label may be indirectly bound, for example, using an avidin-biotin or streptavidin-biotin bridge wherein the avidin or biotin is labeled with a detectable label. In one embodiment, an antibody of the invention is conjugated with avidin and the detectable label is conjugated with biotin.

The term “antibody fragment” refers to a portion of a full-length antibody, generally the antigen binding or variable region. Examples of antibody fragments include Fab, Fab′, F(ab′)₂ and Fv fragments. Papain digestion of antibodies produces two identical antigen binding fragments, called the Fab fragment, each with a single antigen binding site, and a residual “Fc” fragment, so-called for its ability to crystallize readily. Pepsin treatment yields an F(ab′)₂ fragment that has two antigen binding fragments, which are capable of cross-linking antigen, and a residual other fragment (which is termed pFc′). Additional fragments can include diabodies, linear antibodies, single-chain antibody molecules, and multispecific antibodies formed from antibody fragments. As used herein, “functional fragment” with respect to antibodies, refers to Fv, F(ab) and F(ab′)₂ fragments.

Antibody fragments can retain an ability to selectively bind with the antigen or analyte and are defined as follows:

(1) Fab is the fragment that contains a monovalent antigen-binding fragment of an antibody molecule. A Fab fragment can be produced by digestion of whole antibody with the enzyme papain to yield an intact light chain and a portion of one heavy chain.

(2) Fab′ is the fragment of an antibody molecule can be obtained by treating whole antibody with pepsin, followed by reduction, to yield an intact light chain and a portion of the heavy chain. Two Fab′ fragments are obtained per antibody molecule. Fab′ fragments differ from Fab fragments by the addition of a few residues at the carboxyl terminus of the heavy chain CH1 domain including one or more cysteines from the antibody hinge region.

(3) (Fab′)₂ is the fragment of an antibody that can be obtained by treating whole antibody with the enzyme pepsin without subsequent reduction. F(ab′)₂ is a dimer of two Fab′ fragments held together by two disulfide bonds.

(4) Fv is the minimum antibody fragment that contains a complete antigen recognition and binding site. This region consists of a dimer of one heavy and one light chain variable domain in a tight, non-covalent association (V_{H}-V_{L} dimer). It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

(5) Single chain antibody (“SCA”), defined as a genetically engineered molecule containing the variable region of the light chain, the variable region of the heavy chain, linked by a suitable polypeptide linker as a genetically fused single chain molecule. Such single chain antibodies are also referred to as “single-chain Fv” or “sFv” antibody fragments. Generally, the Fv polypeptide further comprises a polypeptide linker between the VH and VL domains that enables the sFv to form the desired structure for antigen binding. For a review of sFv see Pluckthun in The Pharmacology of Monoclonal Antibodies, vol. 113, Rosenburg and Moore eds. Springer-Verlag, N.Y., pp. 269 315 (1994).

Antibodies specific for prognostic antigens of the invention that are used in the methods, arrays, and kits of the invention may be obtained from scientific or commercial sources. Alternatively, isolated native polypeptides or recombinant polypeptides may be utilized to prepare antibodies, monoclonal or polyclonal antibodies, and immunologically active fragments (e.g., a Fab or (Fab)₂ fragment), an antibody heavy chain, an antibody light chain, humanized antibodies, a genetically engineered single chain Fᵥ molecule (Ladne et al., U.S. Pat. No. 4,946,778), or a chimeric antibody, for example, an antibody which contains the binding specificity of a murine antibody, but in which the remaining portions are of human origin. Antibodies, including monoclonal and polyclonal antibodies, fragments and chimeras, may be prepared using methods known to those skilled in the art. In some embodiments, antibodies used in the methods of the invention are reactive against antigens of the invention if they bind with a Kₐ of greater than or equal to 10⁷ M. In a sandwich immunoassay of the invention, mouse polyclonal antibodies and rabbit polyclonal antibodies can be utilized, for example.

In order to produce monoclonal antibodies, a host mammal is inoculated with a protein or peptide representing a prognostic antigen of the invention and then boosted. Spleens are collected from inoculated mammals a few days after the final boost. Cell suspensions from the spleens are fused with a tumor cell in accordance with the general method described by Kohler and Milstein (Nature, 1975, 256:495-497). In order to be useful, a peptide fragment must contain sufficient amino acid residues to define the epitope of the biomarker molecule being detected.

If the fragment is too short to be immunogenic, it may be conjugated to a carrier molecule. Some suitable carrier molecules include keyhole limpet hemocyanin and bovine serum albumin. Conjugation may be carried out by methods known in the art. One such method is to combine a cysteine residue of the fragment with a cysteine residue on the carrier molecule. The peptide fragments may be synthesized by methods known in the art. Some suitable methods are described by Stuart and Young in “Solid Phase Peptide Synthesis,” Second Edition, Pierce Chemical Company (1984).

Purification of the antibodies or fragments can be accomplished by a variety of methods known to those skilled in the art including, precipitation by ammonium sulfate or sodium sulfate followed by dialysis against saline, ion exchange chromatography, affinity or immunoaffinity chromatography as well as gel filtration, zone electrophoresis, etc. (Goding in, Monoclonal Antibodies: Principles and Practice, 2d ed., pp. 104-126, Orlando, Fla., Academic Press). It is preferable to use purified antibodies or purified fragments of the antibodies having at least a portion of an antigenic binding region, including such as Fv, F(ab′)₂, Fab fragments (Harlow and Lane, 1988, Antibody Cold Spring Harbor) for the detection of the prognostic antigens in the samples of subjects.

For use in detection, the purified antibodies can be covalently attached, either directly or via linker, to a compound which serves as a reporter group to permit detection of the presence of the antigen. A variety of different types of substances can serve as the reporter group, including but not limited to enzymes, dyes, radioactive metal and non-metal isotopes, fluorogenic compounds, fluorescent compounds, etc. Methods for preparation of antibody conjugates of the antibodies (or fragments thereof) of the invention useful for detection, monitoring are described in U.S. Pat. Nos. 4,671,958; 4,741,900 and 4,867,973.

In one aspect of the invention, preferred binding epitopes may be identified from a known gene sequence and its encoded amino acid sequence and used to generate antibodies to the prognostic antigen with high binding affinity. Also, identification of binding epitopes on the prognostic antigen can be used in the design and construction of preferred antibodies. For example, a DNA encoding a preferred epitope on a prognostic antigen may be recombinantly expressed and used to select an antibody which binds selectively to that epitope. The selected antibodies then are exposed to the sample under conditions sufficient to allow specific binding of the antibody to the specific binding epitope on the antigen and the amount of complex formed then detected. Specific antibody methodologies are well understood and described in the literature. A more detailed description of their preparation can be found, for example, in Practical Immunology, Butt, W. R., ed., Marcel Dekker, New York, 1984.

The present invention also contemplates the detection of antibodies. Thus, detection of antibodies to the prognostic antigens of the invention in biological samples, such as blood samples or blood derived samples, of a subject is contemplated within the scope of the invention.

### Protein Binding Assays

Antibodies specifically reactive with the prognostic antigens disclosed herein or derivatives, such as enzyme conjugates or labeled derivatives, may be used to the detect antigens in various biological samples, for example they may be used in any known immunoassays which rely on the binding interaction between an antigenic determinant of a protein and the antibodies. Examples of such assays are radioimmunoassays, enzyme immunoassay (e.g., ELISA), immunofluorescence, immunoprecipitation, latex agglutination, hemagglutination, and histochemical tests.

An antibody specific for a prognostic antigen of the invention can be labeled with a detectable substance and localized in biological samples such as blood based upon the presence of the detectable substance. Examples of detectable substances include, but are not limited to, the following radioisotopes (e.g., ³H, ¹⁴C, ³⁵S, ¹²⁵I, ¹³¹I), fluorescent labels (e.g., FITC, rhodamine, lanthanide phosphors), luminescent labels such as luminol; enzymatic labels (e.g., horseradish peroxidase, beta-galactosidase, luciferase, alkaline phosphatase, acetylcholinestease), biotinyl groups (which can be detected by marked avidin, e.g., streptavidin containing a fluorescent marker or enzymatic activity that can be detected by optical or calorimetric methods), predetermined polypeptide epitopes recognized by a secondary reporter (e.g., leucine zipper pair sequences, binding sites for secondary antibodies, metal binding domains, epitope tags). Indirect methods may also be employed in which the primary antigen-antibody reaction is amplified by the introduction of a second antibody, having specificity for the antibody reactive against the prognostic antigen. By way of example, if the antibody having specificity against a prognostic antigen is a rabbit IgG antibody, the second antibody may be goat anti-rabbit gamma-globulin labeled with a detectable substance.

Methods for conjugating or labeling the antibodies discussed above may be readily accomplished by one of ordinary skill in the art. (See, for example, Imman, Methods In Enzymology, Vol. 34, Affinity Techniques, Enzyme Purification: Part B, Jakoby and Wichek (eds.), Academic Press, New York, p. 30, 1974; and Wilchek and Bayer, “The Avidin-Biotin Complex in Bioanalytical Applications,” Anal. Biochem., 1988, 171:1-32, regarding methods for conjugating or labeling the antibodies with an enzyme or ligand binding partner).

Time-resolved fluorometry may be used to detect a signal. For example, the method described in Christopoulos T. K. and Diamandis E. P., Anal. Chem., 1992:64:342-346 may be used with a conventional time-resolved fluorometer.

Therefore, in accordance with an embodiment of the invention, a method is provided wherein an antibody to a prognostic antigen of the invention is labeled with an enzyme, a substrate for the enzyme is added wherein the substrate is selected so that the substrate, or a reaction product of the enzyme and substrate, forms fluorescent complexes with a lanthanide metal. A lanthanide metal is added and the antigen is quantitated in the sample by measuring fluorescence of the fluorescent complexes. The antibodies specific for the antigen may be directly or indirectly labeled with an enzyme. Enzymes are selected based on the ability of a substrate of the enzyme, or a reaction product of the enzyme and substrate, to complex with lanthanide metals such as europium and terbium. Examples of suitable enzymes include alkaline phosphatase and beta-galactosidase. Preferably, the enzyme is alkaline phosphatase. The antibodies may also be indirectly labeled with an enzyme. For example, the antibodies may be conjugated to one partner of a ligand binding pair, and the enzyme may be coupled to the other partner of the ligand binding pair. Representative examples include avidin-biotin, and riboflavin-riboflavin binding protein. Preferably the antibodies are biotinylated, and the enzyme is coupled to streptavidin.

In an embodiment of the invention, antibody bound to a prognostic antigen of the invention in a sample is detected by adding a substrate for the enzyme. The substrate is selected so that in the presence of a lanthanide metal (e.g., europium, terbium, samarium, and dysprosium, preferably europium and terbium), the substrate or a reaction product of the enzyme and substrate, forms a fluorescent complex with the lanthanide metal. Examples of enzymes and substrates for enzymes that provide such fluorescent complexes are described in U.S. Pat. No. 5,312,922 to Diamandis. By way of example, when the antibody is directly or indirectly labeled with alkaline phosphatase, the substrate employed in the method may be 4-methylumbeliferyl phosphate, or 5-fluorpsalicyl phosphate. The fluorescence intensity of the complexes is typically measured using a time-resolved fluorometer, e.g., a CyberFluor 615 Immoanalyzer (Nordion International, Kanata Ontario).

The sample, antibody specific for the prognostic antigen, or the antigen itself, may be immobilized on a substrate. Examples of suitable substrates are agarose, cellulose, dextran, Sephadex, Sepharose, liposomes, carboxymethyl cellulose polystyrene, filter paper, ion-exchange resin, plastic film, plastic tube, glass beads, polyamine-methyl vinyl ether-maleic acid copolymer, amino acid copolymer, ethylene-maleic acid copolymer, nylon, silk, etc. The substrate may be in the shape of, for example, a tube, test plate, well, beads, disc, chip, sphere, etc. The immobilized antibody may be prepared by reacting the material with a suitable insoluble carrier using known chemical or physical methods, for example, cyanogen bromide coupling.

In accordance with an embodiment, the present invention provides a mode for determining the presence and, preferably, the abundance of prognostic antigens, antibodies to the antigens (which antibodies are themselves prognostic), or nucleic acid sequences encoding the antigens, in an appropriate sample such as blood or tumor tissue by measuring the antigen, antibody, or nucleic acids. The tumor antigens and nucleic acids can be removed from tumor tissue using methods known in the art. It will be evident to a skilled artisan that a variety of immunoassay methods can be used to measure these biomolecules. In general, an immunoassay method may be competitive or noncompetitive. Competitive methods typically employ an immobilized or immobilizable antibody to the antigen and a labeled form of the antigen. Sample antigen and labeled antigen compete for binding to the antibody. After separation of the resulting labeled antigen that has become bound to antibody (bound fraction) from that which has remained unbound (unbound fraction), the amount of the label in either bound or unbound fraction is measured and may be correlated with the amount of antigen in the biological sample in any conventional manner, e.g., by comparison to a standard curve.

Preferably, a noncompetitive method is used for the determination of two or more antigens of the invention, with the most common method being the “sandwich” method. In this assay, two anti-antigen antibodies, such as two anti-tumor antigen antibodies, are employed. One of the antibodies is directly or indirectly labeled (also referred to as the “detection antibody”) and the other is immobilized or immobilizable (also referred to as the “capture antibody”). The capture and detection antibodies can be contacted simultaneously or sequentially with the biological sample. Sequential methods can be accomplished by incubating the capture antibody with the sample, and adding the detection antibody at a predetermined time thereafter (sometimes referred to as the “forward” method); or the detection antibody can be incubated with the sample first and then the capture antibody added (sometimes referred to as the “reverse” method). After the necessary incubation(s) have occurred, to complete the assay, the capture antibody is separated from the liquid test mixture, and the label is measured in at least a portion of the separated capture antibody phase or the remainder of the liquid test mixture. Generally, it is measured in the capture antibody phase since it comprises the prognostic antigen bound by (“sandwiched” between) the capture and detection antibodies.

In a typical two-site immunometric assay for an antigen, one or both of the capture and detection antibodies are polyclonal antibodies. The label used in the detection antibody can be selected from any of those known conventionally in the art. As with other embodiments of the protein detection assay, the label can be an enzyme or a chemiluminescent moiety, for example, or a radioactive isotope, a fluorophore, a detectable ligand (e.g., detectable by a secondary binding by a labeled binding partner for the ligand), and the like. Preferably, the antibody is labeled with an enzyme that is detected by adding a substrate that is selected so that a reaction product of the enzyme and substrate forms fluorescent complexes. The capture antibody is selected so that it provides a mode for being separated from the remainder of the test mixture. Accordingly, the capture antibody can be introduced to the assay in an already immobilized or insoluble form, or can be in an immobilizable form, that is, a form which enables immobilization to be accomplished subsequent to introduction of the capture antibody to the assay. An immobilized capture antibody can comprise an antibody covalently or noncovalently attached to a solid phase (substrate) such as a magnetic particle, a latex particle, a microtiter multi-well plate, a bead, a cuvette, chip, slide, or other reaction vessel. An example of an immobilizable capture antibody is an antibody that has been chemically modified with a ligand moiety, e.g., a hapten, biotin, or the like, and that can be subsequently immobilized by contact with an immobilized form of a binding partner for the ligand, e.g., an antibody, avidin, or the like. In an embodiment, the capture antibody can be immobilized using a species specific antibody for the capture antibody that is bound to the solid phase.

A particular sandwich immunoassay method of the invention employs two antibodies reactive against an antigen of the invention, a second antibody having specificity against an antibody reactive against the antigen labeled with an enzymatic label, and a fluorogenic substrate for the enzyme. In an embodiment, the enzyme is alkaline phosphatase (ALP) and the substrate is 5-fluorosalicyl phosphate. ALP cleaves phosphate out of the fluorogenic substrate, 5-fluorosalicyl phosphate, to produce 5-fluorosalicylic acid (FSA). 5-Fluorosalicylic acid can then form a highly fluorescent ternary complex of the form FSA-Tb(3+)-EDTA, which can be quantified by measuring the Tb³⁺ fluorescence in a time-resolved mode. Fluorescence intensity is typically measured using a time-resolved fluorometry as described herein.

The above-described immunoassay methods and formats are intended to be exemplary and are not limiting since, in general, it will be understood that any immunoassay method or format can be used in the present invention.

The detection methods, arrays, and kits of the invention can utilize nanowire sensor technology (Zhen et al., Nature Biotechnology, 2005, 23(10):1294-1301; Lieber et al., Anal. Chem., 2006, 78(13):4260-4269, which are incorporated herein by reference) or microcantilever technology (Lee et al., Biosens. Bioelectron, 2005, 20(10):2157-2162; Wee et al., Biosens. Bioelectron., 2005, 20(10):1932-1938; Campbell and Mutharasan, Biosens. Bioelectron., 2005, 21(3):462-473; Campbell and Mutharasan, Biosens. Bioelectron., 2005, 21(4):597-607; Hwang et al., Lab Chip, 2004, 4(6):547-552; Mukhopadhyay et al., Nano. Lett., 2005, 5(12):2835-2388, which are incorporated herein by reference) for detection of one or more antigens, antibodies, or nucleic acid sequences in samples. In addition, Huang et al. describe a prostate specific antigen immunoassay on a commercially available surface plasmon resonance biosensor (Biosens. Bioelectron., 2005, 21(3):483-490) which may be adapted for detection of one or more antigens of the invention. High-sensitivity miniaturized immunoassays may also be utilized for detection of the antigens (Cesaro-Tadic et al., Lab Chip, 2004, 4(6):563-569; Zimmerman et al., Biomed. Microdevices, 2005, 7(2):99-110).

### Nucleic Acids

Nucleic acids including naturally occurring nucleic acids, oligonucleotides, antisense oligonucleotides, and synthetic oligonucleotides that hybridize to target nucleic acids within target genes or transcripts (e.g., encoding prognostic antigens), are useful as agents to detect the presence of nucleic acids encoding the antigens in biological samples of subjects, such as tumor samples. The present invention contemplates the use of nucleic acid sequences corresponding to the coding sequence of the prognostic antigens and to the complementary sequence thereof, as well as sequences complementary to the antigen transcript sequences occurring further upstream or downstream from the coding sequence (e.g., sequences contained in, or extending into, the 5′ and 3′ untranslated regions) for use as agents for detecting the expression of prognostic antigens in samples of subjects.

The preferred oligonucleotides for detecting the presence of prognostic antigens in samples are those that are complementary to at least part of the cDNA sequence encoding the antigen. These complementary sequences are also known in the art as “antisense” sequences. These oligonucleotides may be oligoribonucleotides or oligodeoxyribonucleotides. In addition, oligonucleotides may be natural oligomers composed of the biologically significant nucleotides, i.e., A (adenine), dA (deoxyadenine), G (guanine), dG (deoxyguanine), C (cytosine), dC (deoxycytosine), T (thymine) and U (uracil), or modified oligonucleotide species, substituting, for example, a methyl group or a sulfur atom for a phosphate oxygen in the inter-nucleotide phosphodiester linkage. Additionally, these nucleotides themselves, and/or the ribose moieties may be modified.

The oligonucleotides may be synthesized chemically, using any of the known chemical oligonucleotide synthesis methods well described in the art. For example, the oligonucleotides can be prepared by using any of the commercially available, automated nucleic acid synthesizers. Alternatively, the oligonucleotides may be created by standard recombinant DNA techniques, for example, inducing transcription of the noncoding strand. The DNA sequence encoding the prognostic antigen may be inverted in a recombinant DNA system, e.g., inserted in reverse orientation downstream of a suitable promoter, such that the noncoding strand now is transcribed.

Although any length oligonucleotide may be utilized to hybridize to a target nucleic acid within antigen genes or transcripts (e.g., to a nucleic acid encoding an antigen), oligonucleotides typically within the range of 8-100 nucleotides are preferred. Most preferable oligonucleotides for use in detecting antigens in biological samples are those within the range of 15-50 nucleotides.

In some embodiments, the substrate (e.g., solid support) of the array of the invention has no more than 500 oligonucleotides attached to it. In some embodiments, the substrate has no more than 100 oligonucleotides attached to it. In some embodiments, the substrate has no more than 50 oligonucleotides attached to it. In some embodiments, the substrate has no more than 20 oligonucleotides attached to it. In some embodiments, the substrate has no more than 10 oligonucleotides attached to it. In some embodiments, the substrate has no more than 5 oligonucleotides attached to it. In some embodiments, the substrate has no more than 4 oligonucleotides attached to it. In some embodiments, the substrate has no more than 3 oligonucleotides attached to it. In some embodiments, the substrate has no more than 2 oligonucleotides attached to it.

When referring to hybridization of one nucleic to another, “low stringency conditions” means in 10% formamide, 5× Denhart's solution, 6×SSPE, 0.2% SDS at 42° C., followed by washing in 1×SSPE, 0.2% SDS, at 50° C.; “moderate stringency conditions” means in 50% formamide, 5×Denhart's solution, 5×SSPE, 0.2% SDS at 42° C., followed by washing in 0.2×SSPE, 0.2% SDS, at 65° C.; and “high stringency conditions” means in 50% formamide, 5×Denhart's solution, 5×SSPE, 0.2% SDS at 42° C., followed by washing in 0.1×SSPE, and 0.1% SDS at 65° C. The phrase “stringent hybridization conditions” means low, moderate, or high stringency conditions.

The oligonucleotide selected for hybridizing to the nucleic acid molecule encoding the prognostic antigen, whether synthesized chemically or by recombinant DNA technology, can be isolated and purified using standard techniques and then preferably labeled (e.g., with ³⁵S or ³²P) using standard labeling protocols. Oligonucleotides can be attached or immobilized to a suitable solid support using methods known in the art.

The present invention also contemplates the use of oligonucleotide pairs (e.g., primers) in polymerize chain reactions (PCR) to detect the expression of the antigen in biological samples. The oligonucleotide pairs include a forward primer and a reverse primer.

The presence of antigen in a sample from a subject may be determined by nucleic acid hybridization, such as but not limited to Northern blot analysis, dot blotting, Southern blot analysis, fluorescence in situ hybridization (FISH), and PCR. Chromatography, preferably HPLC, and other known assays may also be used to determine messenger RNA levels of antigens in a sample.

Nucleic acid molecules encoding prognostic antigens can be found in the biological fluids inside a cancer cell that is present in a biological sample under investigation (e.g., blood or tissue). Nucleic acids encoding antigens may also be found directly (i.e., cell-free) in the fluid or biological sample, e.g., blood.

In one aspect, the present invention contemplates the use of nucleic acids as agents (oligonucleotides) for detecting prognostic antigens in samples, wherein the nucleic acids are labeled. The oligonucleotides may be labeled with a radioactive label, a fluorescent label, an enzyme, a chemiluminescent tag, a colorimetric tag or other labels or tags that are discussed above or that are known in the art.

In another aspect, the present invention contemplates the use of Northern blot analysis to detect the presence of prognostic antigen mRNA in a sample. The first step of the analysis involves separating a sample containing antigen-encoding nucleic acid by gel electrophoresis. The dispersed nucleic acids are then transferred to a nitrocellulose filter or another filter. Subsequently, the labeled oligonucleotide is exposed to the filter under suitable hybridizing conditions, e.g., 50% formamide, 5×SSPE, 2×Denhardt's solution, 0.1% SDS at 42° C., as described in Molecular Cloning: A Laboratory Manual, Maniatis et al. (1982, CSH Laboratory). Other useful procedures known in the art include solution hybridization, dot and slot RNA hybridization, and probe-based microarrays. Measuring the radioactivity of hybridized fragments, using standard procedures known in the art quantitates the amount of nucleic acid present in the sample of a subject.

Dot blotting involves applying samples containing the nucleic acid of interest to a membrane. The nucleic acid can be denatured before or after application to the membrane. The membrane is incubated with a labeled probe. Dot blot procedures are well known to the skilled artisan and are described more fully in U.S. Pat. Nos. 4,582,789 and 4,617,261, the disclosures of which are incorporated herein by reference.

Polymerase chain reaction (PCR) is a process for amplifying one or more target nucleic acid sequences present in a nucleic acid sample using primers and agents for polymerization and then detecting the amplified sequence. The extension product of one primer when hybridized to the other becomes a template for the production of the desired specific nucleic acid sequence, and vice versa, and the process is repeated as often as is necessary to produce the desired amount of the sequence. The skilled artisan to detect the presence of desired sequence (U.S. Pat. No. 4,683,195) routinely uses polymerase chain reaction.

A specific example of PCR that is routinely performed by the skilled artisan to detect desired sequences is reverse transcript PCR (RT-PCR; Saiki et al., Science, 1985, 230:1350; Scharf et al., Science, 1986, 233:1076). RT-PCR involves isolating total RNA from biological fluid, denaturing the RNA in the presence of primers that recognize the desired nucleic acid sequence, using the primers to generate a cDNA copy of the RNA by reverse transcription, amplifying the cDNA by PCR using specific primers, and detecting the amplified cDNA by electrophoresis or other methods known to the skilled artisan.

In a preferred embodiment, the methods of detecting nucleic acids encoding prognostic antigens in samples of subjects include Northern blot analysis, dot blotting, Southern blot analysis, FISH, and PCR.

The methods of the invention can be carried out on a substrate (e.g., solid or semi-solid support). The solid supports used may be those which are conventional for the purpose of assaying an analyte in a biological sample, and are typically constructed of materials such as cellulose, polysaccharide such as Sephadex, and the like, and may be partially surrounded by a housing for protection and/or handling of the solid support. The solid support can be rigid, semi-rigid, flexible, elastic (having shape-memory), etc., depending upon the desired application. Prognostic antigens of the invention can be detected in a sample in vivo or in vitro (ex vivo). When, according to an embodiment of the invention, the amount of antigen in a sample is to be determined without removing the sample from the body (i.e., in vivo, such as with an indwelling catheter or probe), the support should be one which is harmless to the subject and may be in any form convenient for insertion into an appropriate part of the body. For example, the support may be a probe made of polytetrafluoroethylene, polystyrene or other rigid non-harmful plastic material and having a size and shape to enable it to be introduced into a subject. The selection of an appropriate inert support is within the competence of those skilled in the art, as are its dimensions for the intended purpose.

A contacting step made in determining biomarker levels in an assay (method) of the invention can involve contacting, combining, or mixing the biological sample and the solid support, such as a reaction vessel, microvessel, tube, microtube, well, multi-well plate, or other solid support. In an embodiment of the invention, the solid support to be contacted with the biological sample (e.g., blood) has an absorbent pad or membrane for lateral flow of the liquid medium to be assayed, such as those available from Millipore Corp. (Bedford, Mass.), including but not limited to Hi-Flow Plus™ membranes and membrane cards, and SureWick™ pad materials.

Arrays useful in carrying out the methods of the invention can be constructed in any form adapted for the intended use. Thus, in one embodiment, the device can be constructed as a disposable or reusable test strip or stick to be contacted with a sample for which the presence of antigen/antibody/nucleic acid sequence or level thereof is to be determined. In another embodiment, the device can be constructed using art recognized micro-scale manufacturing techniques to produce needle-like embodiments capable of being implanted or injected into an anatomical site, such as a vein or artery, for indwelling diagnostic applications. In other embodiments, devices intended for repeated laboratory use can be constructed in the form of an elongated probe or catheter, for sampling of blood.

In some embodiments, the arrays of the invention comprise a solid support (such as a strip or dipstick), with a surface that functions as a lateral flow matrix defining a flow path for a biological sample such as blood.

Immunochromatographic assays, also known as lateral flow test strips or simply strip tests, for detecting various analytes of interest, have been known for some time, and may be used for detection of prognostic antigens of the invention. The benefits of lateral flow tests include a user-friendly format, rapid results, long-term stability over a wide range of climates, and relatively low cost to manufacture. These features make lateral flow tests ideal for applications involving home testing, rapid point of care testing, and testing in the field for various analytes. The principle behind the test is straightforward. Essentially, any ligand that can be bound to a visually detectable solid support, such as dyed microspheres, can be tested for, qualitatively, and in many cases even semi-quantitatively. For example, a one-step lateral flow immunostrip for the detection of free and total prostate specific antigen in serum is described in Fernandez-Sanchez et al. (J. Immuno. Methods, 2005, 307(1-2):1-12, which is incorporated herein by reference) and may be adapted for detection of prognostic antigens of the invention in a biological sample such as blood.

Some of the more common immunochromatographic assays currently on the market are tests for pregnancy (as an over-the-counter (OTC) test kit), Strep throat, and Chlamydia. Many new tests for well-known antigens have been recently developed using the immunochromatographic assay method. For instance, the antigen for the most common cause of community acquired pneumonia has been known since 1917, but a simple assay was developed only recently, and this was done using this simple test strip method (Murdoch, D. R. et al. J Clin Microbiol, 2001, 39:3495-3498). Human immunodeficiency virus (HIV) has been detected rapidly in pooled blood using a similar assay (Soroka, S. D. et al. J Clin Virol, 2003, 27:90-96). A nitrocellulose membrane card has also been used to diagnose schistosomiasis by detecting the movement and binding of nanoparticles of carbon (van Dam, G. J. et al. J Clin Microbiol, 2004, 42:5458-5461).

The two common approaches to the immunochromatographic assay are the noncompetitive (or direct) and competitive (or competitive inhibition) reaction schemes (TechNote #303, Rev. #001, 1999, Bangs Laboratories, Inc., Fishers, Ind.). The direct (double antibody sandwich) format is typically used when testing for larger analytes with multiple antigenic sites such as luteinizing hormone (LH), human chorionic gonadotropin (hCG), and HIV. In this instance, less than an excess of sample analyte is desired, so that some of the microspheres will not be captured at the capture line, and will continue to flow toward the second line of immobilized antibodies, the control zone. This control line uses species-specific anti-immunoglobulin antibodies, specific for the conjugate antibodies on the microspheres. Free antigen, if present, is introduced onto the device by adding sample (blood, etc.) onto a sample addition pad. Free antigen then binds to antibody-microsphere complexes. Antibody 1, specific for epitope 1 of sample antigen, is coupled to dye microspheres and dried onto the device. When sample is added, microsphere-antibody complex is rehydrated and carried to a capture zone and control lines by liquid. Antibody 2, specific for a second antigenic site (epitope 2) of sample antigen, is dried onto a membrane at the capture line. Antibody 3, a species-specific, anti-immunoglobulin antibody that will react with antibody 1, is dried onto the membrane at the control line. If antigen is present in the sample (i.e., a positive test), it will bind by its two antigenic sites, to both antibody 1 (conjugated to microspheres) and antibody 2 (dried onto membrane at the capture line). Antibody 1-coated microspheres are bound by antibody 3 at the control line, whether antigen is present or not. If antigen is not present in the sample (a negative test), microspheres pass the capture line without being trapped, but are caught by the control line.

The competitive reaction scheme is typically used when testing for small molecules with single antigenic determinants, which cannot bond to two antibodies simultaneously. As with double antibody sandwich assay, free antigen, if present is introduced onto the device by adding sample onto a sample pad. Free antigen present in the sample binds to an antibody-microsphere complex. Antibody 1 is specific for sample antigen and couple to dyed microspheres. An antigen-carrier molecule (typically BSA) conjugate is dried onto a membrane at the capture line. Antibody 2 (Ab2) is dried onto the membrane at the control line, and is a species-specific anti-immunoglobulin that will capture the reagent particles and confirm that the test is complete. If antigen is present in the sample (a positive test), antibody on microspheres (Ab1) is already saturated with antigen from sample and, therefore, antigen conjugate bound at the capture line does not bind to it. Any microspheres not caught by the antigen carrier molecule can be caught by Ab2 on the control line. If antigen is not present in the sample (a negative test), antibody-coated dyed microspheres are allowed to be captured by antigen conjugate bound at the capture line.

Normally, the membranes used to hold the antibodies in place on these devices are made of primary hydrophobic materials, such as nitrocellulose. Both the microspheres used as the solid phase supports and the conjugate antibodies are hydrophobic, and their interaction with the membrane allows them to be effectively dried onto the membrane.

As used herein, the term “ELISA” includes an enzyme-linked immunoabsorbent assay that employs an antibody or antigen bound to a solid phase and an enzyme-antigen or enzyme-antibody conjugate to detect and quantify the amount of an antigen (e.g., biomarker of the invention) or antibody present in a sample. A description of the ELISA technique is found in Chapter 22 of the 4^{th} Edition of Basic and Clinical Immunology by D. P. Sites et al., 1982, published by Lange Medical Publications of Los Altos, Calif. and in U.S. Pat. Nos. 3,654,090; 3,850,752; and 4,016,043, the disclosures of which are herein incorporated by reference. ELISA is an assay that can be used to quantitate the amount of antigen, proteins, or other molecules of interest in a sample. In particular, ELISA can be carried out by attaching on a solid support (e.g., polyvinylchloride) an antibody specific for an antigen or protein of interest. Cell extract or other biological sample of interest such as blood can be added for formation of an antibody-antigen complex, and the extra, unbound sample is washed away. An enzyme-linked antibody, specific for a different site on the antigen is added. The support is washed to remove the unbound enzyme-linked second antibody. The enzyme-linked antibody can include, but is not limited to, alkaline phosphatase. The enzyme on the second antibody can convert an added colorless substrate into a colored product or can convert a non-fluorescent substrate into a fluorescent product. The ELISA-based assay method provided herein can be conducted in a single chamber or on an array of chambers and can be adapted for automated processes.

In these exemplary embodiments, the antibodies can be labeled with pairs of FRET dyes, bioluminescence resonance energy transfer (BRET) protein, fluorescent dye-quencher dye combinations, beta gal complementation assays protein fragments. The antibodies may participate in FRET, BRET, fluorescence quenching or beta-gal complementation to generate fluorescence, colorimetric or enhanced chemiluminescence (ECL) signals, for example.

These methods are routinely employed in the detection of antigen-specific antibody responses, and are well described in general immunology text books such as Immunology by Ivan Roitt, Jonathan Brostoff and David Male (London: Mosby, c1998. 5th ed. and Immunobiology: Immune System in Health and Disease/Charles A. Janeway and Paul Travers. Oxford: Blackwell Sci. Pub., 1994), the contents of which are herein incorporated by reference.

Compounds useful in the treatment and prognostic methods of the subject invention, such as immunotherapies and other therapeutic agents, can be formulated according to known methods for preparing pharmaceutically useful compositions. Formulations are described in detail in a number of sources which are well known and readily available to those skilled in the art. For example, Remington's Pharmaceutical Science by E. W. Martin describes formulations which can be used in connection with the subject invention. In general, the compositions of the subject invention will be formulated such that an effective amount of the compound is combined with a suitable carrier in order to facilitate effective administration of the composition. The compositions used in the present methods can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically acceptable carriers and diluents which are known to those skilled in the art. Examples of carriers or diluents for use with the subject compounds include ethanol, dimethyl sulfoxide, glycerol, alumina, starch, and equivalent carriers and diluents. To provide for the administration of such dosages for the desired therapeutic treatment, pharmaceutical compositions of the invention will advantageously comprise between about 0.1% and 99%, and especially, 1 and 15% by weight of the total of one or more of the subject compounds based on the weight of the total composition including carrier or diluent.

### EXEMPLIFIED EMBODIMENTS

### Embodiment 1

A method for predicting a clinical response (efficacy) and/or adverse event to an immunotherapy for treatment of a malignancy in a subject, comprising:

(a) determining the level of two or more biomarkers in a biological sample taken from the subject before or after initiation of the immunotherapy, and wherein the two or more biomarkers comprise or consist of:
(1) immunoglobulins to two or more antigens selected from among BRAF, CABYR, CRISP3, CSAG2, CTAG2, CXorf48.1, DHFR, FTHL17, GAGE1, GAGE2A, GLUD1, LDHC, MAGEA1, MAGEA3, MAGEA4v2, MAGEA4v3, MAGEA4v4, MAGEB6, MAPK1, MICA, MUC1, NLRP4, NY-ESO-1, PBK, PRAME, SOX2, SILV, SPANXA1, SPANXB1, SSX2A, SSX4, TSGA10, TSSK6, TULP2, TYR, XAGE-2, and ZNF165; or
(2) two or more antigens selected from those set forth in (a)(1); or
(3) nucleic acid sequences that encode two or more antigens selected from those set forth in (a)(1); or
(4) T-cells activated against two or more antigens selected from those set forth in (a)(1); and

(b) correlating the level of the two or more biomarkers in the sample with a predicted clinical response and/or likelihood of an adverse event in the subject.

### Embodiment 2

The method of embodiment 1, wherein the two or more antigens comprise or consist of the group of antigens of example combination A, example combination B, example combination C, example combination D, example combination E, example combination F, example combination G, example combination H, example combination I, or example combination J.

### Embodiment 3

The method of embodiment 1, wherein the two or more antigens comprise or consist of CSAG2, MAGEA1, MAGEA3, MAGEA4v2, MICA, NLRP4, SILV, SSX4, TSSK6, and XAGE-2.

### Embodiment 4

The method of embodiment 1, wherein the two or more antigens comprise or consist of two or more of BRAF, CABYR, CRISP3, CSAG2, CTAG2, DHFR, FTHL17, GAGE1, GLUD1, LDHC, MAGEA1, MAGEB6, MAPK1, FTHL17, SSX2, XAGE2, TULP2, PRAME, SOX2, SPANX-B1, SSX4, TSSK6, and SSX5.

### Embodiment 5

The method of embodiment 1, wherein said correlating of (b) comprises comparing the level of the two or more biomarkers in the sample to a reference level of the two or more biomarkers, wherein the relationship between the level of the two or more biomarkers in the sample and the reference level is indicative of the clinical response and/or the likelihood of an adverse event.

### Embodiment 6

The method of embodiment 5, wherein the reference level is the level of a normal subject, or a normal population of subjects, or a subject having the same malignancy, or a population having the same malignancy.

### Embodiment 7

The method of embodiment 1, wherein said determining of (a) comprises measuring the level of the two or more biomarkers in a biological sample taken from the subject, and said correlating of (b) comprises comparing the measured level of the two or more biomarkers to a reference level of the two or more biomarkers, wherein the relationship between the level of the two or more biomarkers in the sample and the reference level is indicative of the clinical response and/or the likelihood of an adverse event.

### Embodiment 8

The method of embodiment 5, wherein the sample is obtained from the subject after initiation of the immunotherapy, and wherein the reference level is the level of the two or more biomarkers in a sample taken from the subject before initiation of the immunotherapy.

### Embodiment 9

The method of embodiment 1, wherein a significant increase in the level of two, three, or more biomarkers (e.g., 50%+) after immunotherapy is predictive of (correlates with) an adverse event.

### Embodiment 10

The method of embodiment 1, wherein lack of a significant increase (e.g., not having 50%+) in the level of two, three, or more biomarkers after immunotherapy is predictive of (correlates with) an absence of an adverse event.

### Embodiment 11

The method of embodiment 1, wherein a significant increase (e.g., 50%+) in seroreactivity to two, three, or more of the antigens after immunotherapy is predictive of (correlates with) an adverse event.

### Embodiment 12

The method of embodiment 1, wherein lack of a significant increase (e.g., not having 50%+) in seroreactivity to two, three, or more antigens after immunotherapy is predictive of (correlates with) an absence of an adverse event.

### Embodiment 13

The method of embodiment 1, wherein if the level of two, three, four, five, or more biomarkers did not reach a threshold level, the subject is predicted to have a poor clinical response (e.g., survival of 300 days or less).

### Embodiment 14

The method of embodiment 1, wherein if the level of two, three, four, five, or more biomarkers reached a threshold level, the subject is predicted to have a positive clinical response (treatment efficacy), (e.g., survival more than 300 days).

### Embodiment 15

The method of embodiment 1, wherein the biomarkers comprise or consist of (a)(1), and wherein the biological sample is serum.

### Embodiment 16

The method of embodiment 1, wherein the biomarkers comprise or consist of (a)(1) or (a)(2), and wherein the biological sample comprises cells of a malignancy.

### Embodiment 17

The method of embodiment 1, wherein the malignancy is selected from among melanoma, ovarian cancer, breast cancer, lung cancer (small cell or non-small cell), esophageal cancer, sarcoma, or colorectal cancer.

### Embodiment 18

The method of embodiment 1, wherein the adverse event comprises autoimmune toxicity.

### Embodiment 19

The method of embodiment 18, wherein the autoimmune toxicity comprises a gastrointestinal autoimmune side effect (colitis, stomach pain, bloating, constipation, diarrhea), dermatitis, anti-pituitary autoimmune side effect, hepatitis, inflammation of the hormone gland(s), inflammation of the eyes, inflammation of the nerves, or two or more of the foregoing.

### Embodiment 20

The method of embodiment 1, wherein the immunotherapy comprises an agent selected from among a cancer vaccine, immunomodulator, monoclonal antibody, immunostimulant, dendritic cell, viral therapy.

### Embodiment 21

The method of embodiment 20, wherein the immunotherapy comprises an antibody that binds to cytotoxic T lymphocyte-associated antigen 4 (CTLA-4) (e.g., Ipilimumab), a p53 cancer vaccine, 1-methyl-D-tryptophan (1MT), or autologous dendritic cells activated against an antigen of the malignancy (for example prostatic acid phosphatase (PAP), e.g., sipuleucel-T).

### Embodiment 22

The method of embodiment 21, wherein the immunotherapy comprises an antibody that binds to cytotoxic T lymphocyte-associated antigen 4 (CTLA-4) (e.g., Ipilimumab), and wherein the malignancy comprises melanoma, prostate cancer, or lung cancer.

### Embodiment 23

The method of embodiment 1, wherein the two or more antigens comprise or consist of two or more of BRAF, CABYR, CRISPS, CSAG2, CTAG2, DHFR, FTHL17, GAGE1, GLUD1, LDHC, MAGEA1, MAGEB6, MAPK1, FTHL17, SSX2, XAGE2, TULP2, PRAME, SOX2, SPANX-B1, SSX4, TSSK6, and SSX5; wherein the malignancy is selected from among melanoma, ovarian cancer, breast cancer, lung cancer (small cell or non-small cell), esophageal cancer, sarcoma, or colorectal cancer; and wherein the immunotherapy comprises an antibody that binds to cytotoxic T lymphocyte-associated antigen 4 (CTLA-4).

### Embodiment 24

The method of embodiment 1, wherein said correlating of (b) comprises determining a value (score) representative of the number of biomarker levels that meet or exceed a reference threshold level, and comparing the determined score to one or more reference scores, wherein the relationship between the determined score and the one or more reference scores is predictive of (correlates with) an adverse event or absence of an adverse event.

### Embodiment 25

The method of embodiment 24, wherein the method further comprises categorizing the subject (assigning a category) based on the relationship between the determined score and the reference score, wherein the assigned category is representative of the likelihood of positive clinical response to immunotherapy, or likelihood of an adverse event.

### Embodiment 26

The method of embodiment 25, wherein the subject is categorized into one of two categories (e.g., “low” or “high”).

### Embodiment 27

The method of embodiment 25, wherein the determined score is compared to a plurality of scores, and the method further comprises categorizing the subject based on the relationship between the determined score and the plurality of reference scores.

### Embodiment 28

The method of embodiment 27, wherein the subject is categorized into one of three categories (e.g., “low”, “medium”, or “high”).

### Embodiment 29

An immunotherapeutic agent for use in treatment of a malignancy in a subject, the treatment comprising the following prior to administration of the immunotherapeutic agent:

(a) determining the level of two or more biomarkers in a biological sample taken from the subject before or after initiation of the immunotherapy, and wherein the two or more biomarkers comprise or consist of:
(1) immunoglobulins to two or more antigens selected from among BRAF, CABYR, CRISP3, CSAG2, CTAG2, CXorf48.1, DHFR, FTHL17, GAGE1, GAGE2A, GLUD1, LDHC, MAGEA1, MAGEA3, MAGEA4v2, MAGEA4v3, MAGEA4v4, MAGEB6, MAPK1, MICA, MUC1, NLRP4, NY-ESO-1, PBK, PRAME, SOX2, SILV, SPANXA1, SPANXB1, SSX2A, SSX4, TSGA10, TSSK6, TULP2, TYR, RAGE-2, and ZNF165; or
(2) two or more antigens selected from those set forth in (a)(1); or
(3) nucleic acid sequences that encode two or more antigens selected from those set forth in (a)(1); or
(4) T-cells activated against two or more antigens selected from those set forth in (a)(1); and

(b) correlating the level of the two or more biomarkers in the sample with a predicted clinical response and/or likelihood of an adverse event in the subject.

### Embodiment 30

An array comprising a substrate and two or more capture probes disposed thereon, wherein said two or more capture probes comprise or consist of:

(a) at least antigenic epitopes of two or more antigens selected from among BRAF, CABYR, CRISP3, CSAG2, CTAG2, CXorf48.1, DHFR, FTHL17, GAGE1, GAGE2A, GLUD1, LDHC, MAGEA1, MAGEA3, MAGEA4v2, MAGEA4v3, MAGEA4v4, MAGEB6, MAPK1, MICA, MUC1, NLRP4, NY-ESO-1, PBK, PRAME, SOX2, SILV, SPANXA1, SPANXB1, SSX2A, SSX4, TSGA10, TSSK6, TULP2, TYR, XAGE-2, and ZNF165; or

(b) antibodies, or antibody fragments, that specifically bind two or more antigens from those set forth in (a); or

(c) oligonucleotides that are partially or fully complementary to, and bind to, nucleic acid sequences encoding two or more antigens from those set forth in (a).

### Embodiment 31

The array of embodiment 30, wherein the two or more antigens comprise or consist of the group of antigens of example combination A, example combination B, example combination C, example combination D, example combination E, example combination F, example combination G, example combination H, example combination I, or example combination J.

### Embodiment 32

The array of embodiment 30, wherein the two or more antigens comprise or consist of CSAG2, MAGEA1, MAGEA3, MAGEA4v2, MICA, NLRP4, SILV, SSX4, TSSK6, and XAGE-2.

### Embodiment 33

The array of embodiment 30, wherein the two or more antigens comprise or consist of two or more of BRAF, CABYR, CRISP3, CSAG2, CTAG2, DHFR, FTHL17, GAGE1, GLUD1, LDHC, MAGEA1, MAGEB6, MAPK1, FTHL17, SSX2, XAGE2, TULP2, PRAME, SOX2, SPANX-B1, SSX4, TSSK6, and SSX5.

### Embodiment 34

The array of embodiment 30, wherein said two or more capture probes comprise two or more full-length antigens of (a).

### Embodiment 35

The array of embodiment 30, wherein the substrate comprises a particle (e.g., magnetic or latex particle), a microtiter multi-well plate, a bead, a membrane, a cuvette, or a reaction vessel.

### Embodiment 36

The array of embodiment 30, comprising three, four, five, six, seven, eight, nine, ten, eleven, twelve, thirteen, fourteen, fifteen, sixteen, seventeen, eighteen, nineteen, twenty, twenty one, twenty two, twenty three, twenty four, twenty five, twenty six, twenty seven, twenty eight, twenty nine, thirty, thirty one, thirty two, thirty three, thirty four, thirty five, thirty six, or thirty seven of said capture probes.

### Embodiment 37

A kit for predicting a clinical response (efficacy) and/or adverse event to an immunotherapy, comprising two or more capture probes in one or more containers, wherein the capture probes comprise or consist of:

(a) at least antigenic epitopes of two or more antigens selected from among BRAF, CABYR, CRISPS, CSAG2, CTAG2, CXorf48.1, DHFR, FTHL17, GAGE1, GAGE2A, GLUD1, LDHC, MAGEA1, MAGEA3, MAGEA4v2, MAGEA4v3, MAGEA4v4, MAGEB6, MAPK1, MICA, MUC1, NLRP4, NY-ESO-1, PBK, PRAME, SOX2, SILV, SPANXA1, SPANXB1, SSX2A, SSX4, TSGA10, TSSK6, TULP2, TYR, XAGE-2, and ZNF165; or

(b) antibodies, or antibody fragments, that specifically bind two or more antigens from those set forth in (a); or

(c) oligonucleotides that bind to nucleic acid sequences encoding two or more antigens from those set forth in (a).

### Embodiment 38

The kit of embodiment 37, wherein the two or more antigens comprise or consist of the group of antigens of example combination A, example combination B, example combination C, example combination D, example combination E, example combination F, example combination G, example combination H, example combination I, or example combination J.

### Embodiment 39

The kit of embodiment 37, wherein the two or more antigens comprise or consist of CSAG2, MAGEA1, MAGEA3, MAGEA4v2, MICA, NLRP4, SILV, SSX4, TSSK6, and XAGE-2.

### Embodiment 40

The kit of embodiment 37, wherein the two or more antigens comprise or consist of two or more of BRAF, CABYR, CRISP3, CSAG2, CTAG2, DHFR, FTHL17, GAGE1, GLUD1, LDHC, MAGEA1, MAGEB6, MAPK1, FTHL17, SSX2, XAGE2, TULP2, PRAME, SOX2, SPANX-B1, SSX4, TSSK6, and SSX5.

### Embodiment 41

The kit of embodiment 37, wherein the one or more capture probes are disposed on a substrate.

### Embodiment 42

A method for treating or delaying the onset or relapse of a malignancy in a subject, comprising:

(a) predicting the clinical response (efficacy) and/or adverse event to an immunotherapy for treatment of a malignancy in a subject determined by the level of two or more biomarkers comprising or consisting of:
(1) immunoglobulins to two or more antigens selected from among BRAF, CABYR, CRISP3, CSAG2, CTAG2, CXorf48.1, DHFR, FTHL17, GAGE1, GAGE2A, GLUD1, LDHC, MAGEA1, MAGEA3, MAGEA4v2, MAGEA4v3, MAGEA4v4, MAGEB6, MAPK1, MICA, MUC1, NLRP4, NY-ESO-1, PBK, PRAME, SOX2, SILV, SPANXA1, SPANXB1, SSX2A, SSX4, TSGA10, TSSK6, TULP2, TYR, XAGE-2, and ZNF165; or
(2) two or more antigens selected from those set forth in (a)(1); or
(3) nucleic acid sequences that encode two or more antigens selected from those set forth in (a)(1); or
(4) T-cells activated against two or more antigens selected from those set forth in (a)(1); and

(b) administering an immunotherapy to the subject if it is predicted that the immunotherapy will have efficacy and/or will not result in an adverse event; or

(c) withholding the immunotherapy from the subject if it is predicted that the immunotherapy will not have efficacy and/or will result in an adverse event.

### Embodiment 43

The method of embodiment 42, wherein (c) further comprises administering a therapy other than an immunotherapy to the subject if it is predicted that the immunotherapy will not have efficacy and/or will result in an adverse event.

### Embodiment 44

The method of embodiment 43, wherein the therapy other than an immunotherapy comprises chemotherapy, radiation therapy, surgery, or a combination of two or three of the foregoing.

### Embodiment 45

A method for treating or delaying the onset or relapse of a malignancy in a subject, comprising carrying out the method of any one of embodiments 1 to 28, and further comprising:

(c) administering an immunotherapy to the subject if it is predicted that the immunotherapy will have efficacy and/or will not result in an adverse event; or

(d) withholding the immunotherapy from the subject if it is predicted that the immunotherapy will not have efficacy and/or will result in an adverse event.

### Embodiment 46

The method of embodiment 45, wherein (d) further comprises administering a therapy other than an immunotherapy to the subject if it is predicted that the immunotherapy will not have efficacy and/or will result in an adverse event.

### Embodiment 47

The method of embodiment 46, wherein the therapy other than an immunotherapy comprises chemotherapy, radiation therapy, surgery, or a combination of two or three of the foregoing.

The names, National Center for Biotechnology Information (NCBI) Reference Sequence Accession numbers, and nucleic acid sequences of the prognostic antigens (biomarkers) of the invention are as follows:

| 1. CSAG2 NM_004909 | |
|---|---|
| (SEQ ID NO: 1) | |
| 1 | gtgcaatggc tagtactatg tgtcaacttg tctaggctat actgctcagc tgtgtggtca |
| 61 | aacagtagtc tagatgttgc tgtgaaggta ttttgtagat gtgatcaaca tttacaatca |
| 121 | gttgatttta agtaaagcag tttaacttcc ataatgtgga tgggcctcat ccaattagtt |
| 181 | gaaggtgtta agagaaaaga ccaaggtttc ctggaaaagg aattctacca caagactaac |
| 241 | ataaaaatgc gctgtgagtt tctagcctgc tggcctgcct tcactgtcct gggggaggct |
| 301 | tggagagacc aggtggactg gagtagactg ttgagagacg ctggtctggt gaagatgtcc |
| 361 | aggaaaccac gagcctccag cccattgtcc aacaaccacc caccaacacc aaagaggttc |
| 421 | ccaagacaac ccggaaggga aaagggaccc atcaaggaag ttccaggaac aaaaggctct |
| 481 | ccctaaaaga ccgccgcttc aaaaaaacct gaggaatgga gtgggccaac actatccagc |
| 541 | cactctgacc agccgaacga ggaactcaat caaaatgagc catagcggga ccacaagggc |
| 601 | aaggagacca ccaccttctc cagtctctct tcggacagcc agtaattccc gggcaaggcc |
| 661 | agagacttca agtctatctg aaaagtctcc agaggtctaa ccccagataa atagccaaca |
| 721 | gggtgtagag tacattttac accccaaaga gtgtgcccca tggtgatgaa aataaagtga |
| 781 | acatgttgca aaatga |
| 2. CTAG2 NM_172377 | |
| (SEQ ID NO: 2) | |
| 1 | tctgcctccg catcctcgtg ggccctgacc ttctctctga gagccgggca gaggctccgg |
| 61 | agccatgcag gccgaaggcc agggcacagg gggttcgacg ggcgatgctg atggcccagg |
| 121 | aggccctggc attcctgatg gcccaggggg caatgctggc ggcccaggag aggcgggtgc |
| 181 | cacgggcggc agaggtcccc ggggcgcagg ggcagcaagg gcctcggggc cgagaggagg |
| 241 | cgccccgcgg ggtccgcatg gcggtgccgc ttctgcgcag gatggaaggt gcccctgcgg |
| 301 | ggccaggagg ccggacagcc gcctgcttca gttgcacatc acgatgcctt tctcgtcgcc |
| 361 | catggaagcg gagctggtcc gcaggatcct gtcccgggat gccgcacctc tcccccgacc |
| 421 | aggggcggtt ctgaaggact tcaccgtgtc cggcaaccta ctgtttatcc gactgactgc |
| 481 | tgcagaccac cgccaactgc agctctccat cagctcctgt ctccagcagc tttccctgtt |
| 541 | gatgtggatc acgcagtgct ttctgcccgt gtttttggct caggctccct cagggcagag |
| 601 | gcgctaagcc cagcctggcg ccccttccta ggtcatgcct cctcccctag ggaatggtcc |
| 661 | cagcacgagt ggccagttca ttgtgggggc ctgattgttt gtcgctggag gaggacggct |
| 721 | tacatgtttg tttctgtaga aaataaagct gagctacgat tccgaaaaaa aaa |
| 3. CXORF48.1 NM_017863 | |
| (SEQ ID NO: 3) | |
| 1 | ggcagtccta gtacacaaga cacgtacttg ccatcactaa aacattgcct acccacatga |
| 61 | tgccctctct ccacaaaatt gatccctggt gaggagtaac tattgaccgc ccactgagtg |
| 121 | gtgactcctt atgctaagca ccacgaatac aagggcagga gcaggatttt tggttgcaca |
| 181 | gccggtgttt caggcaccca tcctcccagg gaacccaaga gaagagccct gtatctcctt |
| 241 | agtaaatgca gaacccaagc tcccaagcca ttttccccag gccatcctaa tggctccctc |
| 301 | agccttgatg ggagaagcca cgatgttccc acccatcgcc ttttagtagc cacaagaacc |
| 361 | tgcccgccca cccactgcac gcccattggt tggacagtgg tagaggcggg ccctgacgag |
| 421 | cgcatgctca gagggagaag tcagcggaga agctgggagc tcctttggag gctgcggtgt |
| 481 | ccctgactct cctgaggggg gctcactaac gggtgggtcc catgccacag tgtctgagga |
| 541 | gaagcaggaa gtgagtccct gaggagacgc cgtgacctga gggcttccct tactgaggag |
| 601 | gcctcgtgct tcatctgcca caagcggtgc ccaggccggt ggtgacaact gggacgatgc |
| 661 | tcaggcttct gagacttgct ttggccttct acgggaggac ggccgaccct gcagagcgac |
| 721 | agggcccaca gcagcagggc ctcccacaag gtgacaccca gttgacaact gtgcagggag |
| 781 | ttgtcacaag tttctgtggt gattatggca tgattgatga gtcgatctac ttcagtagtg |
| 841 | atgttgtgac tggcaacgtg cctctaaaag ttggacaaaa agttaatgtg gttgtggaag |
| 901 | aagataaacc acattatgga ttgagagcaa tcaaggtgga tgttgtgcct cgccatcttt |
| 961 | atggtgctgg accctcagac tcaggaacca gagttttaat tggatgtgtt acttctataa |
| 1021 | atgaagataa tatttatatt agtaacagca tttatttttc catagccatt gtttctgaag |
| 1081 | attttgtgcc ttataagggt gacttgttag aagttgaata ttccactgag ccaggcatct |
| 1141 | caaacatcaa ggcaacttct gtgaagccca tccgttgtat tcatacggaa gaggtctgca |
| 1201 | ttactagcgt acatggaaga aatggggtga tagattatac tatttttttc accttggatt |
| 1261 | ctgtgaaact tcctgatgga tacgtacctc aagtagatga tatcgtcaat gtggtcatgg |
| 1321 | tggagagcat tcagttctgc tttatttgga gagcgatttc tatcacccca gtgcataaat |
| 1381 | cgtaatgaca aagcattttt attctgttta tctttccttt tatgagcagt aaaggggctg |
| 1441 | gtttaactta aaaggttagc ttagtaagcc taaatagtat tttatatatg acttttctgg |
| 1501 | caaatctaat tgagacactg gccagtccaa ctggaccagg aacccagctt agggaaataa |
| 1561 | cttattaatt aaaaagcatg ctaaattagc ttgctagtca ctggaggaaa ggagttctta |
| 1621 | attaaaatga acacggccat taaatttgaa ttccatattt cccattagca gcagcggatt |
| 1681 | ccaggatgac ggaggcctgg gacggcccaa aagggaacgt cggagccaaa gcatttaact |
| 1741 | gaaaaggcat cagggacagc atgttaaagg catgatttaa agttacaatt tgacttcagt |
| 1801 | tttgagcccc gttatgctgc ctgtacaacc tgtattgttc catagcctct ttcatcttct |
| 1861 | gtcacccaca taacttgcgg tgtttgttgt tttacttgtt ccttcttccc catgctagaa |
| 1921 | tgtaaactcc acaatggcag gatgtctttt aatctggttt ttttttttct tttttctttc |
| 1981 | tttttttttt tctgttgtca ttaaagcatc accaagcact tagaagagtg cctggcagac |
| 2041 | tcaatgagca gataaaatgt tattaagttc aatgactgaa tgactgggcc agcaatgata |
| 2101 | cttacctgtg gttcattcat cactggttcc ttaggaataa aattgttgag caatgcacta |
| 2161 | aaaaaaaaaa aaaaaaaaaa aaaaaaaa |
| 4. FTHL17 NM_031894 | |
| (SEQ ID NO: 4) | |
| 1 | cacccgcctt tcactatccg ccattcttgt cacctcagct gctgccctcg ctaccgcacc |
| 61 | gacttcgccc gtgtgctcgc ctgcacttgc gctgcccgcc atggccaccg cccagccgtc |
| 121 | gcaggtgcgc cagaagtacg acaccaactg cgacgccgcc atcaacagcc acatcacgct |
| 181 | ggagctctac acctcctacc tgtacctgtc tatggccttc tacttcaacc gggacgacgt |
| 241 | ggccctggag aacttcttcc gctacttcct gcgcctgtcg gacgacaaaa tggagcatgc |
| 301 | ccagaagctg atgaggctgc agaacctgcg cggtggccac atctgccttc acgatatcag |
| 361 | gaagccagag tgccaaggct gggagagcgg gctcgtggcc atggagtccg ccttccacct |
| 421 | ggagaagaac gtcaaccaga gcctgctgga tctgtaccag ctggccgtgg agaagggcga |
| 481 | cccccagctg tgccacttcc tggagagcca ctacctgcac gagcaagtca agaccatcaa |
| 541 | agagctgggt ggctacgtga gcaacctgcg caagatttgt tccccggaag ccggcctggc |
| 601 | tgagtacctg ttcgacaagc tcaccctggg cggccgcgtc aaagagactt gagcccagat |
| 661 | gggccccaca gccacggggt cccttccctg ggtcaggcca ctaggcgggg cgtgcatgtt |
| 721 | gccctttcag aacgttctct tcagttttat ctttcagttt taccattgtt agcaaaaaag |
| 781 | ttatctggtt ctcaaagcaa taaaggtgtc cataaaaaaa aaaaaaaaaa |
| 5. GAGE1 NM_001468 | |
| (SEQ ID NO: 5) | |
| 1 | actgggcgtc ttctgcccgg ccccttcgcc cacgtgaaga acgccaggga gctgtgaggc |
| 61 | agtgctgtgt ggttcctgcc gtccggactc tttttcctct actgagattc atctgtgtga |
| 121 | aatatgagtt ggcgaggaag atcgacctat tattggccta gaccaaggcg ctatgtacag |
| 181 | cctcctgaaa tgattgggcc tatgcggccc gagcagttca gtgatgaagt ggaaccagca |
| 241 | acacctgaag aaggggaacc agcaactcaa cgtcaggatc ctgcagctgc tcaggaggga |
| 301 | gaggatgagg gagcatctgc aggtcaaggg ccgaagcctg aagctgatag ccaggaacag |
| 361 | ggtcacccac agactgggtg tgagtgtgaa gatggtcctg atgggcagga gatggacccg |
| 421 | ccaaatccag aggaggtgaa aacgcctgaa gaagagatga ggtctcacta tgttgcccag |
| 481 | actgggattc tctggctttt aatgaacaat tgcttcttaa atctttcccc acggaaacct |
| 541 | tgagtgactg aaatatcaaa tggcaagaga ccgtttagtt cctatcatct gtggcatgtg |
| 601 | aagggcaatc acagtgttaa aagaagacat gctgaaatgt tgcaggctgc tcctatgttg |
| 661 | gaaaattctt cattgaagtt ctcccaataa agctttacag ccttctgcaa agaagtcttg |
| 721 | tgaatctttt gtcaatttta tttctagcta tttgatgctg tgaaatgttt cattctttgc |
| 781 | aattttgtat tctatctcct tgagctgtgt gtagaggcat aattctcatg tattgatttt |
| 841 | ctatccagca accttgttaa atatgcttat gaattttaaa agtttacttc taggtttttt |
| 901 | cagttttcaa cctacagaat catatcattt ttgaataaga acaattttgt ttctgccttt |
| 961 | tttgtttgtt ttttcttttg tatttttcgt agaggtggga ttttggcctg tgtcctaggc |
| 1021 | tttttttgaa ctcctgagcg caagtaatcc actctccttg gcctttcaaa gtgttgggat |
| 1081 | tacaggcatg ggccaccgtg ctggtcctgt ttttgccatt ttaaaccctt ttatttcctt |
| 1141 | ttctgatttt atggcattga gcagatctac cggatacaat tgtgatagtg gaaatttttg |
| 1201 | tgttattcct gatgagaaat ggaaaaattt caacatttca cgacaatatt tagtgtactt |
| 1261 | tttttgtaga tggacttttt cagagtaagt caagccattc tgttttagtt tgttgagagt |
| 1321 | gttcattttg aatatatgtt gaatttcatc aaacactgac ctgagtcatc ttaaaacatg |
| 1381 | tgaattgaga tttctttgct actaagaaag tgagcgggca ctctgcttca tgtttacttt |
| 1441 | tgtcatgttg catgaaaaac attttgcttc atgtttgatt ctgtatgttg aaaactgaaa |
| 1501 | tcatctattg tgattaccac agggtttttt cccccagtaa tctgtttatg tagtcaatta |
| 1561 | cgttgataaa tctgtacttt ttaaatttta acaattgaga caggtctcac tctgtcaccc |
| 1621 | atgctgactg cagtagtgtg atcacagctt actgcaacct caacctcctg ggctcaggcg |
| 1681 | atcctcccac ctcaccctcc tcagtagcta ggactatagg tacatgccac catgccaagc |
| 1741 | taatttttct gttttagaga tggtattttt tcatgttgcc caggctggtt ttatactcct |
| 1801 | aatctcaagc catccactag cctcagcctc ccaaagtgct gggattacag gaatgagtca |
| 1861 | cggtgcctgg aaattttgta catttaaacc aacaatttgg tcatgggtaa tctatgtcct |
| 1921 | aataatttat ttaaggattt ttatgtatat cctcatgagt gacattacct gtacttttat |
| 1981 | ttcatatgct catttgttgg acgttgttat caaggttcct ctagcttcat aaaacgggtt |
| 2041 | ggtatgtaaa acctcttttt ccattcactg gaactcaagt ctcctcaagg ctgtgagtaa |
| 2101 | tgcagggcta ggctttccca taatgagctt ttctagaatg cttctctcag atttggaccc |
| 2161 | tacttaaaca gcagtgacca aacgggcagc tccaggtacc tatcccctca aactttgtga |
| 2221 | gggtcgcgtt ctctgaagat gcctcttcaa tttgaaagct atctgttcct gtttctctgc |
| 2281 | tgatattaac tctctgtgca cagaaagtta aacgtcactg gttattaatt tccctagatt |
| 2341 | ttgatctgtg ctgtgtggct gagaatgggc tgactgaccc tagatctgtg tataattatg |
| 2401 | acaatggctc catttatttt taaaataaga ggaattatta taaaattcct atttactgga |
| 2461 | tgtgtactat ctatgaatta cttctttgtg ctaggttgtg tacatgtatg acctctttag |
| 2521 | atcctcacaa gataaggcag aattttcatg aaattgatga ctgactccag taagaagcag |
| 2581 | atttgggggg atttcaattt ctaagctcaa agcccttgca cttttctcaa agtaaagctt |
| 2641 | ttgaaagtgt taaatgtaca cgaactgatg gtgtatatga tgattttagt tgtaatctga |
| 2701 | tgttttcttt aaaaatttac atatacaaaa gtgtttgact caaaaggctt tgttcttccc |
| 2761 | ttaaaggaag catctaccaa aatgtggcac acaaaccttg catggtgtct ctagggcctc |
| 2821 | ctaccccgca tttcccctgc tttttctctt gttctgactg aaaaacaaag tgctttgact |
| 2881 | gtgctgtgac ccagccagct gcatgtttac ccagcatgct tgaacccaag ctggagcctt |
| 2941 | gaacataaag gtgtttaagt tgttgctcaa aatatggaaa gaatctagct ctggccttga |
| 3001 | accaaatccc ttaaactctc ctataaaact ccataacctg accccctcag tgcggatata |
| 3061 | cctaggcatg acattcttgt tgcctgttgc gaggatgctt tagcctactc taagttctcc |
| 3121 | caataaatgc tttggactga taaaaaaaaa aaaaaaa |
| 6. GAGE2A NM_012196 | |
| (SEQ ID NO: 6) | |
| 1 | ctgtgaggca gtgctgtgtg gttcctgccg tccggactct ttttcctcta ctgagattca |
| 61 | tctgtgtgaa atatgagttg gcgaggaaga tcgacctatc ggcctagacc aagacgctac |
| 121 | gtagagcctc ctgaaatgat tgggcctatg cggcccgagc agttcagtga tgaagtggaa |
| 181 | ccagcaacac ctgaagaagg ggaaccagca actcaacgtc aggatcctgc agctgctcag |
| 241 | gagggagagg atgagggagc atctgcaggt caagggccga agcctgaagc tgatagccag |
| 301 | gaacagggtc acccacagac tgggtgtgag tgtgaagatg gtcctgatgg gcaggagatg |
| 361 | gacccgccaa atccagagga ggtgaaaacg cctgaagaag gtgaaaagca atcacagtgt |
| 421 | taaaagaaga cacgttgaaa tgatgcaggc tgctcctatg ttggaaattt gttcattaaa |
| 481 | attctcccaa taaagcttta cagccttctg caaagaaaaa aaaaaaaa |
| 7. LDHC NM_002301 | |
| (SEQ ID NO: 7) | |
| 1 | cgtgcgtgtc tcgagtcgca cggagggcaa ccgtcgacgg gcttagcgcc tcaactgtcg |
| 61 | ttggtgtatt tttctggtgt cacttctgtg ccttccttca aaggtggtgc tttgtccctg |
| 121 | tgggtcatct gtactgattg cgccaagcaa agcatttgtt ctccaaatgt caactgtcaa |
| 181 | ggagcagcta attgagaagc taattgagga tgatgaaaac tcccagtgta aaattactat |
| 241 | tgttggaact ggtgccgtag gcatggcttg tgctattagt atcttactga aggatttggc |
| 301 | tgatgaactt gcccttgttg atgttgcatt ggacaaactg aagggagaaa tgatggatct |
| 361 | tcagcatggc agtcttttct ttagtacttc aaagattact tctggaaaag attacagtgt |
| 421 | atctgcaaac tccagaatag ttattgtcac agcaggtgca aggcagcagg agggagaaac |
| 481 | tcgccttgcc ctggtccaac gtaatgtggc tataatgaaa tcaatcattc ctgccatagt |
| 541 | ccattatagt cctgattgta aaattcttgt tgtttcaaat ccagtggata ttttgacata |
| 601 | tatagtctgg aagataagtg gcttacctgt aactcgtgta attggaagtg gttgtaatct |
| 661 | agactctgcc cgtttccgtt acctaattgg agaaaagttg ggtgtccacc ccacaagctg |
| 721 | ccatggttgg attattggag aacatggtga ttctagtgtg cccttatgga gtggggtgaa |
| 781 | tgttgctggt gttgctctga agactctgga ccctaaatta ggaacggatt cagataagga |
| 841 | acactggaaa aatatccata aacaagttat tcaaagtgcc tatgaaatta tcaagctgaa |
| 901 | ggggtatacc tcttgggcta ttggactgtc tgtgatggat ctggtaggat ccattttgaa |
| 961 | aaatcttagg agagtgcacc cagtttccac catggttaag ggattatatg gaataaaaga |
| 1021 | agaactcttt ctcagtatcc cttgtgtctt ggggcggaat ggtgtctcag atgttgtgaa |
| 1081 | aattaacttg aattctgagg aggaggccct tttcaagaag agtgcagaaa cactttggaa |
| 1141 | tattcaaaag gatctaatat tttaaattaa agccttctaa tgttccactg tttggagaac |
| 1201 | agaagatagc aggctgtgta ttttaaattt tgaaagtatt ttcatttgat ctttaaaaaa |
| 1261 | taaaaacaaa ttggagacct gtgaaaaaaa aaaaaaaaaa aaaaaaaaaa aaaaaaaa |
| 8. MAGEA1 NM_004988 | |
| (SEQ ID NO: 8) | |
| 1 | agagagaagc gaggtttcca ttctgaggga cggcgtagag ttcggccgaa ggaacctgac |
| 61 | ccaggctctg tgaggaggca aggttttcag gggacaggcc aacccagagg acaggattcc |
| 121 | ctggaggcca cagaggagca ccaaggagaa gatctgcctg tgggtcttca ttgcccagct |
| 181 | cctgcccaca ctcctgcctg ctgccctgac gagagtcatc atgtctcttg agcagaggag |
| 241 | tctgcactgc aagcctgagg aagcccttga ggcccaacaa gaggccctgg gcctggtgtg |
| 301 | tgtgcaggct gccacctcct cctcctctcc tctggtcctg ggcaccctgg aggaggtgcc |
| 361 | cactgctggg tcaacagatc ctccccagag tcctcaggga gcctccgcct ttcccactac |
| 421 | catcaacttc actcgacaga ggcaacccag tgagggttcc agcagccgtg aagaggaggg |
| 481 | gccaagcacc tcttgtatcc tggagtcctt gttccgagca gtaatcacta agaaggtggc |
| 541 | tgatttggtt ggttttctgc tcctcaaata tcgagccagg gagccagtca caaaggcaga |
| 601 | aatgctggag agtgtcatca aaaattacaa gcactgtttt cctgagatct tcggcaaagc |
| 661 | ctctgagtcc ttgcagctgg tctttggcat tgacgtgaag gaagcagacc ccaccggcca |
| 721 | ctcctatgtc cttgtcacct gcctaggtct ctcctatgat ggcctgctgg gtgataatca |
| 781 | gatcatgccc aagacaggct tcctgataat tgtcctggtc atgattgcaa tggagggcgg |
| 841 | ccatgctcct gaggaggaaa tctgggagga gctgagtgtg atggaggtgt atgatgggag |
| 901 | ggagcacagt gcctatgggg agcccaggaa gctgctcacc caagatttgg tgcaggaaaa |
| 961 | gtacctggag taccggcagg tgccggacag tgatcccgca cgctatgagt tcctgtgggg |
| 1021 | tccaagggcc ctcgctgaaa ccagctatgt gaaagtcctt gagtatgtga tcaaggtcag |
| 1081 | tgcaagagtt cgctttttct tcccatccct gcgtgaagca gctttgagag aggaggaaga |
| 1141 | gggagtctga gcatgagttg cagccaaggc cagtgggagg gggactgggc cagtgcacct |
| 1201 | tccagggccg cgtccagcag cttcccctgc ctcgtgtgac atgaggccca ttcttcactc |
| 1261 | tgaagagagc ggtcagtgtt ctcagtagta ggtttctgtt ctattgggtg acttggagat |
| 1321 | ttatctttgt tctcttttgg aattgttcaa atgttttttt ttaagggatg gttgaatgaa |
| 1381 | cttcagcatc caagtttatg aatgacagca gtcacacagt tctgtgtata tagtttaagg |
| 1441 | gtaagagtct tgtgttttat tcagattggg aaatccattc tattttgtga attgggataa |
| 1501 | taacagcagt ggaataagta cttagaaatg tgaaaaatga gcagtaaaat agatgagata |
| 1561 | aagaactaaa gaaattaaga gatagtcaat tcttgcctta tacctcagtc tattctgtaa |
| 1621 | aatttttaaa gatatatgca tacctggatt tccttggctt ctttgagaat gtaagagaaa |
| 1681 | ttaaatctga ataaagaatt cttcctgtta aaaaaaaaaa aaaaaaaaaa aaaaaaaaaa |
| 1741 | aaaaaaaaaa aaaaa |
| 9. MAGEA3 NM_005362 | |
| (SEQ ID NO: 9) | |
| 1 | gagattctcg ccctgagcaa cgagcgacgg cctgacgtcg gcggagggaa gccggcccag |
| 61 | gctcggtgag gaggcaaggt tctgagggga caggctgacc tggaggacca gaggcccccg |
| 121 | gaggagcact gaaggagaag atctgccagt gggtctccat tgcccagctc ctgcccacac |
| 181 | tcccgcctgt tgccctgacc agagtcatca tgcctcttga gcagaggagt cagcactgca |
| 241 | agcctgaaga aggccttgag gcccgaggag aggccctggg cctggtgggt gcgcaggctc |
| 301 | ctgctactga ggagcaggag gctgcctcct cctcttctac tctagttgaa gtcaccctgg |
| 361 | gggaggtgcc tgctgccgag tcaccagatc ctccccagag tcctcaggga gcctccagcc |
| 421 | tccccactac catgaactac cctctctgga gccaatccta tgaggactcc agcaaccaag |
| 481 | aagaggaggg gccaagcacc ttccctgacc tggagtccga gttccaagca gcactcagta |
| 541 | ggaaggtggc cgagttggtt cattttctgc tcctcaagta tcgagccagg gagccggtca |
| 601 | caaaggcaga aatgctgggg agtgtcgtcg gaaattggca gtatttcttt cctgtgatct |
| 661 | tcagcaaagc ttccagttcc ttgcagctgg tctttggcat cgagctgatg gaagtggacc |
| 721 | ccatcggcca cttgtacatc tttgccacct gcctgggcct ctcctacgat ggcctgctgg |
| 781 | gtgacaatca gatcatgccc aaggcaggcc tcctgataat cgtcctggcc ataatcgcaa |
| 841 | gagagggcga ctgtgcccct gaggagaaaa tctgggagga gctgagtgtg ttagaggtgt |
| 901 | ttgaggggag ggaagacagt atcttggggg atcccaagaa gctgctcacc caacatttcg |
| 961 | tgcaggaaaa ctacctggag taccggcagg tccccggcag tgatcctgca tgttatgaat |
| 1021 | tcctgtgggg tccaagggcc ctcgttgaaa ccagctatgt gaaagtcctg caccatatgg |
| 1081 | taaagatcag tggaggacct cacatttcct acccacccct gcatgagtgg gttttgagag |
| 1141 | agggggaaga gtgagtctga gcacgagttg cagccagggc cagtgggagg gggtctgggc |
| 1201 | cagtgcacct tccggggccg catcccttag tttccactgc ctcctgtgac gtgaggccca |
| 1261 | ttcttcactc tttgaagcga gcagtcagca ttcttagtag tgggtttctg ttctgttgga |
| 1321 | tgactttgag attattcttt gtttcctgtt ggagttgttc aaatgttcct tttaacggat |
| 1381 | ggttgaatga gcgtcagcat ccaggtttat gaatgacagt agtcacacat agtgctgttt |
| 1441 | atatagttta ggagtaagag tcttgttttt tactcaaatt gggaaatcca ttccattttg |
| 1501 | tgaattgtga cataataata gcagtggtaa aagtatttgc ttaaaattgt gagcgaatta |
| 1561 | gcaataacat acatgagata actcaagaaa tcaaaagata gttgattctt gccttgtacc |
| 1621 | tcaatctatt ctgtaaaatt aaacaaatat gcaaaccagg atttccttga cttctttgag |
| 1681 | aatgcaagcg aaattaaatc tgaataaata attcttcctc ttcaaaaaaa aaaaaaaaaa |
| 1741 | aaaaaaaaaa aaa |
| 10. MAGEA4V2 NM_002362 | |
| (SEQ ID NO: 10) | |
| 1 | ggttagagag aagcgagctg ctctgtctga ccagcagctt gggattggcg gagggaagcg |
| 61 | ggccaggccc tgtgaggagt caaggttctg agcagacagg ccaaccggag gacaggattc |
| 121 | cctggaggcc acagaggagc accaaggaga agatctgcct gtgggtcccc attgcccagc |
| 181 | ttttgcctgc actcttgcct gctgccctga ccagagtcat catgtcttct gagcagaaga |
| 241 | gtcagcactg caagcctgag gaaggcgttg aggcccaaga agaggccctg ggcctggtgg |
| 301 | gtgcacaggc tcctactact gaggagcagg aggctgctgt ctcctcctcc tctcctctgg |
| 361 | tccctggcac cctggaggaa gtgcctgctg ctgagtcagc aggtcctccc cagagtcctc |
| 421 | agggagcctc tgccttaccc actaccatca gcttcacttg ctggaggcaa cccaatgagg |
| 481 | gttccagcag ccaagaagag gaggggccaa gcacctcgcc tgacgcagag tccttgttcc |
| 541 | gagaagcact cagtaacaag gtggatgagt tggctcattt tctgctccgc aagtatcgag |
| 601 | ccaaggagct ggtcacaaag gcagaaatgc tggagagagt catcaaaaat tacaagcgct |
| 661 | gctttcctgt gatcttcggc aaagcctccg agtccctgaa gatgatcttt ggcattgacg |
| 721 | tgaaggaagt ggaccccgcc agcaacacct acacccttgt cacctgcctg ggcctttcct |
| 781 | atgatggcct gctgggtaat aatcagatct ttcccaagac aggccttctg ataatcgtcc |
| 841 | tgggcacaat tgcaatggag ggcgacagcg cctctgagga ggaaatctgg gaggagctgg |
| 901 | gtgtgatggg ggtgtatgat gggagggagc acactgtcta tggggagccc aggaaactgc |
| 961 | tcacccaaga ttgggtgcag gaaaactacc tggagtaccg gcaggtaccc ggcagtaatc |
| 1021 | ctgcgcgcta tgagttcctg tggggtccaa gggctctggc tgaaaccagc tatgtgaaag |
| 1081 | tcctggagca tgtggtcagg gtcaatgcaa gagttcgcat tgcctaccca tccctgcgtg |
| 1141 | aagcagcttt gttagaggag gaagagggag tctgagcatg agttgcagcc agggctgtgg |
| 1201 | ggaaggggca gggctgggcc agtgcatcta acagccctgt gcagcagctt cccttgcctc |
| 1261 | gtgtaacatg aggcccattc ttcactctgt ttgaagaaaa tagtcagtgt tcttagtagt |
| 1321 | gggtttctat tttgttggat gacttggaga tttatctctg tttcctttta caattgttga |
| 1381 | aatgttcctt ttaatggatg gttgaattaa cttcagcatc caagtttatg aatcgtagtt |
| 1441 | aacgtatatt gctgttaata tagtttagga gtaagagtct tgttttttat tcagattggg |
| 1501 | aaatccgttc tattttgtga atttgggaca taataacagc agtggagtaa gtatttagaa |
| 1561 | gtgtgaattc accgtgaaat aggtgagata aattaaaaga tacttaattc ccgccttatg |
| 1621 | cctcagtcta ttctgtaaaa tttaaaaaat atatatgcat acctggattt ccttggcttc |
| 1681 | gtgaatgtaa gagaaattaa atctgaataa ataattcttt ctgttaa |
| 11. MAGEA4V3 NM_001011549 | |
| (SEQ ID NO: 11) | |
| 1 | ttagagagaa gcgagctgct ctgaccagcc gcttgggatt ggcggaggga agcgggccag |
| 61 | gccctgtgag gagtcaaggt tctgagcaga caggccaacc ggaggacagg attccctgga |
| 121 | ggccacagag gagcaccaag gagaagatct gcctgtgggt ccccattgcc cagcttttgc |
| 181 | ctgcactctt gcctgctgcc ctgaccagag tcatcatgtc ttctgagcag aagagtcagc |
| 241 | actgcaagcc tgaggaaggc gttgaggccc aagaagaggc cctgggcctg gtgggtgcac |
| 301 | aggctcctac tactgaggag caggaggctg ctgtctcctc ctcctctcct ctggtccctg |
| 361 | gcaccctgga ggaagtgcct gctgctgagt cagcaggtcc tccccagagt cctcagggag |
| 421 | cctctgcctt acccactacc atcagcttca cttgctggag gcaacccaat gagggttcca |
| 481 | gcagccaaga agaggagggg ccaagcacct cgcctgacgc agagtccttg ttccgagaag |
| 541 | cactcagtaa caaggtggat gagttggctc attttctgct ccgcaagtat cgagccaagg |
| 601 | agctggtcac aaaggcagaa atgctggaga gagtcatcaa aaattacaag cgctgctttc |
| 661 | ctgtgatctt cggcaaagcc tccgagtccc tgaagatgat ctttggcatt gacgtgaagg |
| 721 | aagtggaccc cgccagcaac acctacaccc ttgtcacctg cctgggcctt tcctatgatg |
| 781 | gcctgctggg taataatcag atctttccca agacaggcct tctgataatc gtcctgggca |
| 841 | caattgcaat ggagggcgac agcgcctctg aggaggaaat ctgggaggag ctgggtgtga |
| 901 | tgggggtgta tgatgggagg gagcacactg tctatgggga gcccaggaaa ctgctcaccc |
| 961 | aagattgggt gcaggaaaac tacctggagt accggcaggt acccggcagt aatcctgcgc |
| 1021 | gctatgagtt cctgtggggt ccaagggctc tggctgaaac cagctatgtg aaagtcctgg |
| 1081 | agcatgtggt cagggtcaat gcaagagttc gcattgccta cccatccctg cgtgaagcag |
| 1141 | ctttgttaga ggaggaagag ggagtctgag catgagttgc agccagggct gtggggaagg |
| 1201 | ggcagggctg ggccagtgca tctaacagcc ctgtgcagca gcttcccttg cctcgtgtaa |
| 1261 | catgaggccc attcttcact ctgtttgaag aaaatagtca gtgttcttag tagtgggttt |
| 1321 | ctattttgtt ggatgacttg gagatttatc tctgtttcct tttacaattg ttgaaatgtt |
| 1381 | ccttttaatg gatggttgaa ttaacttcag catccaagtt tatgaatcgt agttaacgta |
| 1441 | tattgctgtt aatatagttt aggagtaaga gtcttgtttt ttattcagat tgggaaatcc |
| 1501 | gttctatttt gtgaatttgg gacataataa cagcagtgga gtaagtattt agaagtgtga |
| 1561 | attcaccgtg aaataggtga gataaattaa aagatactta attcccgcct tatgcctcag |
| 1621 | tctattctgt aaaatttaaa aaatatatat gcatacctgg atttccttgg cttcgtgaat |
| 1681 | gtaagagaaa ttaaatctga ataaataatt ctttctgtta a |
| 12. MAGEA4V4 NM_001011550 | |
| (SEQ ID NO: 12) | |
| 1 | cgagctgctg tctgaccagc agcttgggat tggtggaagg aagcaggcca ggccctgtga |
| 61 | ggagtcaagg ttctgagcag acaggccaac cggaggacag gattccctgg aggccacaga |
| 121 | ggagcaccaa ggagaagatc tgcctgtggg tccccattgc ccagcttttg cctgcactct |
| 181 | tgcctgctgc cctgaccaga gtcatcatgt cttctgagca gaagagtcag cactgcaagc |
| 241 | ctgaggaagg cgttgaggcc caagaagagg ccctgggcct ggtgggtgca caggctccta |
| 301 | ctactgagga gcaggaggct gctgtctcct cctcctctcc tctggtccct ggcaccctgg |
| 361 | aggaagtgcc tgctgctgag tcagcaggtc ctccccagag tcctcaggga gcctctgcct |
| 421 | tacccactac catcagcttc acttgctgga ggcaacccaa tgagggttcc agcagccaag |
| 481 | aagaggaggg gccaagcacc tcgcctgacg cagagtcctt gttccgagaa gcactcagta |
| 541 | acaaggtgga tgagttggct cattttctgc tccgcaagta tcgagccaag gagctggtca |
| 601 | caaaggcaga aatgctggag agagtcatca aaaattacaa gcgctgcttt cctgtgatct |
| 661 | tcggcaaagc ctccgagtcc ctgaagatga tctttggcat tgacgtgaag gaagtggacc |
| 721 | ccgccagcaa cacctacacc cttgtcacct gcctgggcct ttcctatgat ggcctgctgg |
| 781 | gtaataatca gatctttccc aagacaggcc ttctgataat cgtcctgggc acaattgcaa |
| 841 | tggagggcga cagcgcctct gaggaggaaa tctgggagga gctgggtgtg atgggggtgt |
| 901 | atgatgggag ggagcacact gtctatgggg agcccaggaa actgctcacc caagattggg |
| 961 | tgcaggaaaa ctacctggag taccggcagg tacccggcag taatcctgcg cgctatgagt |
| 1021 | tcctgtgggg tccaagggct ctggctgaaa ccagctatgt gaaagtcctg gagcatgtgg |
| 1081 | tcagggtcaa tgcaagagtt cgcattgcct acccatccct gcgtgaagca gctttgttag |
| 1141 | aggaggaaga gggagtctga gcatgagttg cagccagggc tgtggggaag gggcagggct |
| 1201 | gggccagtgc atctaacagc cctgtgcagc agcttccctt gcctcgtgta acatgaggcc |
| 1261 | cattcttcac tctgtttgaa gaaaatagtc agtgttctta gtagtgggtt tctattttgt |
| 1321 | tggatgactt ggagatttat ctctgtttcc ttttacaatt gttgaaatgt tccttttaat |
| 1381 | ggatggttga attaacttca gcatccaagt ttatgaatcg tagttaacgt atattgctgt |
| 1441 | taatatagtt taggagtaag agtcttgttt tttattcaga ttgggaaatc cgttctattt |
| 1501 | tgtgaatttg ggacataata acagcagtgg agtaagtatt tagaagtgtg aattcaccgt |
| 1561 | gaaataggtg agataaatta aaagatactt aattcccgcc ttatgcctca gtctattctg |
| 1621 | taaaatttaa aaaatatata tgcatacctg gatttccttg gcttcgtgaa tgtaagagaa |
| 1681 | attaaatctg aataaataat tctttctgtt aa |
| // | |
| 13. MAGEB6 NM_173523 | |
| (SEQ ID NO: 13) | |
| 1 | aataaagggg tctgagccgg tcgcctgagc ctgaaaagtg ctgtcacgtc agcggaagga |
| 61 | ggcgtcccag atcttctcag ctgtcttggt gccagccttc ctagtcttcc tacccacact |
| 121 | cctacctgct gtcacaggcc acagccatca tgcctcgggg tcacaagagt aagctccgta |
| 181 | cctgtgagaa acgccaagag accaatggtc agccacaggg tctcacgggt ccccaggcca |
| 241 | ctgcagagaa gcaggaagag tcccactctt cctcatcctc ttctcgcgct tgtctgggtg |
| 301 | attgtcgtag gtcttctgat gcctccattc ctcaggagtc tcagggagtg tcacccactg |
| 361 | ggtctcctga tgcagttgtt tcatattcaa aatccgatgt ggctgccaac ggccaagatg |
| 421 | agaaaagtcc aagcacctcc cgtgatgcct ccgttcctca ggagtctcag ggagcttcac |
| 481 | ccactggctc tcctgatgca ggtgtttcag gctcaaaata tgatgtggct gccaacggcc |
| 541 | aagatgagaa aagtccaagc acttcccatg atgtctccgt tcctcaggag tctcagggag |
| 601 | cttcacccac tggctcgcct gatgcaggtg tttcaggctc aaaatatgat gtggctgccg |
| 661 | agggtgaaga tgaggaaagt gtaagcgcct cacagaaagc catcattttt aagcgcttaa |
| 721 | gcaaagatgc tgtaaagaag aaggcgtgca cgttggcgca attcctgcag aagaagtttg |
| 781 | agaagaaaga gtccattttg aaggcagaca tgctgaagtg tgtccgcaga gagtacaagc |
| 841 | cctacttccc tcagatcctc aacagaacct cccaacattt ggtggtggcc tttggcgttg |
| 901 | aattgaaaga aatggattcc agcggcgagt cctacaccct tgtcagcaag ctaggcctcc |
| 961 | ccagtgaagg aattctgagt ggtgataatg cgctgccgaa gtcgggtctc ctgatgtcgc |
| 1021 | tcctggttgt gatcttcatg aacggcaact gtgccactga agaggaggtc tgggagttcc |
| 1081 | tgggtctgtt ggggatatat gatgggatcc tgcattcaat ctatggggat gctcggaaga |
| 1141 | tcattactga agatttggtg caagataagt acgtggttta ccggcaggtg tgcaacagtg |
| 1201 | atcctccatg ctatgagttc ctgtggggtc cacgagccta tgctgaaacc accaagatga |
| 1261 | gagtcctgcg tgttttggcc gacagcagta acaccagtcc cggtttatac ccacatctgt |
| 1321 | atgaagacgc tttgatagat gaggtagaga gagcattgag actgagagct taaggcaggg |
| 1381 | ctggcactat ttccttggcc agggtacctt atggggccat atcctacaga tcctcccatt |
| 1441 | tctagggagg tctgaagtag aattttcact ttatgttaga agagagtagt gagctttcta |
| 1501 | agtagtgcag tatagtagag gctggaggga acaagatatg tatctttctt ttgttacaca |
| 1561 | tgagtaactt gcagatttat gttttatctc tgtcagttat caacattgtt cctgttaagt |
| 1621 | gaaggtttat tttgcttcag attatacaat tatcaataac atagctctca cattcatggc |
| 1681 | tgtttaacca atctgaaagt tacggtttgg gaattaataa aacaaagtca tacaacacat |
| 1741 | tttctttgta attgagaact agataacatg gtaacagaga attgattttc atatgaatct |
| 1801 | taactccaca gtaaaatagt tgacatcata atatgaagag aaagaaaagg aaaaacagaa |
| 1861 | atgtaaaagt tgtttaattc ttggtttgcc taattcgttt tcctatttct tttcatacaa |
| 1921 | ataaaggata cctggattta tttaggtta |
| // | |
| 14. MICA NM_000247 | |
| (SEQ ID NO: 14) | |
| 1 | cactgcttga gccgctgaga gggtggcgac gtcggggcca tggggctggg cccggtcttc |
| 61 | ctgcttctgg ctggcatctt cccttttgca cctccgggag ctgctgctga gccccacagt |
| 121 | cttcgttata acctcacggt gctgtcctgg gatggatctg tgcagtcagg gtttctcact |
| 181 | gaggtacatc tggatggtca gcccttcctg cgctgtgaca ggcagaaatg cagggcaaag |
| 241 | ccccagggac agtgggcaga agatgtcctg ggaaataaga catgggacag agagaccaga |
| 301 | gacttgacag ggaacggaaa ggacctcagg atgaccctgg ctcatatcaa ggaccagaaa |
| 361 | gaaggcttgc attccctcca ggagattagg gtctgtgaga tccatgaaga caacagcacc |
| 421 | aggagctccc agcatttcta ctacgatggg gagctcttcc tctcccaaaa cctggagact |
| 481 | aaggaatgga caatgcccca gtcctccaga gctcagacct tggccatgaa cgtcaggaat |
| 541 | ttcttgaagg aagatgccat gaagaccaag acacactatc acgctatgca tgcagactgc |
| 601 | ctgcaggaac tacggcgata tctaaaatcc ggcgtagtcc tgaggagaac agtgcccccc |
| 661 | atggtgaatg tcacccgcag cgaggcctca gagggcaaca ttaccgtgac atgcagggct |
| 721 | tctggcttct atccctggaa tatcacactg agctggcgtc aggatggggt atctttgagc |
| 781 | cacgacaccc agcagtgggg ggatgtcctg cctgatggga atggaaccta ccagacctgg |
| 841 | gtggccacca ggatttgcca aggagaggag cagaggttca cctgctacat ggaacacagc |
| 901 | gggaatcaca gcactcaccc tgtgccctct gggaaagtgc tggtgcttca gagtcattgg |
| 961 | cagacattcc atgtttctgc tgttgctgct gctgctattt ttgttattat tattttctat |
| 1021 | gtccgttgtt gtaagaagaa aacatcagct gcagagggtc cagagctcgt gagcctgcag |
| 1081 | gtcctggatc aacacccagt tgggacgagt gaccacaggg atgccacaca gctcggattt |
| 1141 | cagcctctga tgtcagatct tgggtccact ggctccactg agggcgccta gactctacag |
| 1201 | ccaggcagct gggattcaat tccctgcctg gatctcacga gcactttccc tcttggtgcc |
| 1261 | tcagtttcct gacctatgaa acagagaaaa taaaagcact tatttattgt tgttggaggc |
| 1321 | tgcaaaatgt tagtagatat gaggcgtttg cagctgtacc atatt |
| // | |
| 15. NLRP4 NM_134444 | |
| (SEQ ID NO: 15) | |
| 1 | gtgctgggct gttcgtctct tctatgtgct gatttcctgg gttactttgg gtcttccttt |
| 61 | tctttctccc ttttaccctg tctcctttct tgaggctgat cgatcacagc caggcctctc |
| 121 | cattctattt acccagcgtt ttccttctct ccagttagtg gggtagatga acgccctgtg |
| 181 | tttataaggt gcctcccagg agcctgagac ctgtgagaag aatggggggt ggaggtgggg |
| 241 | gagactcgtc acgaagggag accttggagc ttcgagggtg ggaatgttct tattagattc |
| 301 | ttcatctctg ttgacacaaa catgtaggag aagctggaga acatagacag ggatgaggtt |
| 361 | ttatttattt attgttcctg gtcactgtct ctttgaggat tggtatctct gctccagaaa |
| 421 | agatggcagc ctctttcttc tctgattttg gtcttatgtg gtatctggag gagctcaaaa |
| 481 | aggaggagtt caggaaattt aaagaacatc tcaagcaaat gactttgcag cttgaactca |
| 541 | agcagattcc ctggactgag gtcaaaaaag catcccggga agaacttgca aacctcttga |
| 601 | tcaagcacta tgaagaacaa caagcttgga acataacctt aagaatcttt caaaagatgg |
| 661 | atagaaagga tctctgcatg aaggtcatga gggagagaac aggatacaca aagacctatc |
| 721 | aagctcacgc aaagcagaaa ttcagccgct tatggtccag caagtctgtc actgagattc |
| 781 | acctatactt tgaggaggaa gtcaagcaag aagaatgtga ccatttggac cgcctttttg |
| 841 | ctcccaagga agctgggaaa cagccacgta cagtgatcat tcaaggacca caaggaattg |
| 901 | gaaaaacgac actcctgatg aagctgatga tggcctggtc ggacaacaag atctttcggg |
| 961 | ataggttcct gtacacgttc tatttctgct gcagagaact gagggagttg ccgccaacga |
| 1021 | gtttggctga cttgatttcc agagagtggc ctgaccccgc tgctcctata acagagatcg |
| 1081 | tgtctcaacc ggagagactc ttgttcgtca tcgacagctt cgaagagctg cagggcggct |
| 1141 | tgaacgaacc cgattcggat ctgtgtggtg acttgatgga gaaacggccg gtgcaggtgc |
| 1201 | ttctgagcag tttgctgagg aagaagatgc tcccggaggc ctccctgctc atcgctatca |
| 1261 | aacccgtgtg cccgaaggag ctccgggatc aggtgacgat ctcagaaatc taccagcccc |
| 1321 | ggggattcaa cgagagtgat aggttagtgt atttctgctg tttcttcaaa gacccgaaaa |
| 1381 | gagccatgga agccttcaat cttgtaagag aaagtgaaca gctgttttcc atatgccaaa |
| 1441 | tcccgctcct ctgctggatc ctgtgtacca gtctgaagca agagatgcag aaaggaaaag |
| 1501 | acctggccct gacctgccag agcactacct ctgtgtactc ctctttcgtc tttaacctgt |
| 1561 | tcacacctga gggtgccgag ggcccgactc cgcaaaccca gcaccagctg aaggccctgt |
| 1621 | gctccctggc tgcagagggt atgtggacag acacatttga gttttgtgaa gacgacctcc |
| 1681 | ggagaaatgg ggttgttgac gctgacatcc ctgcgctgct gggcaccaag atacttctga |
| 1741 | agtacgggga gcgtgagagc tcctacgtgt tcctccacgt gtgtatccag gagttctgtg |
| 1801 | ccgccttgtt ctatttgctc aagagccacc ttgatcatcc tcacccagct gtgagatgtg |
| 1861 | tacaggaatt gctagttgcc aattttgaaa aagcaaggag agcacattgg atttttttgg |
| 1921 | ggtgttttct aactggcctt ttaaataaaa aggaacaaga aaaactggat gcgttttttg |
| 1981 | gcttccaact gtcccaagag ataaagcagc aaattcacca gtgcctgaag agcttagggg |
| 2041 | agcgtggcaa tcctcaggga caggtggatt ccttggcgat attttactgt ctctttgaaa |
| 2101 | tgcaggatcc tgcctttgtg aagcaggcag tgaacctcct ccaagaagct aactttcata |
| 2161 | ttattgacaa cgtggacttg gtggtttctg cctactgctt aaaatactgc tccagcttga |
| 2221 | ggaaactctg tttttccgtt caaaatgtct ttaagaaaga ggatgaacac agctctacgt |
| 2281 | cggattacag cctcatctgt tggcatcaca tctgctctgt gctcaccacc agcgggcacc |
| 2341 | tcagagagct ccaggtgcag gacagcaccc tcagcgagtc gacctttgtg acctggtgta |
| 2401 | accagctgag gcatcccagc tgtcgccttc agaagcttgg aataaataac gtttcctttt |
| 2461 | ctggccagag tgttctgctc tttgaggtgc tcttttatca gccagacttg aaatacctga |
| 2521 | gcttcaccct cacgaaactc tctcgtgatg acatcaggtc cctctgtgat gccttgaact |
| 2581 | acccagcagg caacgtcaaa gagctagcgc tggtaaattg tcacctctca cccattgatt |
| 2641 | gtgaagtcct tgctggcctt ctaaccaaca acaagaagct gacgtatctg aatgtatcct |
| 2701 | gcaaccagtt agacacaggc gtgccccttt tgtgtgaagc cctgtgcagc ccagacacgg |
| 2761 | tcctggtata cctgatgttg gctttctgcc acctcagcga gcagtgctgc gaatacatct |
| 2821 | ctgaaatgct tctgcgtaac aagagcgtgc gctatctaga cctcagtgcc aatgtcctga |
| 2881 | aggacgaagg actgaaaact ctctgcgagg ccttgaaaca tccggactgc tgcctggatt |
| 2941 | cactgtgttt ggtaaaatgt tttatcactg ctgctggctg tgaagacctc gcctctgctc |
| 3001 | tcatcagcaa tcaaaacctg aagattctgc aaattgggtg caatgaaatc ggagatgtgg |
| 3061 | gtgtgcagct gttgtgtcgg gctctgacgc atacggattg ccgcttagag attcttgggt |
| 3121 | tggaagaatg tgggttaacg agcacctgct gtaaggatct cgcgtctgtt ctcacctgca |
| 3181 | gtaagaccct gcagcagctc aacctgacct tgaacacctt ggaccacaca ggggtggttg |
| 3241 | tactctgtga ggccctgaga cacccagagt gtgccctgca ggtgctcggg ctgagaaaaa |
| 3301 | ctgattttga tgaggaaacc caggcacttc tgacggctga ggaagagaga aatcctaacc |
| 3361 | tgaccatcac agacgactgt gacacaatca caagggtaga gatctgattg cgaggaacct |
| 3421 | gggctctgac tcgaacacct gcaaaggaca gggactggga ccgttactta catgacactg |
| 3481 | cacccaggag atacaaatca ttgatactct gagttgtgag atttctggca ccccattcat |
| 3541 | agatttgata tgatacacgt ggtttttatg tgctctgtgg ccttggatga gtcactgaaa |
| 3601 | ggccttcatg gtctctcggt ctcacaagga cctcttaacc cctcaataaa gtgttacatt |
| 3661 | tctaaacatt ggaaaaaaaa aaaaaaaaaa |
| // | |
| 16. NY-ESO-1 NM_001327 | |
| (SEQ ID NO: 16) | |
| 1 | atcctcgtgg gccctgacct tctctctgag agccgggcag aggctccgga gccatgcagg |
| 61 | ccgaaggccg gggcacaggg ggttcgacgg gcgatgctga tggcccagga ggccctggca |
| 121 | ttcctgatgg cccagggggc aatgctggcg gcccaggaga ggcgggtgcc acgggcggca |
| 181 | gaggtccccg gggcgcaggg gcagcaaggg cctcggggcc gggaggaggc gccccgcggg |
| 241 | gtccgcatgg cggcgcggct tcagggctga atggatgctg cagatgcggg gccagggggc |
| 301 | cggagagccg cctgcttgag ttctacctcg ccatgccttt cgcgacaccc atggaagcag |
| 361 | agctggcccg caggagcctg gcccaggatg ccccaccgct tcccgtgcca ggggtgcttc |
| 421 | tgaaggagtt cactgtgtcc ggcaacatac tgactatccg actgactgct gcagaccacc |
| 481 | gccaactgca gctctccatc agctcctgtc tccagcagct ttccctgttg atgtggatca |
| 541 | cgcagtgctt tctgcccgtg tttttggctc agcctccctc agggcagagg cgctaagccc |
| 601 | agcctggcgc cccttcctag gtcatgcctc ctcccctagg gaatggtccc agcacgagtg |
| 661 | gccagttcat tgtgggggcc tgattgtttg tcgctggagg aggacggctt acatgtttgt |
| 721 | ttctgtagaa aataaaactg agctacgaaa aa |
| // | |
| 17. PBK NM_018492 | |
| (SEQ ID NO: 17) | |
| 1 | agcgcgcgac tttttgaaag ccaggagggt tcgaattgca acggcagctg ccgggcgtat |
| 61 | gtgttggtgc tagaggcagc tgcagggtct cgctgggggc cgctcgggac caattttgaa |
| 121 | gaggtacttg gccacgactt attttcacct ccgacctttc cttccaggcg gtgagactct |
| 181 | ggactgagag tggctttcac aatggaaggg atcagtaatt tcaagacacc aagcaaatta |
| 241 | tcagaaaaaa agaaatctgt attatgttca actccaacta taaatatccc ggcctctccg |
| 301 | tttatgcaga agcttggctt tggtactggg gtaaatgtgt acctaatgaa aagatctcca |
| 361 | agaggtttgt ctcattctcc ttgggctgta aaaaagatta atcctatatg taatgatcat |
| 421 | tatcgaagtg tgtatcaaaa gagactaatg gatgaagcta agattttgaa aagccttcat |
| 481 | catccaaaca ttgttggtta tcgtgctttt actgaagcca atgatggcag tctgtgtctt |
| 541 | gctatggaat atggaggtga aaagtctcta aatgacttaa tagaagaacg atataaagcc |
| 601 | agccaagatc cttttccagc agccataatt ttaaaagttg ctttgaatat ggcaagaggg |
| 661 | ttaaagtatc tgcaccaaga aaagaaactg cttcatggag acataaagtc ttcaaatgtt |
| 721 | gtaattaaag gcgattttga aacaattaaa atctgtgatg taggagtctc tctaccactg |
| 781 | gatgaaaata tgactgtgac tgaccctgag gcttgttaca ttggcacaga gccatggaaa |
| 841 | cccaaagaag ctgtggagga gaatggtgtt attactgaca aggcagacat atttgccttt |
| 901 | ggccttactt tgtgggaaat gatgacttta tcgattccac acattaatct ttcaaatgat |
| 961 | gatgatgatg aagataaaac ttttgatgaa agtgattttg atgatgaagc atactatgca |
| 1021 | gcgttgggaa ctaggccacc tattaatatg gaagaactgg atgaatcata ccagaaagta |
| 1081 | attgaactct tctctgtatg cactaatgaa gaccctaaag atcgtccttc tgctgcacac |
| 1141 | attgttgaag ctctggaaac agatgtctag tgatcatctc agctgaagtg tggcttgcgt |
| 1201 | aaataactgt ttattccaaa atatttacat agttactatc agtagttatt agactctaaa |
| 1261 | attggcatat ttgaggacca tagtttcttg ttaacatatg gataactatt tctaatatga |
| 1321 | aatatgctta tattggctat aagcacttgg aattgtactg ggttttctgt aaagttttag |
| 1381 | aaactagcta cataagtact ttgatactgc tcatgctgac ttaaaacact agcagtaaaa |
| 1441 | cgctgtaaac tgtaacatta aattgaatga ccattacttt tattaatgat ctttcttaaa |
| 1501 | tattctatat tttaatggat ctactgacat tagcactttg tacagtacaa aataaagtct |
| 1561 | acatttgttt aaaacactga accttttgct gatgtgttta tcaaatgata actggaagct |
| 1621 | gaggagaata tgcctcaaaa agagtagctc cttggatact tcagactctg gttacagatt |
| 1681 | gtcttgatct cttggatctc ctcagatctt tggtttttgc tttaatttat taaatgtatt |
| 1741 | ttccatactg agtttaaaat ttattaattt gtaccttaag catttcccag ctgtgtaaaa |
| 1801 | acaataaaac tcaaatagga tgataaagaa taaaggacac tttgggtacc agaaaaaaaa |
| 1861 | aaaaaaaaaa aaaaaaaaaa aaaaaaaaaa aaaaaaaaa |
| // | |
| 18. SILV NM_006928 | |
| (SEQ ID NO: 18) | |
| 1 | cccagcgctc ctccccgcaa atgatcccgc cccaggggcc tatcccagtc cccccagtgc |
| 61 | ctttggttgc tggagggaag aacacaatgg atctggtgct aaaaagatgc cttcttcatt |
| 121 | tggctgtgat aggtgctttg ctggctgtgg gggctacaaa agtacccaga aaccaggact |
| 181 | ggcttggtgt ctcaaggcaa ctcagaacca aagcctggaa caggcagctg tatccagagt |
| 241 | ggacagaagc ccagagactt gactgctgga gaggtggtca agtgtccctc aaggtcagta |
| 301 | atgatgggcc tacactgatt ggtgcaaatg cctccttctc tattgccttg aacttccctg |
| 361 | gaagccaaaa ggtattgcca gatgggcagg ttatctgggt caacaatacc atcatcaatg |
| 421 | ggagccaggt gtggggagga cagccagtgt atccccagga aactgacgat gcctgcatct |
| 481 | tccctgatgg tggaccttgc ccatctggct cttggtctca gaagagaagc tttgtttatg |
| 541 | tctggaagac ctggggccaa tactggcaag ttctaggggg cccagtgtct gggctgagca |
| 601 | ttgggacagg cagggcaatg ctgggcacac acaccatgga agtgactgtc taccatcgcc |
| 661 | ggggatcccg gagctatgtg cctcttgctc attccagctc agccttcacc attactgacc |
| 721 | aggtgccttt ctccgtgagc gtgtcccagt tgcgggcctt ggatggaggg aacaagcact |
| 781 | tcctgagaaa tcagcctctg acctttgccc tccagctcca tgaccccagt ggctatctgg |
| 841 | ctgaagctga cctctcctac acctgggact ttggagacag tagtggaacc ctgatctctc |
| 901 | gggcacttgt ggtcactcat acttacctgg agcctggccc agtcactgcc caggtggtcc |
| 961 | tgcaggctgc cattcctctc acctcctgtg gctcctcccc agttccaggc accacagatg |
| 1021 | ggcacaggcc aactgcagag gcccctaaca ccacagctgg ccaagtgcct actacagaag |
| 1081 | ttgtgggtac tacacctggt caggcgccaa ctgcagagcc ctctggaacc acatctgtgc |
| 1141 | aggtgccaac cactgaagtc ataagcactg cacctgtgca gatgccaact gcagagagca |
| 1201 | caggtatgac acctgagaag gtgccagttt cagaggtcat gggtaccaca ctggcagaga |
| 1261 | tgtcaactcc agaggctaca ggtatgacac ctgcagaggt atcaattgtg gtgctttctg |
| 1321 | gaaccacagc tgcacaggta acaactacag agtgggtgga gaccacagct agagagctac |
| 1381 | ctatccctga gcctgaaggt ccagatgcca gctcaatcat gtctacggaa agtattacag |
| 1441 | gttccctggg ccccctgctg gatggtacag ccaccttaag gctggtgaag agacaagtcc |
| 1501 | ccctggattg tgttctgtat cgatatggtt ccttttccgt caccctggac attgtccagg |
| 1561 | gtattgaaag tgccgagatc ctgcaggctg tgccgtccgg tgagggggat gcatttgagc |
| 1621 | tgactgtgtc ctgccaaggc gggctgccca aggaagcctg catggagatc tcatcgccag |
| 1681 | ggtgccagcc ccctgcccag cggctgtgcc agcctgtgct acccagccca gcctgccagc |
| 1741 | tggttctgca ccagatactg aagggtggct cggggacata ctgcctcaat gtgtctctgg |
| 1801 | ctgataccaa cagcctggca gtggtcagca cccagcttat catgcctggt caagaagcag |
| 1861 | gccttgggca ggttccgctg atcgtgggca tcttgctggt gttgatggct gtggtccttg |
| 1921 | catctctgat atataggcgc agacttatga agcaagactt ctccgtaccc cagttgccac |
| 1981 | atagcagcag tcactggctg cgtctacccc gcatcttctg ctcttgtccc attggtgaga |
| 2041 | acagccccct cctcagtggg cagcaggtct gagtactctc atatgatgct gtgattttcc |
| 2101 | tggagttgac agaaacacct atatttcccc cagtcttccc tgggagacta ctattaactg |
| 2161 | aaataaatac tcagagcctg aaaaaaaaaa aaaaa |
| // | |
| 19. SPANXA1 NM_013453 | |
| (SEQ ID NO: 19) | |
| 1 | aagcctgcca ctgacattga agaaccaata tatacaatgg acaaacaatc cagtgccggc |
| 61 | ggggtgaaga ggagcgtccc ctgtgattcc aacgaggcca acgagatgat gccggagacc |
| 121 | ccaactgggg actcagaccc gcaacctgct cctaaaaaaa tgaaaacatc tgagtcctcg |
| 181 | accatactag tggttcgcta caggaggaac tttaaaagaa catctccaga ggaactgctg |
| 241 | aatgaccacg cccgagagaa cagaatcaac cccctccaaa tggaggagga ggaattcatg |
| 301 | gaaataatgg ttgaaatacc tgcaaagtag caagaagcta catctctcaa ccttgggcaa |
| 361 | tgaaaataaa gtttgagaag ctga |
| // | |
| 20. SPANXB1 NM_032461 | |
| (SEQ ID NO: 20) | |
| 1 | gtcaccagga gggtatgcat agggagggca agagctctgg gccactgcga agattcaaaa |
| 61 | gctccaaaaa cctactgtag acatcgaaga accaatatat acaatgggcc aacaatccag |
| 121 | tgtccgcagg ctgaagagga gcgtcccctg tgaatccaac gaggccaacg aggccaatga |
| 181 | ggccaacaag acgatgccgg agaccccaac tggggactca gacccgcaac ctgctcctaa |
| 241 | aaaaatgaaa acatctgagt cctcgaccat actagtggtt cgctacagga ggaacgtgaa |
| 301 | aagaacatct ccagaggaac tggtgaatga ccacgcccga gagaacagaa tcaaccccga |
| 361 | ccaaatggag gaggaggaat tcatagaaat aacgactgaa agacctaaaa agtagcaaga |
| 421 | agctacatcc ctcaaacttc ggcaatgaaa ataaagtttg agaagctgaa aa |
| // | |
| 21. SSX2A NM_003147 | |
| (SEQ ID NO: 21) | |
| 1 | gcatgctctg actttctctc tctttcgatt cttccatact cagagtacgc acggtctgat |
| 61 | tttctctttg gattcttcca aaatcagagt cagactgctc ccggtgccat gaacggagac |
| 121 | gacgcctttg caaggagacc cacggttggt gctcaaatac cagagaagat ccaaaaggcc |
| 181 | ttcgatgata ttgccaaata cttctctaag gaagagtggg aaaagatgaa agcctcggag |
| 241 | aaaatcttct atgtgtatat gaagagaaag tatgaggcta tgactaaact aggtttcaag |
| 301 | gccaccctcc cacctttcat gtgtaataaa cgggccgaag acttccaggg gaatgatttg |
| 361 | gataatgacc ctaaccgtgg gaatcaggtt gaacgtcctc agatgacttt cggcaggctc |
| 421 | cagggaatct ccccgaagat catgcccaag aagccagcag aggaaggaaa tgattcggag |
| 481 | gaagtgccag aagcatctgg cccacaaaat gatgggaaag agctgtgccc cccgggaaaa |
| 541 | ccaactacct ctgagaagat tcacgagaga tctggaaata gggaggccca agaaaaggaa |
| 601 | gagagacgcg gaacagctca tcggtggagc agtcagaaca cacacaacat tggtcgattc |
| 661 | agtttgtcaa cttctatggg tgcagttcat ggtaccccca aaacaattac acacaacagg |
| 721 | gacccaaaag gggggaacat gcctggaccc acagactgcg tgagagaaaa cagctggtga |
| 781 | tttatgaaga gatcagcgac cctgaggaag atgacgagta actcccctca gggatacgac |
| 841 | acatgcccat gatgagaagc agaacgtggt gacctttcac gaacatgggc atggctgcgg |
| 901 | acccctcgtc atcaggtgca tagcaagtga aagcaagtgt tcacaacagt gaaaagttga |
| 961 | gcgtcatttt tcttagtgtg ccaagagttc gatgttagcg tttacgttgt attttcttac |
| 1021 | actgtgtcat tctgttagat actaacattt tcattgatga gcaagacata cttaatgcat |
| 1081 | attttggttt gtgtatccat gcacctacct tagaaaacaa gtattgtcgg ttacctctgc |
| 1141 | atggaacagc attaccctcc tctctcccca gatgtgacta ctgagggcag ttctgagtgt |
| 1201 | ttaatttcag attttttcct ctgcatttac acacacacgc acacaaacca caccacacac |
| 1261 | acacacacac acacacacac acacacacac acacaccaag taccagtata agcatctgcc |
| 1321 | atctgctttt cccattgcca tgcgtcctgg tcaagctccc ctcactctgt ttcctggtca |
| 1381 | gcatgtactc ccctcatccg attcccctgt agcagtcact gacagttaat aaacctttgc |
| 1441 | aaacgttcaa aaaaaaaaaa aaaaaa |
| // | |
| 22. SSX4 NM_005636 | |
| (SEQ ID NO: 22) | |
| 1 | acacgccgat ttgccctttt gattcttcca caatcagggt gagactgctc ccagtgccat |
| 61 | gaacggagac gacgcctttg caaggagacc cagggatgat gctcaaatat cagagaagtt |
| 121 | acgaaaggcc ttcgatgata ttgccaaata cttctctaag aaagagtggg aaaagatgaa |
| 181 | atcctcggag aaaatcgtct atgtgtatat gaagctaaac tatgaggtca tgactaaact |
| 241 | aggtttcaag gtcaccctcc cacctttcat gcgtagtaaa cgggctgcag acttccacgg |
| 301 | gaatgatttt ggtaacgatc gaaaccacag gaatcaggtt gaacgtcctc agatgacttt |
| 361 | cggcagcctc cagagaatct tcccgaagat catgcccaag aagccagcag aggaagaaaa |
| 421 | tggtttgaag gaagtgccag aggcatctgg cccacaaaat gatgggaaac agctgtgccc |
| 481 | cccgggaaat ccaagtacct tggagaagat taacaagaca tctggaccca aaagggggaa |
| 541 | acatgcctgg acccacagac tgcgtgagag aaagcagctg gtggtttatg aagagatcag |
| 601 | cgaccctgag gaagatgacg agtaactccc ctcggggata tgacacatgc ccatgatgag |
| 661 | aagcagaacg tggtgacctt tcacgaacat gggcatggct gcggacccct cgtcatcagg |
| 721 | tgcatagcaa gtgaaagcaa gtgttcacaa cagtgaaaag ttgagcgtca tttttcttag |
| 781 | tgtgccaaga gttcgatgtt ggcgtttccg ctgtattttc ttgcagtgtg ccattctgtt |
| 841 | agacattagc gttttcgttg atgagcaaga catgcttaat gcatatttcg gcttgtgtat |
| 901 | ccatgcacct acctcagaaa acaagtattg tcaggtattc tctccataga acagcactac |
| 961 | cctcctctct ccccagatgt gactactgag gggaggtctg agtgtttaat ttccgatttt |
| 1021 | ttcctctgca tttacacaca caccacacac gcacacacac acaccaagta ccagtataag |
| 1081 | catctcccat ctgcttttct ccattgccat gcgacctggt caagcccccc tcactctgtt |
| 1141 | tcctgttcag catgtactcc cctcatccga ttccgttgta tcagtcactg acagttaata |
| 1201 | aacctttgca aacgttcaaa aaaaaaaaaa aaaaaaaaaa aaaaaaaaaa |
| // | |
| 23. TSGA10 NM_025244 | |
| (SEQ ID NO: 23) | |
| 1 | agcacagaga taacggccag ccctgggcga aggttaccac caaagaatat gattttggtc |
| 61 | taggggctcc agtttctgaa gctgaaaact accagaatac tctccagcta gaacaagaag |
| 121 | tgagaaacca agatagattc atctcgacac tgaaattaca ggcatttgga tacctttgtg |
| 181 | atagaaaagg cccttttctt catccattga tggtttgata gtgggctggg aaggaaagct |
| 241 | gtgttcctcc acattaggca gcaaatactt gattgatttg atatttagct tagtcaaatt |
| 301 | gaagatctca aacagacaaa tcatggcttg gaagaatatg ttaggaaact cttggatagt |
| 361 | aaggaggtgg taagcagtca agtagatgat ttaaccagcc acaatgagca tctttgtaaa |
| 421 | gaattgatta aaattgacca actagcagag caactcgaaa aagagaaaaa ttttgtggtg |
| 481 | gattccgcca acaaggaact tgaagaagcc aagattgatc tcatttgcca gcaaaataat |
| 541 | attatagtat tggaagatac aataaaaagg cttaaatcta cttgattcct ttgtcaagac |
| 601 | tttggaagca gacaaagatc actataagag tgaagctcaa catttgagaa agatgatgcg |
| 661 | aagtaggtct aaaagtccaa gacgcccatc accaactgcc cggggtgcaa actgtgatgt |
| 721 | agaacttttg aagacaacaa caagagatcg tgaagaactt aaatgcatgc tggaaaaata |
| 781 | tgagcgccat ttggcagaaa ttcagggtaa tgtcaaggtt cttaaatctg agagagacaa |
| 841 | gatcttcctt ctttatgaac aggcacagga agaaattacc cgacttcgac gagaaatgat |
| 901 | gaaaagctgt aagagtccta aatcaacaac ggcacatgct attctccggc gagtggagac |
| 961 | tgaaagagat gtagccttta ctgatttacg aagaatgacc acagaacgag atagtctaag |
| 1021 | ggagaggcta aagattgctc aagagacagc atttaatgag aaggctcacc tggaacaaag |
| 1081 | gatagaggag ctggagtgta cagttcataa tcttgatgat gaacgtatgg agcaaatgtc |
| 1141 | aaatatgact ttgatgaagg aaaccataag cactgtggaa aaagaaatga aatcactagc |
| 1201 | aagaaaggca atggataccg aaagtgaact tggcagacaa aaagcagaga ataattcttt |
| 1261 | gagacttttg tatgaaaaca cagaaaaaga tctttctgat actcagcgac accttgctaa |
| 1321 | gaaaaaatat gagctacagc ttactcagga gaaaattatg tgcttggatg aaaaaattga |
| 1381 | taactttaca aggcaaaata ttgcacagcg agaagaaatc agcattcttg gtggaaccct |
| 1441 | caatgatctg gctaaagaaa aggaatgcct gcaagcatgt ttggataaaa aatctgagaa |
| 1501 | tattgcatcc cttggagaga gtttggcaat gaaagaaaag accatttcag gcatgaagaa |
| 1561 | tatcattgct gagatggaac aggcatcaag acagtgtact gaggccctaa ttgtgtgtga |
| 1621 | acaagacgtt tccagaatgc gtcggcaatt ggatgagaca aatgatgagc tggcccagat |
| 1681 | cgccagggaa agagatatct tggctcatga caatgacaat ctccaggaac agtttgctaa |
| 1741 | agctaaacaa gaaaaccagg cactgtccaa aaaattgaat gacactcata atgaacttaa |
| 1801 | tgacataaaa cagaaggttc aagatactaa tttggaggtt aacaagctga agaatatatt |
| 1861 | aaagtctgaa gaatctgaga accggcaaat gatggaacaa cttcgaaaag ccaatgaaga |
| 1921 | tgctgaaaac tgggaaaata aagcccgtca atcagaggca gataacaata ccctcaaact |
| 1981 | ggaacttatc actgctgagg cagagggtaa cagattaaaa gaaaaagtag attccctcaa |
| 2041 | cagagaggtt gagcaacact taaatgcaga aaggtcttac aagtcccaga tttctacctt |
| 2101 | acataaatct gttgtaaaaa tggaagagga gcttcagaag gttcagtttg aaaaagtgtc |
| 2161 | cgctcttgca gatttgtctt ctactaggga actctgtatt aaacttgact caagcaaaga |
| 2221 | acttcttaat cgacagctgg ttgctaaaga tcaagaaata gaaatgaggg agaatgagtt |
| 2281 | agattctgct cattctgaaa ttgaactcct gaggagtcag atggcaaatg agagaatctc |
| 2341 | catgcagaat ctagaagctt tgctggtggc caatcgagac aaagaatatc agtctcagat |
| 2401 | agcacttcaa gaaaaagaat ctgaaattca gcttcttaaa gaacaccttt gtttggcaga |
| 2461 | aaataaaatg gccatccaga gtagagatgt ggcccagttc agaaatgttg tcacacaatt |
| 2521 | ggaagctgat ttagacatta ccaaaagaca actaggaaca gagcgctttg aaagggagag |
| 2581 | ggccgtacaa gaacttcgcc gccaaaatta ttcaagtaat gcttatcata tgagttctac |
| 2641 | aatgaagcca aatacaaaat gtcattcacc agaacgtgct caccatcgat ctcctgaccg |
| 2701 | aggcctagat cgatcattag aagagaatct ttgctacaga gatttctgac acctgaaatg |
| 2761 | attcttcaca tccctgagaa aggtcaaagt tacaaactga tttttttttt tttgctacat |
| 2821 | gagtgcattt atcttttaaa tgcttggcaa tgttaaatgt atttattaac tttgtgtctc |
| 2881 | tgaatctctg ttctaatgtg ccatgttgca gtgatctgag atgacttata aaaacaaaaa |
| 2941 | tgtatatggc tctttctatc catgcagtga tagtgagtgt aaaatctgct tacttcacta |
| 3001 | ttgaacacta ttctgttcac tatctggagt aaataaagaa gcttattaaa acagggaaaa |
| 3061 | gtgtttttac aaaactgctt tcctttcctt tcttggtatc tcaaataatt ggttggttaa |
| 3121 | ttttttttct ttgtgatttg tgttttcatg gctggaaaat ctgtgccaaa atgggtacaa |
| 3181 | cttactgata ctggtctggg atctgatatt ccattttgga accaacctta catgaattcc |
| 3241 | atgccataat ctgatattga agcagcagaa agtaggagtg ttgtcagggt tttaaaacct |
| 3301 | atagtattgt accactcaaa gactaagtaa catacatatt ctacaattgt gtttactact |
| 3361 | actgctttta gaaaggtagg tgatggtata tctgtcaaga atgtaagatt ttattaaaaa |
| 3421 | tctttatttt taataagttt tctttcaagt actgcaatca acttaattgc gattttattt |
| 3481 | tattgtctgt ggcagctgtc tgtaatctgg agtaataaga taagtgatgt ttccttcctc |
| 3541 | ccccaatttc ctaaatctct taccttatct atccataggc tttgatgtaa tgttcatatt |
| 3601 | agagcctgaa tgctactgta ttttactgat ataaactatt tgtattcttg ccattttaat |
| 3661 | aaaaaaaggc ttgtcattaa tagta |
| // | |
| 24. TSSK6 NM_032037 | |
| (SEQ ID NO: 24) | |
| 1 | ccggggcggg gtcccggacc cccaccctaa ggcggagtga cgcccgcagt cacttcacaa |
| 61 | ggcaaaaatt gttacggggc aataaaaggc acaacagcgg ccaatgtctg gcagtgggca |
| 121 | catgggggtg cgggggtgta ggtgccaagc gccatggctt agacccgaga ttggagtccg |
| 181 | gccgcccccc gacagcagcc gcctcctgcc ccccgtgcgc cctaggcgcc accatgtcgg |
| 241 | gagacaaact tctgagcgaa ctcggttata agctgggccg cacaattgga gagggcagct |
| 301 | actccaaggt gaaggtggcc acatccaaga agtacaaggg taccgtggcc atcaaggtgg |
| 361 | tggaccggcg gcgagcgccc ccggacttcg tcaacaagtt cctgccgcga gagctgtcca |
| 421 | tcctgcgggg cgtgcgacac ccgcacatcg tgcacgtctt cgagttcatc gaggtgtgca |
| 481 | acgggaaact gtacatcgtg atggaagcgg ccgccaccga cctgctgcaa gccgtgcagc |
| 541 | gcaacgggcg catccccgga gttcaggcgc gcgacctctt tgcgcagatc gccggcgccg |
| 601 | tgcgctacct gcacgatcat cacctggtgc accgcgacct caagtgcgaa aacgtgctgc |
| 661 | tgagcccgga cgagcgccgc gtcaagctca ccgacttcgg cttcggccgc caggcccatg |
| 721 | gctacccaga cctgagcacc acctactgcg gctcagccgc ctacgcgtca cccgaggtgc |
| 781 | tcctgggcat cccctacgac cccaagaagt acgatgtgtg gagcatgggc gtcgtgctct |
| 841 | acgtcatggt caccgggtgc atgcccttcg acgactcgga catcgccggc ctgccccggc |
| 901 | gccagaaacg cggcgtgctc tatcccgaag gcctcgagct gtccgagcgc tgcaaggccc |
| 961 | tgatcgccga gctgctgcag ttcagcccgt ccgccaggcc ctccgcgggc caggtagcgc |
| 1021 | gcaactgctg gctgcgcgcc ggggactccg gctagaagcc gggtggttcc agccattcct |
| 1081 | gcagccaagg gcactgggcc agggcggcgc acgcgcaaga ggcgcgcttc gagggaatat |
| 1141 | gcgaagctgc cgcgtgctgc tgcacatgcg ctttttccct tccgcttccc tccctttctt |
| 1201 | cccacggggg agtccgcagt tgcccttgtt cggaatccac gttccccgcg atcccgggag |
| 1261 | ctggaggcgc atgcgcatcc gcgattccct gcgaccaggc cccgagaggg cgagaccaga |
| 1321 | ggggacggaa gcattgcgcc tgcgcggaac tctcagcctc tgcgcggagg gcgtcccttc |
| 1381 | ccaaccagcc gtgggtgcca ggttcccggt tggaacctgc aataaactcg ctgttcctcg |
| 1441 | caaaaaaaaa aaaaaaaaa |
| // | |
| 25. TYR NM_000372 | |
| (SEQ ID NO: 25) | |
| 1 | atcactgtag tagtagctgg aaagagaaat ctgtgactcc aattagccag ttcctgcaga |
| 61 | ccttgtgagg actagaggaa gaatgctcct ggctgttttg tactgcctgc tgtggagttt |
| 121 | ccagacctcc gctggccatt tccctagagc ctgtgtctcc tctaagaacc tgatggagaa |
| 181 | ggaatgctgt ccaccgtgga gcggggacag gagtccctgt ggccagcttt caggcagagg |
| 241 | ttcctgtcag aatatccttc tgtccaatgc accacttggg cctcaatttc ccttcacagg |
| 301 | ggtggatgac cgggagtcgt ggccttccgt cttttataat aggacctgcc agtgctctgg |
| 361 | caacttcatg ggattcaact gtggaaactg caagtttggc ttttggggac caaactgcac |
| 421 | agagagacga ctcttggtga gaagaaacat cttcgatttg agtgccccag agaaggacaa |
| 481 | attttttgcc tacctcactt tagcaaagca taccatcagc tcagactatg tcatccccat |
| 541 | agggacctat ggccaaatga aaaatggatc aacacccatg tttaacgaca tcaatattta |
| 601 | tgacctcttt gtctggatgc attattatgt gtcaatggat gcactgcttg ggggatctga |
| 661 | aatctggaga gacattgatt ttgcccatga agcaccagct tttctgcctt ggcatagact |
| 721 | cttcttgttg cggtgggaac aagaaatcca gaagctgaca ggagatgaaa acttcactat |
| 781 | tccatattgg gactggcggg atgcagaaaa gtgtgacatt tgcacagatg agtacatggg |
| 841 | aggtcagcac cccacaaatc ctaacttact cagcccagca tcattcttct cctcttggca |
| 901 | gattgtctgt agccgattgg aggagtacaa cagccatcag tctttatgca atggaacgcc |
| 961 | cgagggacct ttacggcgta atcctggaaa ccatgacaaa tccagaaccc caaggctccc |
| 1021 | ctcttcagct gatgtagaat tttgcctgag tttgacccaa tatgaatctg gttccatgga |
| 1081 | taaagctgcc aatttcagct ttagaaatac actggaagga tttgctagtc cacttactgg |
| 1141 | gatagcggat gcctctcaaa gcagcatgca caatgccttg cacatctata tgaatggaac |
| 1201 | aatgtcccag gtacagggat ctgccaacga tcctatcttc cttcttcacc atgcatttgt |
| 1261 | tgacagtatt tttgagcagt ggctccgaag gcaccgtcct cttcaagaag tttatccaga |
| 1321 | agccaatgca cccattggac ataaccggga atcctacatg gttcctttta taccactgta |
| 1381 | cagaaatggt gatttcttta tttcatccaa agatctgggc tatgactata gctatctaca |
| 1441 | agattcagac ccagactctt ttcaagacta cattaagtcc tatttggaac aagcgagtcg |
| 1501 | gatctggtca tggctccttg gggcggcgat ggtaggggcc gtcctcactg ccctgctggc |
| 1561 | agggcttgtg agcttgctgt gtcgtcacaa gagaaagcag cttcctgaag aaaagcagcc |
| 1621 | actcctcatg gagaaagagg attaccacag cttgtatcag agccatttat aaaaggctta |
| 1681 | ggcaatagag tagggccaaa aagcctgacc tcactctaac tcaaagtaat gtccaggttc |
| 1741 | ccagagaata tctgctggta tttttctgta aagaccattt gcaaaattgt aacctaatac |
| 1801 | aaagtgtagc cttcttccaa ctcaggtaga acacacctgt ctttgtcttg ctgttttcac |
| 1861 | tcagcccttt taacattttc ccctaagccc atatgtctaa ggaaaggatg ctatttggta |
| 1921 | atgaggaact gttatttgta tgtgaattaa agtgctctta ttttaaaaaa ttgaaataat |
| 1981 | tttgattttt gccttctgat tatttaaaga tctatatatg ttttattggc cccttcttta |
| 2041 | ttttaataaa acagtgagaa atctaaaaaa aaaaaaaaaa aa |
| // | |
| 26. RAGE-2 NM_130777 | |
| (SEQ ID NO: 26) | |
| 1 | accagggagg ggagggagga ctgcatgacg cgggatgggg tggggcgagg cggggcactg |
| 61 | cagcacacaa cgcaggcacc gacttcagtg tgcatgttcc ttggacacct gcctcagtgt |
| 121 | gcatgttcac tgggcatctt cccttcgacc cctttgccca cgtggtgacc gctggggagc |
| 181 | tgtgagagtg tgaggggcac gttccagccg tctggactct ttctctccta ctgagacgca |
| 241 | gcctataggt ccgcaggcca gtcctcccag gaactgaaat agtgaaatat gagttggcga |
| 301 | ggaagatcaa catataggcc taggccaaga agaagtttac agcctcctga gctgattggg |
| 361 | gctatgcttg aacccactga tgaagagcct aaagaagaga aaccacccac taaaagtcgg |
| 421 | aatcctacac ctgatcagaa gagagaagat gatcagggtg cagctgagat tcaagtgcct |
| 481 | gacctggaag ccgatctcca ggagctatgt cagacaaaga ctggggatgg atgtgaaggt |
| 541 | ggtactgatg tcaaggggaa gattctacca aaagcagagc actttaaaat gccagaagca |
| 601 | ggtgaaggga aatcacaggt ttaaaggaag ataagctgaa acaacacaaa ctgtttttat |
| 661 | attagatatt ttactttaaa atatcttaat aaagttttaa gcttttctcc aaaaaaaaaa |
| // | |
| 27. ZNF165 NM_003447 | |
| (SEQ ID NO: 27) | |
| 1 | cttttcagga gtcccttctg ttatttagca agacacacta ctacaatgga gaaaaaagat |
| 61 | gccccctcct tcaagccctg agatcttcag gactttggcg aaaagtctgc gcccgaagag |
| 121 | acccaggaag gattcttgga attgtagtcc aaaggcatcc cgccttctgc gcagactcac |
| 181 | aagtccctgt ggacggaatt cttgaagtgt agcgccgctc agtccttcca ccggaagtgt |
| 241 | ccgatcggaa tcagccctgt ccgagaggtg agtccgggtt tggggatcca gatgtccagc |
| 301 | cccgtgtccc cctccaaaca tccagtccct ctcatattgc ctttgaaatt agcagcctct |
| 361 | gggtgaccag accttggccc tcagaggaat cccggagaaa ggtagaacca gcttcggcgt |
| 421 | tgggaacgca ggcgcgctta cgcatttagt gagggtttgg cggtctccat agttaccgcc |
| 481 | gccgcgcgtg acgtcatagt ggagcgctga gggcttggtg gcgtggggtg ggggctgtcc |
| 541 | tactgatcct gaatttgggg tcactggtaa gaggagttgc ccattccagc caggtggaac |
| 601 | ggggaggggt gccacatgtc tcagatctgc cattgtctgc gaaaagaaac tgctgcgagg |
| 661 | accatcccca atcccctgct tcccttggga agagtaaccg ccgttttgta ggacacttgg |
| 721 | ggacaacccc gcttgtcctg aaatttattg acacggtaaa tagtatttcc tgtgtgccga |
| 781 | ggatgcagtt aaaccaacac tgaccccctg cccttgagaa acacaagatg gctacagaac |
| 841 | caaagaaagc tgcagcccag aactctccag aggatgaagg acttctgata gtgaagatag |
| 901 | aagaggaaga atttatccat gggcaggaca cttgcttaca gagaagtgaa ctccttaagc |
| 961 | aggagctctg caggcagctt tttaggcagt tctgctacca ggattctcct ggacctcgcg |
| 1021 | aggcactgag ccgcctccgg gagctctgct gtcagtggct gaagccagag atccatacca |
| 1081 | aggaacagat tctggaactg ctggtgctag agcagttcct gaccatcctg ccaggagatt |
| 1141 | tgcaggcctg ggtacatgaa cattacccag agagtggaga ggaggcagtg accatactag |
| 1201 | aagatttgga gagaggcact gatgaagcag tactccaggt tcaagcccat gaacatggac |
| 1261 | aagaaatatt ccagaaaaaa gtgtcacctc ctggaccagc acttaatgtc aagttacagc |
| 1321 | cagtggagac caaggcccat tttgattcat cagaacccca gctcctatgg gactgtgata |
| 1381 | atgagagtga aaacagtaga tccatgccaa agctggaaat ttttgaaaaa attgaatcac |
| 1441 | agagaattat atctggaaga atctcaggat acatatcaga agcatctggt gagtctcaag |
| 1501 | acatctgtaa gtctgcaggc agggtaaaga gacaatggga aaaagaatca ggggagtctc |
| 1561 | agagactctc gtctgcccag gatgaaggtt ttggtaaaat cctcacccac aaaaatacag |
| 1621 | tcagaggtga aataataagc cacgatggat gtgagaggag attaaatctg aactcaaatg |
| 1681 | aattcacaca ccagaaatct tgtaaacatg gtacctgtga ccagagcttc aaatggaact |
| 1741 | cagattttat taaccatcaa ataatttatg ctggagaaaa aaatcaccaa tatggaaaat |
| 1801 | ctttcaagag cccaaaactt gctaaacatg cagcagtttt cagtggagat aaaactcatc |
| 1861 | agtgtaatga atgtgggaaa gctttcaggc acagctcaaa acttgctagg catcagagaa |
| 1921 | tccacactgg agagagatgc tatgaatgta atgaatgtgg gaaaagcttt gcagagagct |
| 1981 | cagatcttac tagacatcgg cgaattcaca ctggggaaag accctttggt tgcaaagaat |
| 2041 | gtgggagagc attcaacctg aactcacatc ttatcaggca tcagagaatt cacaccagag |
| 2101 | agaaacccta cgagtgtagt gaatgtggga aaaccttccg agtgagctca catcttattc |
| 2161 | gacactttag aattcacact ggagaaaaac cctatgaatg cagtgagtgt ggaagagcct |
| 2221 | tcagtcagag ctcaaacctt agtcaacacc agagaattca catgagggaa aacctattaa |
| 2281 | tgtaaggaac ttaaatttgt aagtaaatgc tgaggaaatg gcacaatatg aaaaatatta |
| 2341 | aataaaaaat aaatattggg caagatggaa gactgaaaaa aaaaaaaaaa aaaaaaaaaa |
| 2401 | aaaaaaaaaa a |
| // | |
| 28. MAPK1 NM_002745 | |
| (SEQ ID NO: 28) | |
| 1 | gcccctccct ccgcccgccc gccggcccgc ccgtcagtct ggcaggcagg caggcaatcg |
| 61 | gtccgagtgg ctgtcggctc ttcagctctc ccgctcggcg tcttccttcc tcctcccggt |
| 121 | cagcgtcggc ggctgcaccg gcggcggcgc agtccctgcg ggaggggcga caagagctga |
| 181 | gcggcggccg ccgagcgtcg agctcagcgc ggcggaggcg gcggcggccc ggcagccaac |
| 241 | atggcggcgg cggcggcggc gggcgcgggc ccggagatgg tccgcgggca ggtgttcgac |
| 301 | gtggggccgc gctacaccaa cctctcgtac atcggcgagg gcgcctacgg catggtgtgc |
| 361 | tctgcttatg ataatgtcaa caaagttcga gtagctatca agaaaatcag cccctttgag |
| 421 | caccagacct actgccagag aaccctgagg gagataaaaa tcttactgcg cttcagacat |
| 481 | gagaacatca ttggaatcaa tgacattatt cgagcaccaa ccatcgagca aatgaaagat |
| 541 | gtatatatag tacaggacct catggaaaca gatctttaca agctcttgaa gacacaacac |
| 601 | ctcagcaatg accatatctg ctattttctc taccagatcc tcagagggtt aaaatatatc |
| 661 | cattcagcta acgttctgca ccgtgacctc aagccttcca acctgctgct caacaccacc |
| 721 | tgtgatctca agatctgtga ctttggcctg gcccgtgttg cagatccaga ccatgatcac |
| 781 | acagggttcc tgacagaata tgtggccaca cgttggtaca gggctccaga aattatgttg |
| 841 | aattccaagg gctacaccaa gtccattgat atttggtctg taggctgcat tctggcagaa |
| 901 | atgctttcta acaggcccat ctttccaggg aagcattatc ttgaccagct gaaccacatt |
| 961 | ttgggtattc ttggatcccc atcacaagaa gacctgaatt gtataataaa tttaaaagct |
| 1021 | aggaactatt tgctttctct tccacacaaa aataaggtgc catggaacag gctgttccca |
| 1081 | aatgctgact ccaaagctct ggacttattg gacaaaatgt tgacattcaa cccacacaag |
| 1141 | aggattgaag tagaacaggc tctggcccac ccatatctgg agcagtatta cgacccgagt |
| 1201 | gacgagccca tcgccgaagc accattcaag ttcgacatgg aattggatga cttgcctaag |
| 1261 | gaaaagctca aagaactaat ttttgaagag actgctagat tccagccagg atacagatct |
| 1321 | taaatttgtc aggacaaggg ctcagaggac tggacgtgct cagacatcgg tgttcttctt |
| 1381 | cccagttctt gacccctggt cctgtctcca gcccgtcttg gcttatccac tttgactcct |
| 1441 | ttgagccgtt tggaggggcg gtttctggta gttgtggctt ttatgctttc aaagaatttc |
| 1501 | ttcagtccag agaattcctc ctggcagccc tgtgtgtgtc acccattggt gacctgcggc |
| 1561 | agtatgtact tcagtgcacc tactgcttac tgttgcttta gtcactaatt gctttctggt |
| 1621 | ttgaaagatg cagtggttcc tccctctcct gaatcctttt ctacatgatg ccctgctgac |
| 1681 | catgcagccg caccagagag agattcttcc ccaattggct ctagtcactg gcatctcact |
| 1741 | ttatgatagg gaaggctact acctagggca ctttaagtca gtgacagccc cttatttgca |
| 1801 | cttcaccttt tgaccataac tgtttcccca gagcaggagc ttgtggaaat accttggctg |
| 1861 | atgttgcagc ctgcagcaag tgcttccgtc tccggaatcc ttggggagca cttgtccacg |
| 1921 | tcttttctca tatcatggta gtcactaaca tatataaggt atgtgctatt ggcccagctt |
| 1981 | ttagaaaatg cagtcatttt tctaaataaa aaggaagtac tgcacccagc agtgtcactc |
| 2041 | tgtagttact gtggtcactt gtaccatata gaggtgtaac acttgtcaag aagcgttatg |
| 2101 | tgcagtactt aatgtttgta agacttacaa aaaaagattt aaagtggcag cttcactcga |
| 2161 | catttggtga gagaagtaca aaggttgcag tgctgagctg tgggcggttt ctggggatgt |
| 2221 | cccagggtgg aactccacat gctggtgcat atacgccctt gagctacttc aaatgtgggt |
| 2281 | gtttcagtaa ccacgttcca tgcctgagga tttagcagag aggaacactg cgtctttaaa |
| 2341 | tgagaaagta tacaattctt tttccttcta cagcatgtca gcatctcaag ttcatttttc |
| 2401 | aacctacagt ataacaattt gtaataaagc ctccaggagc tcatgacgtg aagcactgtt |
| 2461 | ctgtcctcaa gtactcaaat atttctgata ctgctgagtc agactgtcag aaaaagctag |
| 2521 | cactaactcg tgtttggagc tctatccata ttttactgat ctctttaagt atttgttcct |
| 2581 | gccactgtgt actgtggagt tgactcggtg ttctgtccca gtgcggtgcc tcctcttgac |
| 2641 | ttccccactg ctctctgtgg tgagaaattt gccttgttca ataattactg taccctcgca |
| 2701 | tgactgttac agctttctgt gcagagatga ctgtccaagt gccacatgcc tacgattgaa |
| 2761 | atgaaaactc tattgttacc tctgagttgt gttccacgga aaatgctatc cagcagatca |
| 2821 | tttaggaaaa ataattctat ttttagcttt tcatttctca gctgtccttt tttcttgttt |
| 2881 | gatttttgac agcaatggag aatgggttat ataaagactg cctgctaata tgaacagaaa |
| 2941 | tgcatttgta attcatgaaa ataaatgtac atcttctatc ttcacattca tgttaagatt |
| 3001 | cagtgttgct ttcctctgga tcagcgtgtc tgaatggaca gtcaggttca ggttgtgctg |
| 3061 | aacacagaaa tgctcacagg cctcactttg ccgcccaggc actggcccag cacttggatt |
| 3121 | tacataagat gagttagaaa ggtacttctg tagggtcctt tttacctctg ctcggcagag |
| 3181 | aatcgatgct gtcatgttcc tttattcaca atcttaggtc tcaaatattc tgtcaaaccc |
| 3241 | taacaaagaa gccccgacat ctcaggttgg attccctggt tctctctaaa gagggcctgc |
| 3301 | ccttgtgccc cagaggtgct gctgggcaca gccaagagtt gggaagggcc gccccacagt |
| 3361 | acgcagtcct caccacccag cccagggtgc tcacgctcac cactcctgtg gctgaggaag |
| 3421 | gatagctggc tcatcctcgg aaaacagacc cacatctcta ttcttgccct gaaatacgcg |
| 3481 | cttttcactt gcgtgctcag agctgccgtc tgaaggtcca cacagcattg acgggacaca |
| 3541 | gaaatgtgac tgttaccgga taacactgat tagtcagttt tcatttataa aaaagcattg |
| 3601 | acagttttat tactcttgtt tctttttaaa tggaaagtta ctattataag gttaatttgg |
| 3661 | agtcctcttc taaatagaaa accatatcct tggctactaa catctggaga ctgtgagctc |
| 3721 | cttcccattc cccttcctgg tactgtggag tcagattggc atgaaaccac taacttcatt |
| 3781 | ctagaatcat tgtagccata agttgtgtgc tttttattaa tcatgccaaa cataatgtaa |
| 3841 | ctgggcagag aatggtccta accaaggtac ctatgaaaag cgctagctat catgtgtagt |
| 3901 | agatgcatca ttttggctct tcttacattt gtaaaaatgt acagattagg tcatcttaat |
| 3961 | tcatattagt gacacggaac agcacctcca ctatttgtat gttcaaataa gctttcagac |
| 4021 | taatagcttt tttggtgtct aaaatgtaag caaaaaattc ctgctgaaac attccagtcc |
| 4081 | tttcatttag tataaaagaa atactgaaca agccagtggg atggaattga aagaactaat |
| 4141 | catgaggact ctgtcctgac acaggtcctc aaagctagca gagatacgca gacattgtgg |
| 4201 | catctgggta gaagaatact gtattgtgtg tgcagtgcac agtgtgtggt gtgtgcacac |
| 4261 | tcattccttc tgctcttggg cacaggcagt gggtgtagag gtaaccagta gctttgagaa |
| 4321 | gctacatgta gctcaccagt ggttttctct aaggaatcac aaaagtaaac tacccaacca |
| 4381 | catgccacgt aatatttcag ccattcagag gaaactgttt tctctttatt tgcttatatg |
| 4441 | ttaatatggt ttttaaattg gtaactttta tatagtatgg taacagtatg ttaatacaca |
| 4501 | catacatacg cacacatgct ttgggtcctt ccataatact tttatatttg taaatcaatg |
| 4561 | ttttggagca atcccaagtt taagggaaat atttttgtaa atgtaatggt tttgaaaatc |
| 4621 | tgagcaatcc ttttgcttat acatttttaa agcatttgtg ctttaaaatt gttatgctgg |
| 4681 | tgtttgaaac atgatactcc tgtggtgcag atgagaagct ataacagtga atatgtggtt |
| 4741 | tctcttacgt catccacctt gacatgatgg gtcagaaaca aatggaaatc cagagcaagt |
| 4801 | cctccagggt tgcaccaggt ttacctaaag cttgttgcct tttcttgtgc tgtttatgcg |
| 4861 | tgtagagcac tcaagaaagt tctgaaactg ctttgtatct gctttgtact gttggtgcct |
| 4921 | tcttggtatt gtaccccaaa attctgcata gattatttag tataatggta agttaaaaaa |
| 4981 | tgttaaagga agattttatt aagaatctga atgtttattc attatattgt tacaatttaa |
| 5041 | cattaacatt tatttgtggt atttgtgatt tggttaatct gtataaaaat tgtaagtaga |
| 5101 | aaggtttata tttcatctta attcttttga tgttgtaaac gtacttttta aaagatggat |
| 5161 | tatttgaatg tttatggcac ctgacttgta aaaaaaaaaa actacaaaaa aatccttaga |
| 5221 | atcattaaat tgtgtccctg tattaccaaa ataacacagc accgtgcatg tatagtttaa |
| 5281 | ttgcagtttc atctgtgaaa acgtgaaatt gtctagtcct tcgttatgtt ccccagatgt |
| 5341 | cttccagatt tgctctgcat gtggtaactt gtgttagggc tgtgagctgt tcctcgagtt |
| 5401 | gaatggggat gtcagtgctc ctagggttct ccaggtggtt cttcagacct tcacctgtgg |
| 5461 | gggggggggt aggcggtgcc cacgcccatc tcctcatcct cctgaacttc tgcaacccca |
| 5521 | ctgctgggca gacatcctgg gcaacccctt ttttcagagc aagaagtcat aaagatagga |
| 5581 | tttcttggac atttggttct tatcaatatt gggcattatg taatgactta tttacaaaac |
| 5641 | aaagatactg gaaaatgttt tggatgtggt gttatggaaa gagcacaggc cttggaccca |
| 5701 | tccagctggg ttcagaacta ccccctgctt ataactgcgg ctggctgtgg gccagtcatt |
| 5761 | ctgcgtctct gctttcttcc tctgcttcag actgtcagct gtaaagtgga agcaatatta |
| 5821 | cttgccttgt atatggtaaa gattataaaa atacatttca actgttcagc atagtacttc |
| 5881 | aaagcaagta ctcagtaaat agcaagtctt tttaaa |
| // | |
| 29. BRAF NM_004333 | |
| (SEQ ID NO: 29) | |
| 1 | cgcctccctt ccccctcccc gcccgacagc ggccgctcgg gccccggctc tcggttataa |
| 61 | gatggcggcg ctgagcggtg gcggtggtgg cggcgcggag ccgggccagg ctctgttcaa |
| 121 | cggggacatg gagcccgagg ccggcgccgg cgccggcgcc gcggcctctt cggctgcgga |
| 181 | ccctgccatt ccggaggagg tgtggaatat caaacaaatg attaagttga cacaggaaca |
| 241 | tatagaggcc ctattggaca aatttggtgg ggagcataat ccaccatcaa tatatctgga |
| 301 | ggcctatgaa gaatacacca gcaagctaga tgcactccaa caaagagaac aacagttatt |
| 361 | ggaatctctg gggaacggaa ctgatttttc tgtttctagc tctgcatcaa tggataccgt |
| 421 | tacatcttct tcctcttcta gcctttcagt gctaccttca tctctttcag tttttcaaaa |
| 481 | tcccacagat gtggcacgga gcaaccccaa gtcaccacaa aaacctatcg ttagagtctt |
| 541 | cctgcccaac aaacagagga cagtggtacc tgcaaggtgt ggagttacag tccgagacag |
| 601 | tctaaagaaa gcactgatga tgagaggtct aatcccagag tgctgtgctg tttacagaat |
| 661 | tcaggatgga gagaagaaac caattggttg ggacactgat atttcctggc ttactggaga |
| 721 | agaattgcat gtggaagtgt tggagaatgt tccacttaca acacacaact ttgtacgaaa |
| 781 | aacgtttttc accttagcat tttgtgactt ttgtcgaaag ctgcttttcc agggtttccg |
| 841 | ctgtcaaaca tgtggttata aatttcacca gcgttgtagt acagaagttc cactgatgtg |
| 901 | tgttaattat gaccaacttg atttgctgtt tgtctccaag ttctttgaac accacccaat |
| 961 | accacaggaa gaggcgtcct tagcagagac tgccctaaca tctggatcat ccccttccgc |
| 1021 | acccgcctcg gactctattg ggccccaaat tctcaccagt ccgtctcctt caaaatccat |
| 1081 | tccaattcca cagcccttcc gaccagcaga tgaagatcat cgaaatcaat ttgggcaacg |
| 1141 | agaccgatcc tcatcagctc ccaatgtgca tataaacaca atagaacctg tcaatattga |
| 1201 | tgacttgatt agagaccaag gatttcgtgg tgatggagga tcaaccacag gtttgtctgc |
| 1261 | taccccccct gcctcattac ctggctcact aactaacgtg aaagccttac agaaatctcc |
| 1321 | aggacctcag cgagaaagga agtcatcttc atcctcagaa gacaggaatc gaatgaaaac |
| 1381 | acttggtaga cgggactcga gtgatgattg ggagattcct gatgggcaga ttacagtggg |
| 1441 | acaaagaatt ggatctggat catttggaac agtctacaag ggaaagtggc atggtgatgt |
| 1501 | ggcagtgaaa atgttgaatg tgacagcacc tacacctcag cagttacaag ccttcaaaaa |
| 1561 | tgaagtagga gtactcagga aaacacgaca tgtgaatatc ctactcttca tgggctattc |
| 1621 | cacaaagcca caactggcta ttgttaccca gtggtgtgag ggctccagct tgtatcacca |
| 1681 | tctccatatc attgagacca aatttgagat gatcaaactt atagatattg cacgacagac |
| 1741 | tgcacagggc atggattact tacacgccaa gtcaatcatc cacagagacc tcaagagtaa |
| 1801 | taatatattt cttcatgaag acctcacagt aaaaataggt gattttggtc tagctacagt |
| 1861 | gaaatctcga tggagtgggt cccatcagtt tgaacagttg tctggatcca ttttgtggat |
| 1921 | ggcaccagaa gtcatcagaa tgcaagataa aaatccatac agctttcagt cagatgtata |
| 1981 | tgcatttgga attgttctgt atgaattgat gactggacag ttaccttatt caaacatcaa |
| 2041 | caacagggac cagataattt ttatggtggg acgaggatac ctgtctccag atctcagtaa |
| 2101 | ggtacggagt aactgtccaa aagccatgaa gagattaatg gcagagtgcc tcaaaaagaa |
| 2161 | aagagatgag agaccactct ttccccaaat tctcgcctct attgagctgc tggcccgctc |
| 2221 | attgccaaaa attcaccgca gtgcatcaga accctccttg aatcgggctg gtttccaaac |
| 2281 | agaggatttt agtctatatg cttgtgcttc tccaaaaaca cccatccagg cagggggata |
| 2341 | tggtgcgttt cctgtccact gaaacaaatg agtgagagag ttcaggagag tagcaacaaa |
| 2401 | aggaaaataa atgaacatat gtttgcttat atgttaaatt gaataaaata ctctcttttt |
| 2461 | ttttaaggtg aaccaaagaa cacttgtgtg gttaaagact agatataatt tttccccaaa |
| 2521 | ctaaaattta tacttaacat tggattttta acatccaagg gttaaaatac atagacattg |
| 2581 | ctaaaaattg gcagagcctc ttctagaggc tttactttct gttccgggtt tgtatcattc |
| 2641 | acttggttat tttaagtagt aaacttcagt ttctcatgca acttttgttg ccagctatca |
| 2701 | catgtccact agggactcca gaagaagacc ctacctatgc ctgtgtttgc aggtgagaag |
| 2761 | ttggcagtcg gttagcctgg gttagataag gcaaactgaa cagatctaat ttaggaagtc |
| 2821 | agtagaattt aataattcta ttattattct taataatttt tctataacta tttcttttta |
| 2881 | taacaatttg gaaaatgtgg atgtctttta tttccttgaa gcaataaact aagtttcttt |
| 2941 | ttataaaaa |
| // | |
| 30. CABYR NM_012189 | |
| (SEQ ID NO: 30) | |
| 1 | tgcggagctt cgtgatgcac gccccgatgc ctgcggggct ataaaaacgc tcgcaagcgc |
| 61 | caagtctcct caggagccgc cggcaagggg gcaacgagga agctcttaag agcgcggccg |
| 121 | gaaagcagtt gagttacaga catcctgcca aaatgatttc ttcaaagccc agacttgtcg |
| 181 | taccctatgg cctcaagact ctgctcgagg gaattagcag agctgttctc aaaaccaacc |
| 241 | catcaaacat caaccagttt gcagcagctt attttcaaga acttactatg tatagaggga |
| 301 | atactactat ggatataaaa gatctggtta aacaatttca tcagattaaa gtagagaaat |
| 361 | ggtcagaagg aacgacacca cagaagaaat tagaatgttt aaaagaacca ggaaaaacat |
| 421 | ctgtagaatc taaagtacct acccagatgg aaaaatctac agacacagac gaggacaatg |
| 481 | taaccagaac agaatatagt gacaaaacca cccagtttcc atcagtttat gctgtgccag |
| 541 | gcactgagca aacggaagca gttggtggtc tttcttccaa accagccacc cctaagacta |
| 601 | ctaccccacc ctcatcacca cctccaacag ctgtctcacc agagtttgcc tacgtcccag |
| 661 | ctgacccagc tcagcttgct gctcagatgt taggtaaagt ttcatctatt cattctgatc |
| 721 | aatctgatgt gttaatggtg gatgtggcaa ccagtatgcc tgttgttatc aaggaggtgc |
| 781 | caagctcaga ggctgctgaa gatgtcatgg tggctgctcc tcttgtgtgt tctggaaagg |
| 841 | tgctagaagt gcaggttgtg aaccaaacat ctgtccatgt agatttgggt tctcaaccta |
| 901 | aagaaaatga ggctgaacca tcaacggctt cctcagtccc cttgcaggat gaacaagaac |
| 961 | ctcctgctta tgatcaagct cctgaggtca ctttgcaggc tgatattgag gttatgtcaa |
| 1021 | ctgttcatat atcatctgtc tataacgatg tgcctgtgac tgaaggagtt gtttatatcg |
| 1081 | agcaactgcc agaacaaata gttatccctt ttactgatca agttgcttgt cttaaagaaa |
| 1141 | atgagcagtc aaaagaaaat gagcagtcac cacgagttag tcccaaatct gtagtagaaa |
| 1201 | agaccacctc tggcatgtct aaaaaatctg tagagtctgt aaaacttgca cagttggagg |
| 1261 | agaatgcaaa atattcctca gtatatatgg aggcagaagc aacagctctg ctctctgaca |
| 1321 | catctttgaa aggtcagcct gaggtacctg cacaactcct ggatgcagaa ggtgctatca |
| 1381 | aaataggctc tgaaaaatct ctgcaccttg aagtggagat cacttcaata gtctctgaca |
| 1441 | atactgggca ggaggagtct ggggaaaact ctgtacccca ggagatggaa ggcaaacctg |
| 1501 | tgctctctgg ggaagctgca gaagcagtgc actcaggtac atctgtaaag tcatctagtg |
| 1561 | gccccttccc tcctgctcca gaaggcctta ctgcaccaga aattgaacca gaaggggaat |
| 1621 | caacagctga ataaggtttg atgaagccag caatggcaac aagtgaacga ggacaaccac |
| 1681 | caccatgttc taacatgtgg accctttatt gtctaactga taagaatcaa caaggtcacc |
| 1741 | catcaccgcc acctgcacct gggccttttc cccaagcaac cctctattta cctaatccta |
| 1801 | aggatccaca gtttcagcag catccaccaa aagtcacttt tccaacttat gtgatgggcg |
| 1861 | acaccaagaa gaccagtgcc ccacctttta tcttagtagg ctcaaatgtt caggaagcac |
| 1921 | agggatggaa acctcttcct ggacatgctg tcgtttcaca gtcagatgtc ttgagatatg |
| 1981 | ttgcaatgca agtgcccatt gctgttcctg cagatgagaa ataccagaaa cataccctaa |
| 2041 | gtccccagaa tgctaatcct ccaagtggac aagatgtccc caggccaaaa agccctgttt |
| 2101 | tcctttctgt tgctttccca gtagaagatg tagctaaaaa aagttcagga tctggtgaca |
| 2161 | aatgtgctcc ctttggaagt tacggtattg ctggggaggt aaccgtgact actgctcaca |
| 2221 | aacgtcgcaa agcagaaact gaaaactgat ccagaaatga cgctgtctgg gtcaacattt |
| 2281 | cagggaggag tctgccacca gtgtaatgta tcaataaact tcatgcaagc ata |
| // | |
| 31. CRISP3 NM_006061 | |
| (SEQ ID NO: 31) | |
| 1 | gcacaaccag aatttgccaa aacaggaaat aggtgtttca tatatacggc tctaaccttc |
| 61 | tctctctgca ccttccttct gtcaatagat gaaacaaata cttcatcctg ctctggaaac |
| 121 | cactgcaatg acattattcc cagtgctgtt gttcctggtt gctgggctgc ttccatcttt |
| 181 | tccagcaaat gaagataagg atcccgcttt tactgctttg ttaaccaccc aaacacaagt |
| 241 | gcaaagggag attgtgaata agcacaatga actgaggaga gcagtatctc cccctgccag |
| 301 | aaacatgctg aagatggaat ggaacaaaga ggctgcagca aatgcccaaa agtgggcaaa |
| 361 | ccagtgcaat tacagacaca gtaacccaaa ggatcgaatg acaagtctaa aatgtggtga |
| 421 | gaatctctac atgtcaagtg cctccagctc atggtcacaa gcaatccaaa gctggtttga |
| 481 | tgagtacaat gattttgact ttggtgtagg gccaaagact cccaacgcag tggttggaca |
| 541 | ttatacacag gttgtttggt actcttcata cctcgttgga tgtggaaatg cctactgtcc |
| 601 | caatcaaaaa gttctaaaat actactatgt ttgccaatat tgtcctgctg gtaattgggc |
| 661 | taatagacta tatgtccctt atgaacaagg agcaccttgt gccagttgcc cagataactg |
| 721 | tgacgatgga ctatgcacca atggttgcaa gtacgaagat ctctatagta actgtaaaag |
| 781 | tttgaagctc acattaacct gtaaacatca gttggtcagg gacagttgca aggcctcctg |
| 841 | caattgttca aacagcattt attaaatacg cattacacac cgagtagggc tatgtagaga |
| 901 | ggagtcagat tatctactta gatttggcat ctacttagat ttaacatata ctagctgaga |
| 961 | aattgtaggc atgtttgata cacatttgat ttcaaatgtt tttcttctgg atctgctttt |
| 1021 | tattttacaa aaatattttt catacaaatg gttaaaaaga aacaaaatct ataacaacaa |
| 1081 | ctttggattt ttatatataa actttgtgat ttaaatttac tgaatttaat tagggtgaaa |
| 1141 | attttgaaag ttgtattctc atatgactaa gttcactaaa accctggatt gaaagtgaaa |
| 1201 | attatgttcc tagaacaaaa tgtacaaaaa gaacaatata attttcacat gaacccttgg |
| 1261 | ctgtagttgc ctttcctagc tccactctaa ggctaagcat cttcaaagac gttttcccat |
| 1321 | atgctgtctt aattcttttc actcattcac ccttcttccc aatcatctgg ctggcatcct |
| 1381 | cacaattgag ttgaagctgt tcctcctaaa acaatcctga cttttatttt gccaaaatca |
| 1441 | atacaatcct ttgaattttt tatctgcata aattttacag tagaatatga tcaaaccttc |
| 1501 | atttttaaac ctctcttctc tttgacaaaa cttccttaaa aaagaataca agataatata |
| 1561 | ggtaaatacc ctccactcaa ggaggtagaa ctcagtcctc tcccttgtga gtcttcacta |
| 1621 | aaatcagtga ctcacttcca aagagtggag tatggaaagg gaaacatagt aactttacag |
| 1681 | gggagaaaaa tgacaaatga cgtcttcacc aagtgatcaa aattaacgtc accagtgata |
| 1741 | agtcattcag atttgttcta gataatcttt ctaaaaattc ataatcccaa tctaattatg |
| 1801 | agctaaaaca tccagcaaac tcaagttgaa ggacattcta caaaatatcc ctggggtatt |
| 1861 | ttagagtatt cctcaaaact gtaaaaatca tggaaaataa gggaatcctg agaaacaatc |
| 1921 | acagaccaca tgagactaag gagacatgtg agccaaatgc aatgtgcttc ttggatcaga |
| 1981 | tcctggaaca gaaaaagatc agtaatgaaa aaactgatga agtctgaata gaatctggag |
| 2041 | tatttttaac agtagtgttg atttcttaat cttgataaat atagcagggt aatgtaagat |
| 2101 | gataacgtta gagaaactga aactgggtga gggctatcta ggaattctct gtactatctt |
| 2161 | accaaatttt cggtaagtct aagaaagcaa tgcaaaataa aaagtgtctt gaaaaaaaa |
| // | |
| 32. DHFR NM_000791 | |
| (SEQ ID NO: 32) | |
| 1 | tcccagacag aacctactat gtgcggcggc agctggggcg ggaaggcggg agctgggggc |
| 61 | gctgggggcg ctgcggccgc tgcggccgct gcagccgctg cagcgccagg gtccacctgg |
| 121 | tcggctgcac ctgtggagga ggaggtggat ttcaggcttc ccgtagactg gaagaatcgg |
| 181 | ctcaaaaccg cttgcctcgc aggggctgag ctggaggcag cgaggccgcc cgacgcaggc |
| 241 | ttccggcgag acatggcagg gcaaggatgg cagcccggcg gcagggcctg gcgaggagcg |
| 301 | cgagcccgcg gccgcagttc ccaggcgtct gcgggcgcga gcacgccgcg accctgcgtg |
| 361 | cgccggggcg ggggggcggg gcctcgcctg cacaaatggg gacgaggggg gcggggcggc |
| 421 | cacaatttcg cgccaaactt gaccgcgcgt tctgctgtaa cgagcgggct cggaggtcct |
| 481 | cccgctgctg tcatggttgg ttcgctaaac tgcatcgtcg ctgtgtccca gaacatgggc |
| 541 | atcggcaaga acggggacct gccctggcca ccgctcagga atgaattcag atatttccag |
| 601 | agaatgacca caacctcttc agtagaaggt aaacagaatc tggtgattat gggtaagaag |
| 661 | acctggttct ccattcctga gaagaatcga cctttaaagg gtagaattaa tttagttctc |
| 721 | agcagagaac tcaaggaacc tccacaagga gctcattttc tttccagaag tctagatgat |
| 781 | gccttaaaac ttactgaaca accagaatta gcaaataaag tagacatggt ctggatagtt |
| 841 | ggtggcagtt ctgtttataa ggaagccatg aatcacccag gccatcttaa actatttgtg |
| 901 | acaaggatca tgcaagactt tgaaagtgac acgttttttc cagaaattga tttggagaaa |
| 961 | tataaacttc tgccagaata cccaggtgtt ctctctgatg tccaggagga gaaaggcatt |
| 1021 | aagtacaaat ttgaagtata tgagaagaat gattaatatg aaggtgtttt ctagtttaag |
| 1081 | ttgttccccc tccctctgaa aaaagtatgt atttttacat tagaaaaggt tttttgttga |
| 1141 | ctttagatct ataattattt ctaagcaact agtttttatt ccccactact cttgtctcta |
| 1201 | tcagatacca tttatgagac attcttgcta taactaagtg cttctccaag accccaactg |
| 1261 | agtccccagc acctgctaca gtgagctgcc attccacacc catcacatgt ggcactcttg |
| 1321 | ccagtccttg acattgtcgg gcttttcaca tgttggtaat atttattaaa gatgaagatc |
| 1381 | cacataccct tcaactgagc agtttcacta gtggaaatac caaaagcttc ctacgtgtat |
| 1441 | atccagaggt ttgtagataa atgttgccac cttgtttgta acagtgaaaa attgaaaaca |
| 1501 | acctggaagt ccagtgatgg gaaaatgagt atgtttctgt cttagattgg ggaacccaaa |
| 1561 | gcagattgca agactgaaat ttcagtgaaa gcagtgtatt tgctaggtca taccagaaat |
| 1621 | catcaattga ggtacggaga aactgaactg agaaggtaag aaaaccaatt taaagtcagc |
| 1681 | gagcaggttc tcattgataa caagctccat actgctgaga tacagggaaa tggagggggg |
| 1741 | aaagctggag tattgatccc gcccccctcc ttggttgtca gctccctgtc ctgtgtgtgg |
| 1801 | gcggaacata gtccagctgc tctatagcaa gtctcaggtg tttgcagtaa gaagctgctg |
| 1861 | gcatgcacgg gaacagtgaa tgccaaacac ttaaagcaat tcgatgttta agtatgtaag |
| 1921 | ttcttttttt tttagacagc gtttcgctct tgttgcccag gctagcatgc aatggtgtga |
| 1981 | cctcggctta ctgcaacctc cgccttccca gattcaagcg attctcctgc ctcaggctcc |
| 2041 | caagtagcta ggaccaggtg cgcgccacca cgcccggcta atttttgtat tttgtatttt |
| 2101 | tagtagagat ggggtttcac catgttggtc aggctagtct cgaactcgtg accgcaagcg |
| 2161 | attcacccac ctcagcctcc caaagtgctg ggattaccgg cttgagccac cacacccggc |
| 2221 | acatcttcat tctttttatg tagtaaaaag tataaggcca cacatggttt atttgaagta |
| 2281 | ttttataatt taaaaaaata cagaagcagg aaaaccaatt ataagttcaa gtgagggatg |
| 2341 | atggttgctt gaaccaaagg gttgcatgta gtaagaaatt gtgatttaag atatatttta |
| 2401 | aagttataag tagcaggata ttctgatgga gtttgacttt ggttttgggc ccagggagtt |
| 2461 | tcagatgcct ttgagaaatg aatgaagtag agagaaaata aaagaaaaac cagccaggca |
| 2521 | cagtggctca cacctgtaat cccagcgctt tgggaggcta aggcaggcag atcacttgag |
| 2581 | accagcttgg gcaacatggc aaagccccat ctctacaaaa aacacaaaaa ttagctgggc |
| 2641 | attgtggcgc acacctgtat tcccatctag tcaggaagct gagatggaag aattaattga |
| 2701 | gcccacgagt tcaaggctgc agtgagtcgt gattgtgcca ctgcactcca gccggggtga |
| 2761 | cagaagagac cttgtctcga aaaggaatct gaaaacaatg gaaccatgcc ttcataattc |
| 2821 | tagaaagtta ttttcaactg ataaatctat attcacccaa ataatcaagg gtgaaggtaa |
| 2881 | aataatacat ttttagacaa gcaaagactc aggggttacc tccatgtgcc ctttttaggg |
| 2941 | aagctgttgg agaaaatact ccagcaaaat gaaggagtac acaaaccaga gaatgacatg |
| 3001 | aatccagcaa ataggatcca acacaggcaa tattccagct atggagctag ctttaaaaag |
| 3061 | gaacagtaaa aatattaatc ggttagctgg gtggaatggc ccatgcctgt agtcccagct |
| 3121 | actcaggagg ctcagcagca ggacgacttg agcccaagag ttccagacca gcctggccac |
| 3181 | cttagtgaga tcccttctct taaaaataat aacttattgc cagatttggg gcatttggaa |
| 3241 | agaagttcat tgaagataaa gcaaaagtaa aaaaaaaaaa aaaaaaaaca aggggaaagg |
| 3301 | gttggttagg caatcattct agggcagaaa gaagtacagg ataggaagag cataatacac |
| 3361 | tgtttttctc aacaaggagc agtatgtaca cagtcataat gatgtgactg cttagcccct |
| 3421 | aaatatggta actactctgg gacaatatgg gaggaaaagt gaagattgtg atggtgtaag |
| 3481 | agctaaatcc tcatctgtca tatccagaaa tcactatata atatataata atgaaatgac |
| 3541 | taagttatgt gaggaaaaaa acagaagaca ttgctaaaag agttaaaagt cattgctctg |
| 3601 | gagaattagg agggatgggg caggggactg ttaggatgca ttataaactg aaaagccttt |
| 3661 | ttaaaatttt atgtattaat atatgcattc acttgaaaaa ctaaaaaaaa acaataattt |
| 3721 | ggaaaaaccc atgaaggtaa ctaacggaag gaaaaactaa gagaatgaaa agtatttgcc |
| 3781 | tctggaaaga acaactggca ggactgttgt tttcattgta agacttttgg agccatttaa |
| 3841 | ttgtacttaa ccattttcat ctatttcttt aataagaaca attccatctt aataaagagt |
| 3901 | tacacttgtt aataagtaaa aaaaaaaaaa aa |
| // | |
| 33. GLUD1 NM_005271 | |
| (SEQ ID NO: 33) | |
| 1 | gcgctgccgc cagcgaggcc cggggaggcc gcggcggagg cggaggcccg gcgccctggg |
| 61 | cggcgccctg tccccgaagt ccgtcctccc cgttaggtgg cgagcgcccg aggggagggg |
| 121 | acagccgggc aagcaggaag ctgcggctta aaagggcaac ccgcgcggga cccttcctcc |
| 181 | ctagtcgcgg ggagtctgag aaagcgcgcc tgtttcgcga ccatcacgca cctcccctcc |
| 241 | gcttgtggcc atgtaccgct acctgggcga agcgctgttg ctgtcccggg ccgggcccgc |
| 301 | tgccctgggc tcggcgtccg ccgactcggc cgcgttgctg ggctgggccc ggggacagcc |
| 361 | cgccgccgcc ccgcagccgg ggctggcatt ggccgcccgg cgccactaca gcgaggcggt |
| 421 | ggccgaccgc gaggacgacc ccaacttctt caagatggtg gagggcttct tcgatcgcgg |
| 481 | cgccagcatc gtggaggaca agctggtgga ggacctgagg acccgggaga gcgaggagca |
| 541 | gaagcggaac cgggtgcgcg gcatcctgcg gatcatcaag ccctgcaacc atgtgctgag |
| 601 | tctctccttc cccatccggc gcgacgacgg ctcctgggag gtcatcgaag gctaccgggc |
| 661 | ccagcacagc cagcaccgca cgccctgcaa gggaggtatc cgttacagca ctgatgtgag |
| 721 | tgtagatgaa gtaaaagctt tggcttctct gatgacatac aagtgtgcag tggttgatgt |
| 781 | gccgtttggg ggtgctaaag ctggtgttaa gatcaatccc aagaactata ctgataatga |
| 841 | attggaaaag atcacaagga ggttcaccat ggagctagca aaaaagggct ttattggtcc |
| 901 | tggcattgat gtgcctgctc cagacatgag cacaggtgag cgggagatgt cctggatcgc |
| 961 | tgatacctat gccagcacca tagggcacta tgatattaat gcacacgcct gtgttactgg |
| 1021 | taaacccatc agccaagggg gaatccatgg acgcatctct gctactggcc gtggtgtctt |
| 1081 | ccatgggatt gaaaatttca tcaatgaagc ttcttacatg agcattttag gaatgacacc |
| 1141 | agggtttgga gataaaacat ttgttgttca gggatttggt aatgtgggcc tacactctat |
| 1201 | gagatattta catcgttttg gtgctaaatg tattgctgtt ggtgagtctg atgggagtat |
| 1261 | atggaatcca gatggtattg acccaaagga actggaagac ttcaaattgc aacatgggtc |
| 1321 | cattctgggc ttccccaagg caaagcccta tgaaggaagc atcttggagg ccgactgtga |
| 1381 | catactgatc ccagctgcca gtgagaagca gttgaccaaa tccaacgcac ccagagtcaa |
| 1441 | agccaagatc attgctgaag gtgccaatgg gccaacaact ccagaagctg acaagatctt |
| 1501 | cctggagaga aacattatgg ttattccaga tctctacttg aatgctggag gagtgacagt |
| 1561 | atcttacttt gagtggctga agaatctaaa tcatgtcagc tatggccgtt tgaccttcaa |
| 1621 | atatgaaagg gattctaact accacttgct catgtctgtt caagagagtt tagaaagaaa |
| 1681 | atttggaaag catggtggaa ctattcccat tgtacccacg gcagagttcc aagacaggat |
| 1741 | atcgggtgca tctgagaaag acatcgtgca ctctggcttg gcatacacaa tggagcgttc |
| 1801 | tgccaggcaa attatgcgca cagccatgaa gtataacctg ggattggacc tgagaacagc |
| 1861 | tgcctatgtt aatgccattg agaaagtctt caaagtgtac aatgaagctg gtgtgacctt |
| 1921 | cacatagatg gatcatggct gacttcctca ctatcctctt cacatgtaac ttctgcagac |
| 1981 | ctatcacaag tttacatgta accacagaaa tccctttctc tcctgactca ttaataatgg |
| 2041 | ataccattct caacaagtca atccaagtca gcccgttaag gagaaagaaa ttaaggttag |
| 2101 | cggatcatgt acaagctgag tgtgaaagta gaaatcacct acaccagaga gccattttgg |
| 2161 | tattttgcct ttaaataaaa agcctccttt atctggctgt gcagccttgc tctgtggctt |
| 2221 | ttcccaacac aatcagtgct agtgctgggg aggaacagtc aagagcagtc agttgcttgc |
| 2281 | ttattttttc tggatgagtc tgggacacac tgtaacttta acacatttaa gaagtaggtg |
| 2341 | tgtggccttt tcagaaggtg gcatggtcct caagtgagtt cttagtattt tatatcagca |
| 2401 | aaataattca attttgcagg ttgcaaacaa atataaaacc tgtttctgtt tatgaatatt |
| 2461 | attcttttag aatagaataa gtacatgctg ctgtaataaa attgccttta atcacttaac |
| 2521 | aagcctaacc ttgactcaaa cagtgaatgc ctatagaaat aataaatgaa aaaaactagt |
| 2581 | atttttatat cataaaacaa tgtcatttat agcttatcat tcatgtattg tccagcagac |
| 2641 | attaaaagcc ctgtggataa ttaagttatc ttcatacctg caaaatggtg gaggctattt |
| 2701 | tcattaaaac tgtcagaatt tgcttactat aattatgata cagtccaaag aatgcagtca |
| 2761 | ctttttatca tgttaactaa ttgttctctt ttgaagatct atggttgact aattaaacaa |
| 2821 | taattcaagt agagtgtccc agaaaaaaac cacttgggct ccctgtttgg agtctggctg |
| 2881 | gctctgagca ttgccaatgg cccctactca cctgactttg tatcctctcc ttttagaggc |
| 2941 | tttgcattct gcacccagct tcactaacag tgggctgaaa acatccttgg gttgagtgtt |
| 3001 | tcatttggga gttatttggc cagggccttt tgaacagtag tgtccccatg aagtgctaga |
| 3061 | taatatatgt gtaagagtca gctttttttt tttttttaac tctaacaccc ttcagaaatt |
| 3121 | tctaactact ttgtaactgc atggcttaac ctggtgataa aagcagttat taaaagtcta |
| 3181 | cgttttccaa aacttacgtt tcttttctgt gtttttacat gtggtagttt ctcttttcat |
| 3241 | aagttataat actgcaattg gatttctgaa atgtttatag cgaccacctg tataacattt |
| 3301 | ccttccactt tattgtgagc tgcccagatt ttattcttga attgtttttt ttttttttgt |
| 3361 | tcggtgcttt acacgttcag agaaacttcc cgagtaacga actatagaaa tgatccctga |
| 3421 | aagcatagtc tttattctcg aattattttg tattttatta aataatatga acagctaaaa |
| 3481 | aaaaaa |
| // | |
| 34. MUC1 NM_002456 | |
| (SEQ ID NO: 34) | |
| 1 | cgctccacct ctcaagcagc cagcgcctgc ctgaatctgt tctgccccct ccccacccat |
| 61 | ttcaccacca ccatgacacc gggcacccag tctcctttct tcctgctgct gctcctcaca |
| 121 | gtgcttacag ttgttacggg ttctggtcat gcaagctcta ccccaggtgg agaaaaggag |
| 181 | acttcggcta cccagagaag ttcagtgccc agctctactg agaagaatgc tttgtctact |
| 241 | ggggtctctt tctttttcct gtcttttcac atttcaaacc tccagtttaa ttcctctctg |
| 301 | gaagatccca gcaccgacta ctaccaagag ctgcagagag acatttctga aatgtttttg |
| 361 | cagatttata aacaaggggg ttttctgggc ctctccaata ttaagttcag gccaggatct |
| 421 | gtggtggtac aattgactct ggccttccga gaaggtacca tcaatgtcca cgacgtggag |
| 481 | acacagttca atcagtataa aacggaagca gcctctcgat ataacctgac gatctcagac |
| 541 | gtcagcgtga gtgatgtgcc atttcctttc tctgcccagt ctggggctgg ggtgccaggc |
| 601 | tggggcatcg cgctgctggt gctggtctgt gttctggttg cgctggccat tgtctatctc |
| 661 | attgccttgg ctgtctgtca gtgccgccga aagaactacg ggcagctgga catctttcca |
| 721 | gcccgggata cctaccatcc tatgagcgag taccccacct accacaccca tgggcgctat |
| 781 | gtgcccccta gcagtaccga tcgtagcccc tatgagaagg tttctgcagg taatggtggc |
| 841 | agcagcctct cttacacaaa cccagcagtg gcagccactt ctgccaactt gtaggggcac |
| 901 | gtcgcccgct gagctgagtg gccagccagt gccattccac tccactcagg ttcttcaggg |
| 961 | ccagagcccc tgcaccctgt ttgggctggt gagctgggag ttcaggtggg ctgctcacag |
| 1021 | cctccttcag aggccccacc aatttctcgg acacttctca gtgtgtggaa gctcatgtgg |
| 1081 | gcccctgagg gctcatgcct gggaagtgtt gtggtggggg ctcccaggag gactggccca |
| 1141 | gagagccctg agatagcggg gatcctgaac tggactgaat aaaacgtggt ctcccactgc |
| 1201 | gccaaaaaaa aaaaaaaaaa |
| // | |
| 35. PRAME NM_006115 | |
| (SEQ ID NO: 35) | |
| 1 | cgagttccgg cgaggcttca gggtacagct cccccgcagc cagaagccgg gcctgcagcg |
| 61 | cctcagcacc gctccgggac accccacccg cttcccaggc gtgacctgtc aacagcaact |
| 121 | tcgcggtgtg gtgaactctc tgaggaaaaa ccattttgat tattactctc agacgtgcgt |
| 181 | ggcaacaagt gactgagacc tagaaatcca agcgttggag gtcctgaggc cagcctaagt |
| 241 | cgcttcaaaa tggaacgaag gcgtttgtgg ggttccattc agagccgata catcagcatg |
| 301 | agtgtgtgga caagcccacg gagacttgtg gagctggcag ggcagagcct gctgaaggat |
| 361 | gaggccctgg ccattgccgc cctggagttg ctgcccaggg agctcttccc gccactcttc |
| 421 | atggcagcct ttgacgggag acacagccag accctgaagg caatggtgca ggcctggccc |
| 481 | ttcacctgcc tccctctggg agtgctgatg aagggacaac atcttcacct ggagaccttc |
| 541 | aaagctgtgc ttgatggact tgatgtgctc cttgcccagg aggttcgccc caggaggtgg |
| 601 | aaacttcaag tgctggattt acggaagaac tctcatcagg acttctggac tgtatggtct |
| 661 | ggaaacaggg ccagtctgta ctcatttcca gagccagaag cagctcagcc catgacaaag |
| 721 | aagcgaaaag tagatggttt gagcacagag gcagagcagc ccttcattcc agtagaggtg |
| 781 | ctcgtagacc tgttcctcaa ggaaggtgcc tgtgatgaat tgttctccta cctcattgag |
| 841 | aaagtgaagc gaaagaaaaa tgtactacgc ctgtgctgta agaagctgaa gatttttgca |
| 901 | atgcccatgc aggatatcaa gatgatcctg aaaatggtgc agctggactc tattgaagat |
| 961 | ttggaagtga cttgtacctg gaagctaccc accttggcga aattttctcc ttacctgggc |
| 1021 | cagatgatta atctgcgtag actcctcctc tcccacatcc atgcatcttc ctacatttcc |
| 1081 | ccggagaagg aagagcagta tatcgcccag ttcacctctc agttcctcag tctgcagtgc |
| 1141 | ctgcaggctc tctatgtgga ctctttattt ttccttagag gccgcctgga tcagttgctc |
| 1201 | aggcacgtga tgaacccctt ggaaaccctc tcaataacta actgccggct ttcggaaggg |
| 1261 | gatgtgatgc atctgtccca gagtcccagc gtcagtcagc taagtgtcct gagtctaagt |
| 1321 | ggggtcatgc tgaccgatgt aagtcccgag cccctccaag ctctgctgga gagagcctct |
| 1381 | gccaccctcc aggacctggt ctttgatgag tgtgggatca cggatgatca gctccttgcc |
| 1441 | ctcctgcctt ccctgagcca ctgctcccag cttacaacct taagcttcta cgggaattcc |
| 1501 | atctccatat ctgccttgca gagtctcctg cagcacctca tcgggctgag caatctgacc |
| 1561 | cacgtgctgt atcctgtccc cctggagagt tatgaggaca tccatggtac cctccacctg |
| 1621 | gagaggcttg cctatctgca tgccaggctc agggagttgc tgtgtgagtt ggggcggccc |
| 1681 | agcatggtct ggcttagtgc caacccctgt cctcactgtg gggacagaac cttctatgac |
| 1741 | ccggagccca tcctgtgccc ctgtttcatg cctaactagc tgggtgcaca tatcaaatgc |
| 1801 | ttcattctgc atacttggac actaaagcca ggatgtgcat gcatcttgaa gcaacaaagc |
| 1861 | agccacagtt tcagacaaat gttcagtgtg agtgaggaaa acatgttcag tgaggaaaaa |
| 1921 | acattcagac aaatgttcag tgaggaaaaa aaggggaagt tggggatagg cagatgttga |
| 1981 | cttgaggagt taatgtgatc tttggggaga tacatcttat agagttagaa atagaatctg |
| 2041 | aatttctaaa gggagattct ggcttgggaa gtacatgtag gagttaatcc ctgtgtagac |
| 2101 | tgttgtaaag aaactgttga aaataaagag aagcaatgtg aagcaaaaaa aaaaaaaaaa |
| 2161 | aa |
| // | |
| 36. SOX2 NM_003106 | |
| (SEQ ID NO: 36) | |
| 1 | ggatggttgt ctattaactt gttcaaaaaa gtatcaggag ttgtcaaggc agagaagaga |
| 61 | gtgtttgcaa aagggggaaa gtagtttgct gcctctttaa gactaggact gagagaaaga |
| 121 | agaggagaga gaaagaaagg gagagaagtt tgagccccag gcttaagcct ttccaaaaaa |
| 181 | taataataac aatcatcggc ggcggcagga tcggccagag gaggagggaa gcgctttttt |
| 241 | tgatcctgat tccagtttgc ctctctcttt ttttccccca aattattctt cgcctgattt |
| 301 | tcctcgcgga gccctgcgct cccgacaccc ccgcccgcct cccctcctcc tctccccccg |
| 361 | cccgcgggcc ccccaaagtc ccggccgggc cgagggtcgg cggccgccgg cgggccgggc |
| 421 | ccgcgcacag cgcccgcatg tacaacatga tggagacgga gctgaagccg ccgggcccgc |
| 481 | agcaaacttc ggggggcggc ggcggcaact ccaccgcggc ggcggccggc ggcaaccaga |
| 541 | aaaacagccc ggaccgcgtc aagcggccca tgaatgcctt catggtgtgg tcccgcgggc |
| 601 | agcggcgcaa gatggcccag gagaacccca agatgcacaa ctcggagatc agcaagcgcc |
| 661 | tgggcgccga gtggaaactt ttgtcggaga cggagaagcg gccgttcatc gacgaggcta |
| 721 | agcggctgcg agcgctgcac atgaaggagc acccggatta taaataccgg ccccggcgga |
| 781 | aaaccaagac gctcatgaag aaggataagt acacgctgcc cggcgggctg ctggcccccg |
| 841 | gcggcaatag catggcgagc ggggtcgggg tgggcgccgg cctgggcgcg ggcgtgaacc |
| 901 | agcgcatgga cagttacgcg cacatgaacg gctggagcaa cggcagctac agcatgatgc |
| 961 | aggaccagct gggctacccg cagcacccgg gcctcaatgc gcacggcgca gcgcagatgc |
| 1021 | agcccatgca ccgctacgac gtgagcgccc tgcagtacaa ctccatgacc agctcgcaga |
| 1081 | cctacatgaa cggctcgccc acctacagca tgtcctactc gcagcagggc acccctggca |
| 1141 | tggctcttgg ctccatgggt tcggtggtca agtccgaggc cagctccagc ccccctgtgg |
| 1201 | ttacctcttc ctcccactcc agggcgccct gccaggccgg ggacctccgg gacatgatca |
| 1261 | gcatgtatct ccccggcgcc gaggtgccgg aacccgccgc ccccagcaga cttcacatgt |
| 1321 | cccagcacta ccagagcggc ccggtgcccg gcacggccat taacggcaca ctgcccctct |
| 1381 | cacacatgtg agggccggac agcgaactgg aggggggaga aattttcaaa gaaaaacgag |
| 1441 | ggaaatggga ggggtgcaaa agaggagagt aagaaacagc atggagaaaa cccggtacgc |
| 1501 | tcaaaaagaa aaaggaaaaa aaaaaatccc atcacccaca gcaaatgaca gctgcaaaag |
| 1561 | agaacaccaa tcccatccac actcacgcaa aaaccgcgat gccgacaaga aaacttttat |
| 1621 | gagagagatc ctggacttct ttttggggga ctatttttgt acagagaaaa cctggggagg |
| 1681 | gtggggaggg cgggggaatg gaccttgtat agatctggag gaaagaaagc tacgaaaaac |
| 1741 | tttttaaaag ttctagtggt acggtaggag ctttgcagga agtttgcaaa agtctttacc |
| 1801 | aataatattt agagctagtc tccaagcgac gaaaaaaatg ttttaatatt tgcaagcaac |
| 1861 | ttttgtacag tatttatcga gataaacatg gcaatcaaaa tgtccattgt ttataagctg |
| 1921 | agaatttgcc aatatttttc aaggagaggc ttcttgctga attttgattc tgcagctgaa |
| 1981 | atttaggaca gttgcaaacg tgaaaagaag aaaattattc aaatttggac attttaattg |
| 2041 | tttaaaaatt gtacaaaagg aaaaaattag aataagtact ggcgaaccat ctctgtggtc |
| 2101 | ttgtttaaaa agggcaaaag ttttagactg tactaaattt tataacttac tgttaaaagc |
| 2161 | aaaaatggcc atgcaggttg acaccgttgg taatttataa tagcttttgt tcgatcccaa |
| 2221 | ctttccattt tgttcagata aaaaaaacca tgaaattact gtgtttgaaa tattttctta |
| 2281 | tggtttgtaa tatttctgta aatttattgt gatattttaa ggttttcccc cctttatttt |
| 2341 | ccgtagttgt attttaaaag attcggctct gtattatttg aatcagtctg ccgagaatcc |
| 2401 | atgtatatat ttgaactaat atcatcctta taacaggtac attttcaact taagttttta |
| 2461 | ctccattatg cacagtttga gataaataaa tttttgaaat atggacactg aaaaaaaaaa |
| // | |
| 37. TULP2 NM_003323 | |
| (SEQ ID NO: 37) | |
| 1 | tttgttggaa gtggagagtg gagggtcaga agggagtgga ccagttcagg tcccagaggg |
| 61 | aatcctccct ccctctgagc cgtctttctt ctcctcccta tttcgcagat atcccgagat |
| 121 | taggtcccca gcttccaaag agaggatcag aatgtctcag gataatgaca cattgatgag |
| 181 | agacatcctg gggcatgagc tcgctgctat gaggctgcag aagctggaac agcagcggcg |
| 241 | gctgtttgaa aagaagcagc gacagaagcg ccaggagctc ctcatggttc aggccaatcc |
| 301 | tgacgcttcc ccgtggcttt ggcgctcttg tctgcgggag gagcgccttt taggtgacag |
| 361 | aggccttggg aaccctttcc tccggaagaa agtgtcagag gcacatctgc cctctggcat |
| 421 | ccacagtgcc ctgggcaccg tgagctgtgg tggagacggc aggggcgagc gcggcctccc |
| 481 | gacaccgcgg acagaagcag tgttcaggaa tctcggtctc cagtcccctt tcttatcctg |
| 541 | gctcccagac aattccgatg cagaattgga ggaagtctcc gtggagaatg gttccgtctc |
| 601 | tcccccacct tttaaacagt ctccgagaat ccgacgcaag ggttggcaag cccaccaacg |
| 661 | acctgggacc cgtgcagagg gtgagagtga ctcccaggat atgggagatg cacacaagtc |
| 721 | acccaatatg ggaccaaacc ctggaatgga tggtgactgt gtatatgaaa acttggcctt |
| 781 | ccaaaaggaa gaagacttgg aaaagaagag agaggcctct gagtctacag ggacgaactc |
| 841 | ctcagcagca cacaacgaag agttgtccaa ggccctgaaa ggcgagggtg gcacggacag |
| 901 | cgaccatatg aggcacgaag cctccttggc aatccgctcc ccctgccctg ggctggagga |
| 961 | ggacatggaa gcctacgtgc tgcggccagc gctcccgggc accatgatgc agtgctacct |
| 1021 | cacccgtgac aagcacggcg tggacaaggg cttgttcccc ctctactacc tctacctgga |
| 1081 | gacctctgac agcctgcagc gcttcctcct ggctgggcga aagagaagaa ggagcaaaac |
| 1141 | ttctaattac ctcatctccc tggatcctac acacctatct cgggacgggg acaatttcgt |
| 1201 | gggcaaagtc agatccaatg tcttcagcac caagttcacc atctttgaca atggggtgaa |
| 1261 | tcctgaccgg gagcatttaa ccaggaatac tgcccggatc agacaggagc tgggggctgt |
| 1321 | gtgttatgag cccaacgtct taggatacct ggggcctcgg aaaatgactg tgattctccc |
| 1381 | aggaaccaac agccagaacc agcgaatcaa tgtccagcca ctaaatgaac aggagtcgct |
| 1441 | actgagtcgt taccaacgtg gggacaaaca agggttgctt ttgttgcaca acaaaacccc |
| 1501 | gtcgtgggac aaggagaacg gtgtctacac gctcaatttc catggtcgag tcactcgggc |
| 1561 | ttcggtgaag aacttccaaa tcgtggatcc caaacaccaa gaacatctgg tgctccagtt |
| 1621 | cggccgagtg ggcccagaca cattcaccat ggacttctgc tttccattta gcccgctcca |
| 1681 | ggccttcagc atctgcttgt ccagtttcaa ttagaagctg gctgttgaat aactcaataa |
| 1741 | aataccatac ccttgccagc aaaaaaaaaa aaaaaaaaaa aaaaaaaaaa a |
| // | |

The terms “isolated” or “biologically pure” refer to material that is substantially or essentially free from components which normally accompany the material as it is found in its native state.

As used in this specification, the singular forms “a”, “an”, and “the” include plural reference unless the context clearly dictates otherwise. Thus, for example, a reference to “a biomarker” includes more than one such biomarker. Reference to an “antibody” includes more than one such antibody. A reference to “an epitope” includes more than one such epitope, and so forth.

The practice of the present invention can employ, unless otherwise indicated, conventional techniques of molecular biology, microbiology, recombinant DNA technology, electrophysiology, and pharmacology that are within the skill of the art. Such techniques are explained fully in the literature (see, e.g., Sambrook, Fritsch & Maniatis, Molecular Cloning: A Laboratory Manual, Second Edition (1989); DNA Cloning, Vols. I and II (D. N. Glover Ed. 1985); Perbal, B., A Practical Guide to Molecular Cloning (1984); the series, Methods In Enzymology (S. Colowick and N. Kaplan Eds., Academic Press, Inc.); Transcription and Translation (Hames et al. Eds. 1984); Gene Transfer Vectors For Mammalian Cells (J. H. Miller et al. Eds. (1987) Cold Spring Harbor Laboratory, Cold Spring Harbor, N.Y.); Scopes, Protein Purification: Principles and Practice (2nd ed., Springer-Verlag); and PCR: A Practical Approach (McPherson et al. Eds. (1991) IRL Press)), each of which are incorporated herein by reference in their entirety.

Experimental controls are considered fundamental in experiments designed in accordance with the scientific method. It is routine in the art to use experimental controls in scientific experiments to prevent factors other than those being studied from affecting the outcome.

Following are examples which illustrate procedures for practicing the invention. These examples should not be construed as limiting. All percentages are by weight and all solvent mixture proportions are by volume unless otherwise noted.

### Example 1

### Changes in Antibody Profiles after Immunotherapy Predict Adverse Events

The treatment of cancer with immunotherapy is associated with higher risk of autoimmune side effects that can be life threatening. This study shows that such events can be predicted by comparing patient immunity to panel antigens before and after treatment. In this study, the immunotherapy 1-methyl-D-tryptophan (1MT) was used to treat 12 patients with various cancer types. Serum antibody levels to panel antigens were measured before and after treatment. It was found that elevated immunity to panel antigens after treatment (50%+ increase in antibody levels) was a risk factor for anti-pituitary autoimmune side effects (hypophysis).

Methods:

Serum (100 μL) was collected from 1MT-treated cancer patients (n=12) before treatment with 1 MT immunotherapy. A second sample was collected at week 12 after therapy. Each serum sample was tested for antibodies using a protein microarray. The array contained full-length human recombinant proteins corresponding to a panel of 10 tumor antigens (CTAG2, MAGEA1, MAGEA3, MAGEAv2, MICA, NLRP4, SILV, SSX4, TSSK6, and XAGE-2). After incubating the serum on the array, unbound antibody was removed by washing, and bound antibody was detected using a fluorescent secondary antibody. Signals (RFU) were recorded for each antigen and the level of increase or decrease after drug treatment was calculated and compared to clinical data regarding the development of autoimmune adverse effect.

Results:

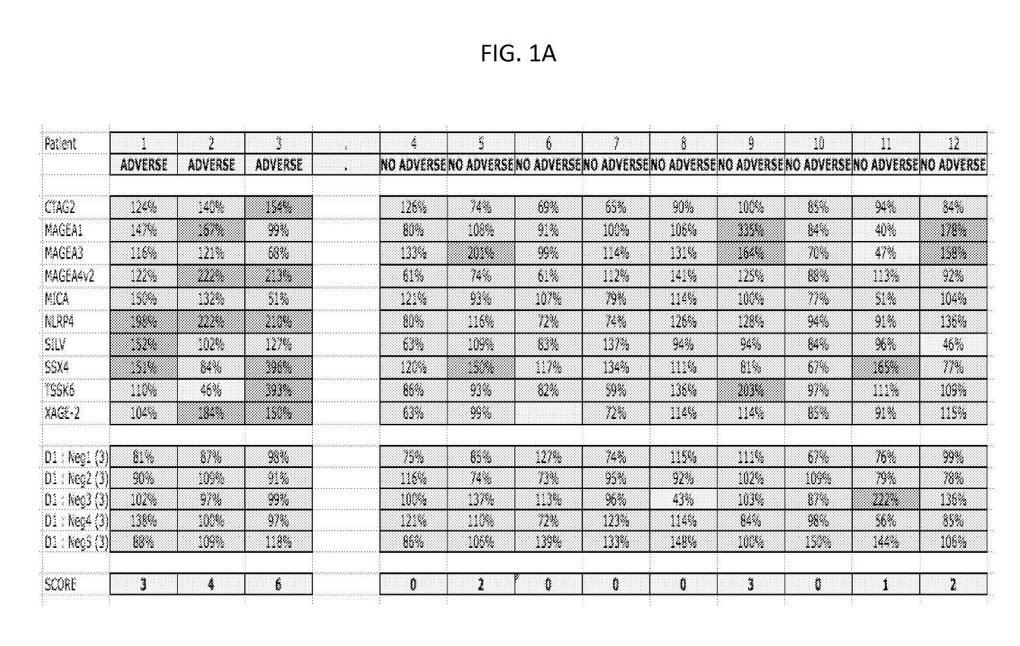

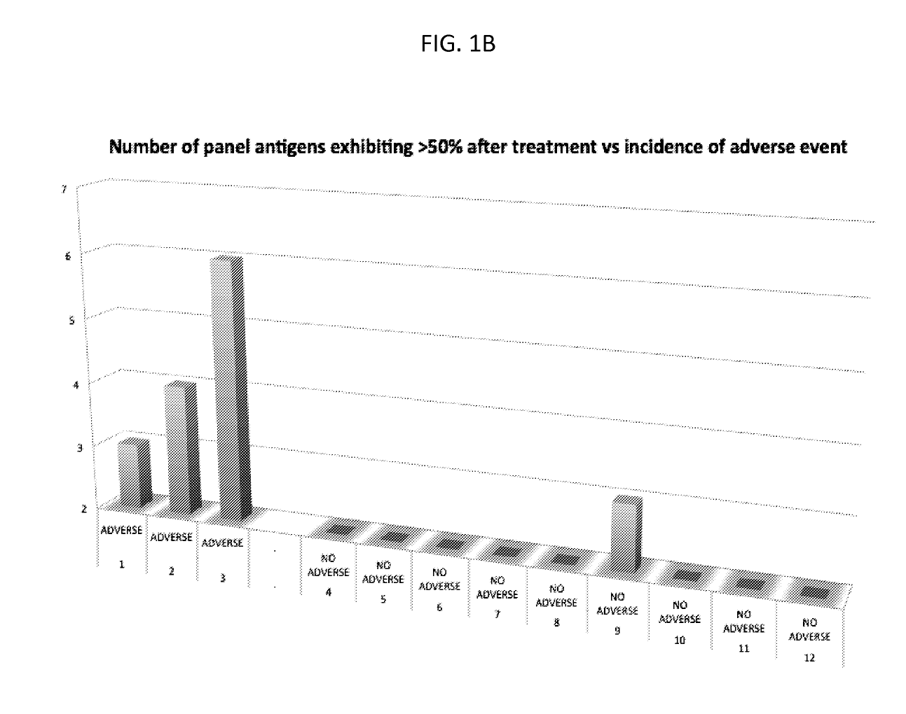

As shown by the table in FIG. 1A, there was a positive relationship between an increase in reactivity to panel antigens and the incidence of autoimmune side effect. Patients who did have a 50%+ increase in reactivity to 3+(three or more) panel antigens did suffer autoimmune side effect (100%, n=3). Patients who did not have a 50%+ increase in reactivity to 3+(three or more) panel antigens did not suffer autoimmune side effect (89%, n=9). FIG. 1B is a graph showing the number of panel antigens exhibiting greater than 50% after treatment versus incidence of adverse event.

### Example 2

### Baseline Antibody Profiles to Predict Response to Immunotherapy

This study demonstrates the positive relationship between baseline immunity to panel antigens and survival following immunotherapy. Patients (n=8) with lung cancer (SCLC) were treated with immunotherapy (p53 cancer vaccine) then chemotherapy (cisplatin or carboplatin). Prior to chemotherapy, patient serum was tested for serum antibody to a panel of tumor antigens. The number of reactive antigens was compared to the length of survival following first vaccination. The survival time of patients was found to correlate with baseline immunity to panel antigens—patients who tested positive (reactive to more than 10 panel antigens) lived longer than those who tested negative (reactive to less than 10 panel antigens).

Methods:

Serum (100 μL) was collected from p53-vaccine-treated SCLC patients (n=8) prior to chemotherapy. Each serum sample was tested for antibodies using a protein microarray. The array contained full-length human recombinant proteins corresponding to a panel of 10 tumor antigens (CTAG2, MAGEA1, MAGEA3, MAGEA4v3, MICA, NURP4, SILV, SSX4, TSSK6, and XAGE-2). After incubating the serum on the array, unbound antibody was removed by washing, and bound antibody detected using a fluorescent secondary antibody. Signals (RFU) were recorded for each antigen and determined positive if greater than the antigen-specific cut-off value (1.2×average value for antigen). Survival time from the first dose of vaccine was recorded and compared with the number of antigens to which patients were seropositive at baseline.

Results:

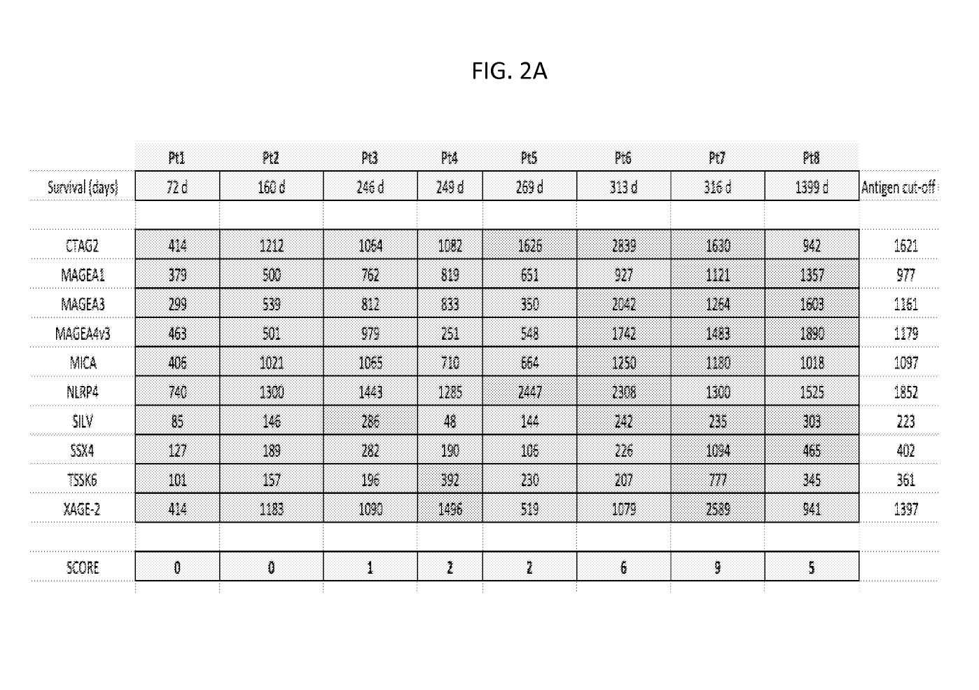

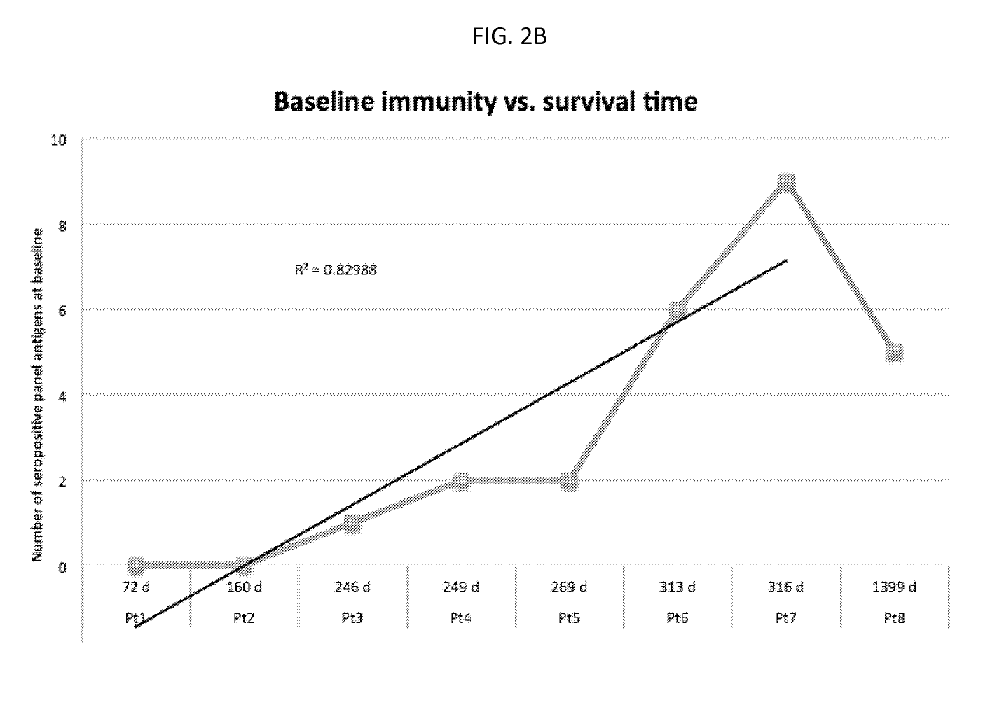

As shown by the table in FIG. 2A, there was a positive relationship between the number of seropositive events at baseline and the number of months' survival following immunotherapy. Patients who did not test positive to 5+ panel antigens did not survive more than 300 days (100%, n=5). Patients who did test positive to 5+ panel antigens did survive more than 300 days (66%, n=3). FIG. 2B is a graph showing baseline immunity versus survival time. With the removal of one outlier, the R-squared value of the trend line was 0.82.

### Example 3

### Antigen Profiles Predictive of Clinical Response and Adverse Events Following Immunotherapy

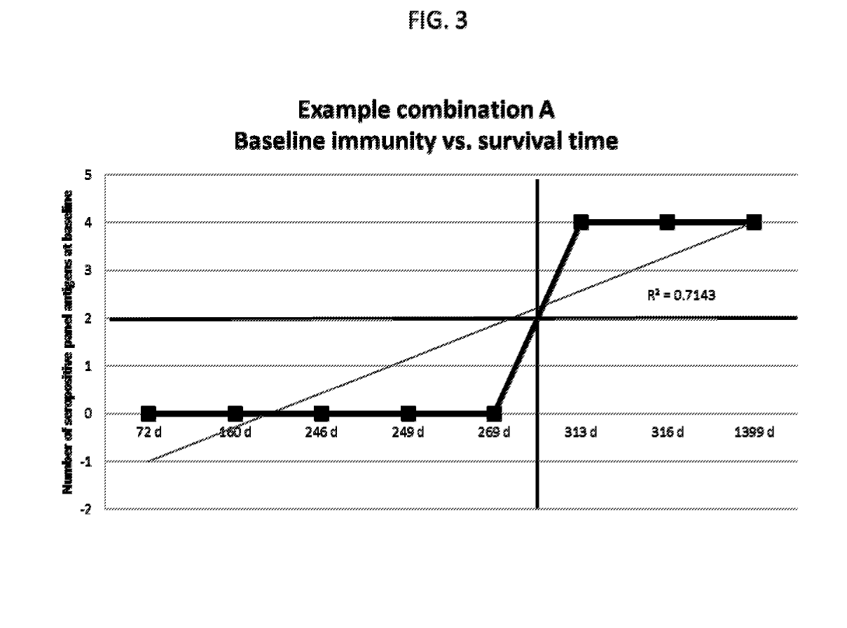

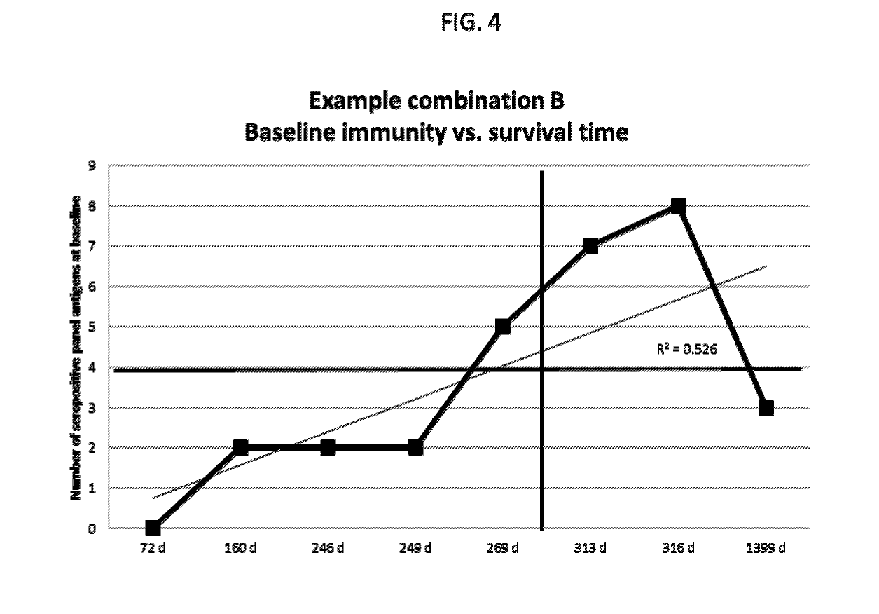

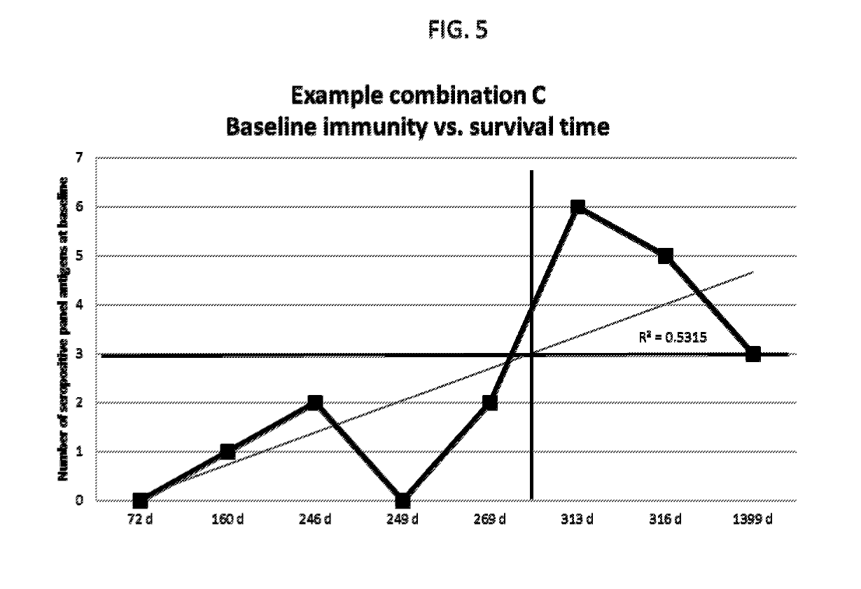

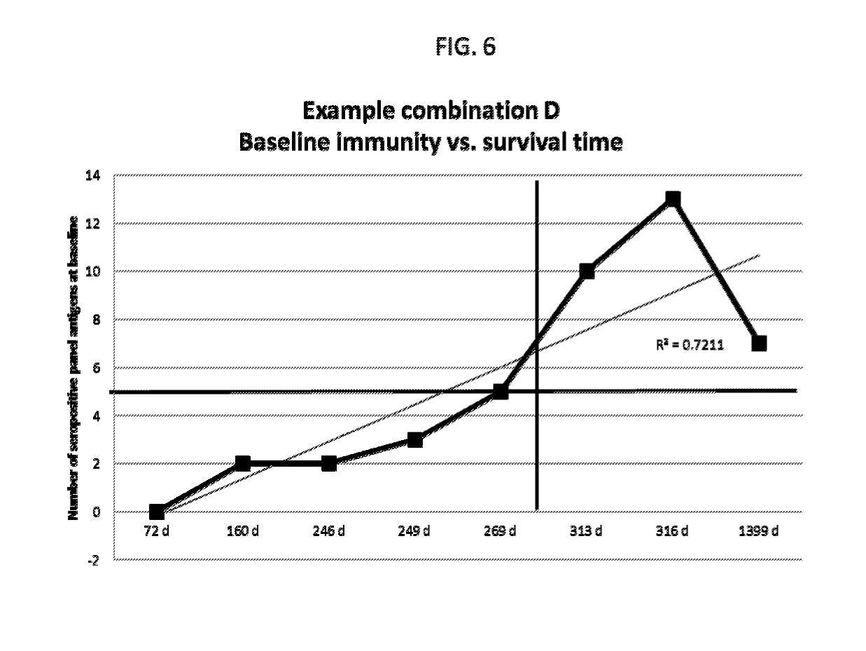

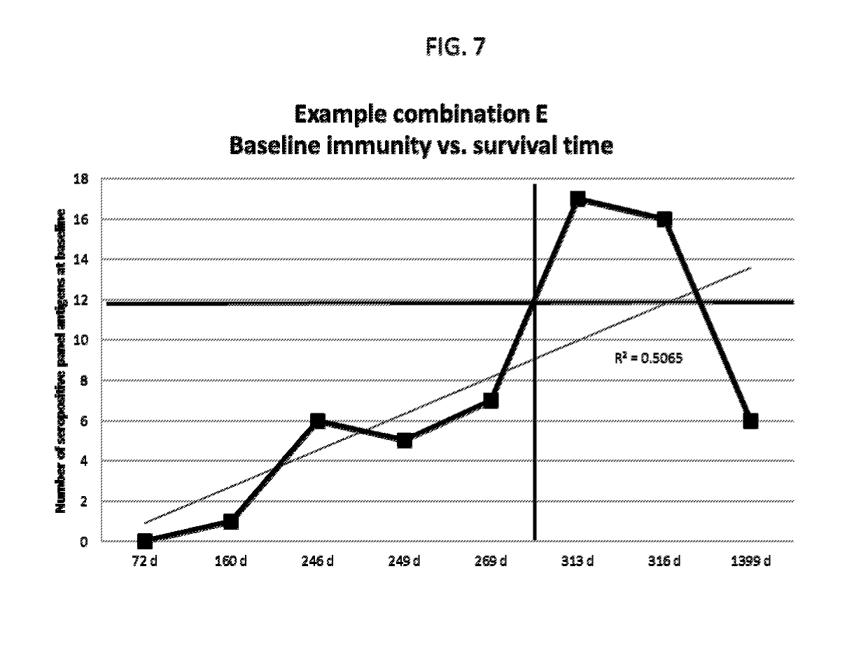

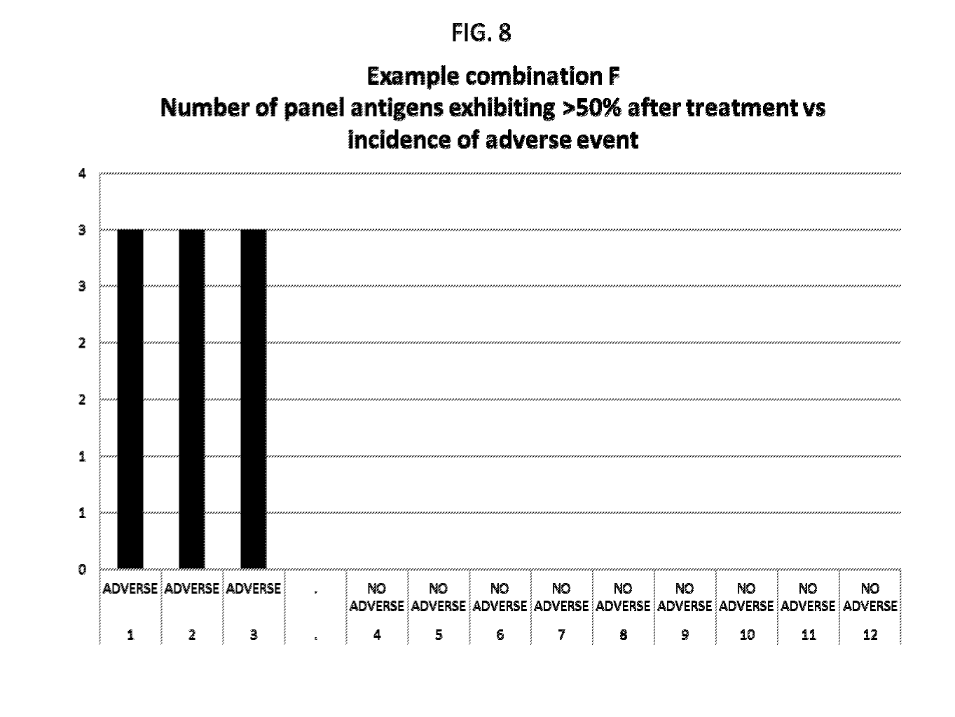

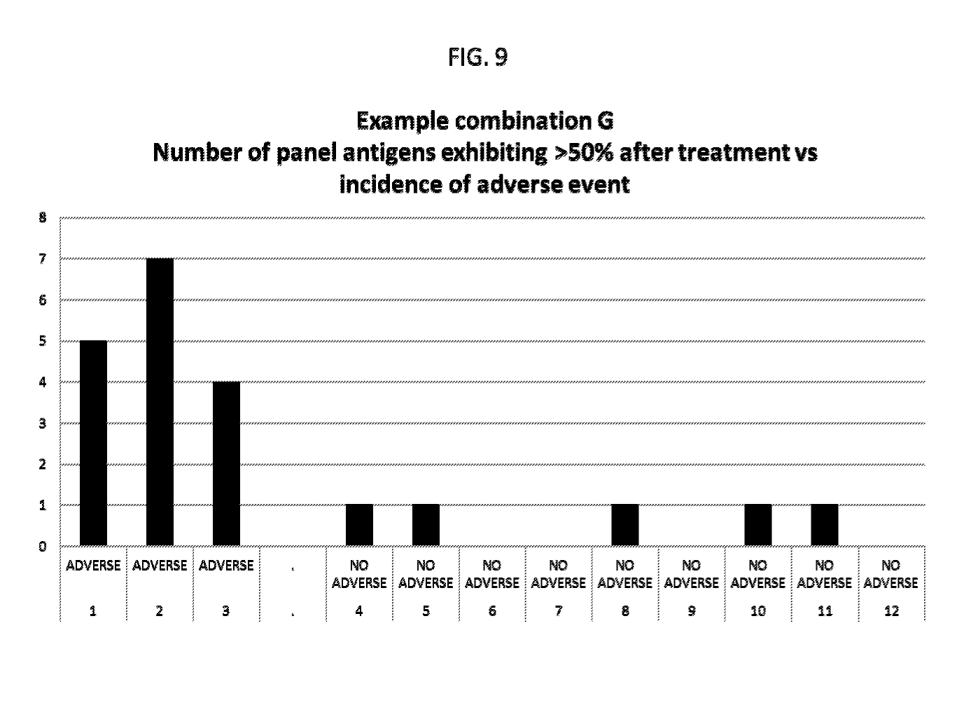

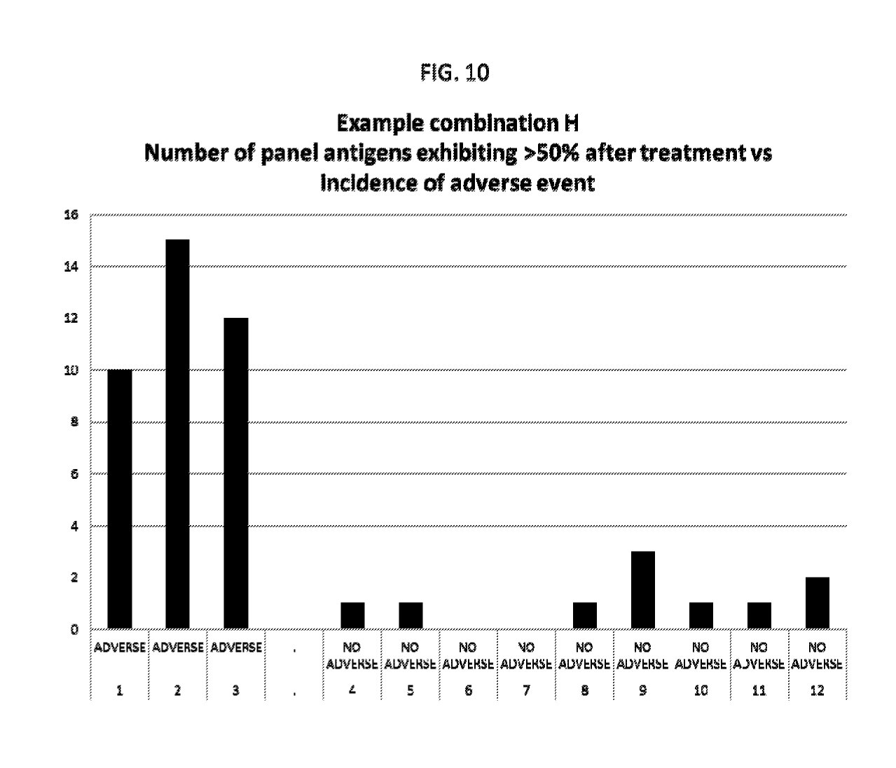

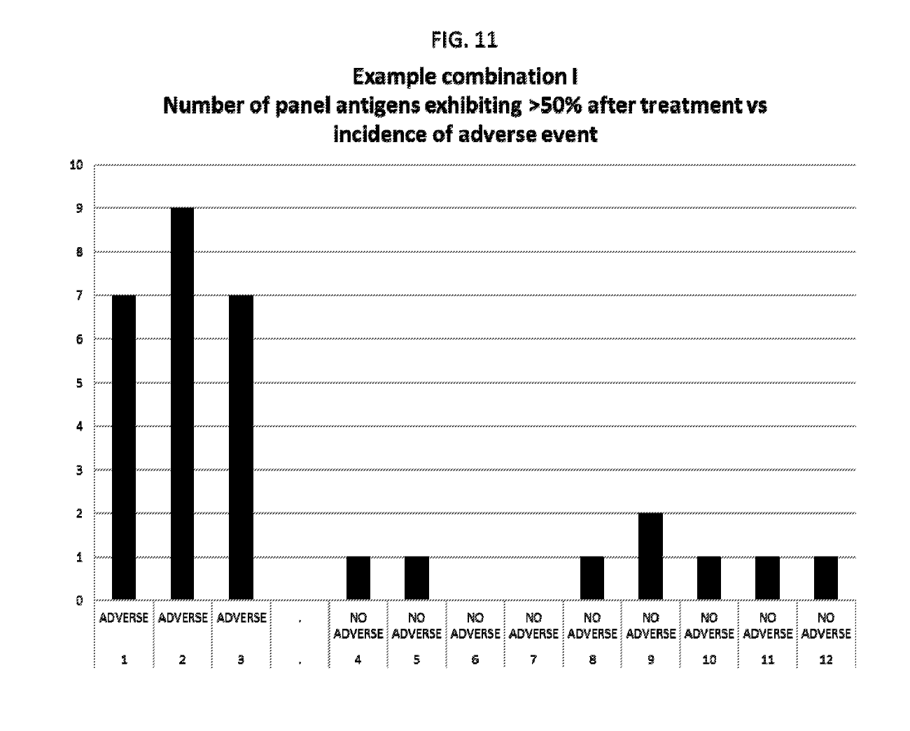

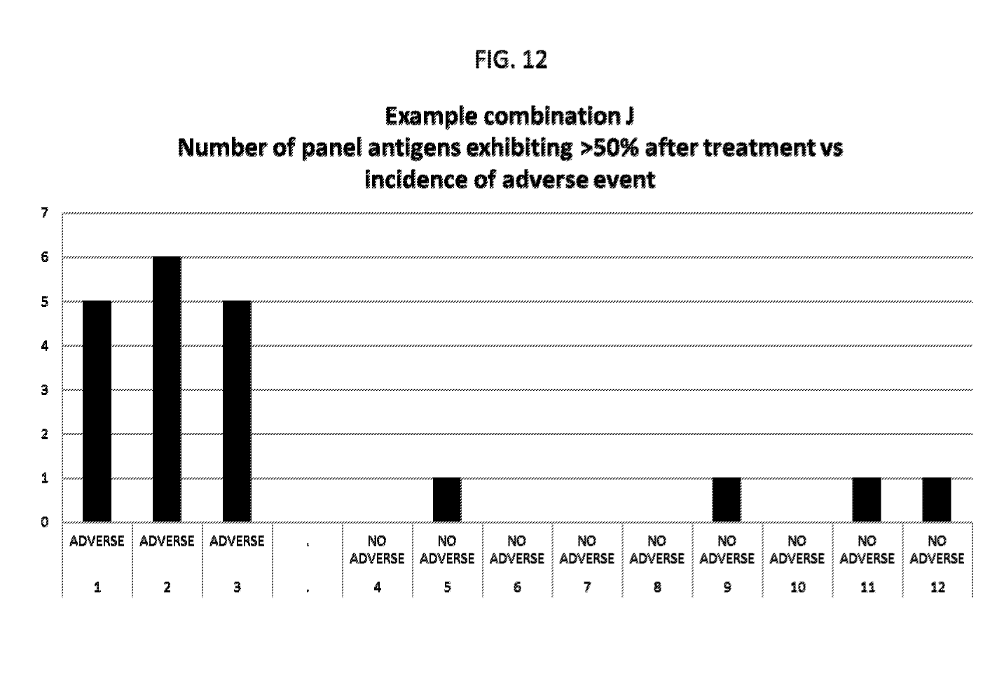

The results in Examples 1 and 2 were analyzed on the basis of ten different exemplified combinations of antigens (combinations A-J) ranging in number from 3 antigens to 24 antigens. Tables 2 and 3 below show performance of each antigen combination in predicting clinical response to immunotherapy (Table 2; combinations A-E) and predicting adverse events following immunotherapy (Table 3; combinations F-J). The score threshold refers to the minimum number of antigens that must be reactive in that patient to be deemed positive for the test. For example, referring to Combination A, any 2-antigen signature out of the five antigens would have predictive value. The performance of example combinations A-J are shown in the graphs of FIGS. 3-12, respectively.

### Performance Characteristics of Exemplified Antigen Combinations:

TABLE 2
Combinations A-E, predicting response to immunotherapy

| | # of antigens | score threshold | Non-Resp +ve (n) | Resp +ve (n) | Non-Resp +ve (%) | Resp +ve (%) |
|---|---|---|---|---|---|---|
| Combination A | 5 | 2 | 0 | 3 | 0% | 100% |
| Combination B | 11 | 4 | 1 | 2 | 20% | 67% |
| Combination C | 7 | 3 | 0 | 3 | 0% | 100% |
| Combination D | 16 | 5 | 1 | 3 | 20% | 100% |
| Combination E | 24 | 12 | 0 | 2 | 0% | 67% |

TABLE 3
Combinations F-J, predicting adverse events following immunotherapy

| | # of antigens | score threshold | AE +ve (n) | Non-AE +ve (n) | AE +ve (%) | Non-AE +ve (%) |
|---|---|---|---|---|---|---|
| Combination F | 3 | 2 | 3 | 0 | 100% | 0% |
| Combination G | 8 | 4 | 3 | 0 | 100% | 0% |
| Combination H | 23 | 10 | 3 | 0 | 100% | 0% |
| Combination I | 16 | 6 | 3 | 0 | 100% | 0% |
| Combination J | 7 | 3 | 3 | 0 | 100% | 0% |

The antigens of each exemplified combination are as follows:

### Combination A

### 1 GAGE2A

### 2 MAGEA1

### 3 MAGEA3

### 4 MAGEA4v2

### 5 MAGEA4v3

### Combination B

### 1 CSAG2

### 2 CTAG2

### 3 CXorf48.1

### 4 FTHL17

### 5 GAGE1

### 6 MAGEA3

### 7 SSX4

### 8 TSGA10

### 9 XAGE-2

### 10 ZNF165

### 11 MAPK1

### Combination C

### 1 CSAG2

### 2 CTAG2

### 3 MICA

### 4 NLRP4

### 5 SILV

### 6 SSX4

### 7 TSGA10

### Combination D

### 1 CSAG2

### 2 CTAG2

### 3 CXorf48.1

### 4 FTHL17

### 5 GAGE2A

### 6 MAGEA1

### 7 MAGEA3

### 8 MAGEA4v2

### 9 MAGEA4v3

### 10 SILV

### 11 SSX4

### 12 TSGA10

### 13 TSSK6

### 14 XAGE-2

### 15 ZNF165

### 16 MAPK1

### Combination E

### 1 CSAG2

### 2 CTAG2

### 3 CXorf48.1

### 4 FTHL17

### 5 GAGE1

### 6 GAGE2A

### 7 LDHC

### 8 MAGEA1

### 9 MAGEA3

### 10 MAGEA4v2

### 11 MAGEA4v3

### 12 MAGEB6

### 13 MICA

### 14 NLRP4

### 15 NY-ESO-1

### 16 PBK

### 17 SILV

### 18 SPANXA1

### 19 SPANXB1

### 20 SSX2A

### 21 SSX4

### 22 TSSK6

### 23 TYR

### 24 XAGE-2

### Combination F

### 1 MAGEB6

### 2 NLRP4

### 3 SPANXB1

### Combination G

### 1 GAGE2A

### 2 LDHC

### 3 MAGEA4v2

### 4 NY-ESO-1

### 5 PBK

### 6 SSX4

### 7 XAGE-2

### 8 ZNF165

### Combination H

### 1 CSAG2

### 2 CTAG2

### 3 CXorf48.1

### 4 GAGE2A

### 5 LDHC

### 6 MAGEA1

### 7 MAGEA4v2

### 8 MAGEA4v3

### 9 MAGEB6

### 10 MICA

### 11 NLRP4

### 12 NY-ESO-1

### 13 PBK

### 14 SILV

### 15 SPANXA1

### 16 SPANXB1

### 17 SSX2A

### 18 SSX4

### 19 TSGA10

### 20 TSSK6

### 21 TYR

### 22 XAGE-2

### 23 ZNF165

### Combination I

### 1 CXorf48.1

### 2 FTHL17

### 3 GAGE1

### 4 GAGE2A

### 5 LDHC

### 6 MICA

### 7 NLRP4

### 8 NY-ESO-1

### 9 PBK

### 10 SILV

### 11 SSX2A

### 12 SSX4

### 13 TSGA10

### 14 TSSK6

### 15 TYR

### 16 XAGE-2

### Combination J

### 1 MAGEB6

### 2 SPANXB1

### 3 SSX2A

### 4 SSX4

### 5 TSGA10

### 6 XAGE-2

### 7 ZNF165

### Example 4

### Predicting Response to Ipilimumab

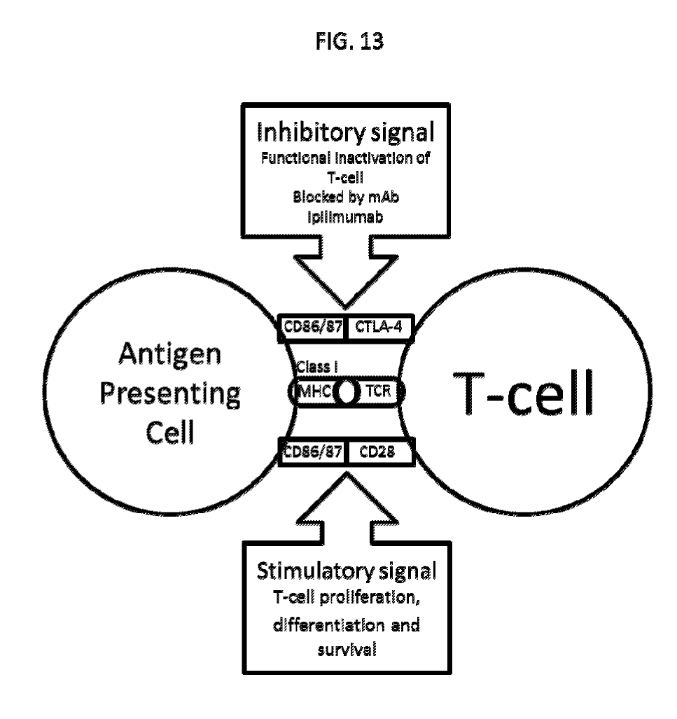

Checkpoint blockade through CTLA-4 with Ipilimumab was the first treatment to improve survival for patients with advanced melanoma, with a significant subgroup of patients benefiting long term. Ipilimumab's mechanism of action is shown in FIG. 13. Immune-mediated toxicity and increase in humoral and T-cell anti-NY-ESO-1 immune responses after treatment are both linked with benefit. However, there is currently no broadly relevant, immunological biomarker for predicting outcome prior to initiation of therapy. Thus, there is a need to identify the subset of patients who stand to gain a survival benefit from Ipilimumab treatment, given the high treatment cost and risks of significant toxicity. As Ipilimumab is an immunomodulatory agent, such biomarkers are most likely to be identified through the study of the immunological events in the patient. There is currently no immunological biomarker applicable to all melanoma patients available that predicts outcome prior to initiation of treatment.

Seropositivity in combination with a corresponding T-cell immune response to the Cancer/Testis antigen (CTA) NY-ESO-1 correlates with clinical benefit in a small subgroup of patients. However, monitoring a panel of antigens is likely to be preferable, since not all melanomas may express any single tumor antigen.

A panel of tumor-associated antigens (TAAs) was used to identify potential anti-TAA humoral responses that could predict clinical outcome in a patient population after treatment with Ipilimumab. The purpose of this study was to identify a broadly relevant immunological biomarker for predicting outcome prior to initiation of Ipilimumab therapy in patients with advanced melanoma.

Patients with advanced melanoma had variable levels of humoral immunity to a large number of TAAs. The presence of antibody response to 2 or more TAA correlated with longer survival following treatment with Ipilimumab, providing a biomarker for identifying those patients that are most likely to respond to checkpoint blockade with anti-CTLA-4 therapies such as Ipilimumab.

Methods:

All consecutive patients with metastatic melanoma treated in the expanded access program at Southampton University Hospitals between July 2010 and July 2011 were included in this study.

Patients received Ipilimumab at 3 mg/kg IV 3 weekly, for up to 4 cycles. Serum was stored frozen at baseline, prior to each subsequent cycle of Ipilimumab and at follow up. Sample analysis was blinded. Survival time from the first dose of Ipilumumab was recorded and compared with the number of antigens to which patients were seropositive at baseline.

Protein array analysis was performed using a multiplex immunoassay (described in Examples 1 and 2). The antigen panel included the following 23 tumor antigens:

1. BRAF

2. CABYR

3. CRISP3

4. CSAG2

5. CTAG2

6. DHFR

7. FTHL17

8. GAGE1

9. GLUD1

10. LDHC

11. MAGEA1

12. MAGEB6

13. MAPK1

14. FTHL17

15. SSX2

16. XAGE2

17. TULP2

18. PRAME

19. SOX2

20. SPANX-B1

21. SSX4

22. TSSK6

23. SSX5

### Results:

Patient demographics and treatment information are shown in Table 4.

TABLE 4
Patient Demographics

| Total No. of Patients in cohort | | 34 |
|---|---|---|
| Gender | Male | 13 |
| | Female | 22 |

| Median Age | 63 years | |
|---|---|---|
| Age Range | 37-84 years | |
| Disease Stage | IV | |
| | Cycle No. | No. of Patients |
| No. of Cycles Ipilumumab Received by | 1 | 34 |
| each patient (3 mg/kg) | 2 | 27 |
| | 3 | 24 |
| | 4 | 20 |
| | Line No. | No. of Patients |
| Line of treatment | 1st | 4 |
| | 2nd | 25 |
| | 3rd | 5 |

The median overall survival in this patient cohort was ˜24 weeks. However, patient survival was significantly different according to baseline immunity to the antigen panel.

Among the 34 patients, 12 had no detectable immunity, 6 had an antibody response to a single antigen from the panel, 5 had an antibody response to 2 antigens from the panel, and 11 patients had antibody responses to 3 or more of the panel antigens.

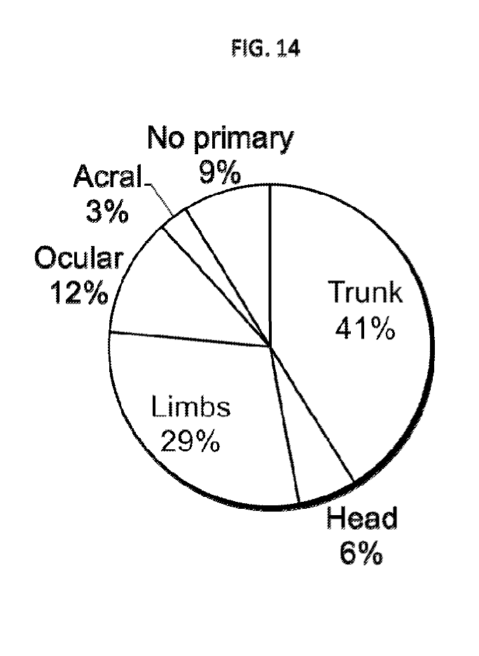

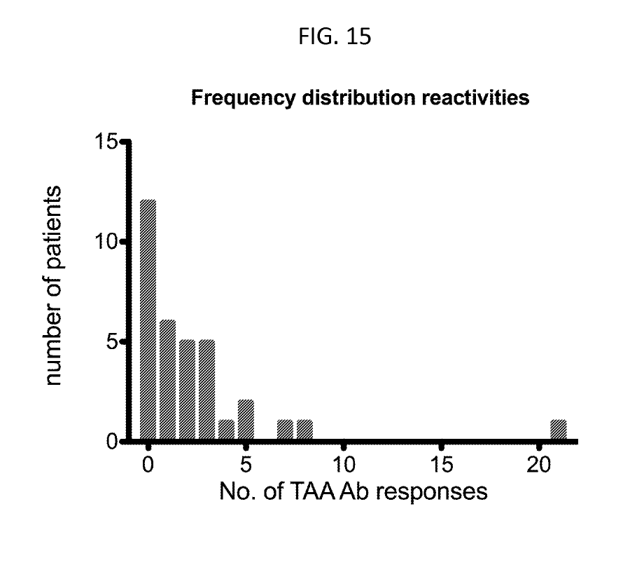

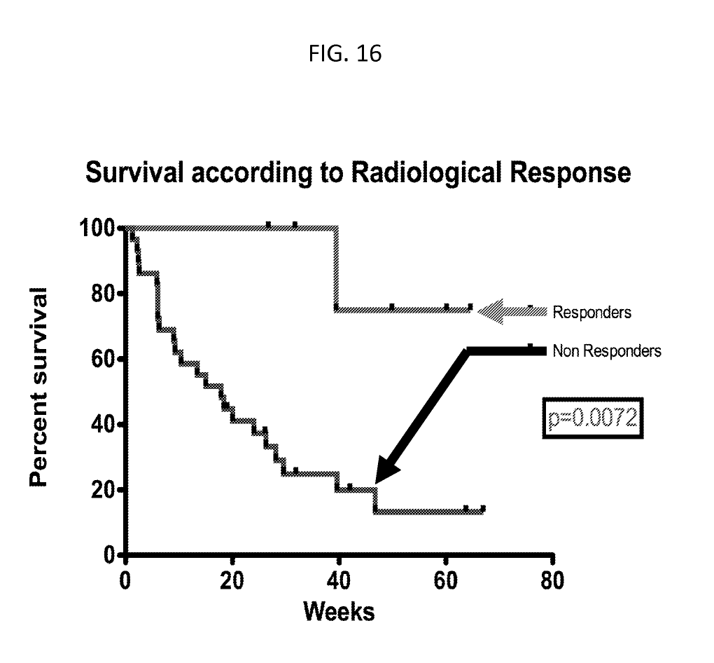

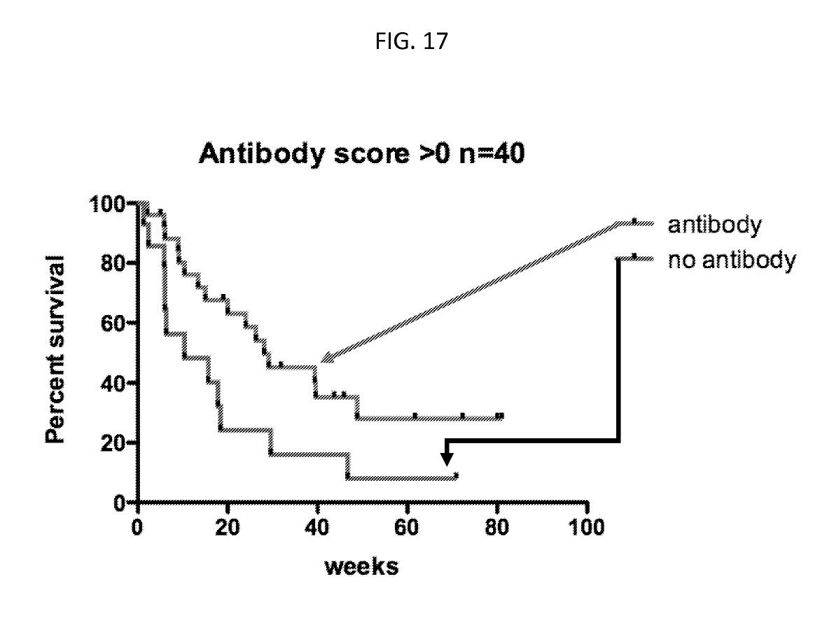

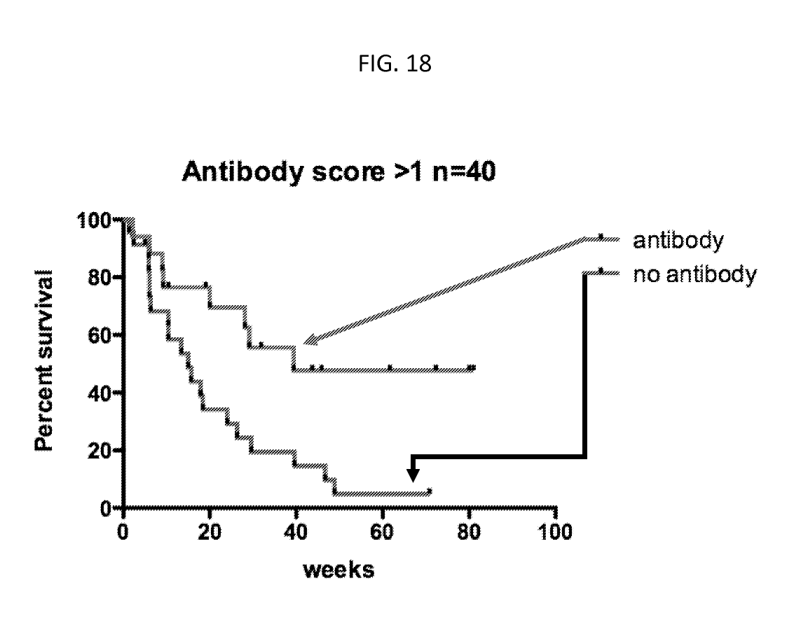

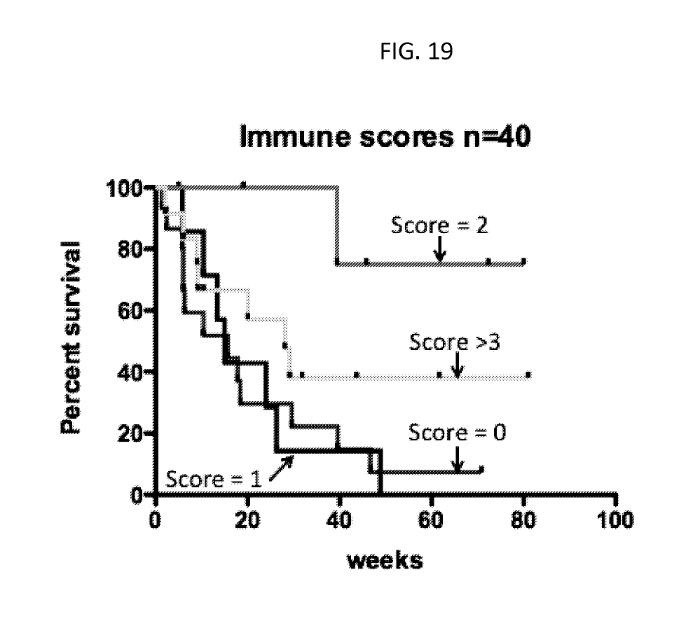

FIG. 14 is a chart showing sites of primary melanoma. FIG. 15 is a bar graph showing frequency distribution of antibody responses detected at baseline in the study cohort. FIG. 16 is a Kaplan-Meier survival curve, comparing survival of radiological responders (stable disease (SD)/partial response (PR)) to non-responders (partial disease (PD)). FIG. 17 shows Kaplan-Meier survival curves comparing survival in Ipilimumab-treated patients with an antibody response to 0 panel antigens to patients with an antibody response to 1 or more antigens. FIG. 18 shows Kaplan-Meier survival curves comparing survival in Ipilimumab-treated patients with an antibody response to 0 panel antigens to patients with an antibody response to 2 or more antigens. FIG. 19 shows Kaplan-Meier survival curves comparing survival in Ipilimumab-treated patients with an antibody response to 0 panel antigens, an antibody response to 1 panel antigen, an antibody response to 2 panel antigens, and an antibody response to 3 or more panel antigens.

Patients with antibody responses to at least two tumor antigens had significantly longer overall survival, compared to those patients with antibody responses to either zero or one antigen only. The median survival in the group of patients with antibody responses to at least two tumor antigens at baseline was 39.4 weeks. In contrast, the median survival in the group of patients with antibody responses to zero or one tumor antigen was 16.4 weeks. The difference in the two populations was statistically significant (p=0.02).

6/34 patients (17.6%) had an objective response to Ipilumumab 2×SD, 4×PR were observed, median survival not yet reached. Patients with antibody responses to equal to or greater than 2 tumor antigens had a significantly longer overall survival, compared to those with 0 or 1 specificity (median survival 39.4 vs 16.4 weeks, p=0.02). All patients with PR were in the equal to or greater than 2 specificity group.

Checkpoint blockade activates antigen specific T-cell responses and boosts pre-existing anti-tumour immunity. Protective antigens that are recognized by tumor-specific T-cells remain unknown. CTA or melanonocytic antigens are likely candidates for specific immune attack of melanoma cells. Using the described immunoassay, the inventors found that patients have a broad range of antibody reactivities to melanoma-associated antigens, ranging from 0 to 21 reactivities, with a median of 1.5.

Reactivity to a single antigen did not have the power to predict survival; however, patients with antibodies against two or more panel antigens were significantly more likely to survive when treated with Ipilimumab. All objective radiological responders fell into the group of patients with equal to or greater than 2 antibody specificities, supporting the identification of a biologically significant link between humoral immunity and benefit from checkpoint blockade. The inventors have identified a group of patients in which the melanomas are more immunologically visible and, therefore, more likely to benefit from untargeted attack by generalized activation of cellular immunity.

In conclusion, the antigen set of the invention is the first immunological biomarker to predict outcome prior to Ipilumumab treatment, representing a test positive as having above-threshold level of antibody to any two (i.e., two or more) antigens in the panel (irrespective of which antigens in the panel were reactive). Unlike T cell analyses, the antigen set of the invention is attractive because it does not require complex sample processing and storage. Examination of T-cell responses may also be used to assess how cellular TAA specific immune responses correlate with the observed antibody specificities.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

1. A method for predicting a clinical response (efficacy) and/or adverse event to an immunotherapy for treatment of a malignancy in a subject, comprising:
(a) measuring the level of two or more biomarkers in a biological sample taken from the subject before or after initiation of the immunotherapy, and wherein the two or more biomarkers comprise:
(1) immunoglobulins to two or more antigens selected from among BRAF, CABYR, CRISP3, CSAG2, CTAG2, CXorf48.1, DHFR, FTHL17, GAGE1, GAGE2A, GLUD1, LDHC, MAGEA1, MAGEA3, MAGEA4v2, MAGEA4v3, MAGEA4v4, MAGEB6, MAPK1, MICA, MUC1, NLRP4, NY-ESO-1, PBK, PRAME, SOX2, SILV, SPANXA1, SPANXB1, SSX2A, SSX4, TSGA10, TSSK6, TULP2, TYR, XAGE-2, and ZNF165; or
(2) two or more antigens selected from those set forth in (a)(1); or
(3) nucleic acid sequences that encode two or more antigens selected from those set forth in (a)(1); or
(4) T-cells activated against two or more antigens selected from those set forth in (a)(1); and
(b) correlating the level of the two or more biomarkers in the sample with a predicted clinical response and/or likelihood of an adverse event in the subject.

2. The method of claim 1, wherein the two or more antigens comprise the group of antigens of example combination A, example combination B, example combination C, example combination D, example combination E, example combination F, example combination G, example combination H, example combination I, or example combination J.

3. The method of claim 1, wherein the two or more antigens comprise CSAG2, MAGEA1, MAGEA3, MAGEA4v2, MICA, NLRP4, SILV, SSX4, TSSK6, and XAGE-2.

4. The method of claim 1, wherein the two or more antigens comprise two or more of BRAE, CABYR, CRISP3, CSAG2, CTAG2, DHFR, FTHL17, GAGE1, GLUD1, LDHC, MAGEA1, MAGEB6, MAPK1, FTHL17, SSX2, XAGE2, TULP2, PRAME, SOX2, SPANX-B1, SSX4, TSSK6, and SSX5.

5. The method of claim 1, wherein said correlating of (b) comprises comparing the level of the two or more biomarkers in the sample to a reference level of the two or more biomarkers, wherein the relationship between the level of the two or more biomarkers in the sample and the reference level is indicative of the clinical response and/or the likelihood of an adverse event.

6. (canceled)

7. The method of claim 1, wherein said measuring of (a) comprises measuring the level of the two or more biomarkers in a biological sample taken from the subject, and said correlating of (b) comprises comparing the measured level of the two or more biomarkers to a reference level of the two or more biomarkers, wherein the relationship between the level of the two or more biomarkers in the sample and the reference level is indicative of the clinical response and/or the likelihood of an adverse event.

8. The method of claim 5, wherein the sample is obtained from the subject after initiation of the immunotherapy, and wherein the reference level is the level of the two or more biomarkers in a sample taken from the subject before initiation of the immunotherapy.

9-14. (canceled)

15. The method of claim 1, wherein the biomarkers comprise or consist of (a)(1), and wherein the biological sample is serum.

16. The method of claim 1, wherein the biomarkers comprise or consist of (a)(1) or (a)(2), and wherein the biological sample comprises cells of a malignancy.

17. The method of claim 1, wherein the malignancy is selected from among melanoma, ovarian cancer, breast cancer, lung cancer (small cell or non-small cell), esophageal cancer, sarcoma, or colorectal cancer.

18. The method of claim 1, wherein the adverse event comprises autoimmune toxicity.

19. (canceled)

20. The method of claim 1, wherein the immunotherapy comprises an agent selected from among a cancer vaccine, immunomodulator, monoclonal antibody, immunostimulant, dendritic cell, viral therapy.

21-22. (canceled)

23. The method of claim 1, wherein the two or more antigens comprise two or more of BRAF, CABYR, CRISP3, CSAG2, CTAG2, DHFR, FTHL17, GAGE1, GLUD1, LDHC, MAGEA1, MAGEB6, MAPK1, FTHL17, SSX2, XAGE2, TULP2, PRAME, SOX2, SPANX-B1, SSX4, TSSK6, and SSX5; wherein the malignancy is selected from among melanoma, ovarian cancer, breast cancer, lung cancer (small cell or non-small cell), esophageal cancer, sarcoma, or colorectal cancer; and wherein the immunotherapy comprises an antibody that binds to cytotoxic T lymphocyte-associated antigen 4 (CTLA-4).

24-29. (canceled)

30. A composition of matter, comprising:
(a) an array comprising a substrate and two or more capture probes disposed thereon, wherein said two or more capture probes comprise:
(i) at least antigenic epitopes of two or more antigens selected from among BRAF, CABYR, CRISP3, CSAG2, CTAG2, CXorf48.1, DHFR, FTHL17, GAGE1, GAGE2A, GLUD1, LDHC, MAGEA1, MAGEA3, MAGEA4v2, MAGEA4v3, MAGEA4v4, MAGEB6, MAPK1, MICA, MUC1, NLRP4, NY-ESO-1, PBK, PRAMS, SOX2, SILV, SPANXA1, SPANXB1, SSX2A, SSX4, TSGA10, TSSK6, TULP2, TYR, XAGE-2, and ZNF165; or
(ii) antibodies, or antibody fragments, that specifically bind two or more antigens from those set forth in (i); or
(iii) oligonucleotides that are partially or fully complementary to, and bind to, nucleic acid sequences encoding two or more antigens from those set forth in (i); or
(b) a kit for predicting a clinical response (efficacy) and/or adverse event to an immunotherapy, comprising two or more capture probes in one or more containers, wherein the capture probes comprise or consist of:
(i) at least antigenic epitopes of two or more antigens selected from among BRAE, CABYR, CRISP3, CSAG2, CTAG2, CXorf48.1, DHFR, FTHL17, GAGE1, GAGE2A, GLUD1, LDHC, MAGEA1, MAGEA3, MAGEA4v2, MAGEA4v3, MAGEA4v4, MAGEB6, MAPK1, MICA, MUC1, NLRP4, NY-ESO-1, PBK, PRAME, SOX2, SILV, SPANXA1, SPANXB1, SSX2A, SSX4, TSGA10, TSSK6, TULP2, TYR, XAGE-2, and ZNF165; or
(ii) antibodies, or antibody fragments, that specifically bind two or more antigens from those set forth in (i); or
(iii) oligonucleotides that bind to nucleic acid sequences encoding two or more antigens from those set forth in (i).

31. The composition of matter of claim 30, wherein the two or more antigens of the array of (a) comprise the group of antigens of example combination A, example combination B, example combination C, example combination D, example combination E, example combination F, example combination G, example combination H, example combination I, or example combination J.

32-37. (canceled)

38. The composition of matter of claim 30, wherein the two or more antigens of the kit of (b) comprise the group of antigens of example combination A, example combination B, example combination C, example combination D, example combination E, example combination F, example combination G, example combination H, example combination I, or example combination J.

39-41. (canceled)

42. A method for treating or delaying the onset or relapse of a malignancy in a subject, comprising:
(a) predicting the clinical response (efficacy) and/or adverse event to an immunotherapy for treatment of a malignancy in a subject determined by the level of two or more biomarkers comprising or consisting of:
(1) immunoglobulins to two or more antigens selected from among BRAF, CABYR, CRISP3, CSAG2, CTAG2, CXorf48.1, DHFR, FTHL17, GAGE1, GAGE2A, GLUD1, LDHC, MAGEA1, MAGEA3, MAGEA4v2, MAGEA4v3, MAGEA4v4, MAGEB6, MAPK1, MICA, MUC1, NLRP4, NY-ESO-1, PBK, PRAMS, SOX2, SILV, SPANXA1, SPANXB1, SSX2A, SSX4, TSGA10, TSSK6, TULP2, TYR, XAGE-2, and ZNF165; or
(2) two or more antigens selected from those set forth in (a)(1); or
(3) nucleic acid sequences that encode two or more antigens selected from those set forth in (a)(1); or
(4) T-cells activated against two or more antigens selected from those set forth in (a)(1); and
(b) administering an immunotherapy to the subject if it is predicted that the immunotherapy will have efficacy and/or will not result in an adverse event; or
(c) withholding the immunotherapy from the subject if it is predicted that the immunotherapy will not have efficacy and/or will result in an adverse event.

43. The method of claim 42, wherein (c) further comprises administering a therapy other than an immunotherapy to the subject if it is predicted that the immunotherapy will not have efficacy and/or will result in an adverse event.

44. (canceled)

45. A method for treating or delaying the onset or relapse of a malignancy in a subject, comprising carrying out the method of claim 1, and further comprising:
(c) administering an immunotherapy to the subject if it is predicted that the immunotherapy will have efficacy and/or will not result in an adverse event; or
(d) withholding the immunotherapy from the subject if it is predicted that the immunotherapy will not have efficacy and/or will result in an adverse event.

46. The method of claim 45, wherein (d) further comprises administering a therapy other than an immunotherapy to the subject if it is predicted that the immunotherapy will not have efficacy and/or will result in an adverse event.

47. (canceled)
